(19) United States
(12) Patent Application Publication
(10) Pub. No.: US 2026/0365610 A1
(43) Pub. Date: Nov. 19, 2026

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Sung Kil HONG, Daejeon (KR); Sung Jae LEE, Daejeon (KR); Jongsoo SONG, Daejeon (KR); Hyeon Jin MUN, Daejeon (KR)

(21) Appl. No.: 19/163,292

(22) PCT Filed: Jun. 5, 2024
(86) PCT No.: PCT/KR2024/007741

§ 371 Date: Sep. 8, 2025

(30) Foreign Application Priority Data
Jun. 5, 2023 (KR) 10-2023-0072234
Jun. 4, 2024 (KR) 10-2024-0073292

(51) Int. Cl. C07C 211/54; C07C 211/57; C07D 307/91; C07D 333/76; H10K 85/60

(52) U.S. Cl. CPC C07C 211/54; C07C 211/57; C07D 307/91; C07D 333/76; H10K 85/633; H10K 85/6574; H10K 85/6576

(57) ABSTRACT

An organic light-emitting material including a compound of Chemical Formula 1:

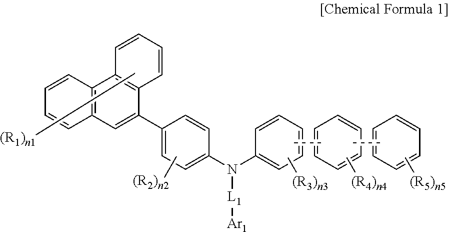

where Ar₁ is a substituted or unsubstituted C₆₋₆₀ aryl, a substituted or unsubstituted dibenzofuranyl, or a substituted or unsubstituted dibenzothiophenyl; L₁ is a single bond, a substituted or unsubstituted C₆₋₆₀ arylene, a substituted or unsubstituted dibenzofuranylene, or a substituted or unsubstituted dibenzothiophenylene; R₁ is deuterium; R₂ to R₄ are each independently hydrogen or deuterium; R₅ is hydrogen, deuterium, or a substituted or unsubstituted C₁₋₆₀ alkyl; n1 is an integer of 1 to 9; n2 to n4 are each independently an integer of 1 to 4; and n5 is an integer of 1 to 5, wherein the compound has a deuterium substitution rate of 50% or more. Also provided is an organic light-emitting device including the same.

### CROSS-REFERENCE TO RELATED APPLICATION(S)

This application is a National Stage Application of International Application No. PCT/KR2024/007741 filed on Jun. 5, 2024, which claims priority benefit of Korean Patent Application No. 10-2023-0072234 filed on Jun. 5, 2023 and Korean Patent Application No. 10-2024-0073292 filed on Jun. 4, 2024 in the Korean Intellectual Property Office, the disclosures of which are incorporated herein by reference in their entirety.

### TECHNICAL FIELD

The present disclosure relates to a novel compound and an organic light emitting device comprising the same.

### BACKGROUND ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continued need to develop a new material for the organic material used in the organic light emitting device as described above.

### PRIOR ART LITERATURE

### Patent Literature

(Patent Literature 1) Korean Unexamined Patent Publication No. 10-2000-0051826

### DETAILED DESCRIPTION OF THE INVENTION

### Technical Problem

It is an object of the present disclosure to provide a novel organic light emitting material and an organic light emitting device comprising the same.

### Technical Solution

Provided herein is a compound represented by the following Chemical Formula 1:

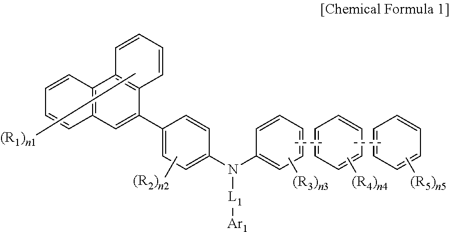

in Chemical Formula 1,
Ar₁ is a substituted or unsubstituted C₆₋₆₀ aryl; a substituted or unsubstituted dibenzofuranyl; or a substituted or unsubstituted dibenzothiophenyl,
L₁ is a single bond; a substituted or unsubstituted C₆₋₆₀ arylene; a substituted or unsubstituted dibenzofuranylene; or a substituted or unsubstituted dibenzothiophenylene,
R₁ to R₄ are each independently hydrogen or deuterium,
R₅ is hydrogen, deuterium, or a substituted or unsubstituted C₁₋₆₀ alkyl,
n1 is an integer of 1 to 9,
n2 to n4 are each independently an integer of 1 to 4,
n5 is an integer of 1 to 5,
wherein the compound represented by Chemical Formula 1 has a deuterium substitution rate of 50% or more.

Also provided herein is an organic light emitting device comprising: a first electrode; a second electrode that is provided opposite to the first electrode; and one or more organic material layers that are provided between the first electrode and the second electrode, wherein at least one layer of the organic material layers comprises the compound represented by Chemical Formula 1.

### Advantageous Effects

The compound represented by Chemical Formula 1 described above can be used as a material of an organic material layer of an organic light emitting device, and can improve the efficiency, achieve low driving voltage and/or improve lifetime characteristics in the organic light emitting device. In particular, the compound represented by Chemical Formula 1 can be used as a hole transport material, a hole transport auxiliary material, and/or light emitting material.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a hole transport auxiliary layer 7, a light emitting layer 3, a hole blocking layer 8, an electron transport and injection layer 9, and cathode 4.

### DETAILED DESCRIPTION OF THE EMBODIMENTS

Hereinafter, embodiments of the present disclosure will be described to help understanding of the disclosed subject matter.

The present disclosure provides the compound represented by Chemical Formula 1.

In the present disclosure, the notation

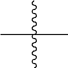

or [symbol] means a bond linked to another substituent group.

In the present disclosure, the term “substituted or unsubstituted” means being unsubstituted or substituted with one or more substituents selected from the group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heteroaryl group containing at least one of N, O and S atoms, or being unsubstituted or substituted with a substituent to which two or more substituents of the above-exemplified substituents are linked. For example, “a substituent in which two or more substituents are linked” may be a biphenyl group. Namely, a biphenyl group may be an aryl group, or it may be interpreted as a substituent formed by linking two phenyl groups. In one example, the term “substituted or unsubstituted” may be understood as meaning “being unsubstituted or substituted with one or more substituents selected from the group consisting of deuterium, halogen, a C₁₋₁₀ alkyl, a C₁₋₁₀ alkoxy, a C₆₋₂₀ aryl, and one or more substituents, for example 1 to 5 substituents, selected from the group consisting of a C₂₋₂₀ heteroaryl containing at least one heteroatom of N, O and S”. Further, the term “substituted with one or more substituents” as used herein may be understood as meaning “being substituted with 1 to 5 substituents”, or “being substituted with one or two substituents”.

In the present disclosure, “when a substituent is not indicated in the chemical formula or compound structure” may mean that hydrogen and deuterium mixedly exist in the chemical formula or compound structure unless deuterium is explicitly excluded, such as “the content of deuterium is 0%,” “the content of hydrogen is 100%,”.

In the present disclosure, the carbon number of a carbonyl group is not particularly limited, but is preferably 1 to 40. Specifically, it may be a substituent group having the following structure, but is not limited thereto.

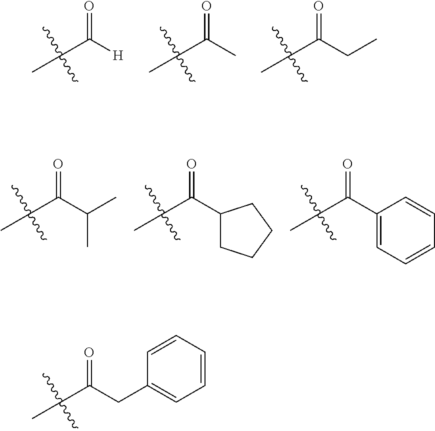

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent group having the following structural formulas, but is not limited thereto.

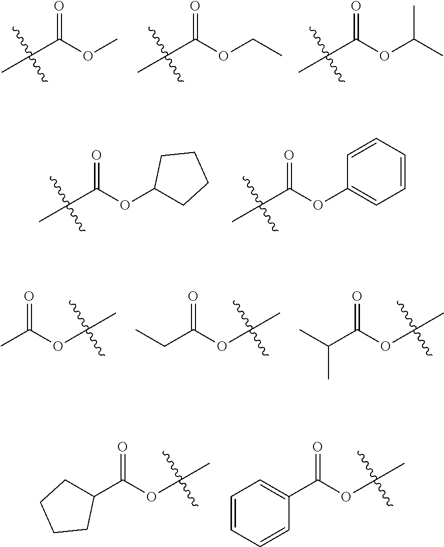

In the present disclosure, the carbon number of an imide group is not particularly limited, but is preferably 1 to 25. Specifically, the imide group may be a substituent group having the following structural formulas, but is not limited thereto.

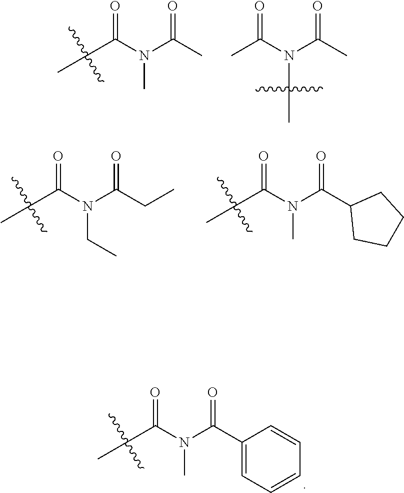

In the present disclosure, specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, of the cycloalkyl group is not particularly limited, but the carbon number thereof is preferably 3 to 60. According to another embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, a fluorenyl group, or the like, but is not limited thereto.

In the present disclosure, the fluorenyl group may be substituted, and two substituent groups may be linked with each other to form a spiro structure. In the case where the fluorenyl group is substituted,

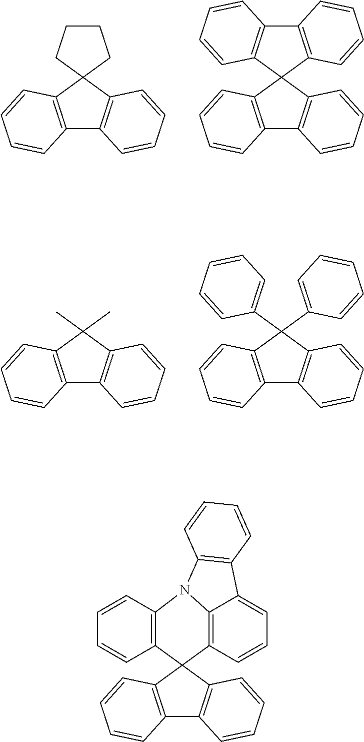

and the like can be formed. However, the structure is not limited thereto.

In the present disclosure, a heteroaryl group is a heteroaryl group containing one or more of O, N, Si and S as a heteroatom, and the carbon number thereof is not particularly limited, but is preferably 2 to 60. According to one embodiment, the carbon number of the heteroaryl group is 6 to 30. According to one embodiment, the carbon number of the heteroaryl group is 6 to 20. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the examples of the aryl group as defined above. In the present disclosure, the alkyl group in the aralkyl group, the alkylaryl group and the alkylamine group is the same as the examples of the alkyl group as defined above. In the present disclosure, the heteroaryl in the heteroarylamine can be applied to the description of the heteroaryl as defined above. In the present disclosure, the alkenyl group in the aralkenyl group is the same as the examples of the alkenyl group as defined above. In the present disclosure, the description of the aryl group as defined above may be applied except that the arylene is a divalent group. In the present disclosure, the description of the heteroaryl as defined above can be applied except that the heteroarylene is a divalent group. In the present disclosure, the description of the aryl group or cycloalkyl group as defined above can be applied except that the hydrocarbon ring is not a monovalent group but formed by combining two substituent groups. In the present disclosure, the description of the heteroaryl as defined above can be applied, except that the heteroaryl is not a monovalent group but formed by combining two substituent groups.

In the present disclosure, the term “deuterated or substituted with deuterium” means that at least one of the substitutable hydrogens in a compound, a divalent linking group, or a monovalent substituent has been substituted with deuterium.

Further, the term “unsubstituted or substituted with deuterium” or “substituted or unsubstituted with deuterium” means that “mono to the maximum number of unsubstituted or substitutable hydrogens have been substituted with deuterium.” In one example, the term “phenanthryl unsubstituted or substituted with deuterium” may be understood as meaning “phenanthryl unsubstituted or substituted with 1 to 9 deuterium atoms”, considering that the maximum number of hydrogens that can be substituted with deuterium in the phenanthryl structure is 9.

Further, “deuterated structure” means to include compounds, divalent linking groups, or monovalent substituents of all structures in which at least one hydrogen is substituted with deuterium. As an example, the deuterated structure of phenyl can be understood to refer to monovalent substituents of all structures in which at least one substitutable hydrogen in the phenyl group is substituted with deuterium, as follows.

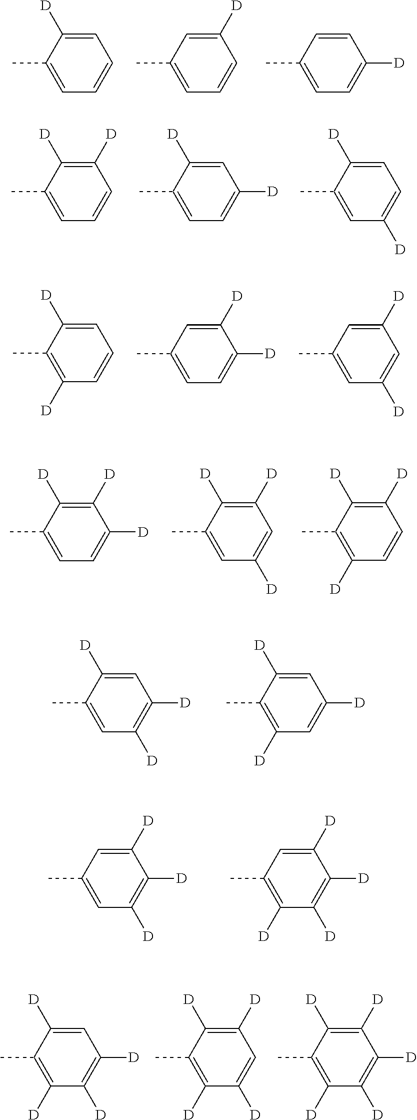

In addition, the “deuterium substitution rate” of a compound means that the ratio of the number of substituted deuterium atoms to the total number of hydrogen atoms (the sum of the number of hydrogen atoms substitutable with deuterium and the number of substituted deuterium atoms in a compound) that can exist in the compound is calculated as a percentage. Therefore, when the “deuterium substitution rate” of a compound is “K %”, it means that K % of the hydrogen atoms substitutable with deuterium in the compound are substituted with deuterium.

At this time, the “deuterium substitution rate” or “degree of deuteration” can be determined according to a commonly known method using MALDI-TOF MS (Matrix-Assisted Laser Desorption/Ionization Time-of-Flight Mass Spectrometer), a nuclear magnetic resonance spectroscopy (¹H NMR), TLC/MS (Thin-Layer Chromatography/Mass Spectrometry), GC/MS (Gas Chromatography/Mass Spectrometry), or the like.

More specifically, when using MALDI-TOF MS, the “deuterium substitution rate” or “degree of deuteration” may be obtained by determining the number of substituted deuterium in the compound through MALDI-TOF MS analysis, and then calculating the ratio of the number of substituted deuterium to the total number of hydrogen atoms that can exist in the compound as a percentage. Therefore, when the “deuterium substitution rate” of a compound is “K %”, it means that K % of the hydrogen atoms substitutable with deuterium in the compound are substituted with deuterium.

In addition, when analyzing the “deuterium substitution rate” or “degree of deuteration” through TLC/MS (thin-layer chromatography/mass spectrometry), the substitution rate can be calculated based on the maximum value (max. value) of distribution that molecular weights form at the end of the reaction.

Further, when analyzing the “deuterium substitution rate” or “degree of deuteration” using nuclear magnetic resonance (¹H NMR), the deuterium substitution rate or degree of deuteration can be calculated from the integration quantity of total peaks through the integration ratio in ¹H NMR.

Meanwhile, in the present disclosure, “deuterium does not exist at a specific position” means that the deuterium substitution rate at that position is 10% or less, and does not mean that the deuterium substitution rate is 0%. In addition, in the present disclosure, “deuterium exists at a specific position” means that the deuterium substitution rate at that position is more than 1%, and does not mean that the deuterium substitution rate at that position is 100%. In this manner, the “deuterium substitution rate at a specific position” may be calculated by comparing the ¹H NMR spectrum of a compound not substituted with deuterium with the ¹H NMR spectrum of a compound substituted with deuterium, and confirming the rate at which the integration quantity of the peak for each hydrogen (proton) position decreases.

In the compound represented by Chemical Formula 1, R₁ is deuterium and n1 is 1 to 9, so that phenanthrenyl in Chemical Formula 1 is substituted with at least one hydrogen. Herein, ‘phenanthrenyl is substituted with at least one deuterium’ means that ‘deuterium (R₁), which is not hydrogen, is located on at least one of the nine carbons of phenanthrenyl of Chemical Formula 1 which can be substituted with deuterium. More specifically, it means that one carbon of each carbon of phenanthrenyl included in the compound represented by Chemical Formula 1 is substituted with deuterium, wherein the deuterium substitution rate of each of the remaining eight carbons is greater than 1%. The deuterium substitution rate at each carbon of the phenanthrenyl can be determined by comparing the ¹H NMR spectrum of the compound in which deuterium is not substituted with the ¹H NMR spectrum of the compound in which deuterium is substituted, as described above.

Preferably, the Chemical Formula 1 may be represented by any one of the following Chemical Formulas 1A to 1I.

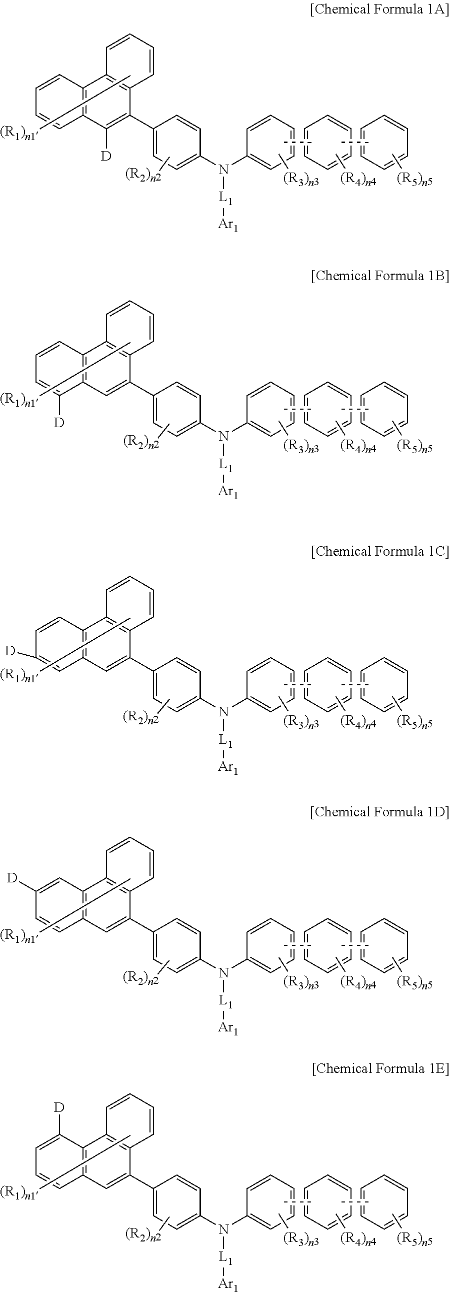

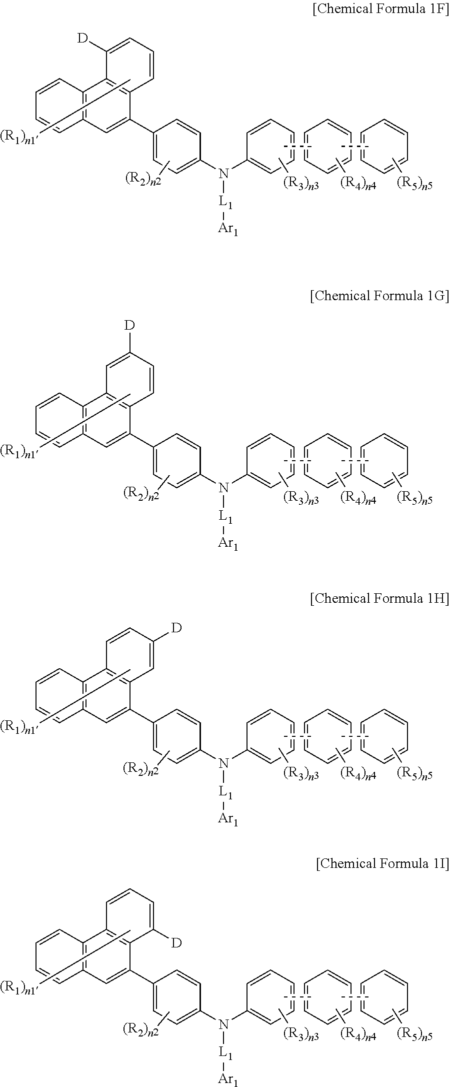

in Chemical Formulas 1A to 1I,
D is deuterium,
n1′ is an integer of 0 to 8,
Ar₁, L₁, R₁ to R₅ and n2 to n5 are as defined in Chemical Formula 1.

Preferably, the Chemical Formula 1 may be represented by any one of the following Chemical Formulas 1-1 to 1-8:

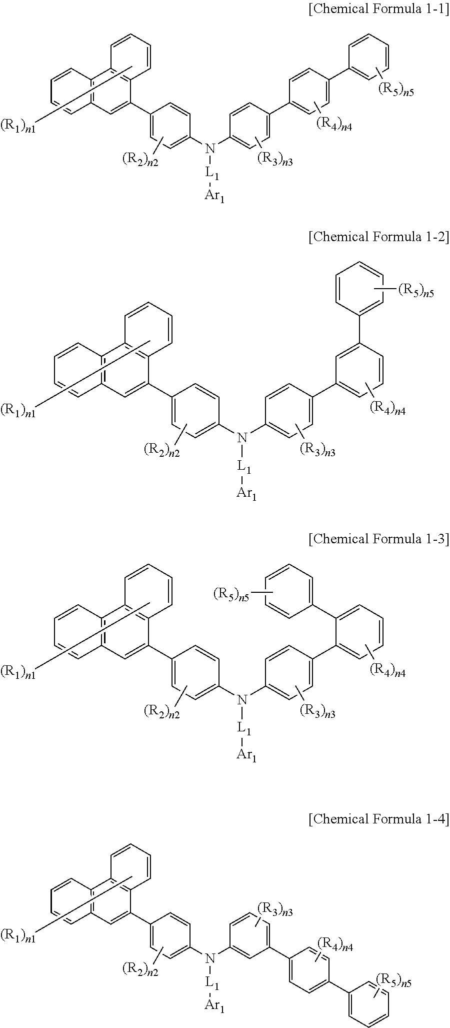

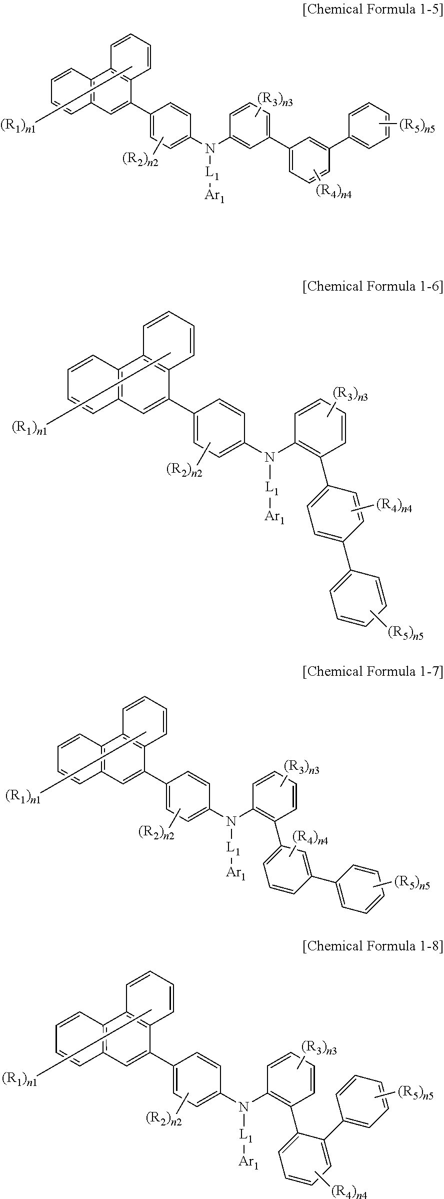

in Chemical Formulas 1-1 to 1-8,
Ar₁, L₁, R₁ to R₅ and n1 to n5 are as defined in Chemical Formula 1.

Preferably, Ar₁ may be a substituted or unsubstituted C₆₋₂₀ aryl; a substituted or unsubstituted dibenzofuranyl; or a substituted or unsubstituted dibenzothiophenyl,

More preferably, Ar₁ may be phenyl, biphenylyl, terphenylyl, naphthyl, phenanthrenyl, tetrahydronaphthyl, tetramethyltetrahydronaphthyl, phenyl substituted with one or two methyl groups, phenyl substituted with one or two isopropyl groups, phenyl substituted with one or two tertbutyl groups, dibenzofuranyl, or dibenzothiophenyl, wherein the phenyl, biphenylyl, terphenylyl, naphthyl, phenanthrenyl, tetrahydronaphthyl, tetramethyltetrahydronaphthyl, phenyl substituted with one or two methyl groups, phenyl substituted with one or two isopropyl groups, phenyl substituted with one or two tertbutyl groups, dibenzofuranyl, or dibenzothiophenyl may be each independently unsubstituted, or substituted with at least one deuterium.

Most preferably, Ar₁ may be any one selected from the group consisting of the following compounds, wherein Ar₁ of the following groups may be further unsubstituted, or substituted with at least one deuterium:

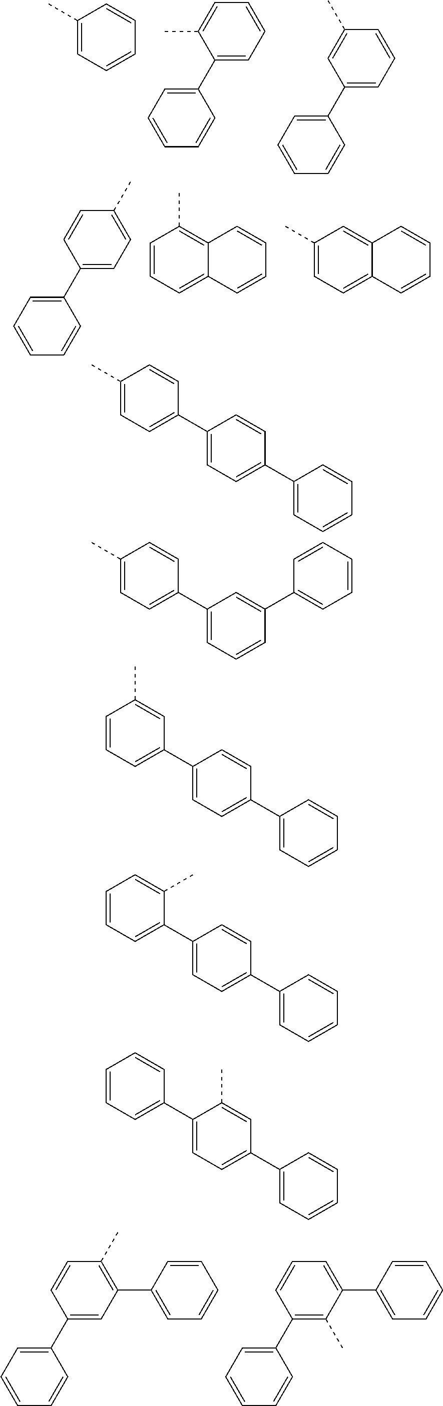

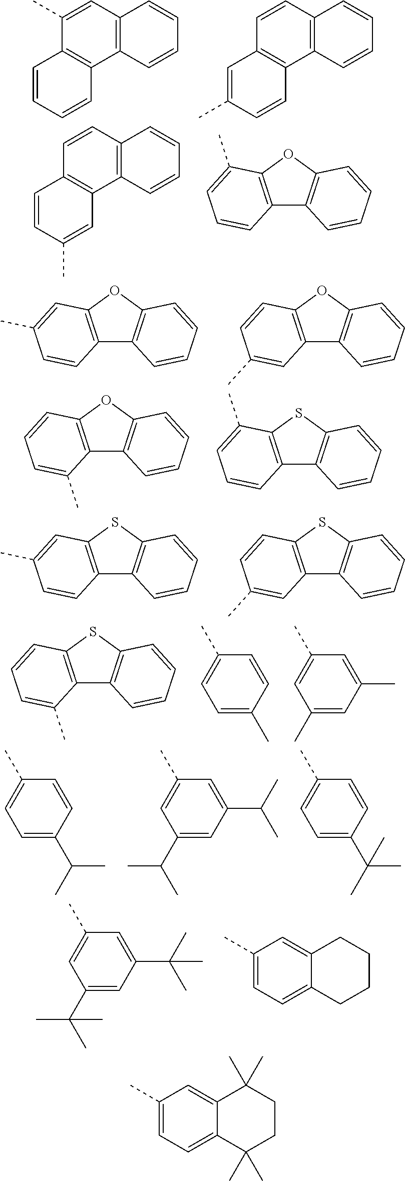

Preferably, L₁ may be a single bond; a substituted or unsubstituted C₆₋₂₀ arylene; a substituted or unsubstituted dibenzofuranylene; or a substituted or unsubstituted dibenzothiophenylene.

More preferably, L₁ may be a single bond, phenylene, biphenyldiyl, naphthalenediyl, or phenyl naphthalenediyl, wherein the phenylene, biphenyldiyl, naphthalenediyl, and phenyl naphthalenediyl may be each independently unsubstituted, or substituted with one or more deuterium.

Preferably, R₅ may be hydrogen, deuterium, or a substituted or unsubstituted C₁₋₂₀ alkyl.

More preferably, R₅ may be hydrogen, deuterium, or a substituted or unsubstituted C₁₋₁₀ alkyl.

More preferably, R₅ may be hydrogen, deuterium, a substituted or unsubstituted methyl, a substituted or unsubstituted isopropyl, or a substituted or unsubstituted tertbutyl.

Most preferably, R₅ may be hydrogen, deuterium, methyl, isopropyl, or tertbutyl, wherein the methyl, isopropyl, and tertbutyl may be each independently unsubstituted, or substituted with at least one deuterium.

The deuterium substitution rate of the compound may be 50% to 100%. Specifically, the deuterium substitution rate of the compound may be 50% or more, 60% or more, 70% or more, 75% or more, 80% or more, or 90% or more, and 100% or less.

Furthermore, the compound may contain 16 or more deuterium atoms. As an example, the compound may contain 16 to 50 deuterium atoms, but is not limited thereto. More specifically, the compounds may contain 16 or more, 17 or more, or 18 or more, and 50 or less, 45 or less, 40 or less, 38 or less, 36 or less, 34 or less, 32 or less, 30 or less, 28 or less, 26 or less, 24 or less, 23 or less, 22 or less, 21 or less, or 20 or less deuterium atoms.

At this time, if the number of deuterium substitutions in the compound is to be represented, it can be represented as the following Chemical Formula 1-D:

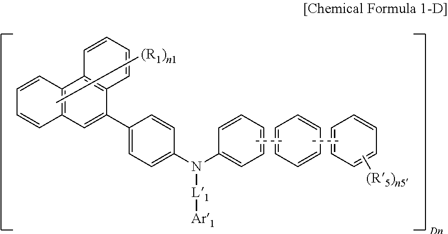

in Chemical Formula 1-D,
Dn means that the total number of substituted deuterium (D) in the entire compound is n,
R₁ and n1 is as defined in Chemical Formula 1,
the n is a value including n1,
R′₅ is a C₁₋₆₀ alkyl that is not substituted with deuterium,
n5′ is an integer of 0 to 5,
L′₁ and Ar′₁ mean L₁ and Ar₁ substituents that are not substituted with deuterium, respectively.

That is, in the Chemical Formula 1-D, n is the total number of deuterium substituted in the compound, which is an integer whose deuterium substitution rate of the compound is 50% or more, and is a value including n1, which is the number of deuterium substituted in phenanthrenyl. The compound represented by Chemical Formula 1-D means a compound in which phenanthrenyl is substituted with n1 deuterium atoms and the entire compound is substituted with n deuterium atoms.

As an example, in Chemical Formula 1D, n of Dn may be 16 or more, 17 or more, or 18 or more, and 50 or less, 45 or less, 40 or less, 38 or less, 36 or less, 34 or less, 32 or less, 30 or less, 28 or less, 26 or less, 24 or less, 23 or less, 22 or less, 21 or less, or 20 or less.

Representative examples of the compound represented by Chemical Formula 1 are as follows:

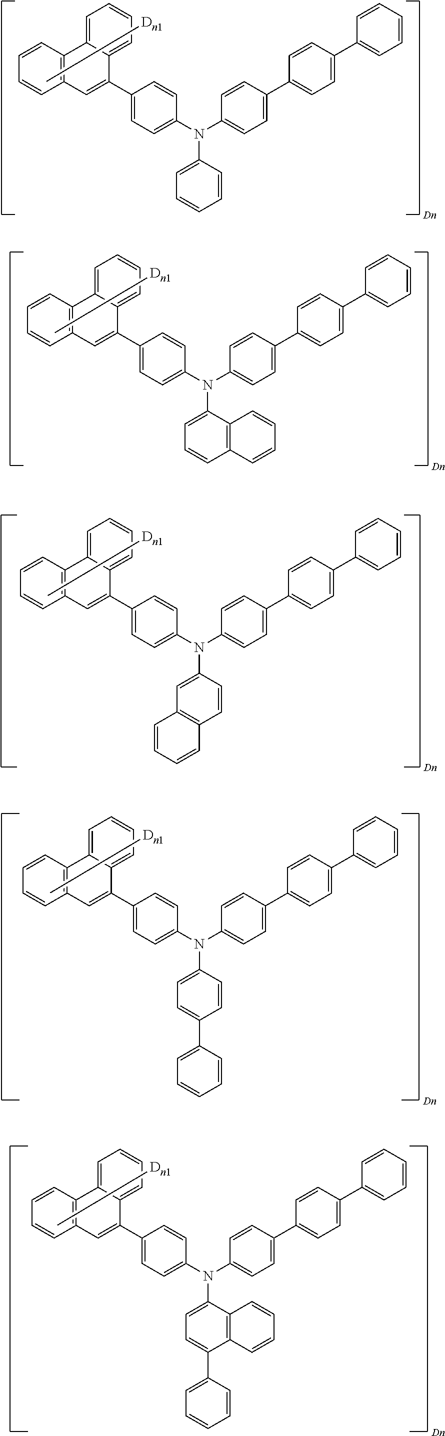

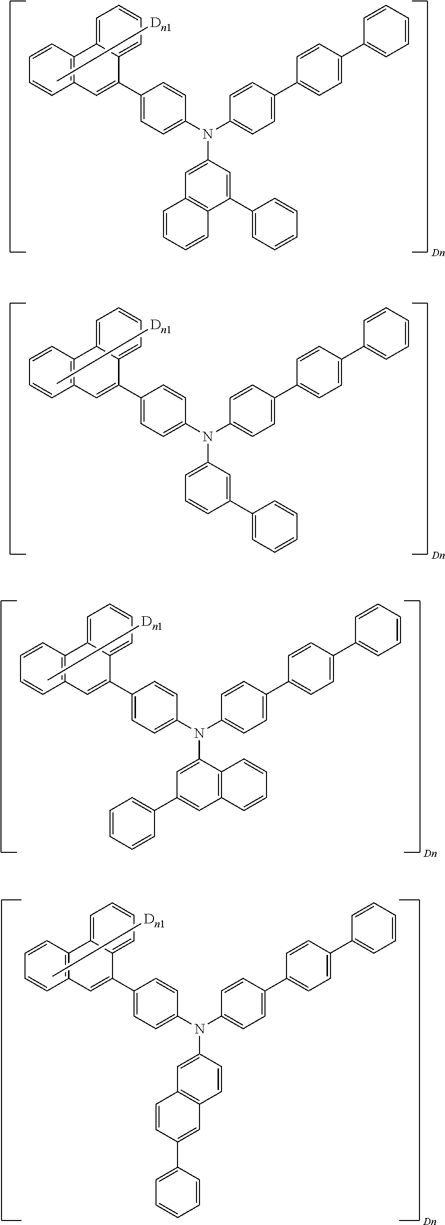

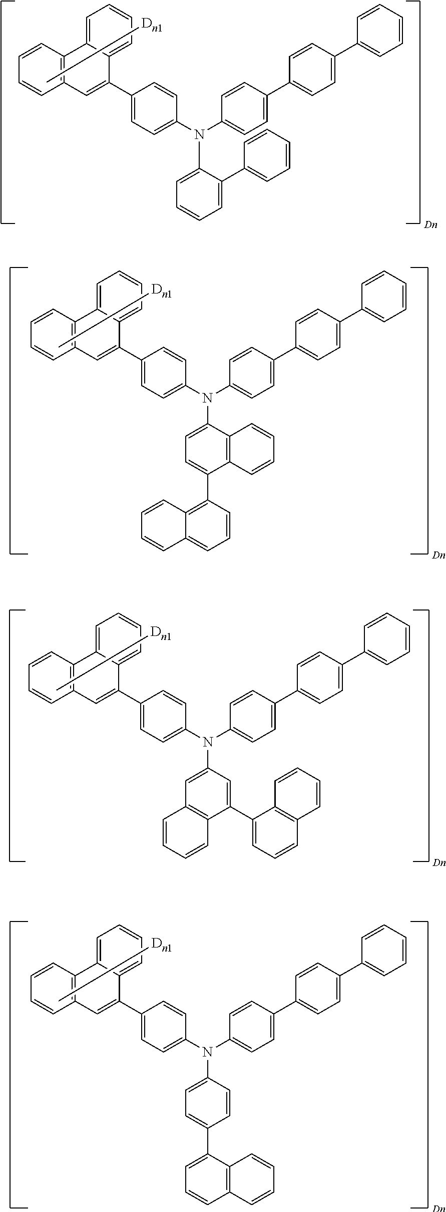

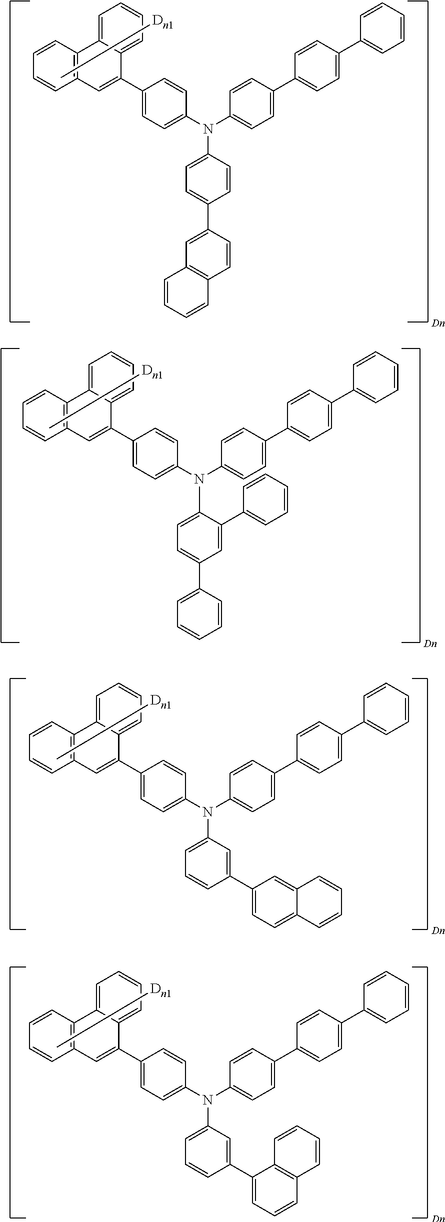

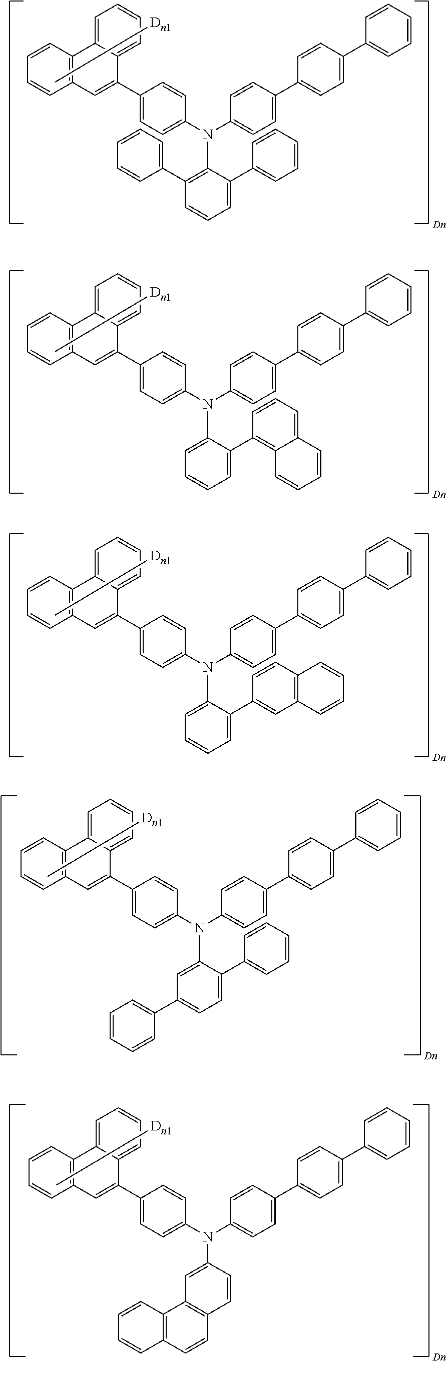

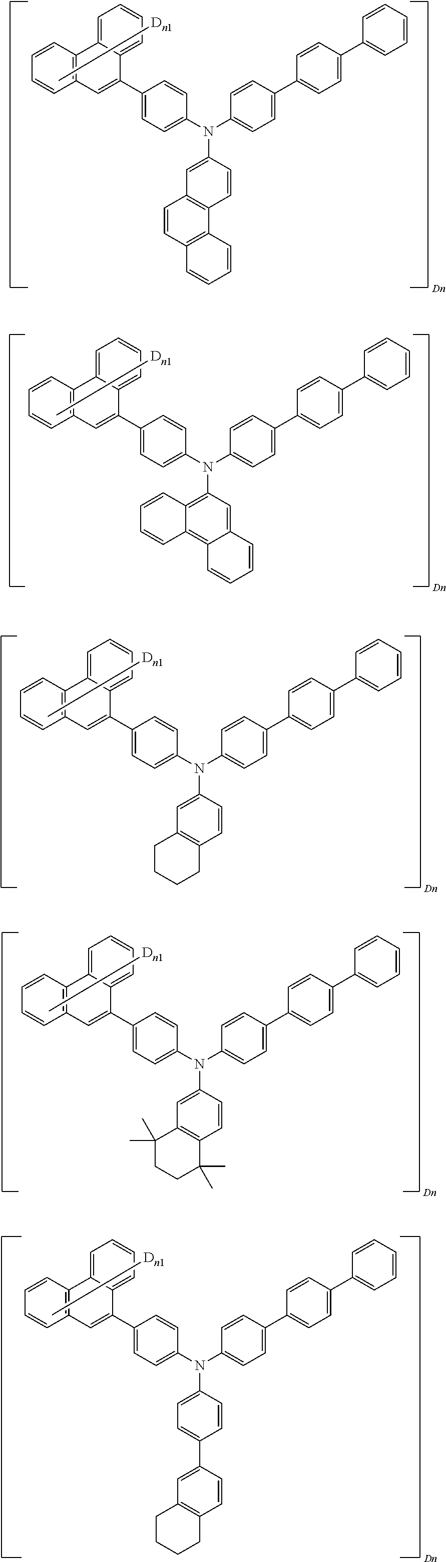

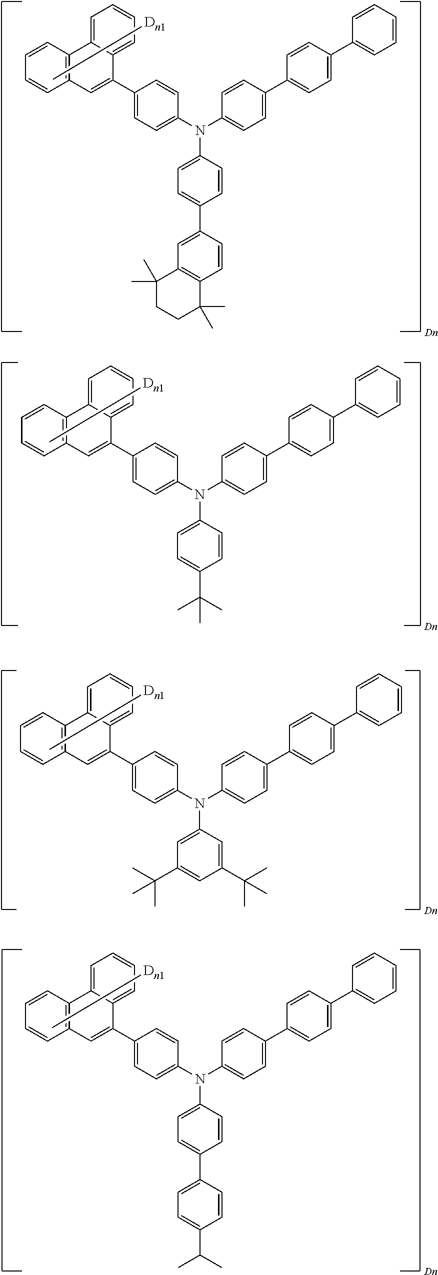

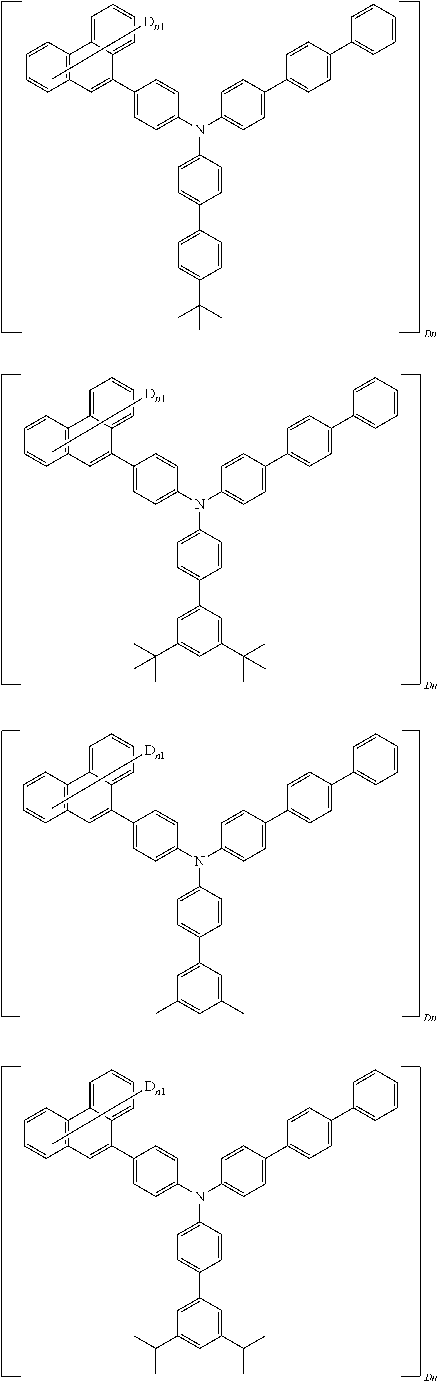

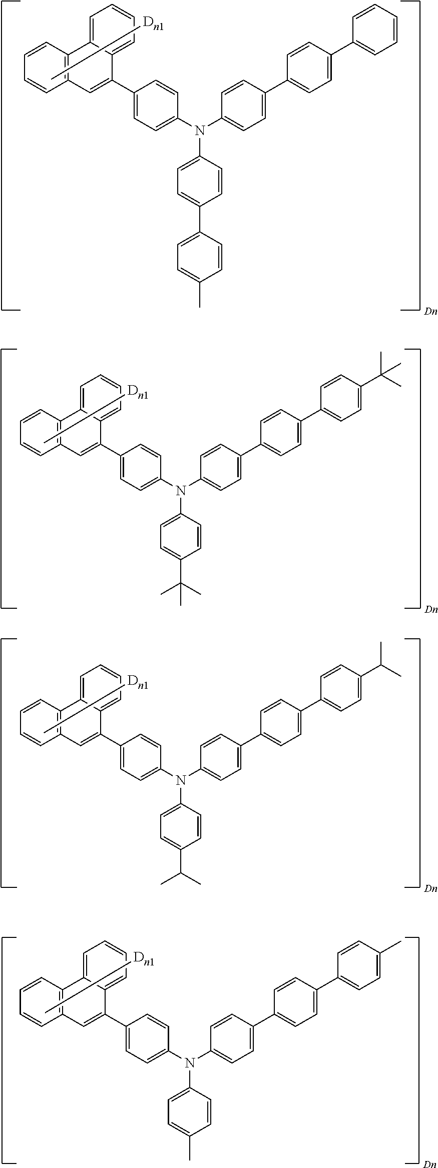

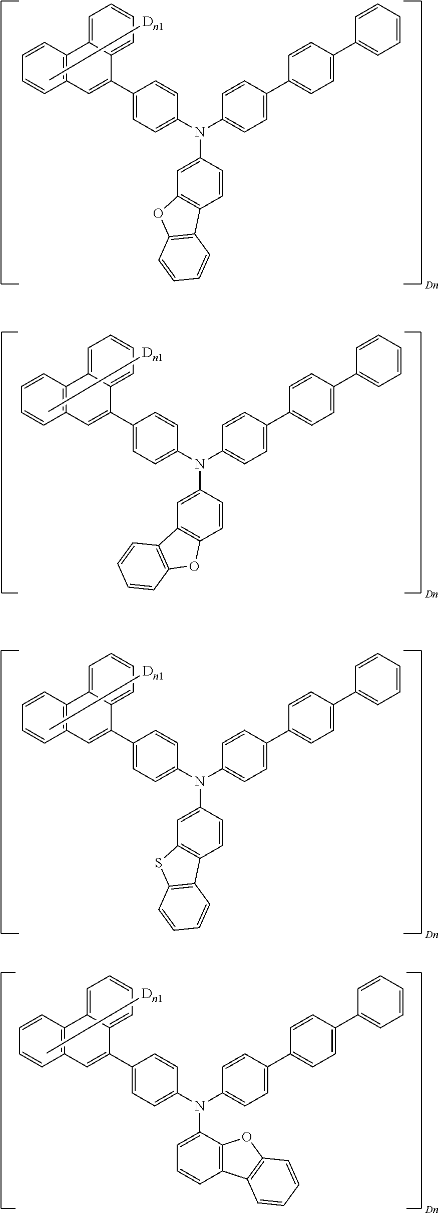

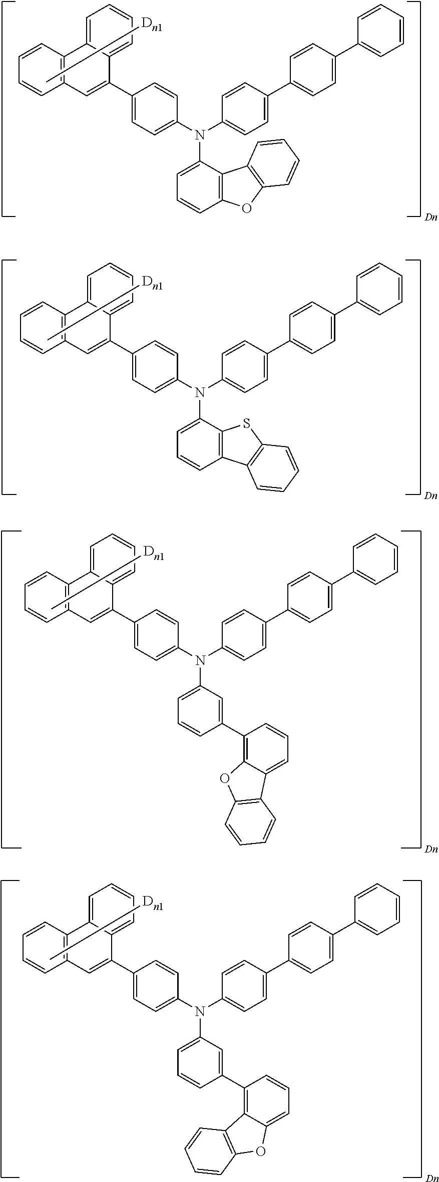

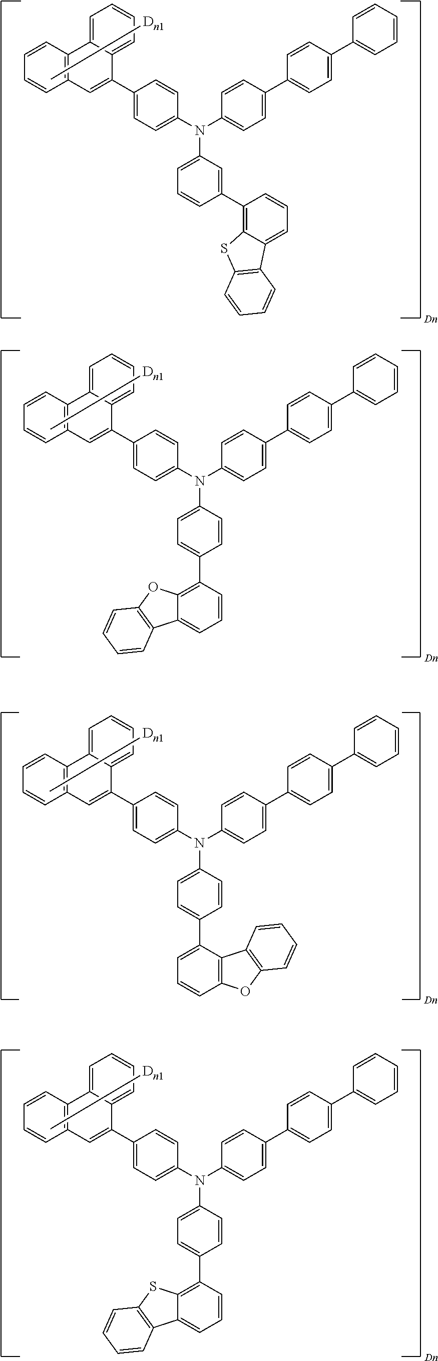

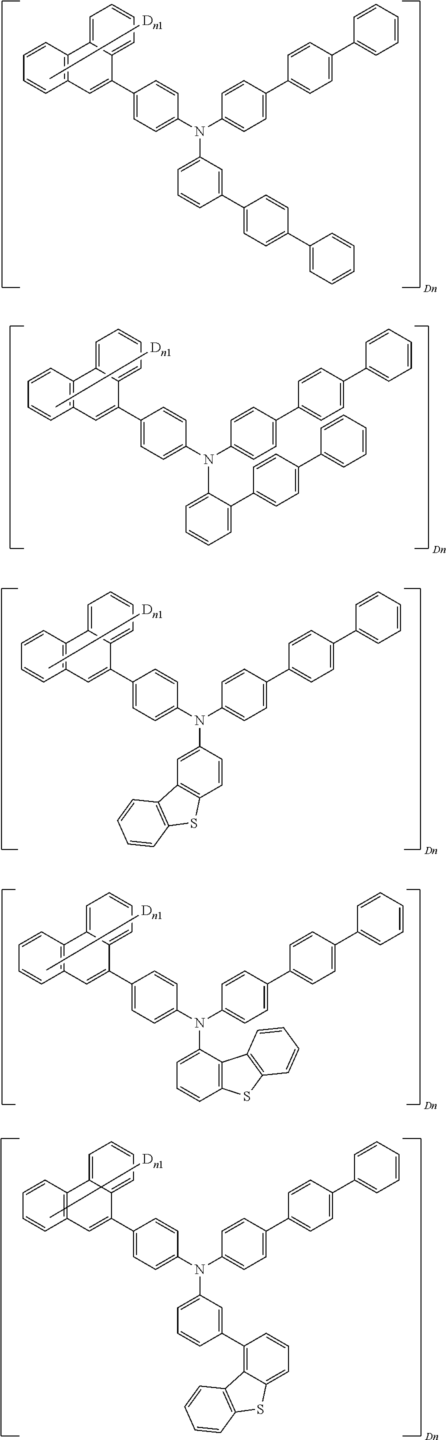

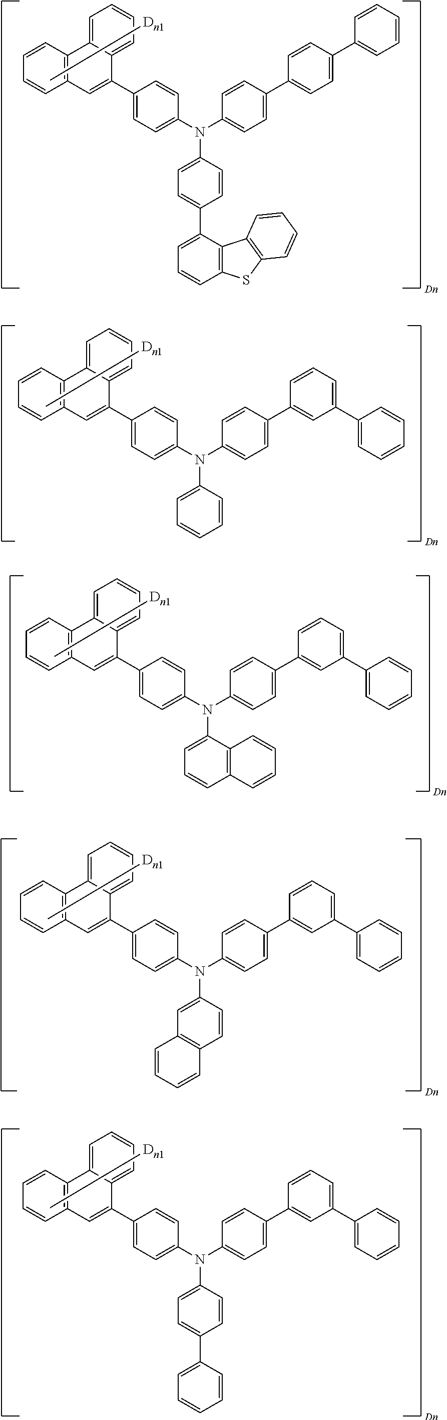

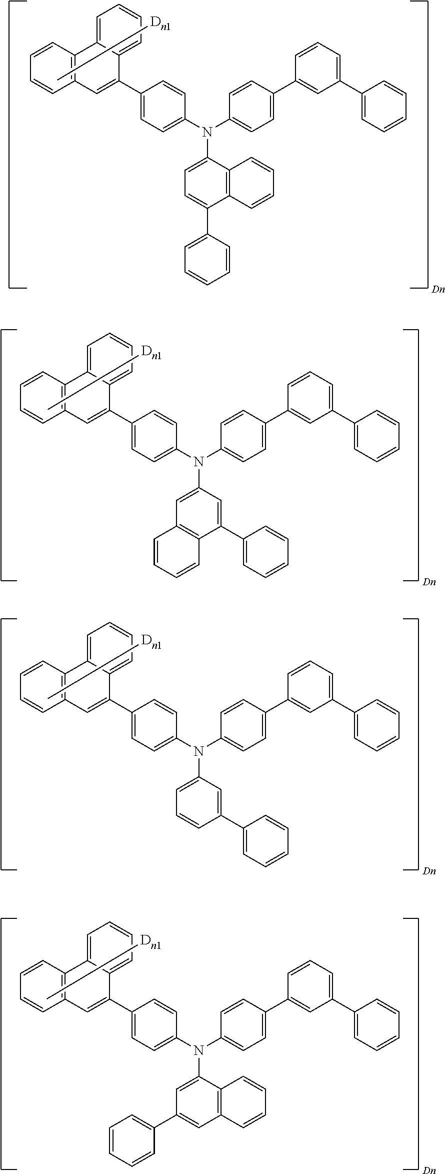

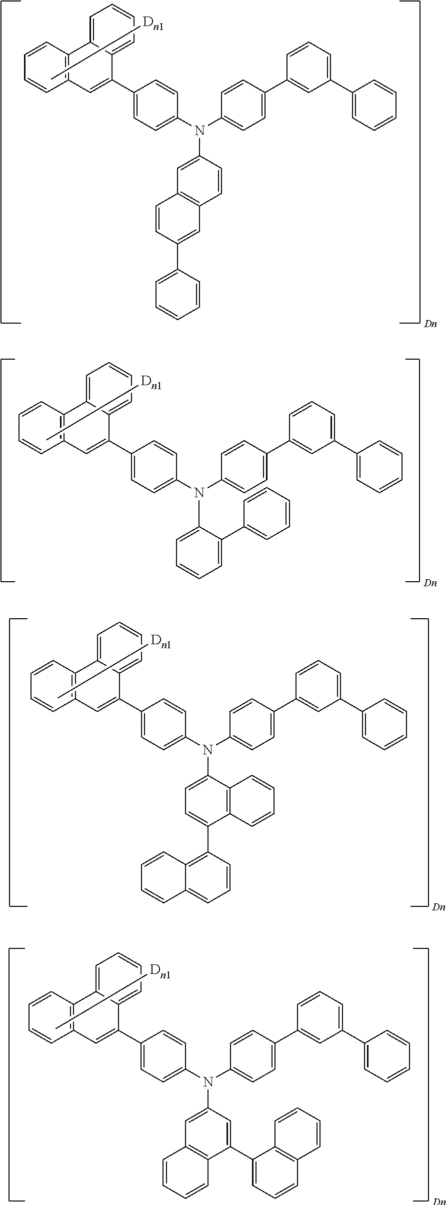

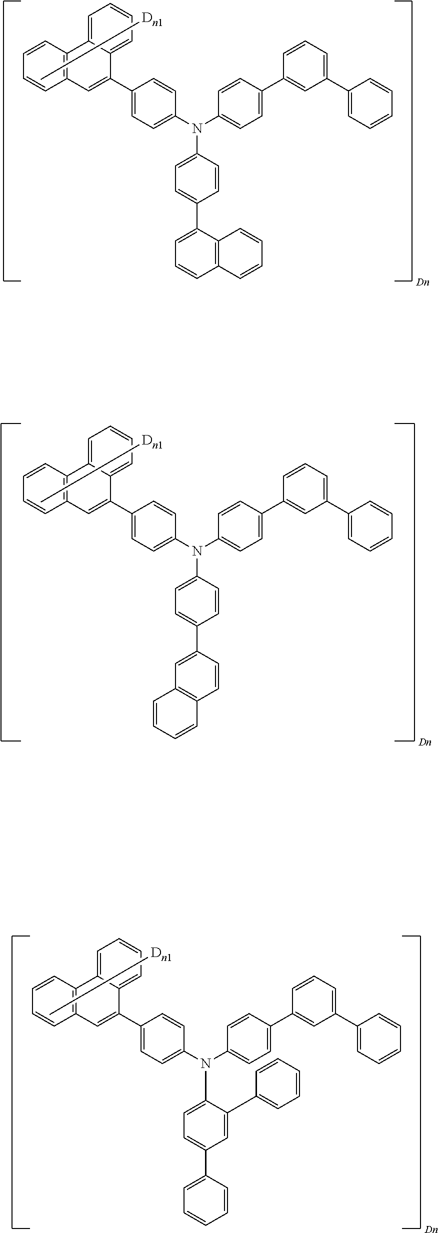

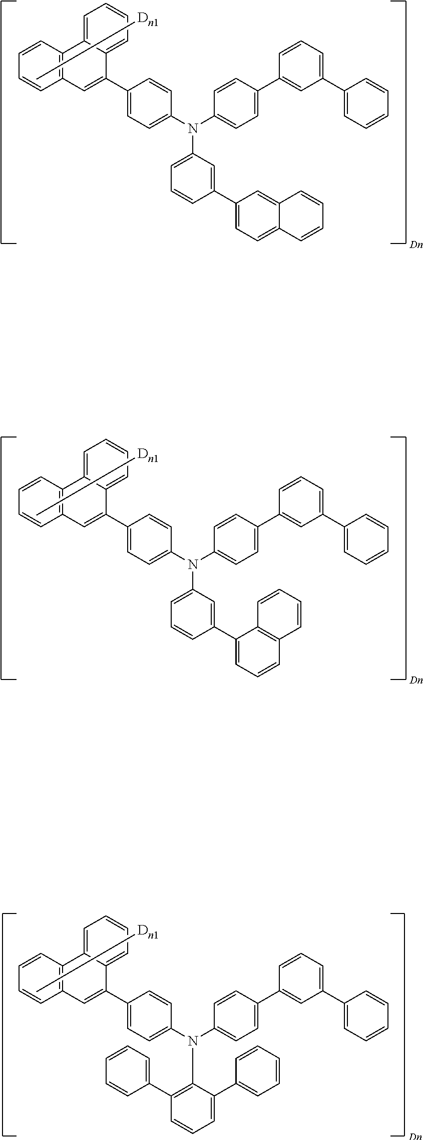

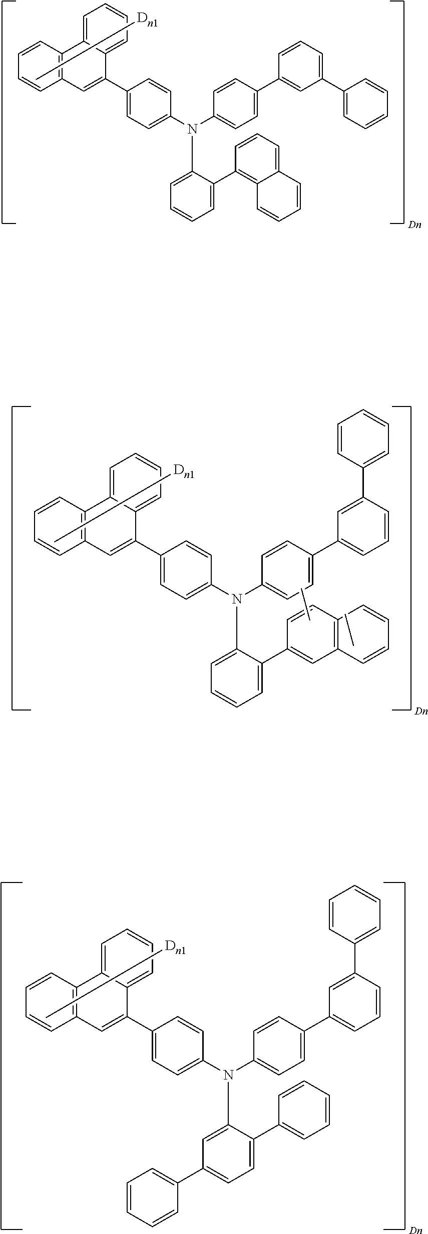

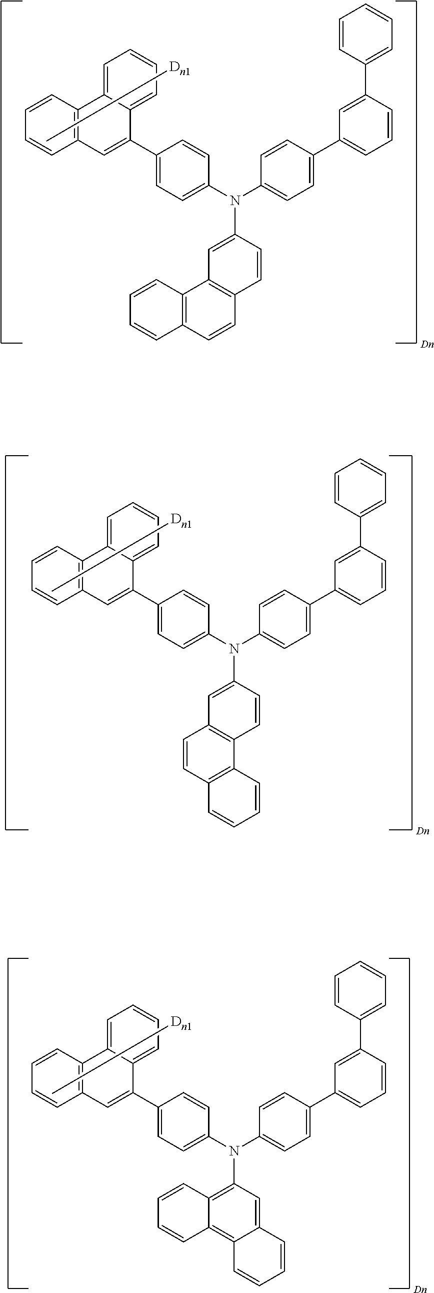

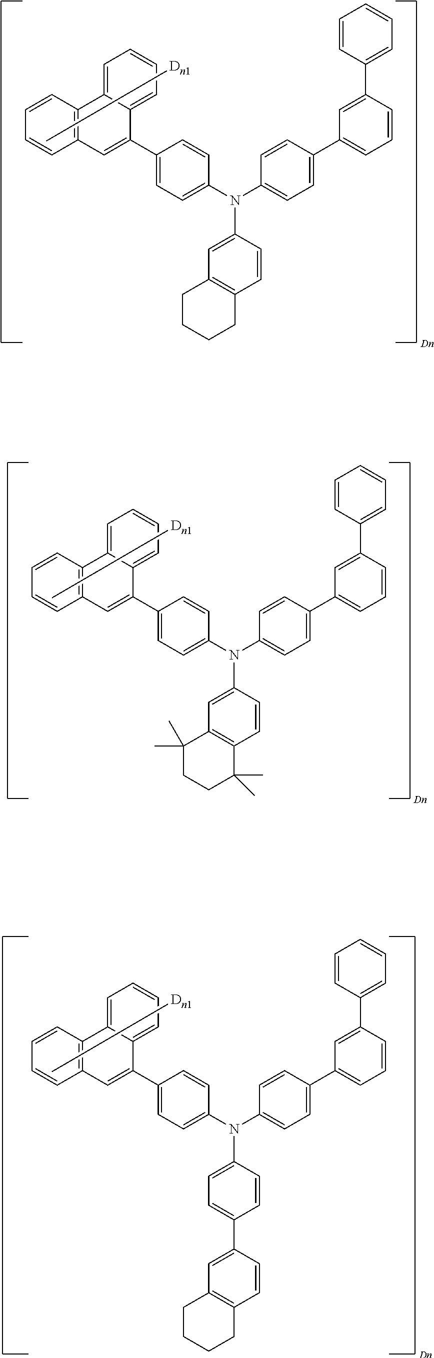

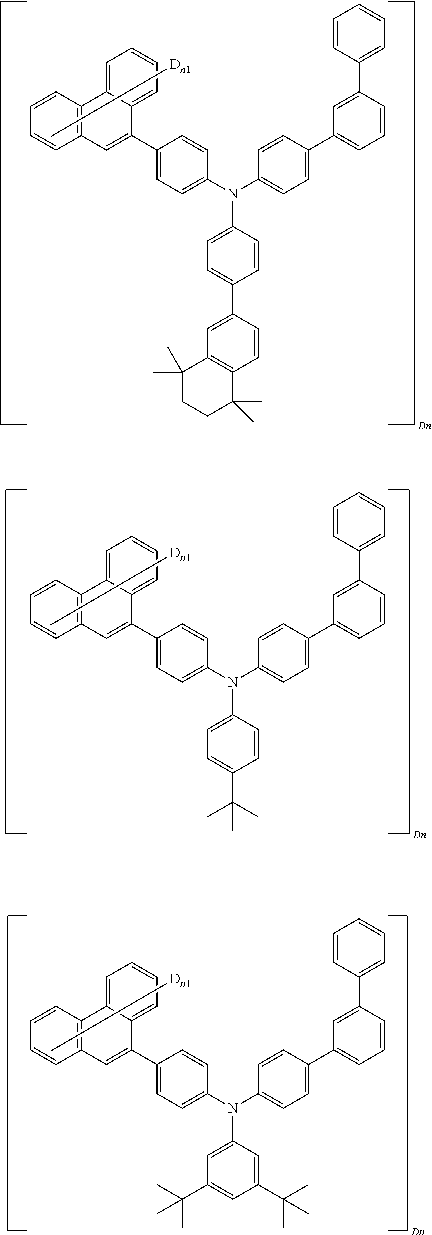

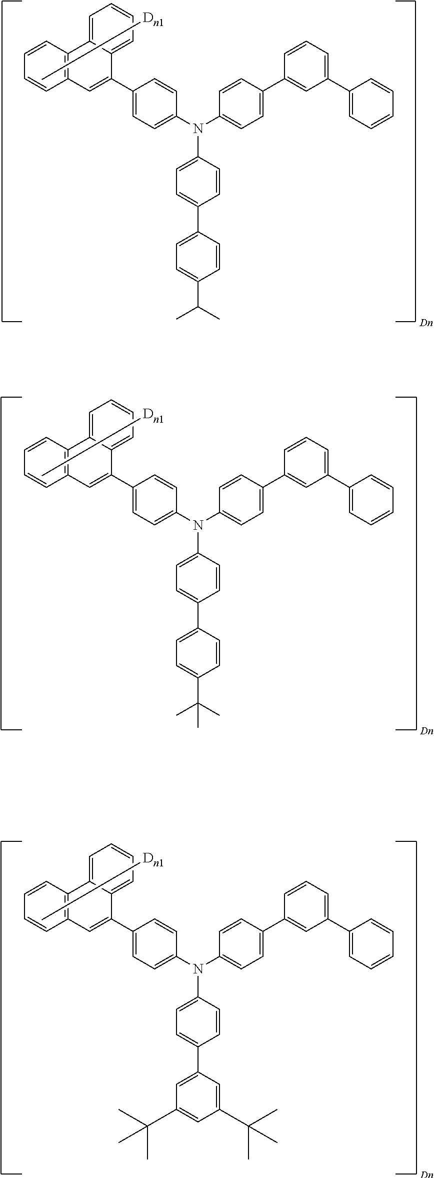

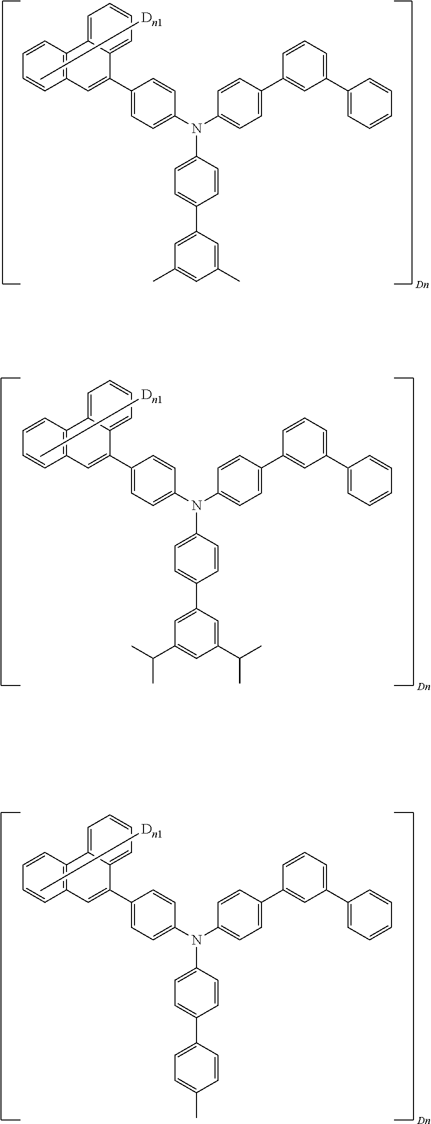

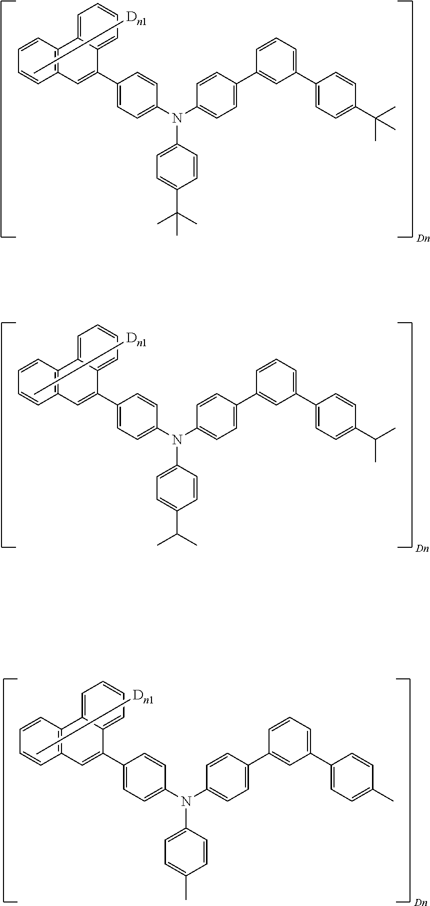

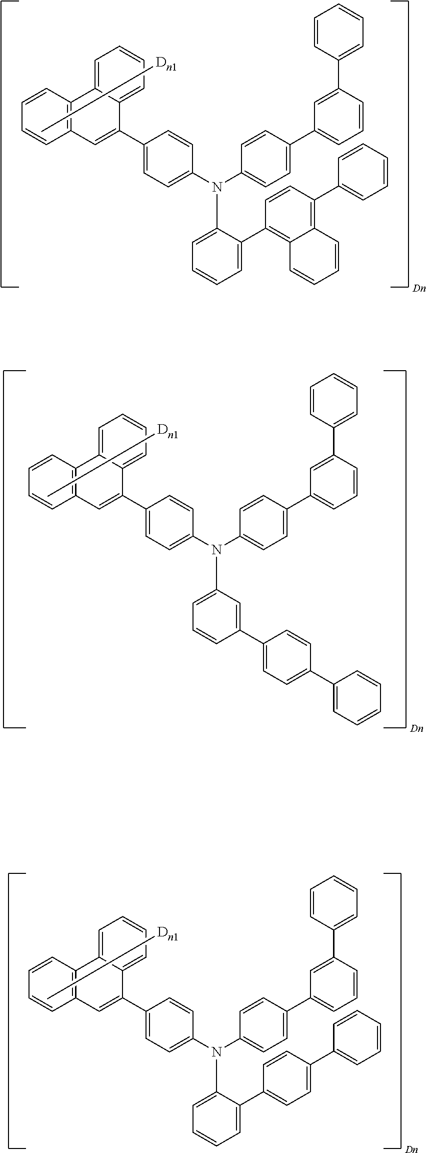

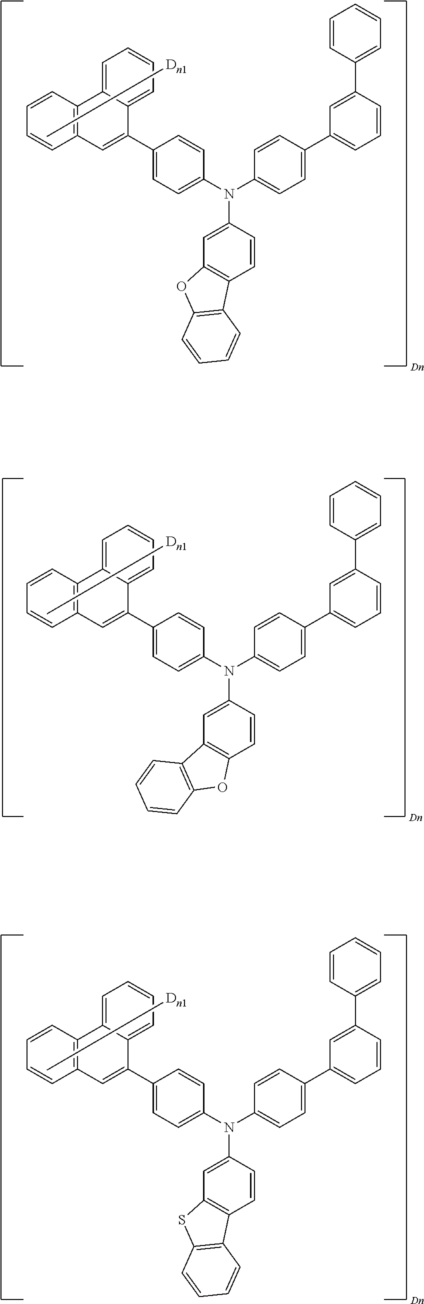

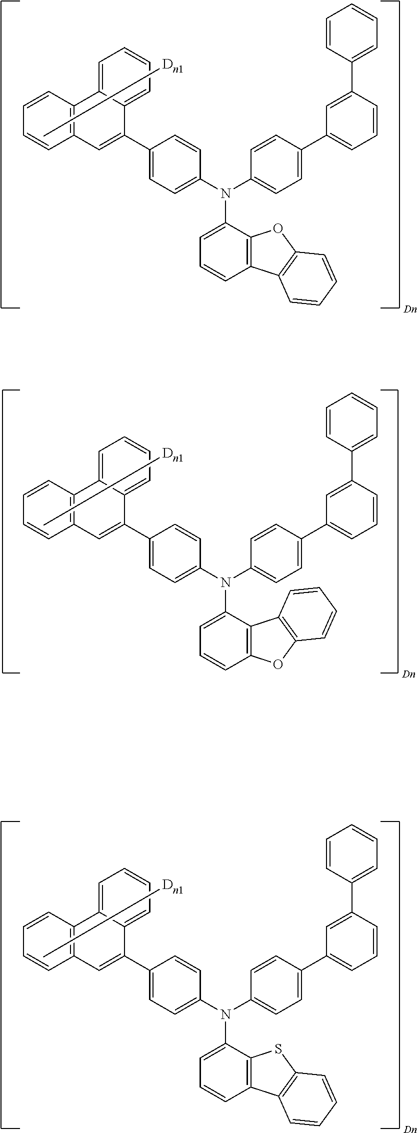

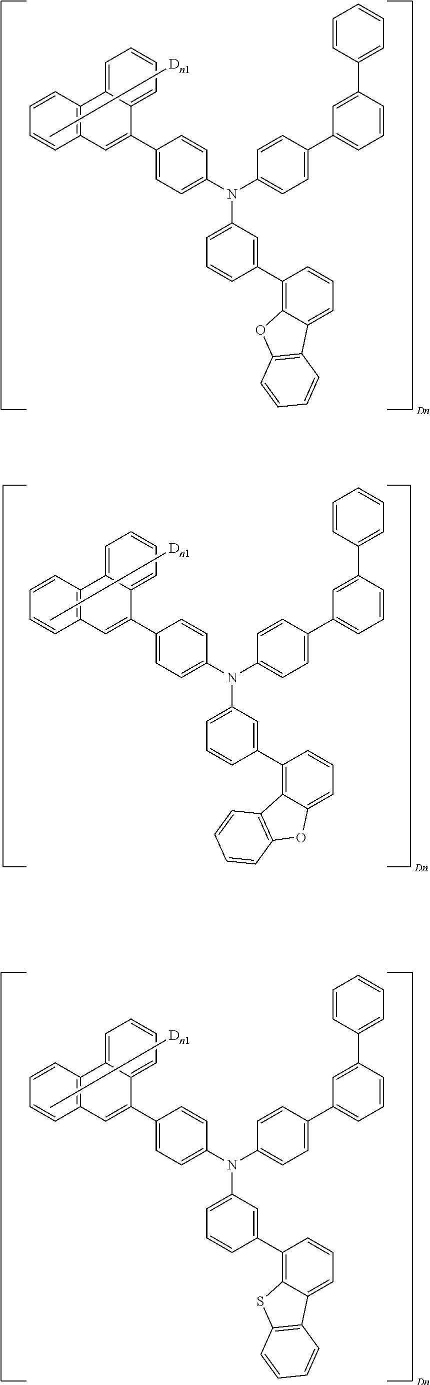

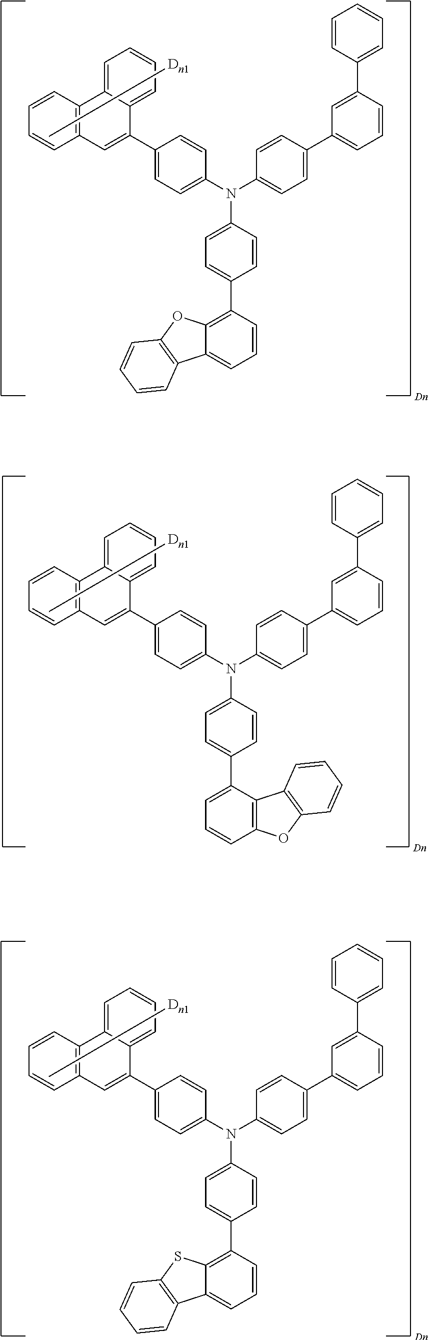

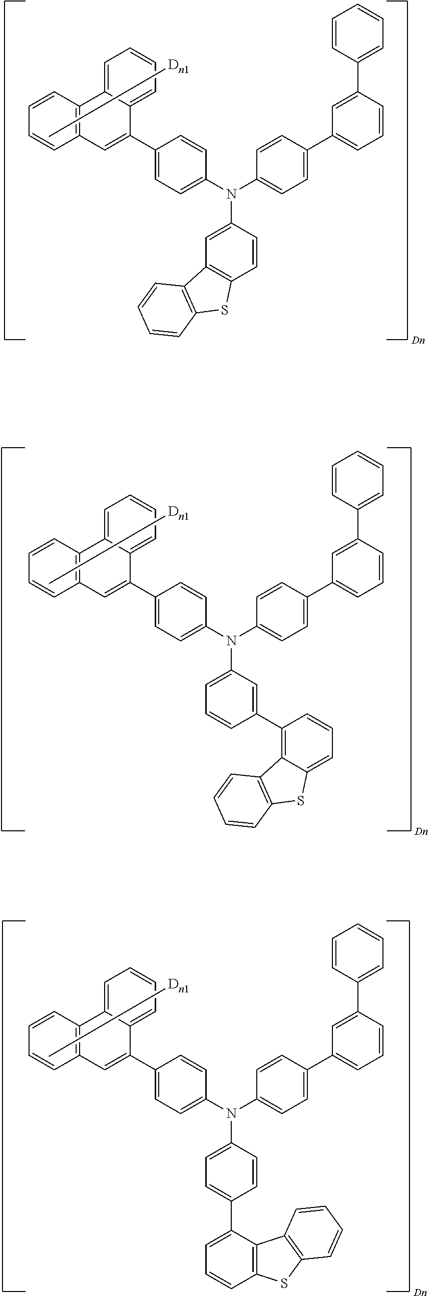

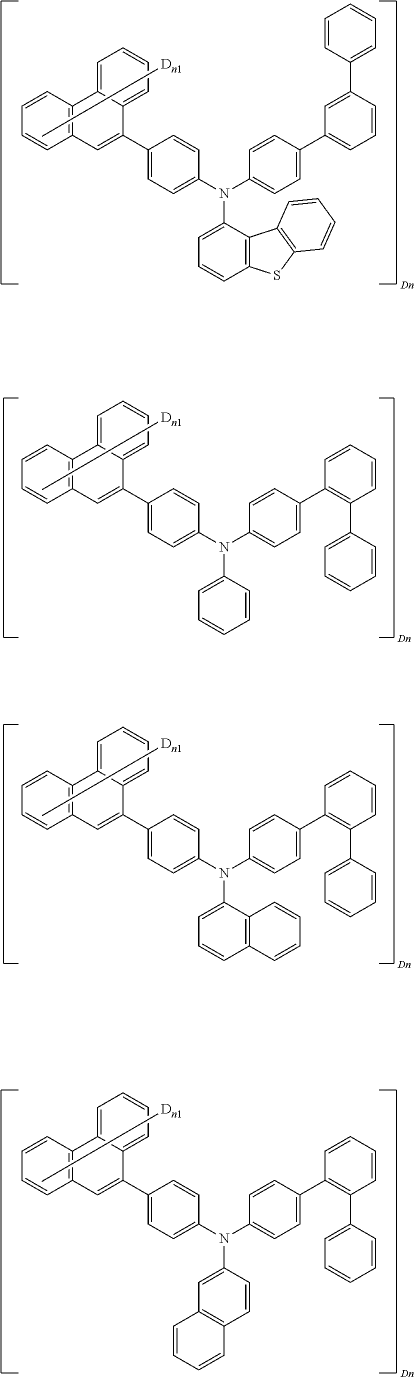

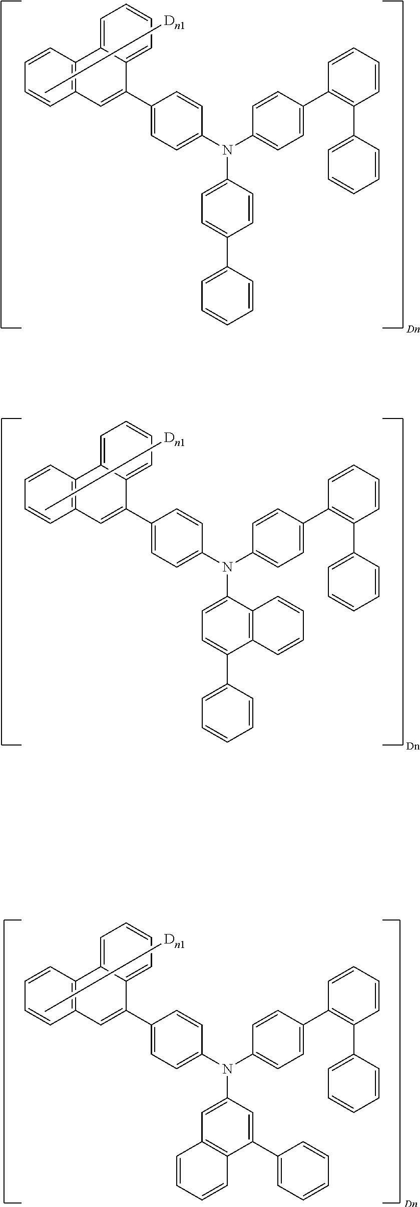

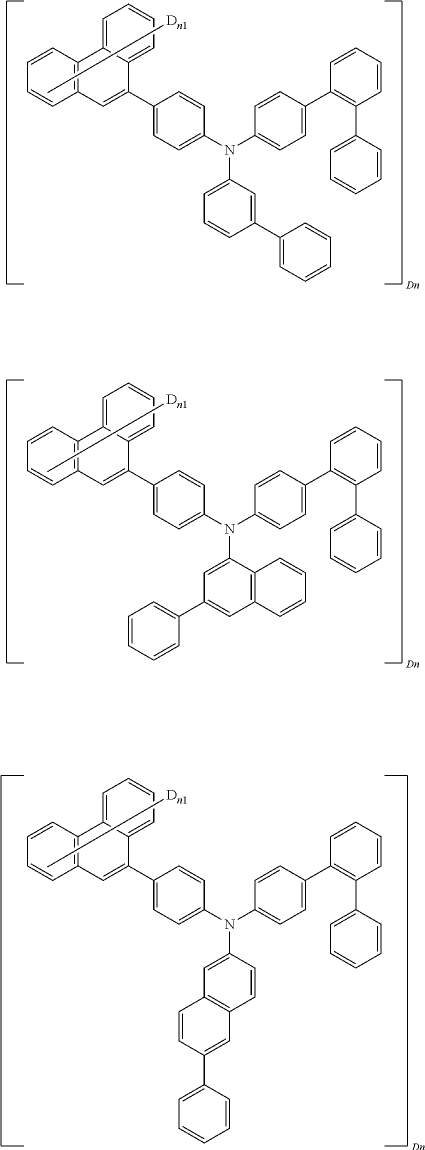

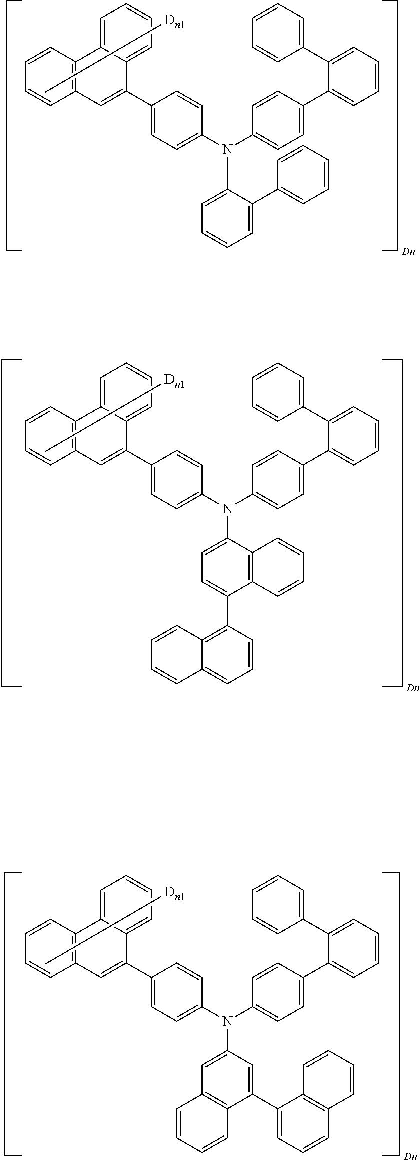

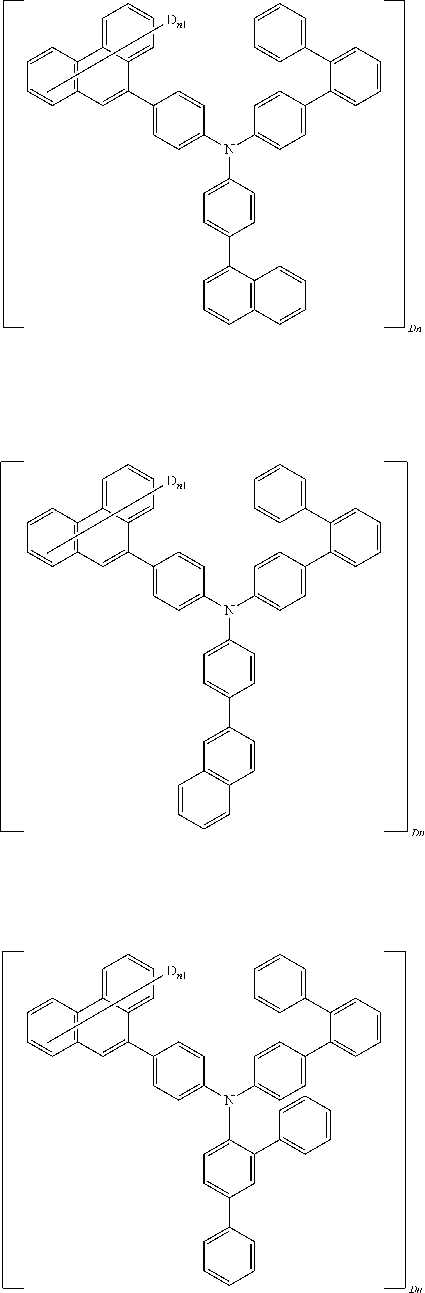

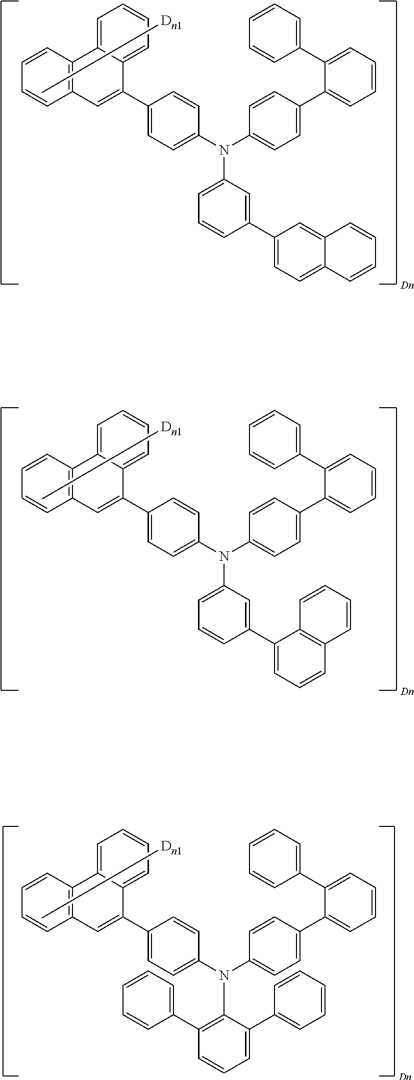

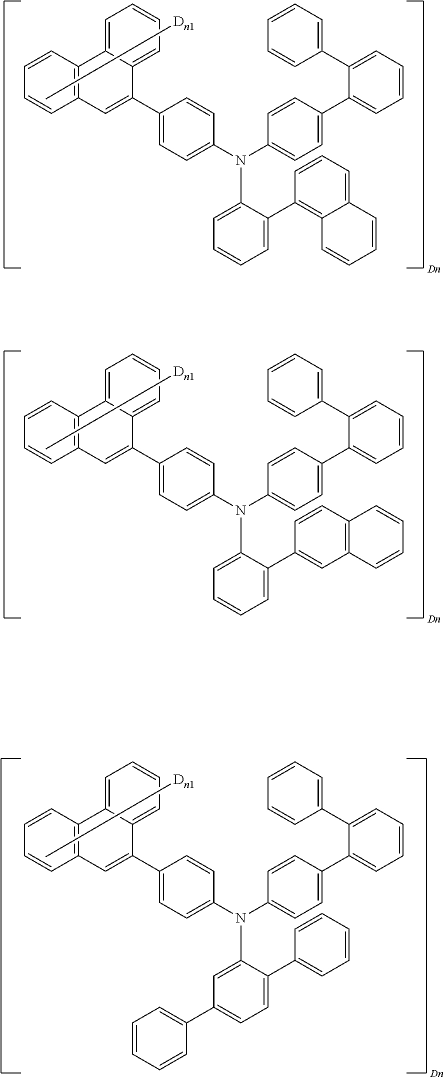

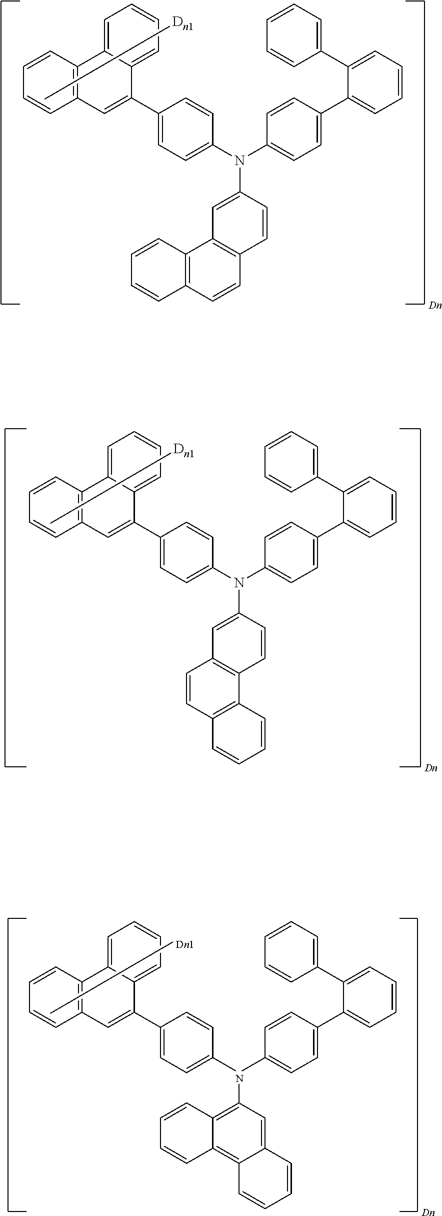

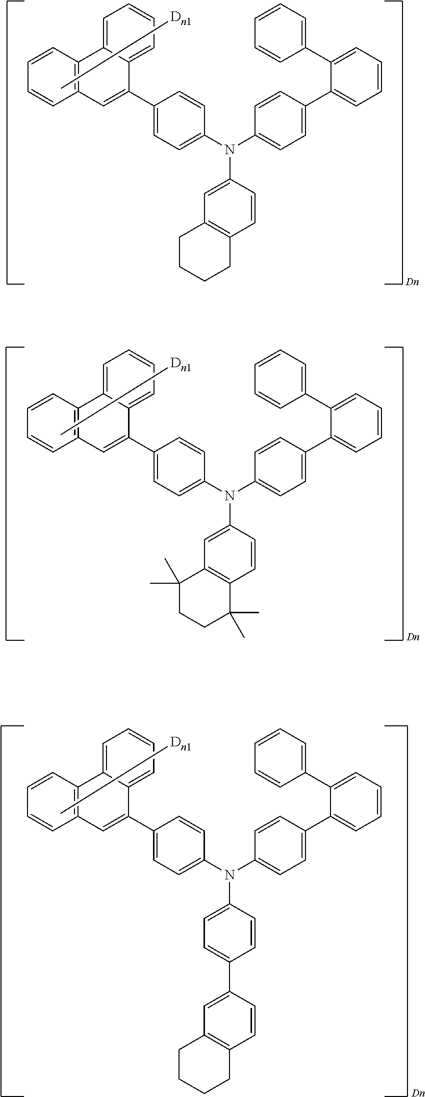

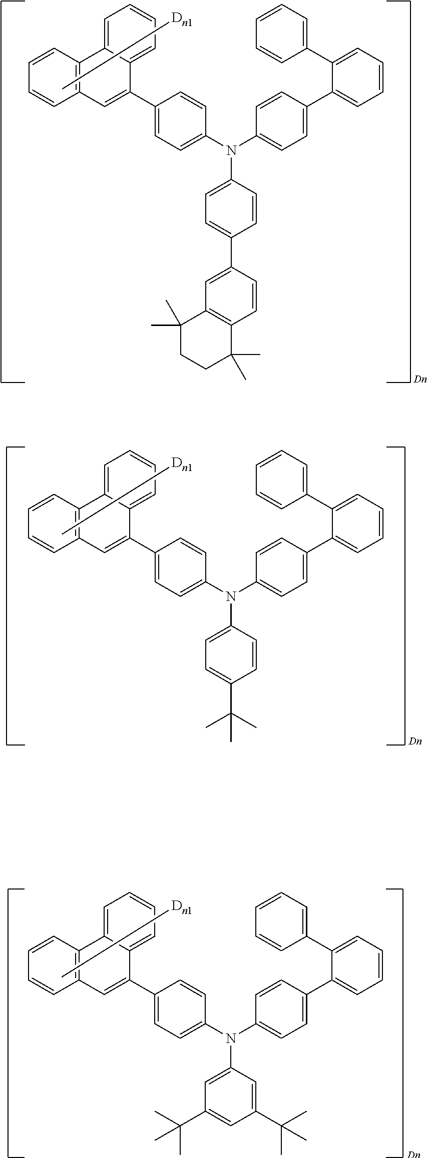

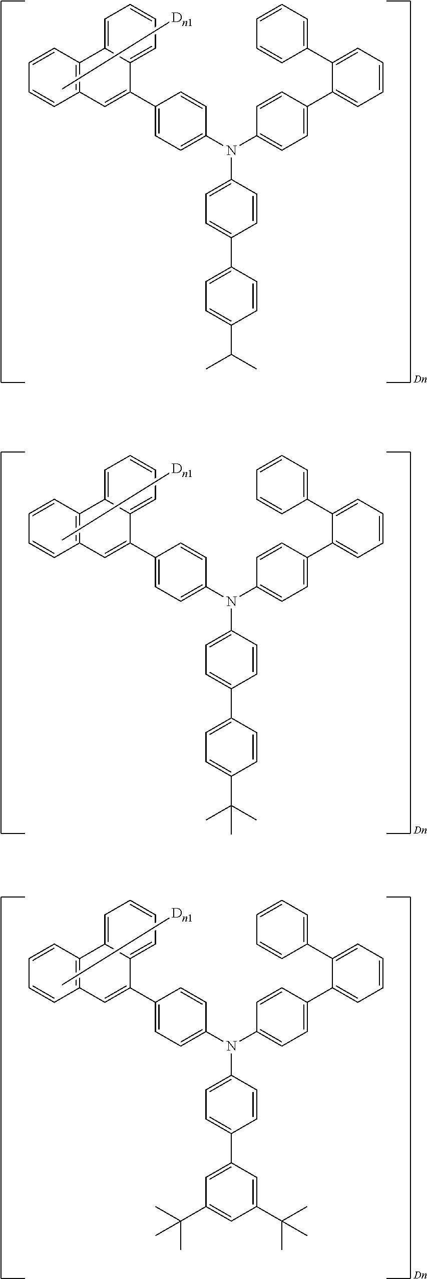

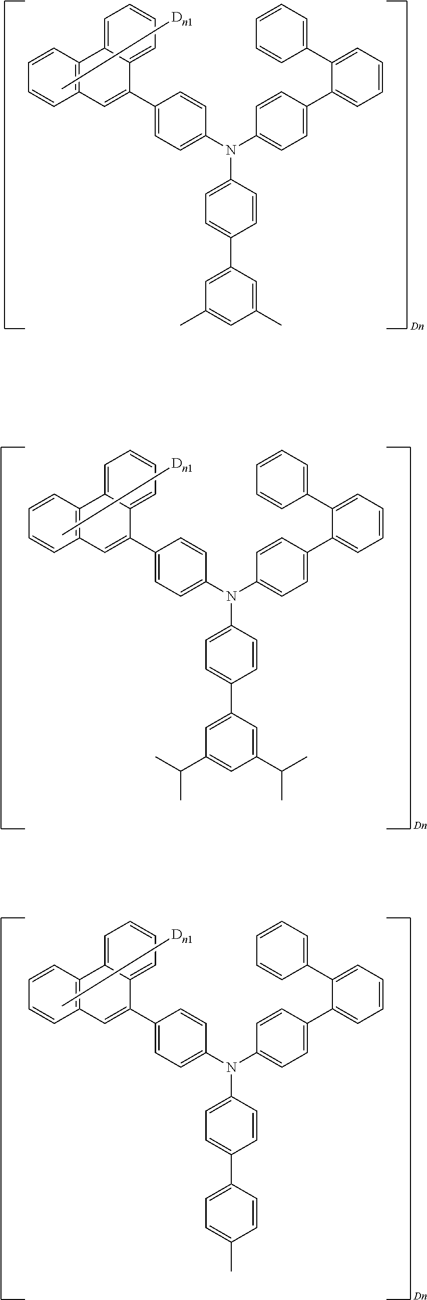

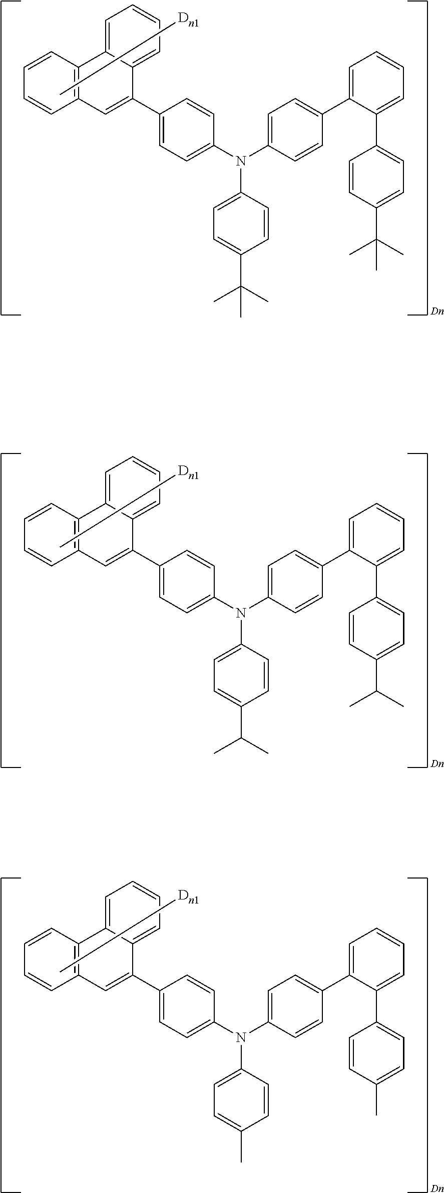

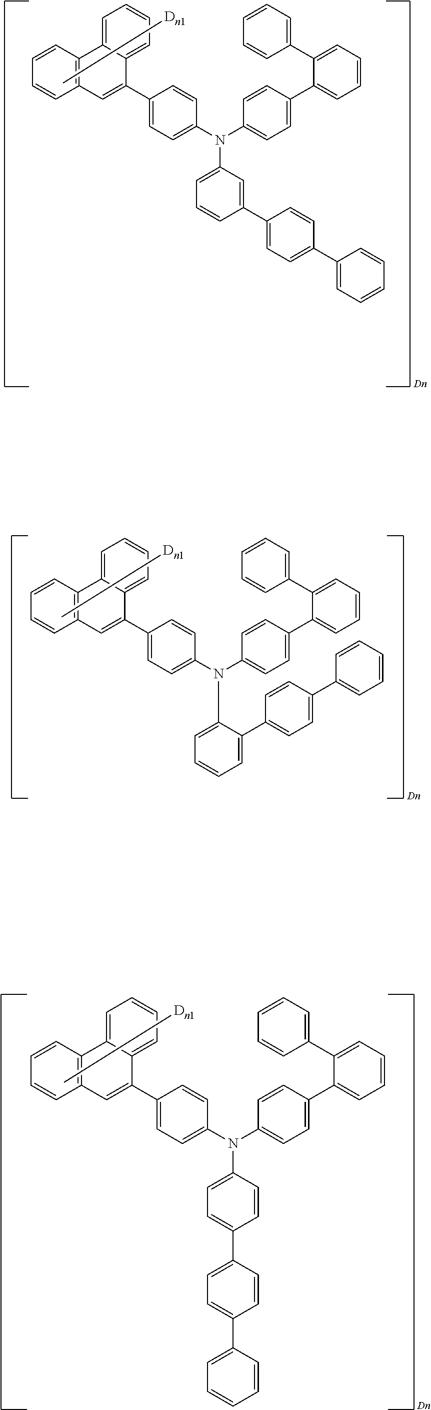

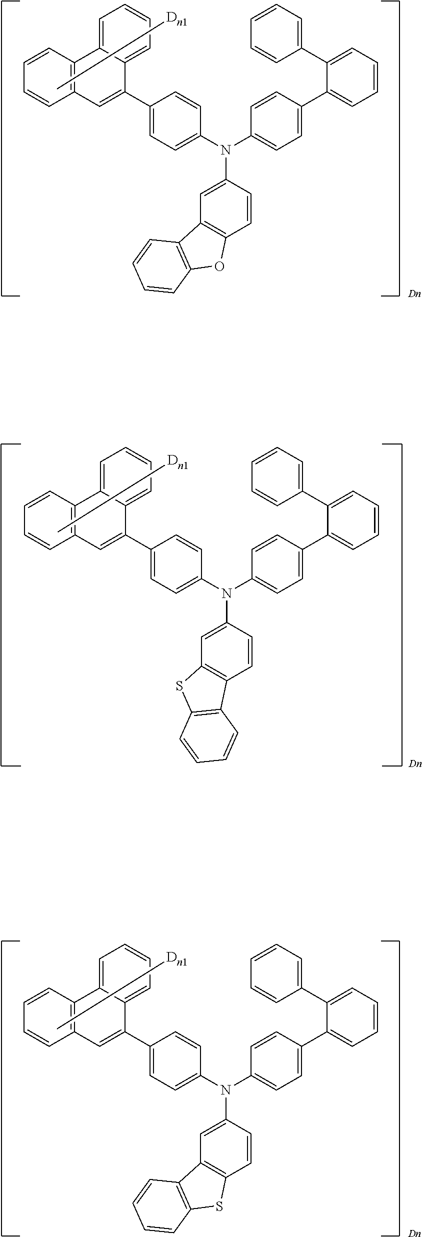

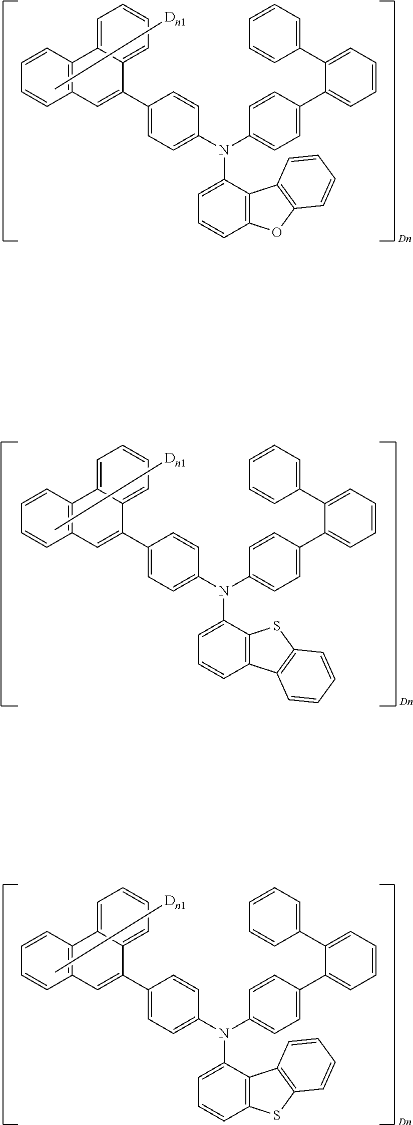

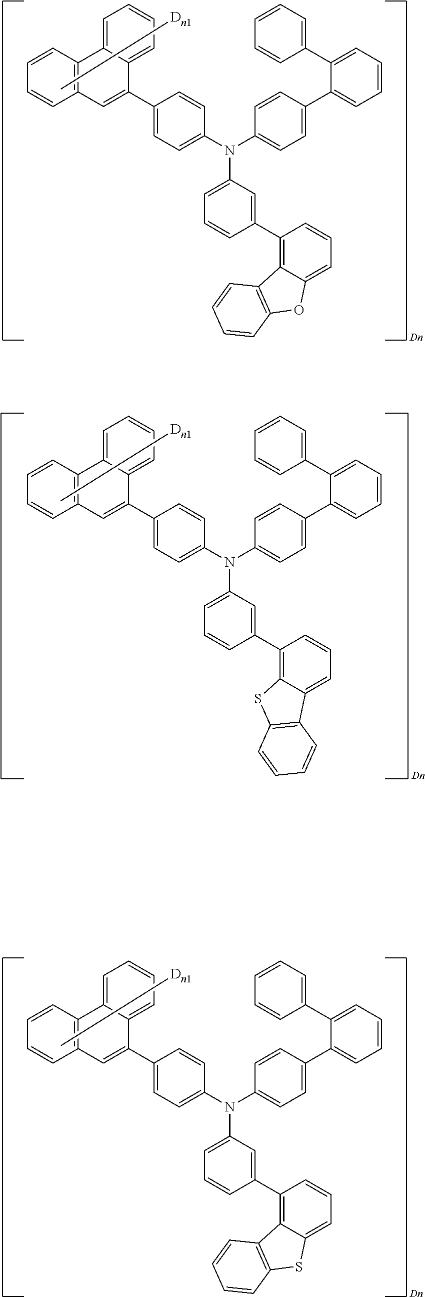

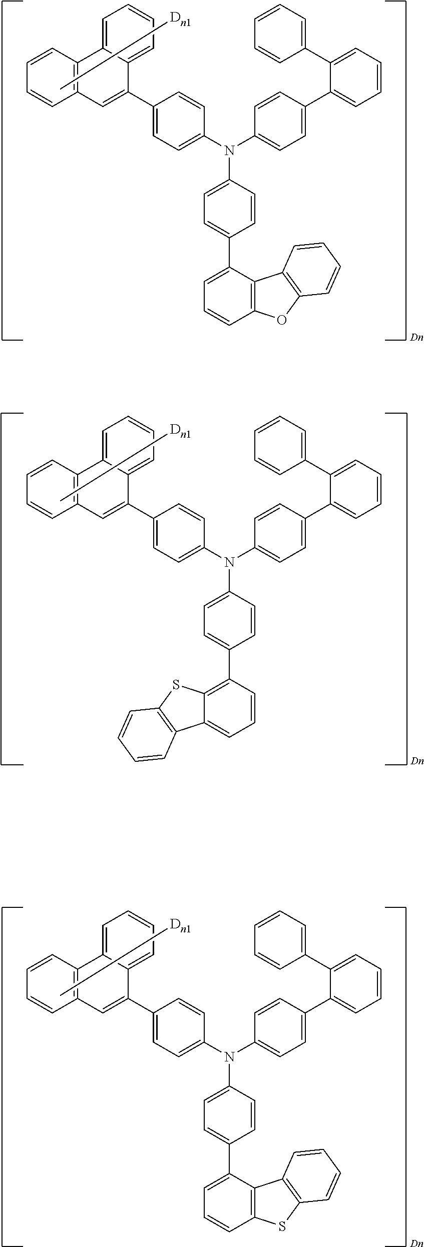

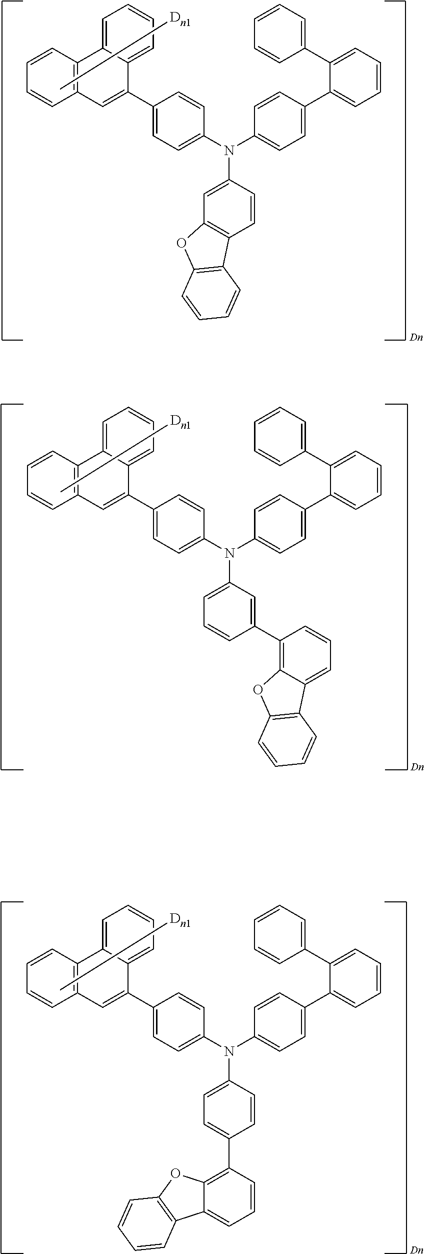

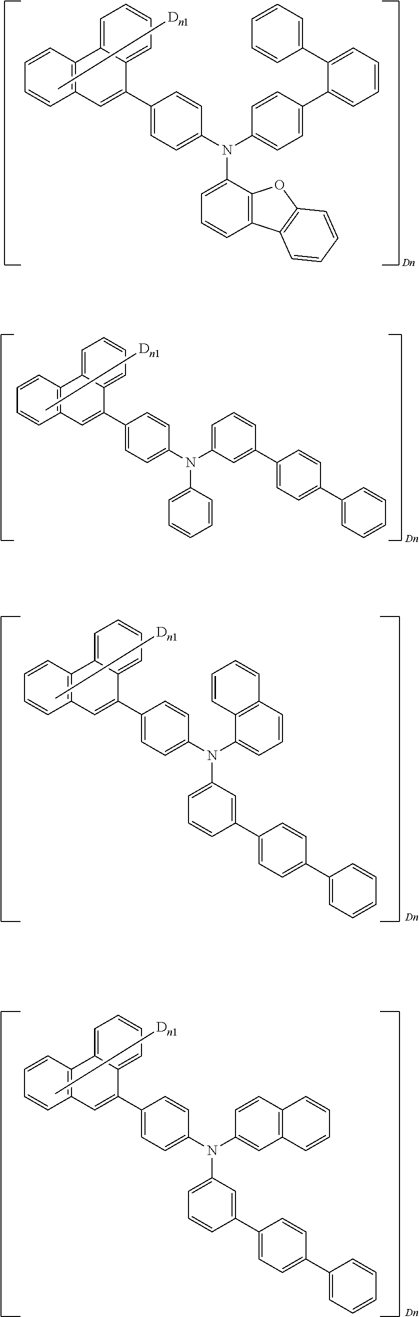

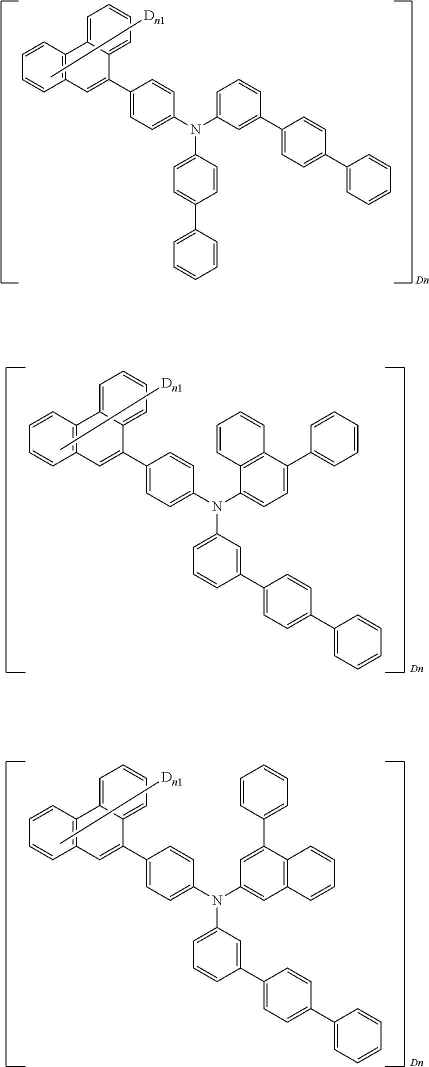

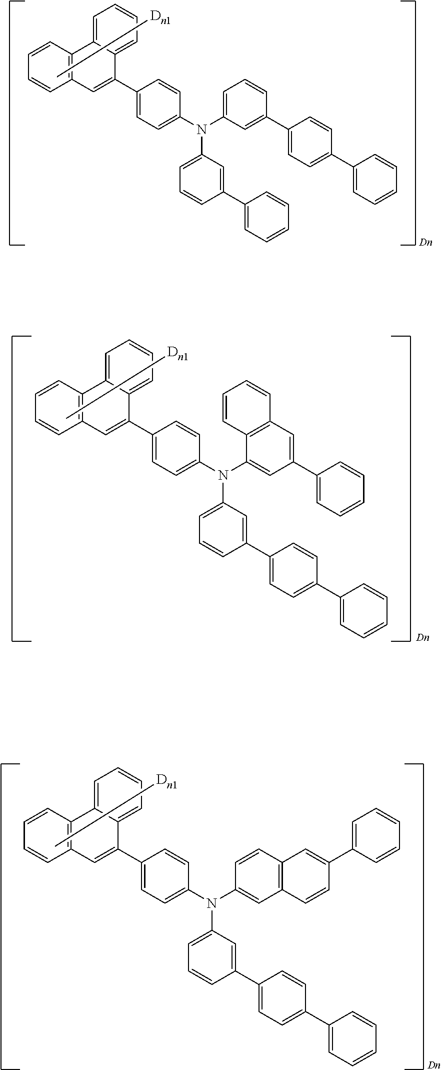

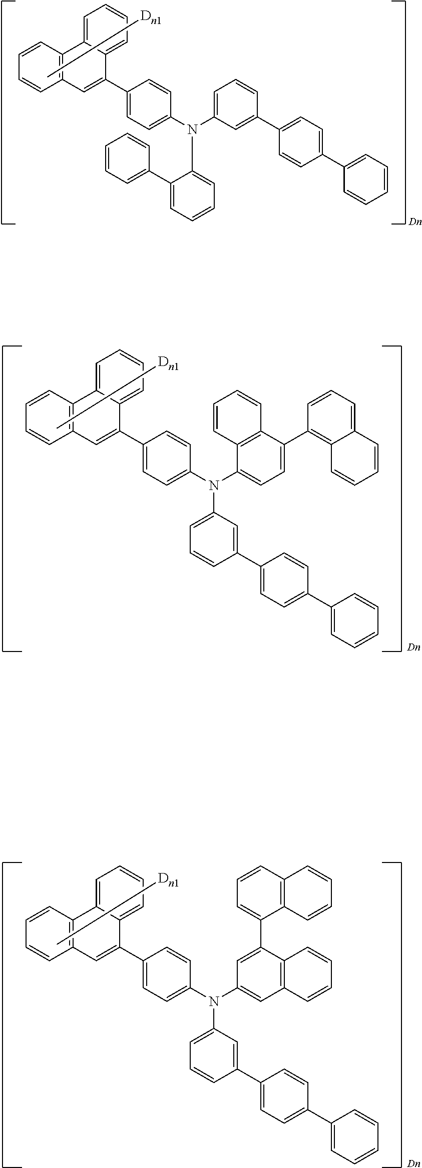

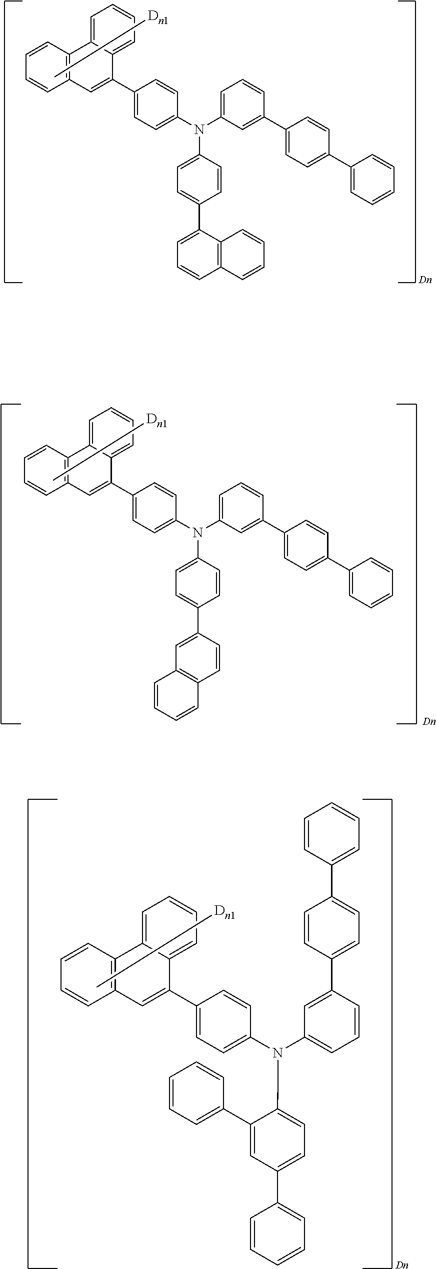

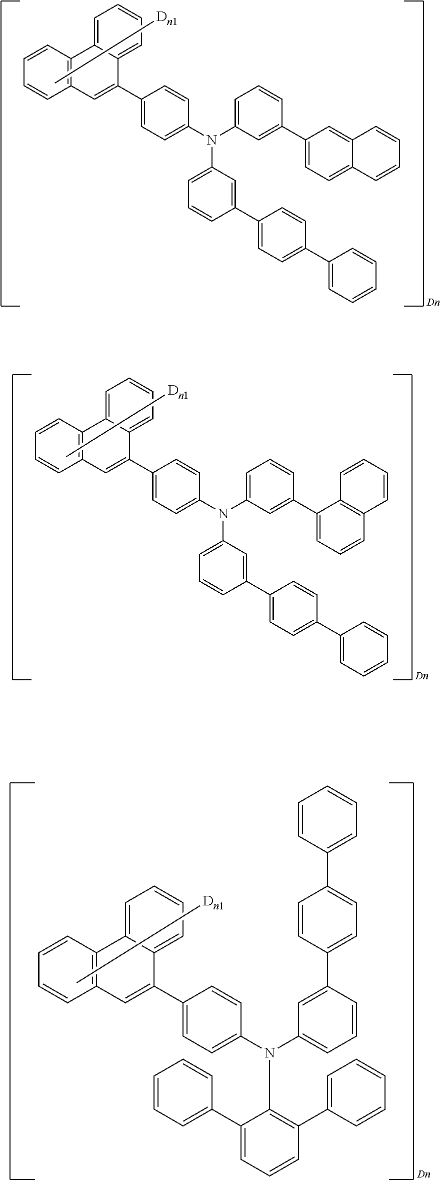

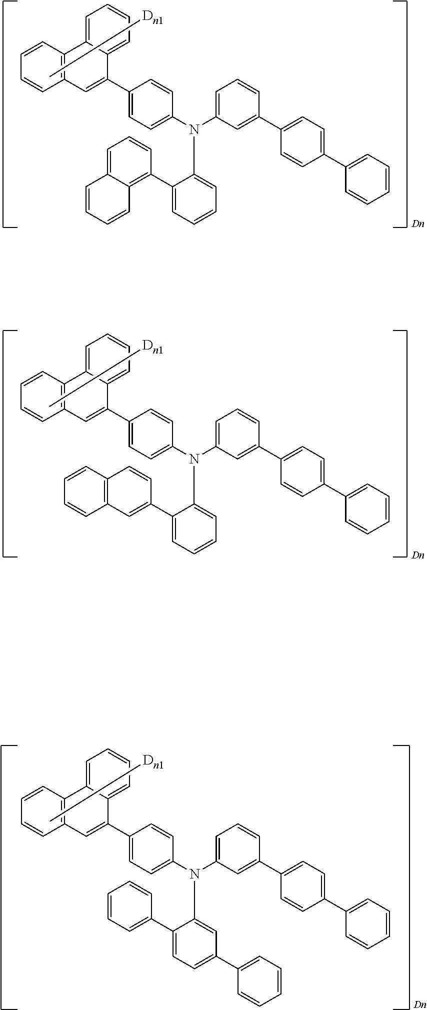

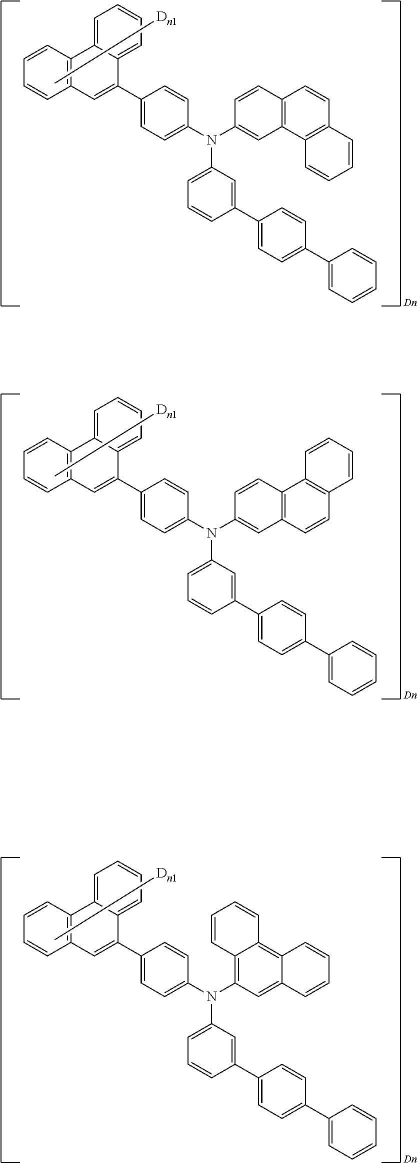

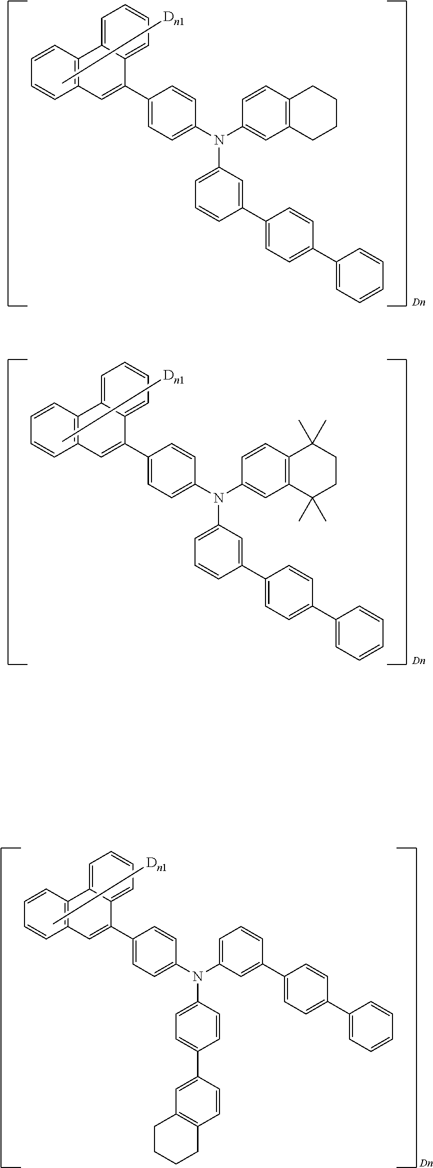

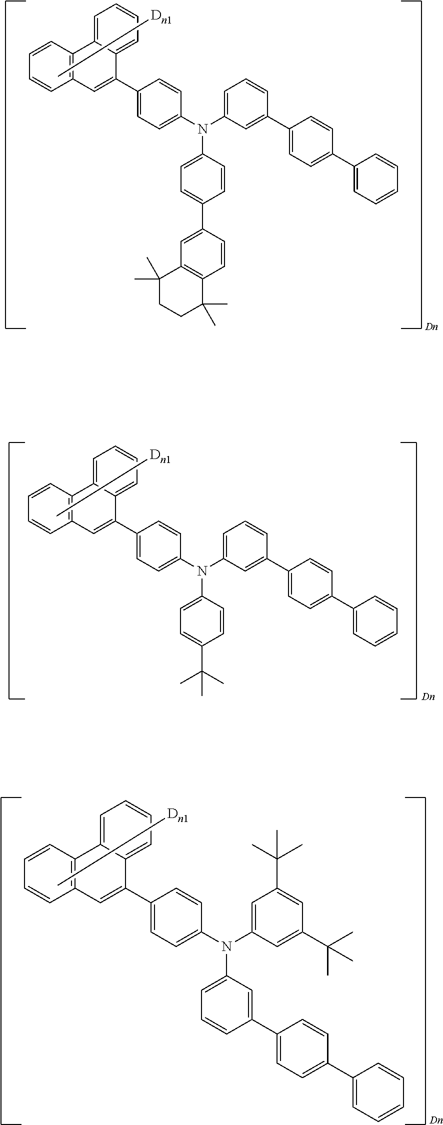

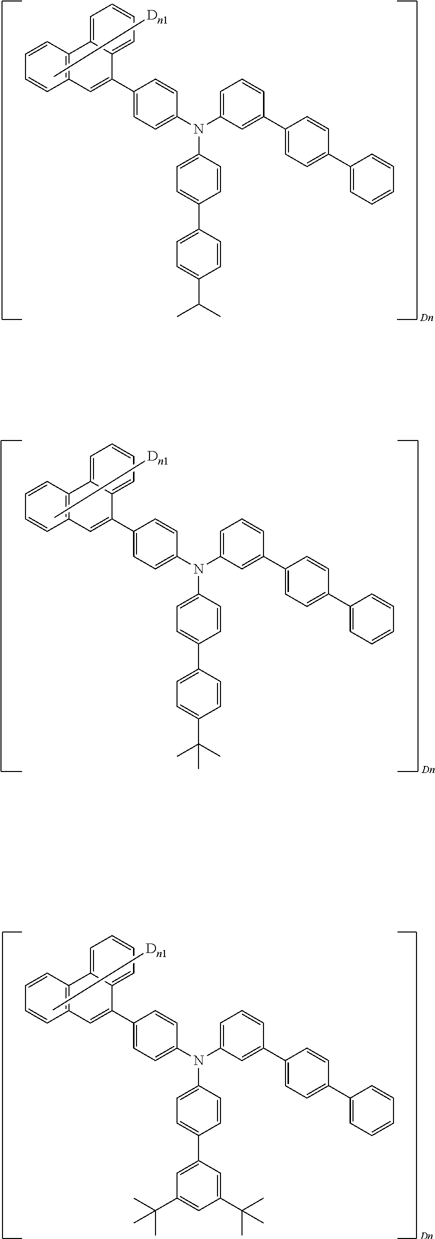

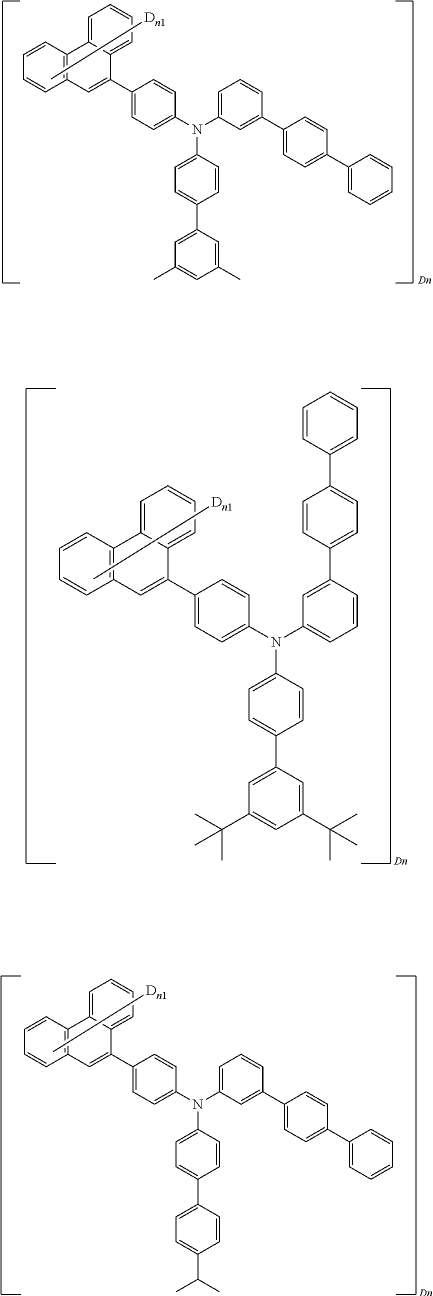

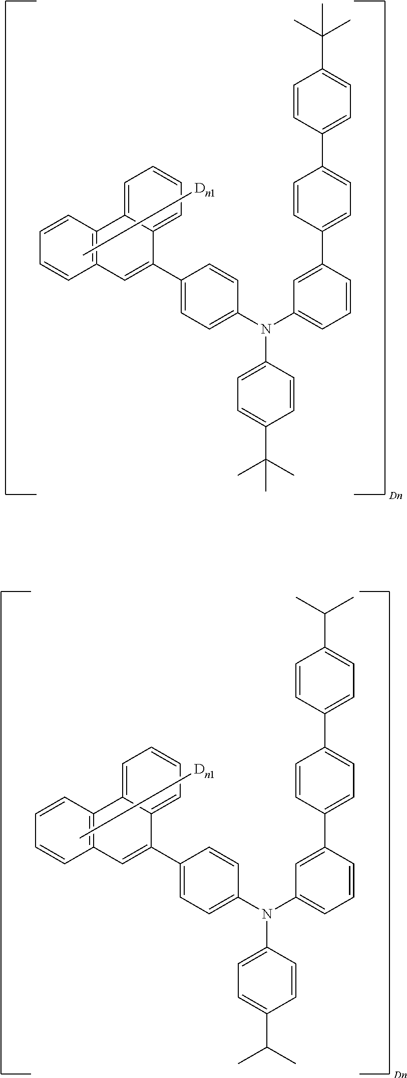

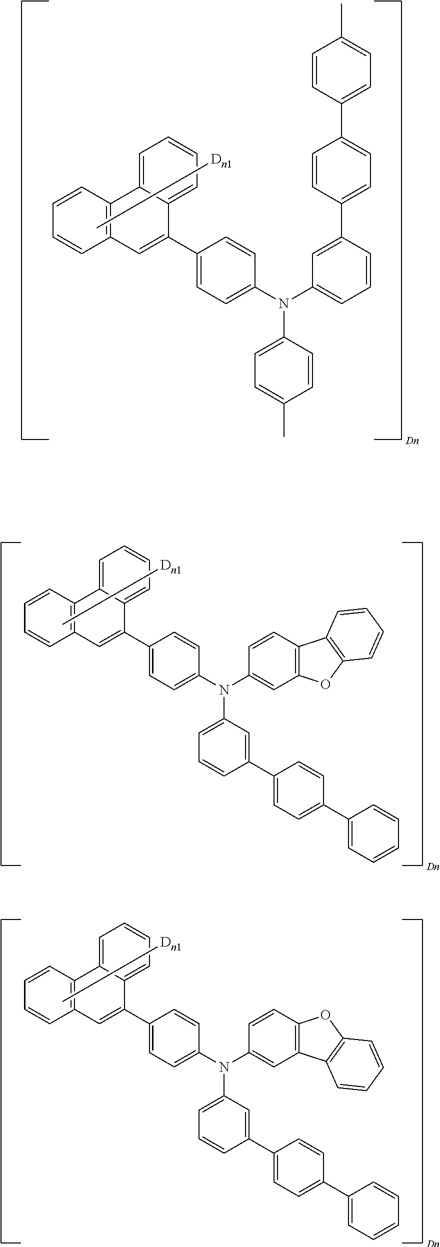

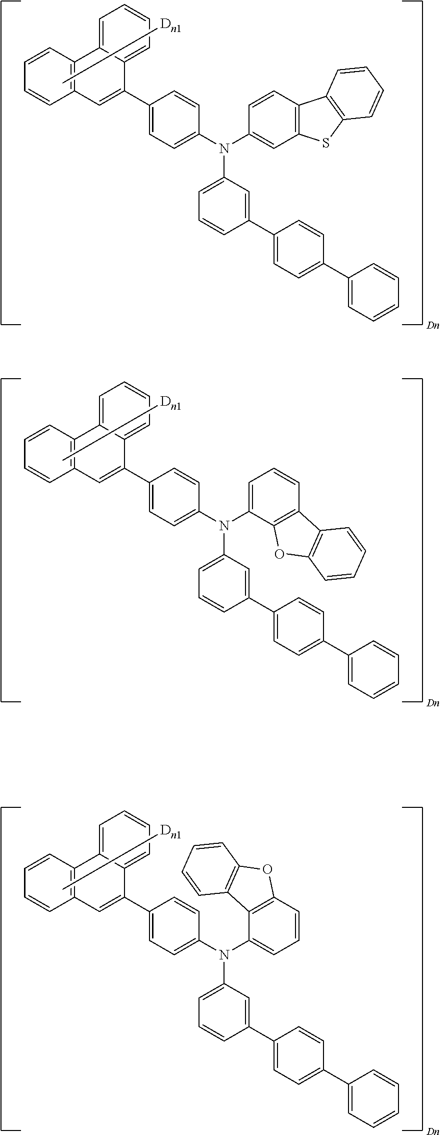

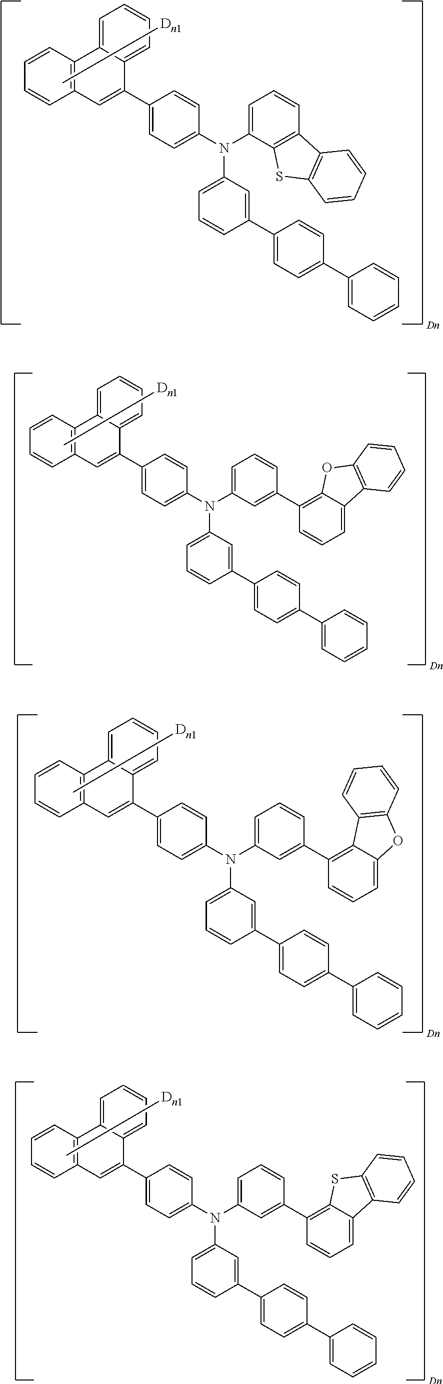

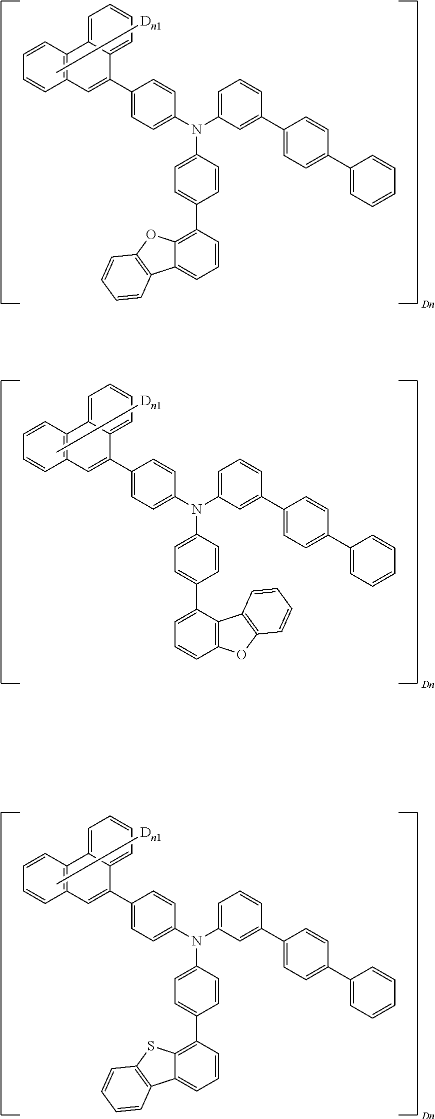

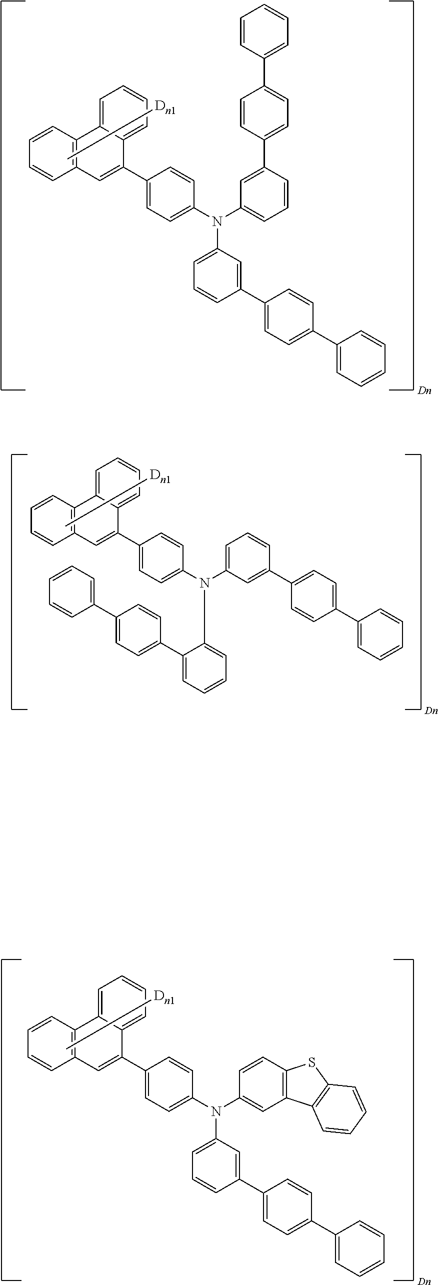

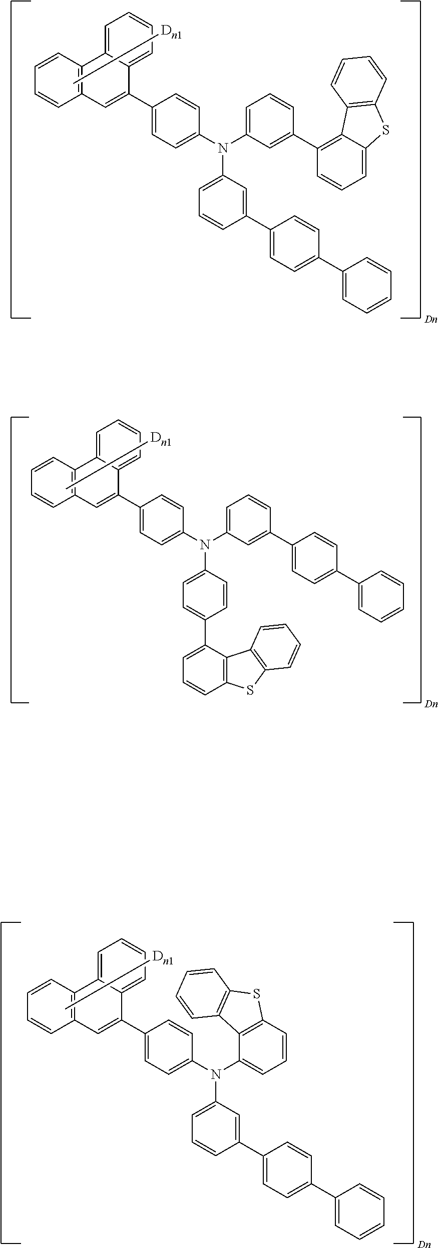

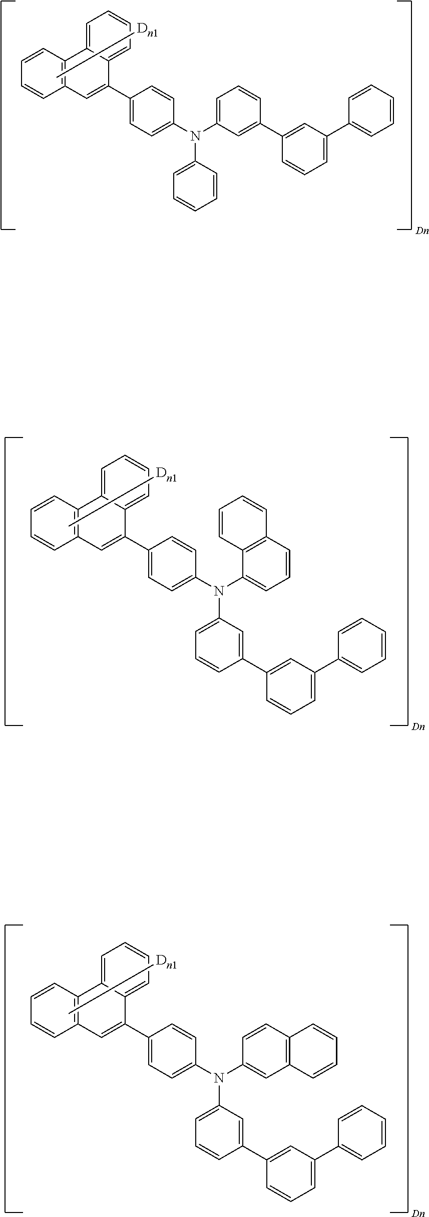

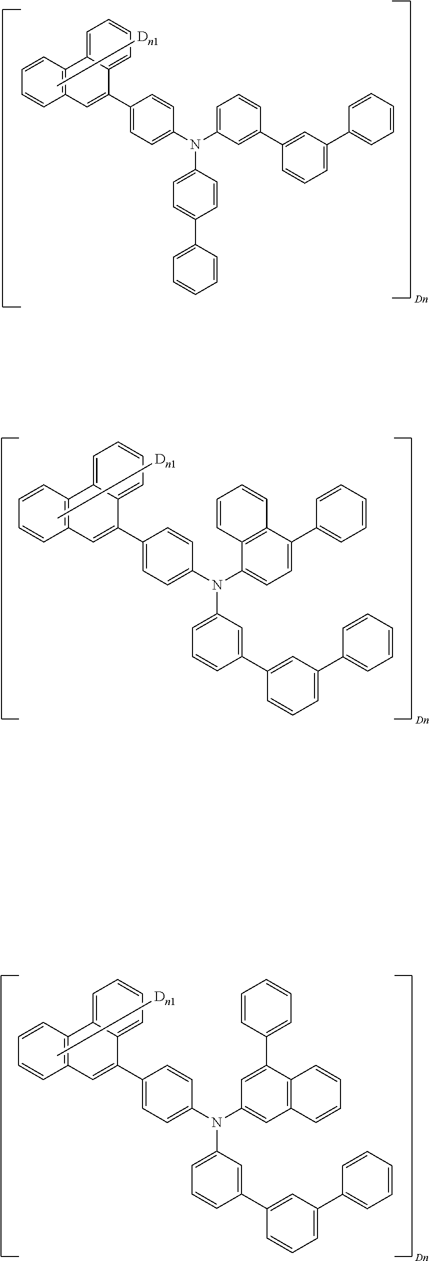

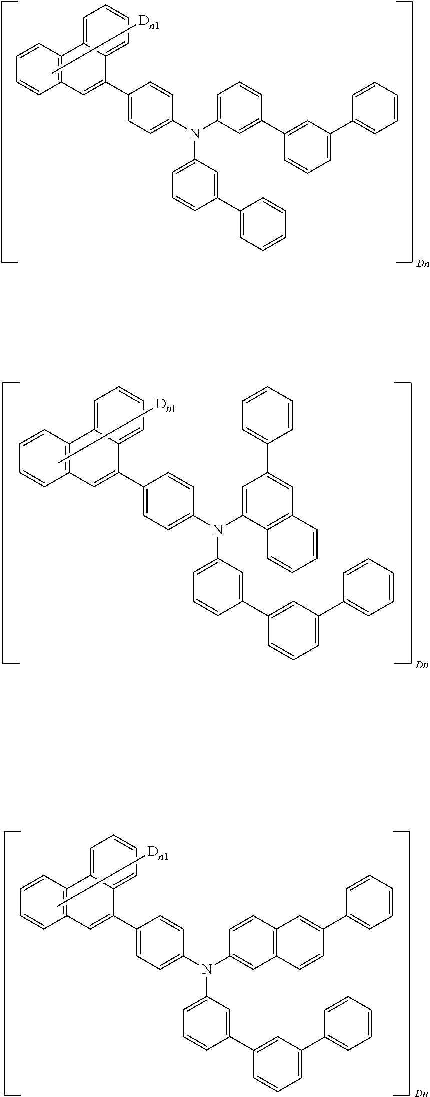

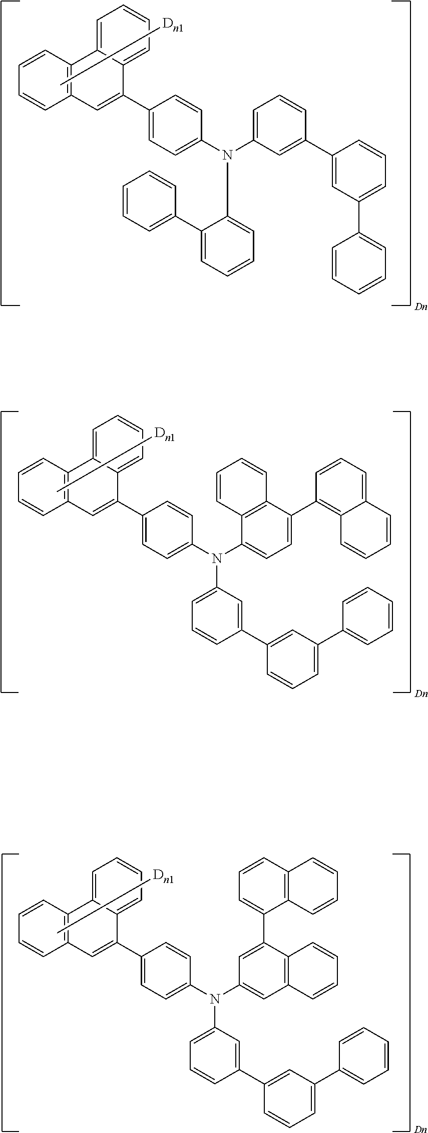

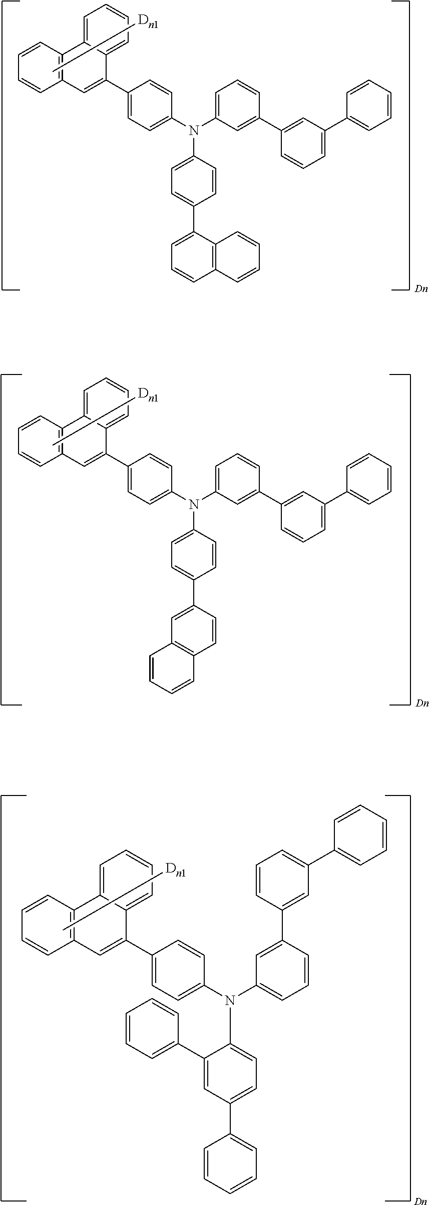

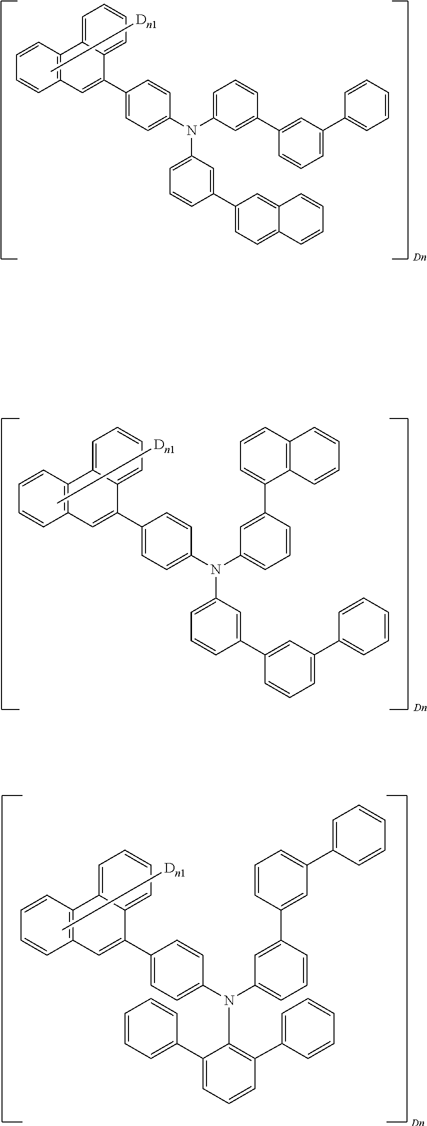

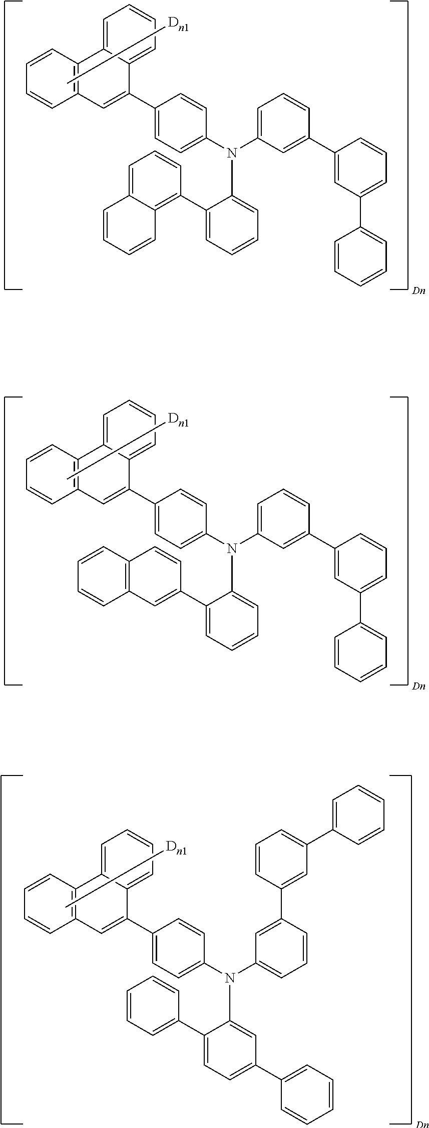

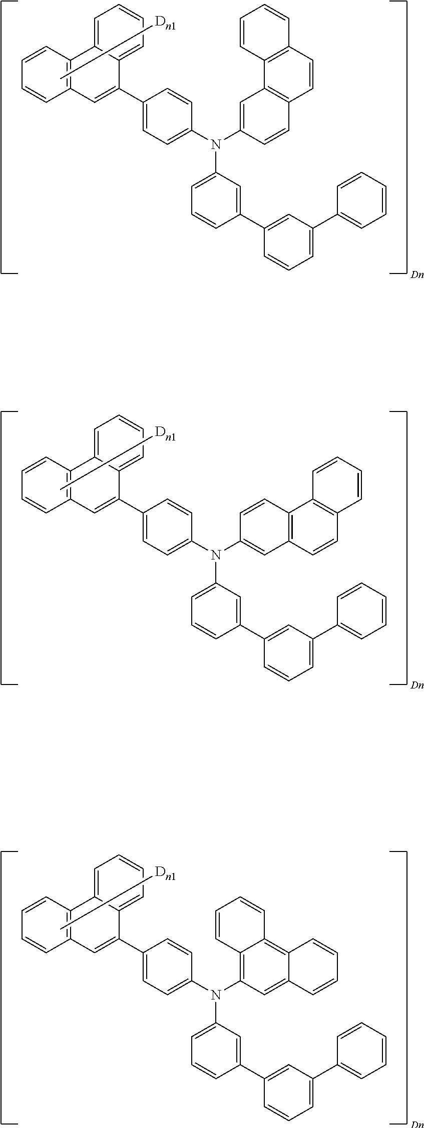

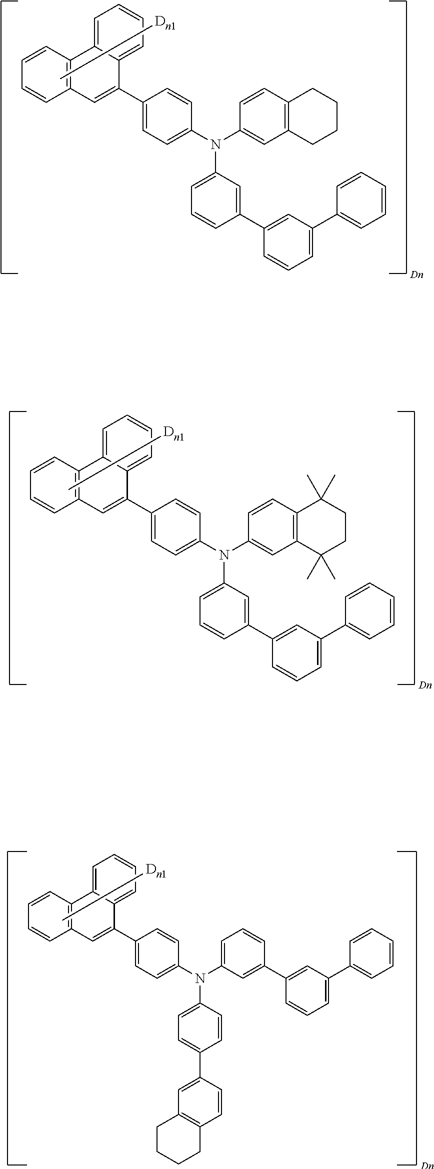

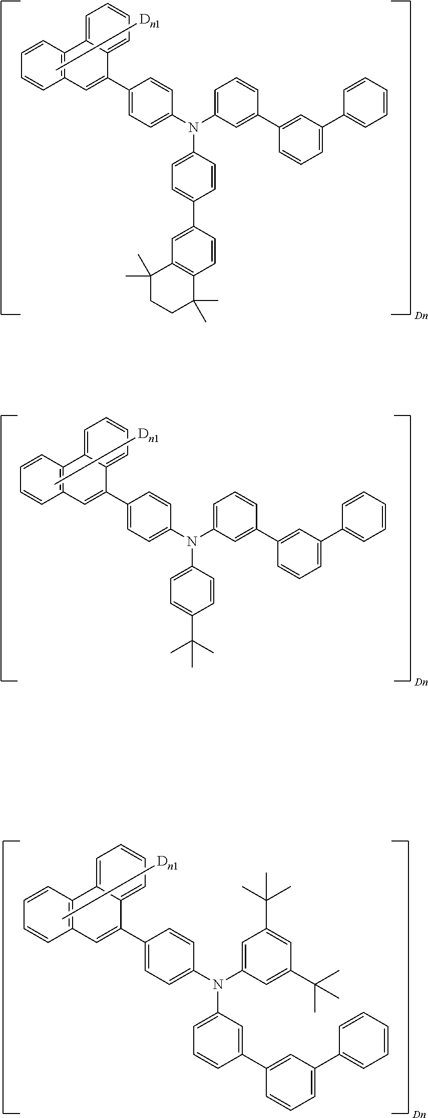

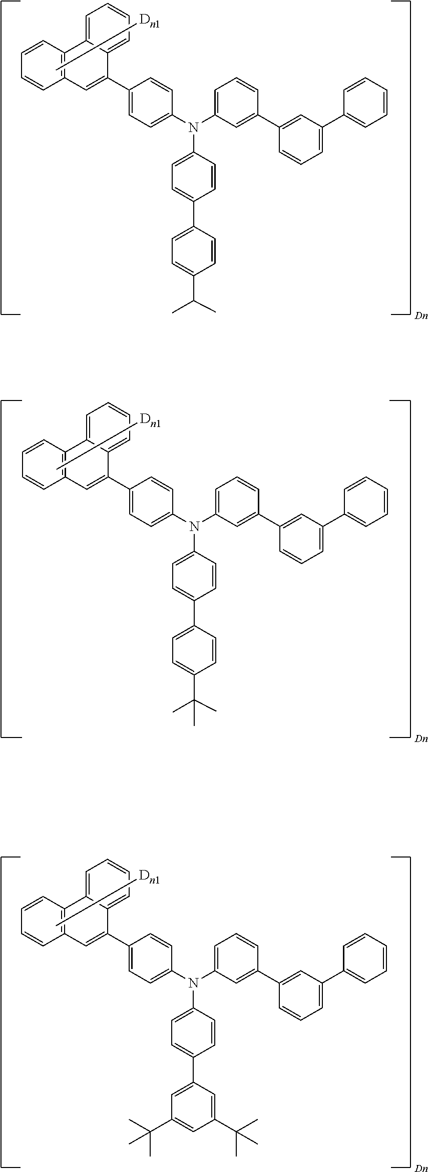

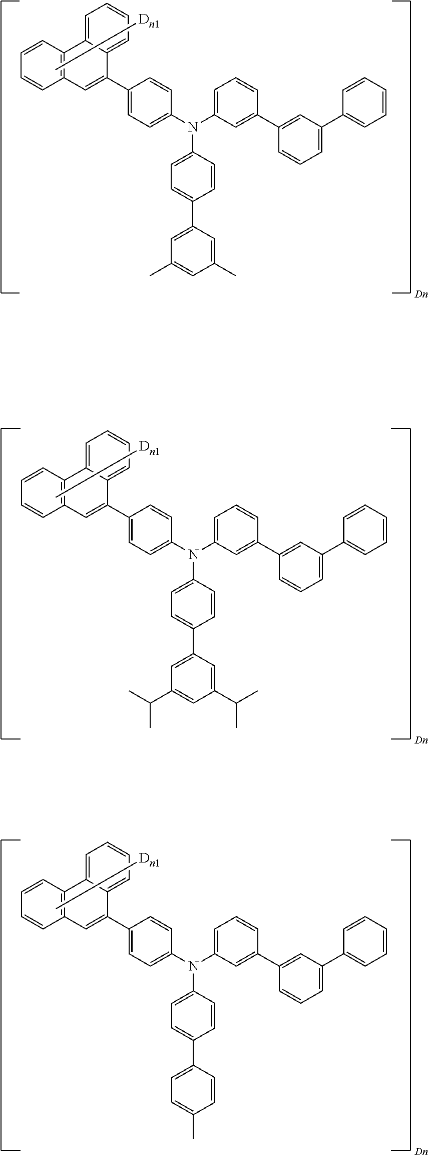

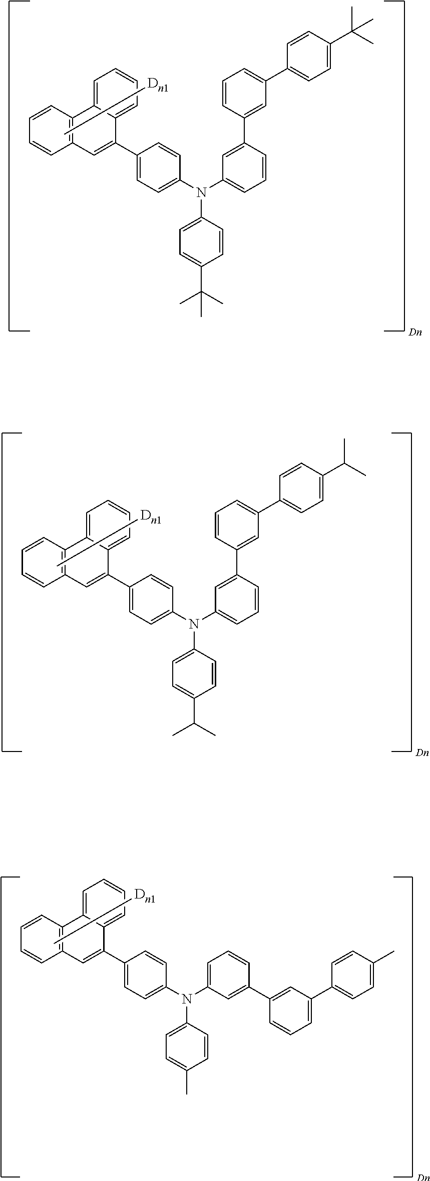

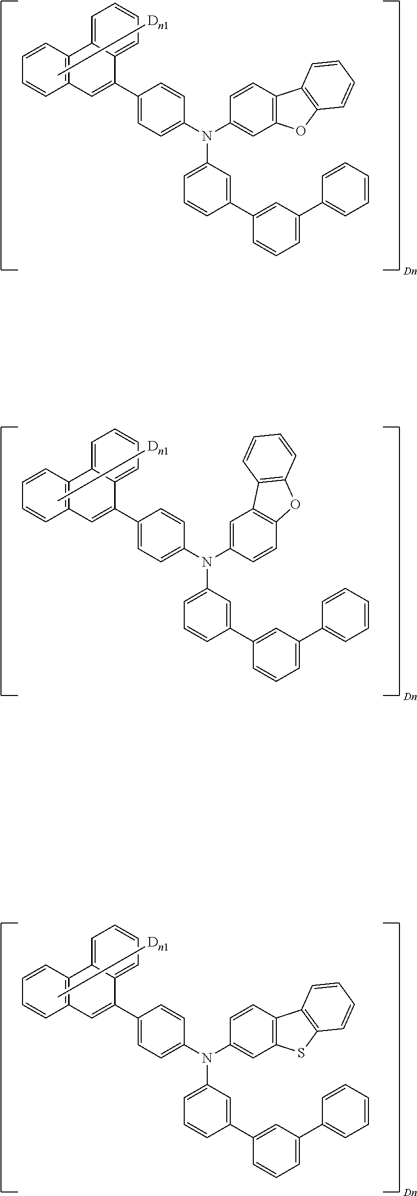

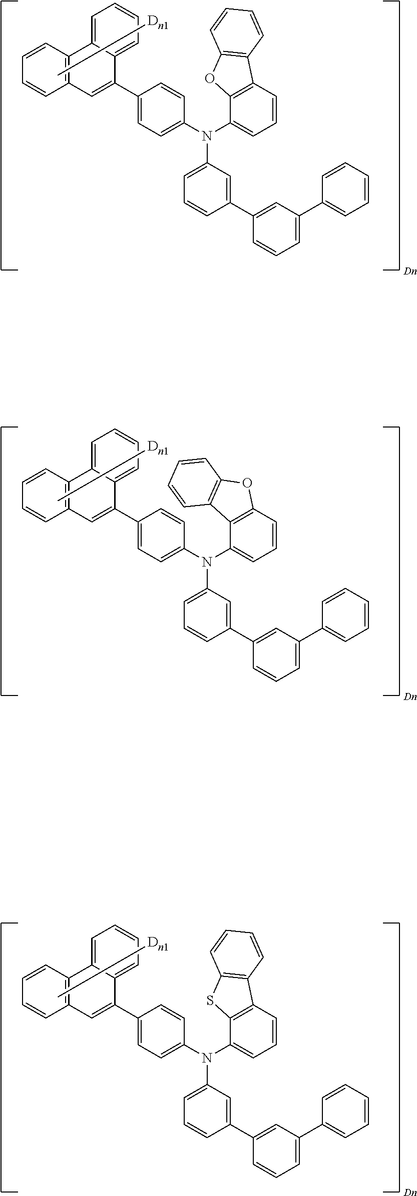

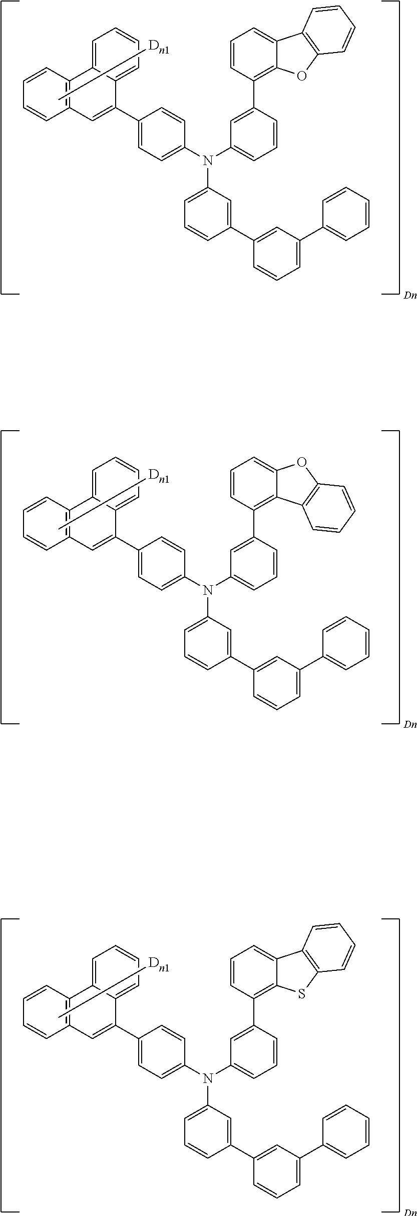

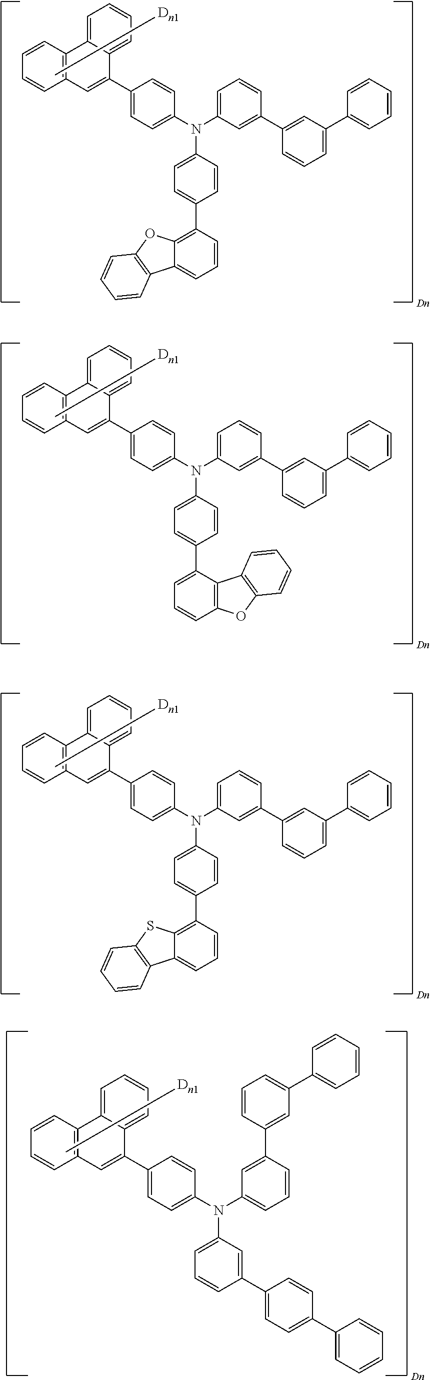

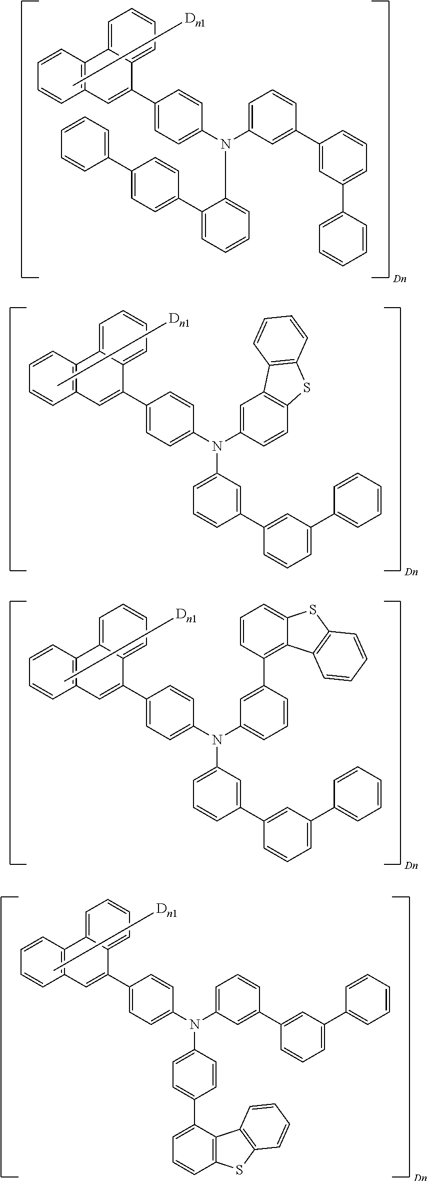

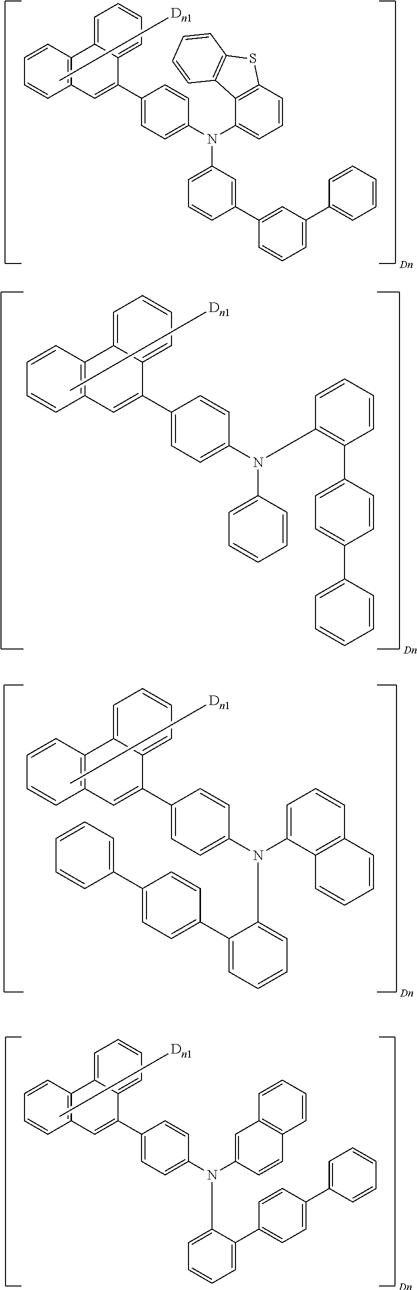

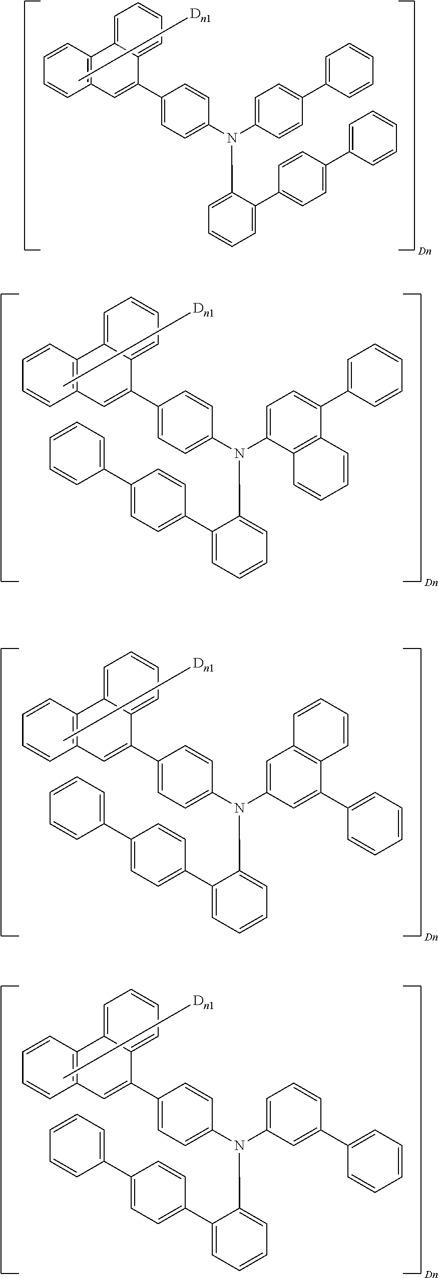

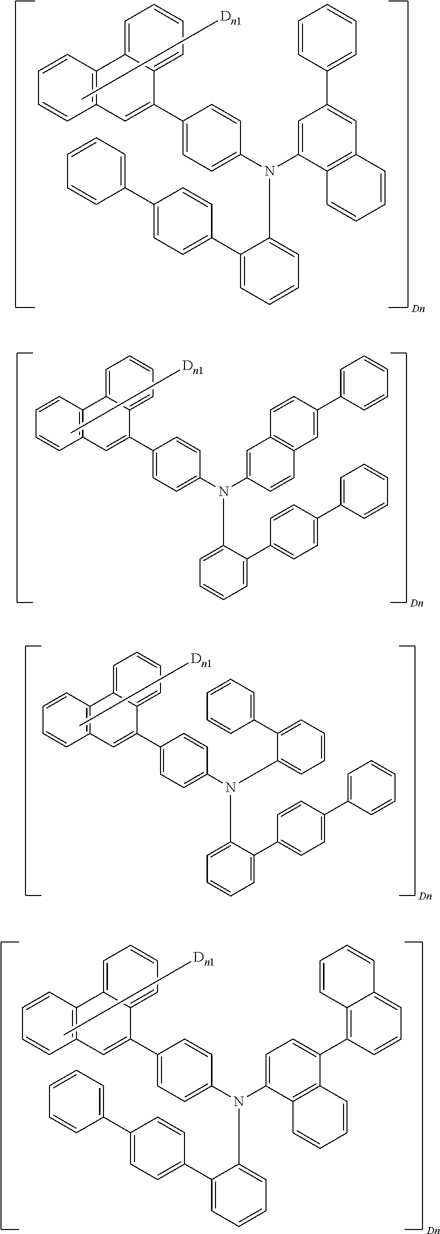

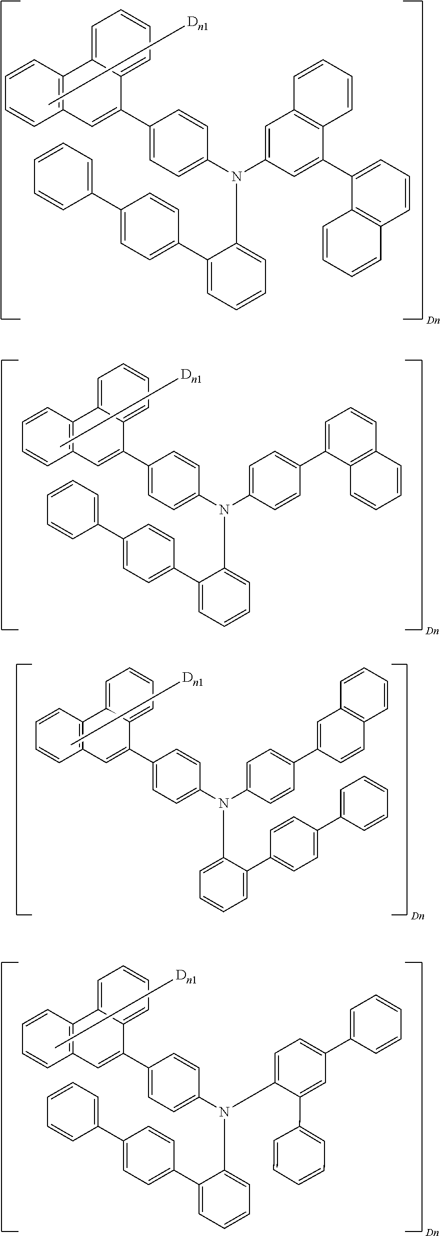

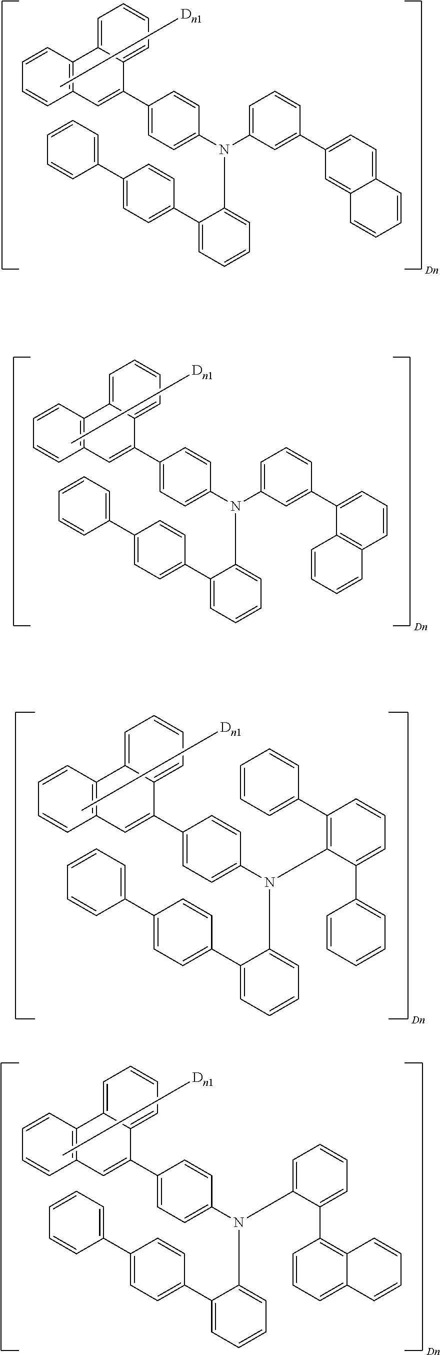

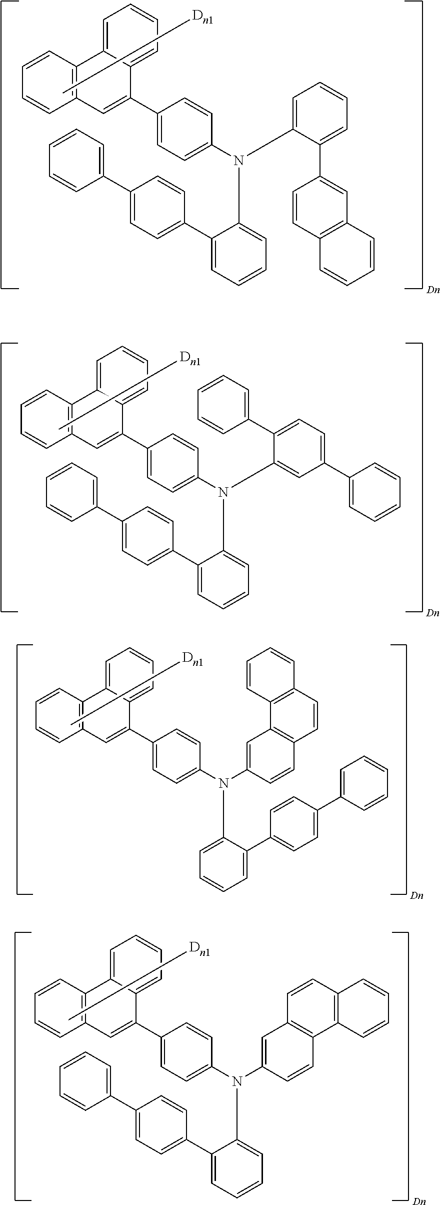

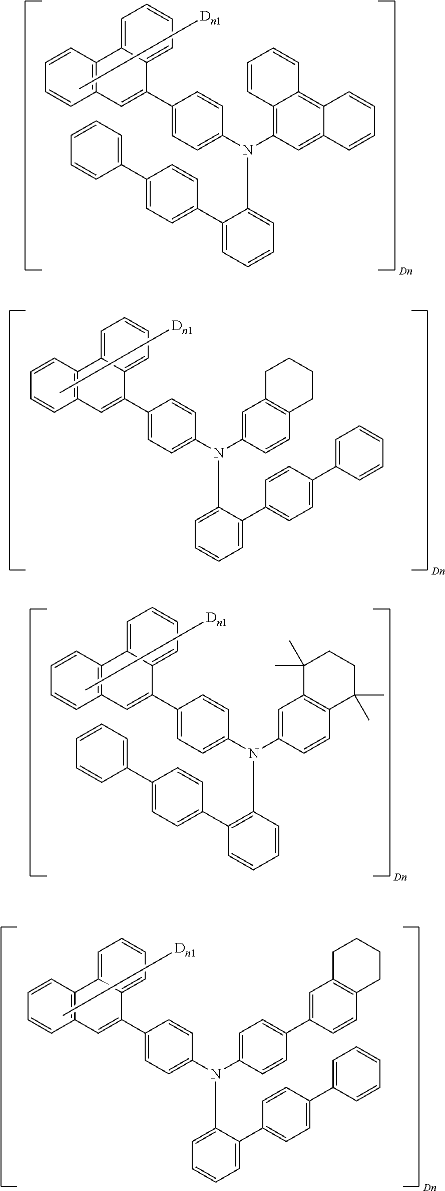

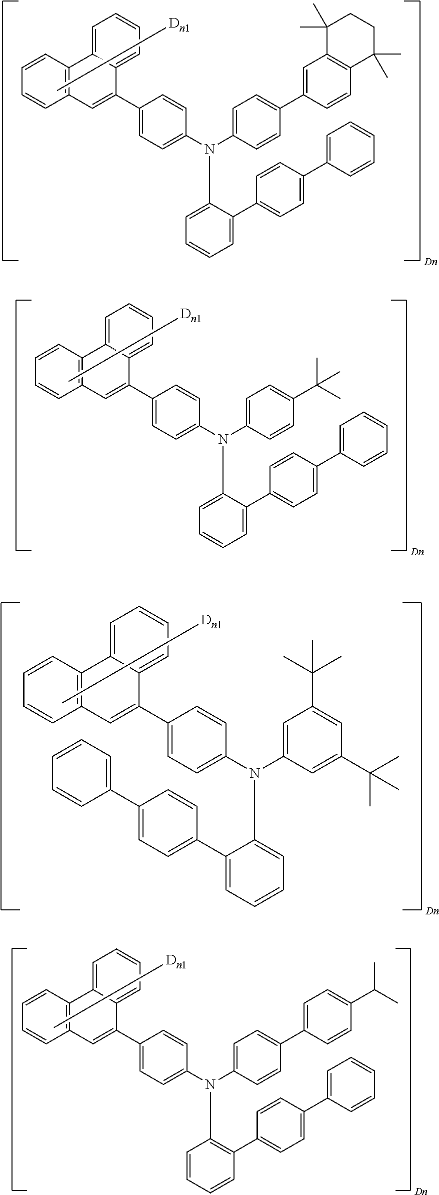

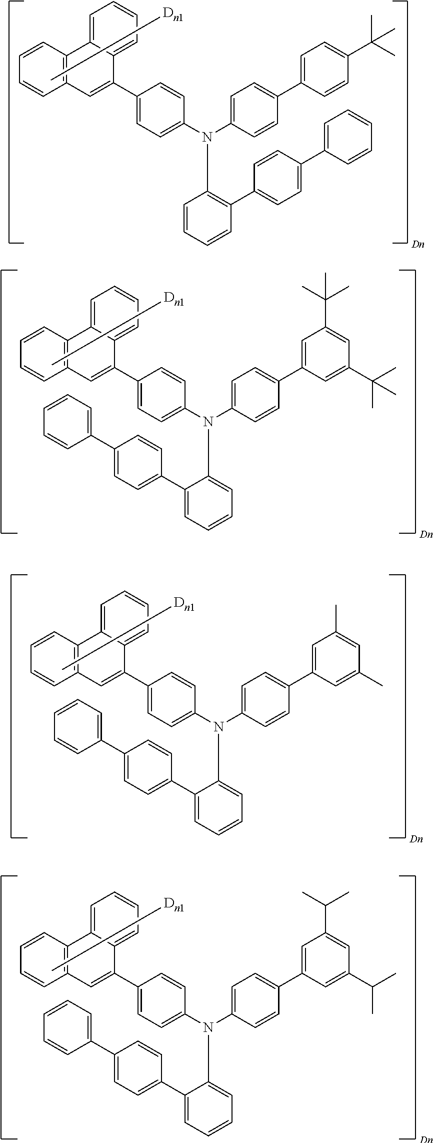

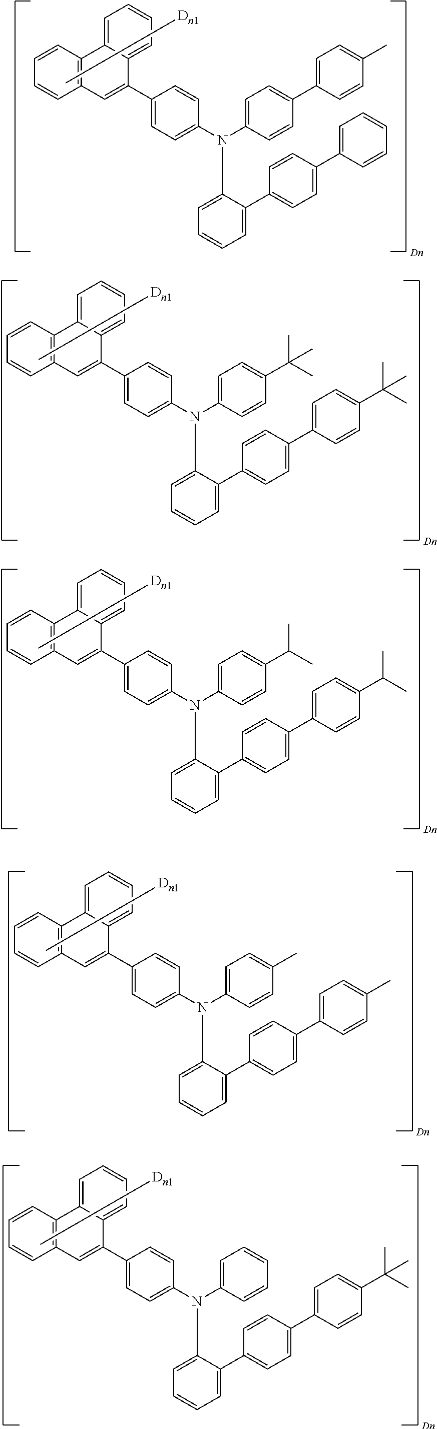

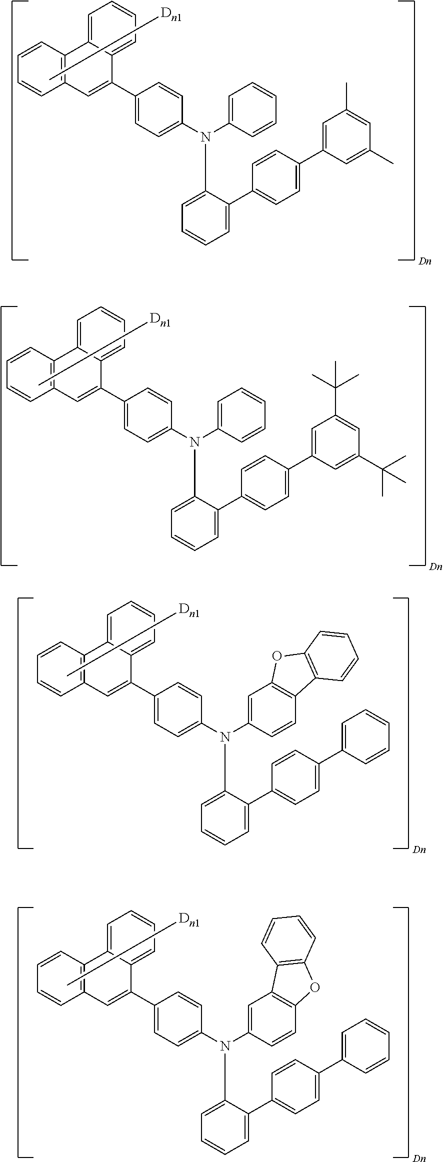

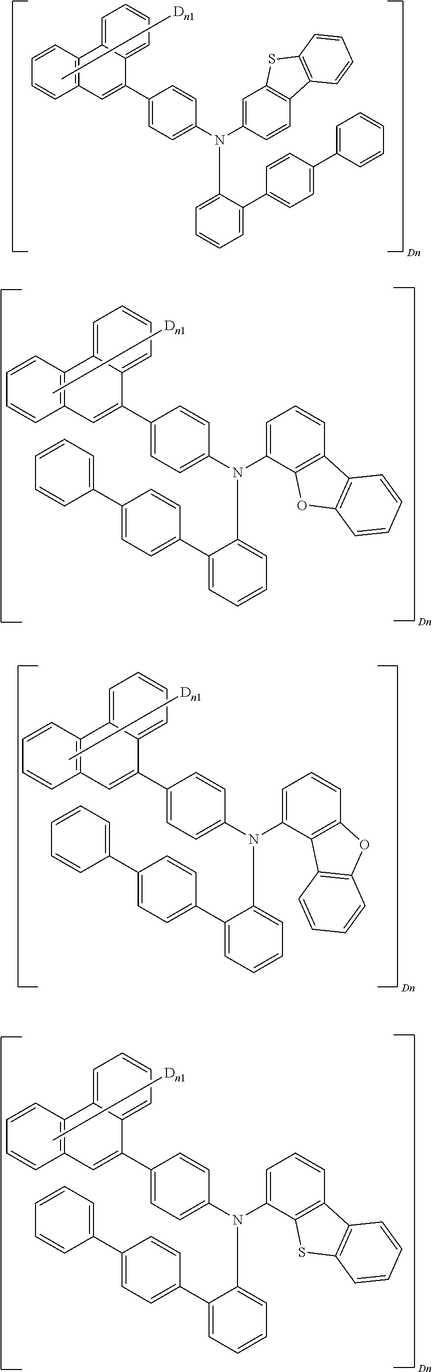

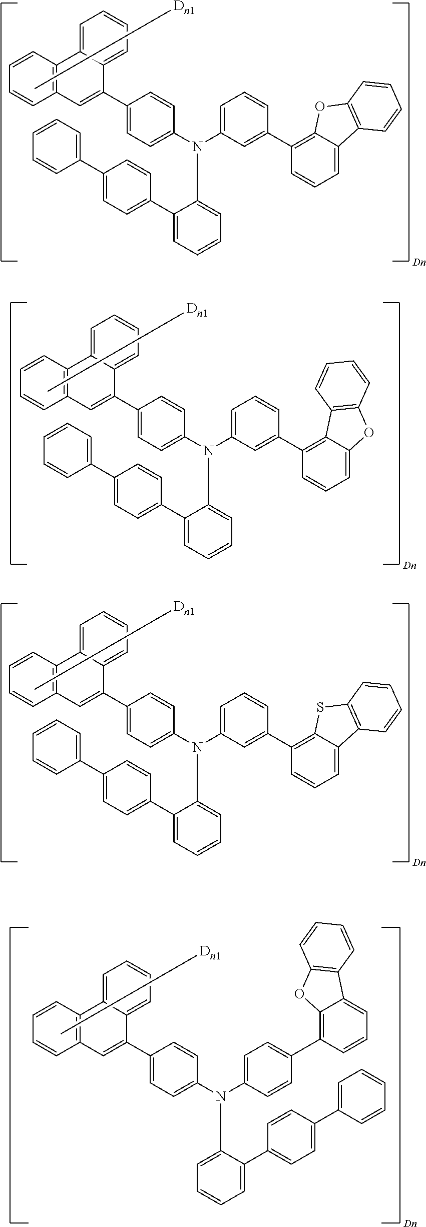

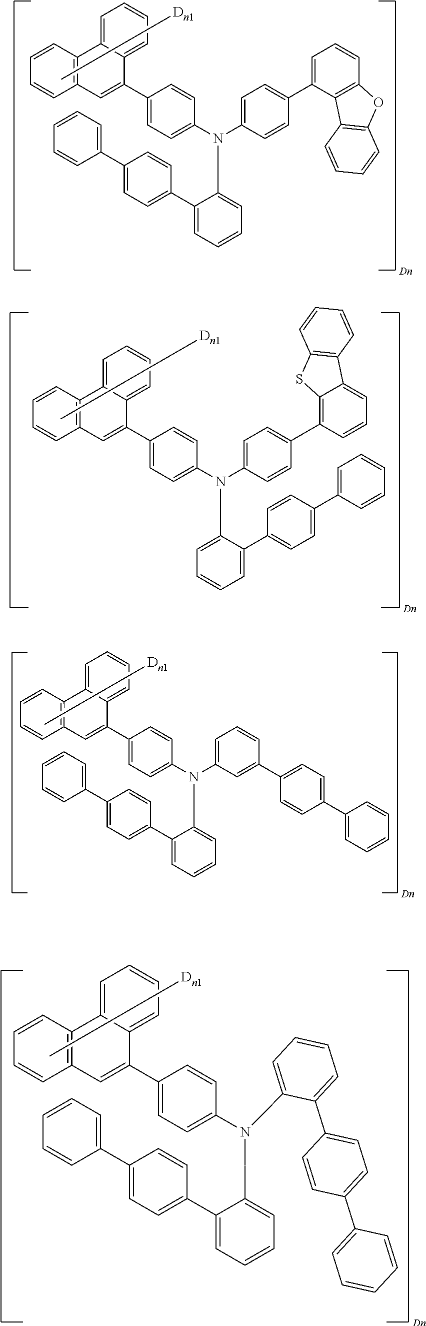

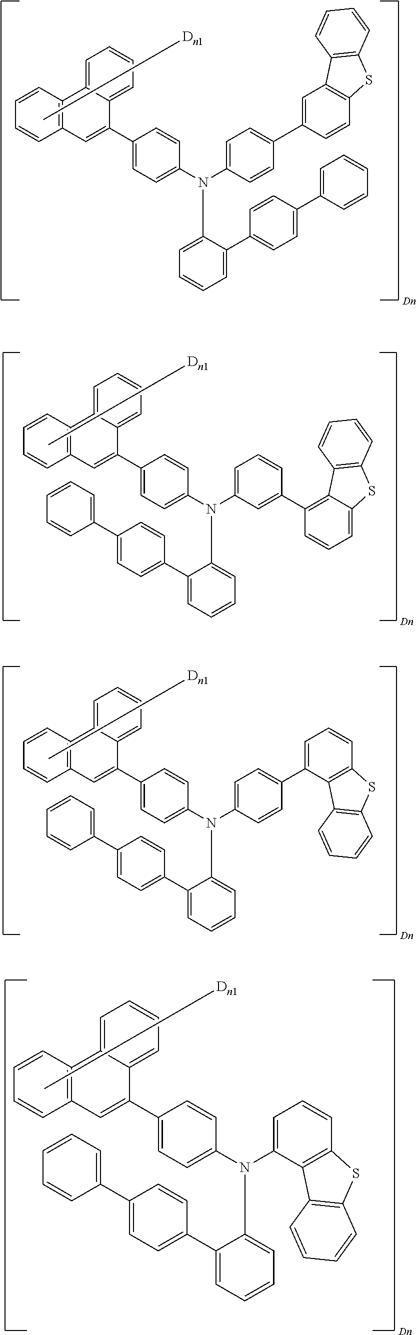

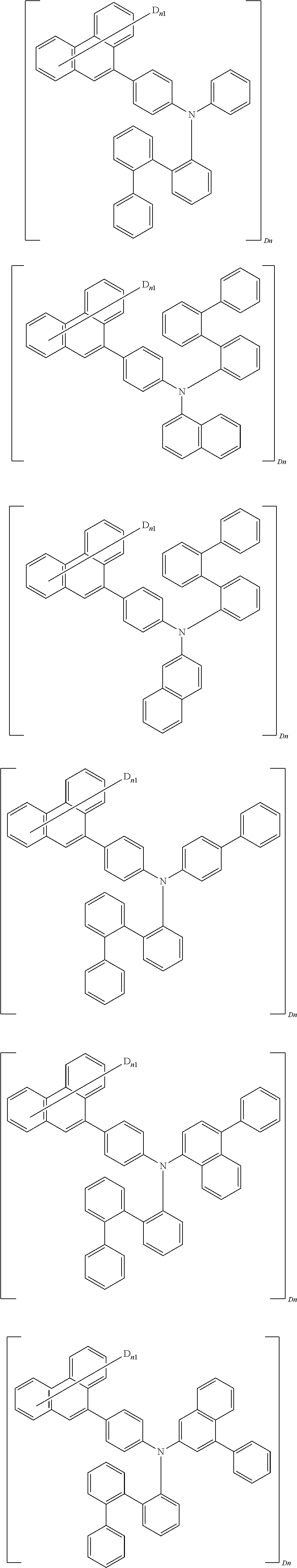

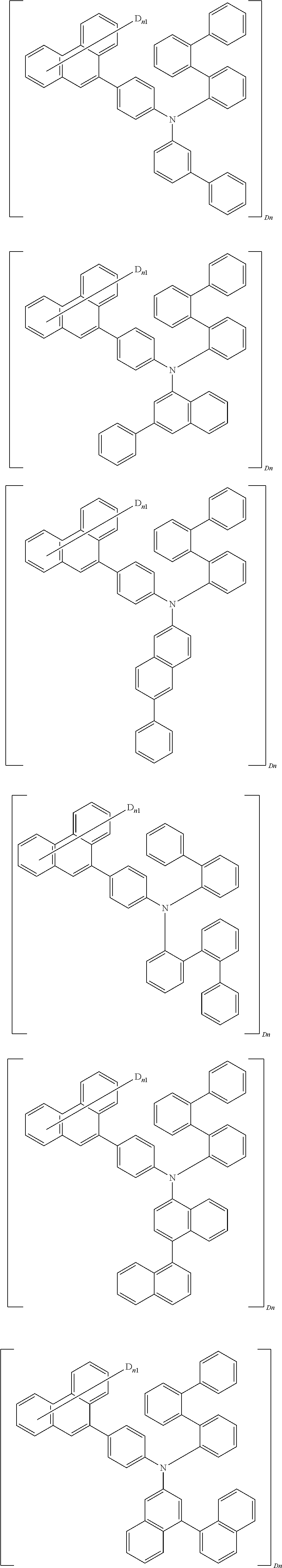

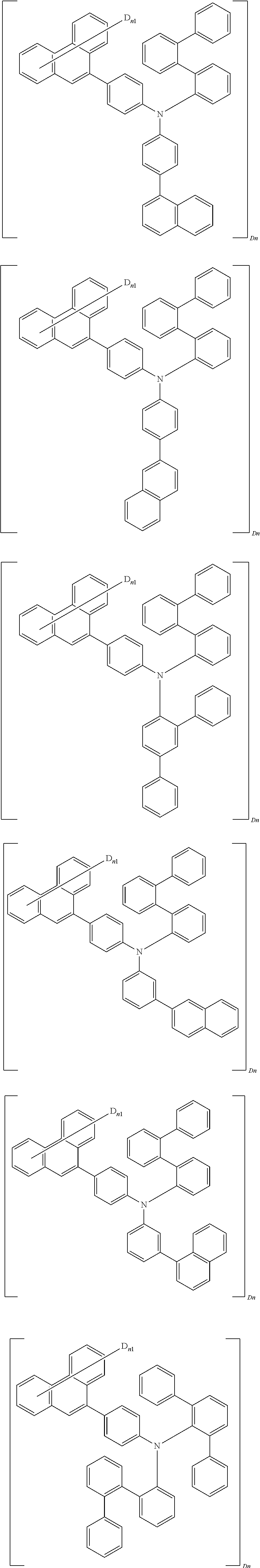

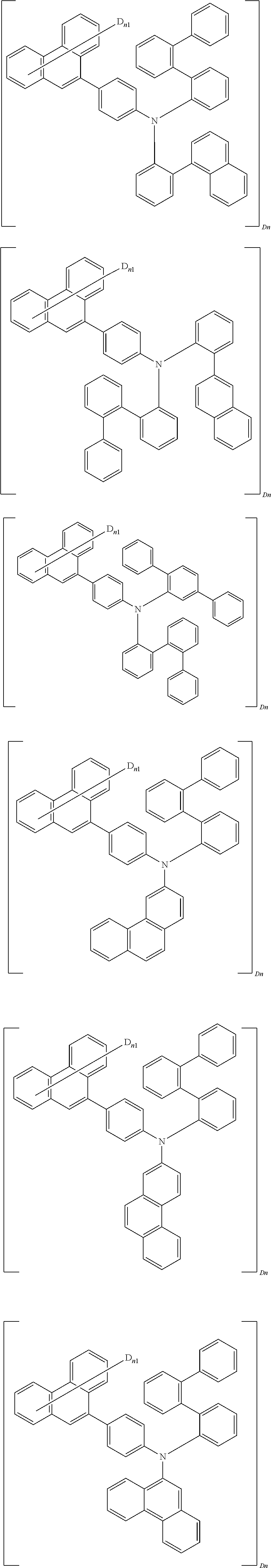

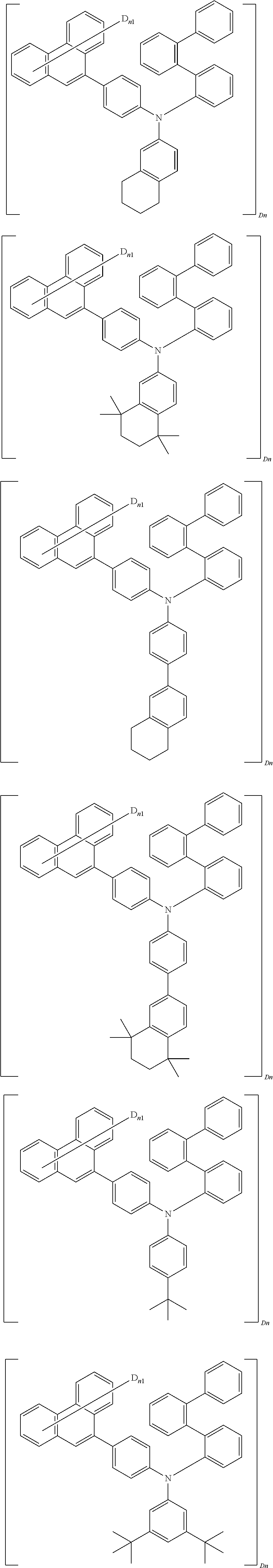

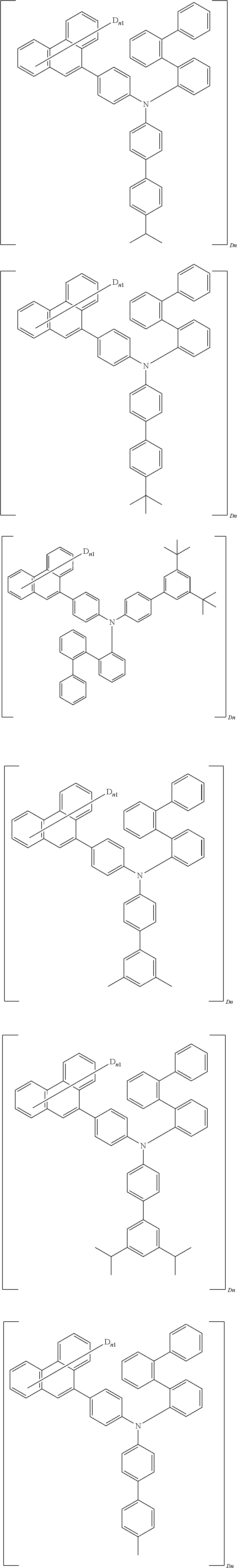

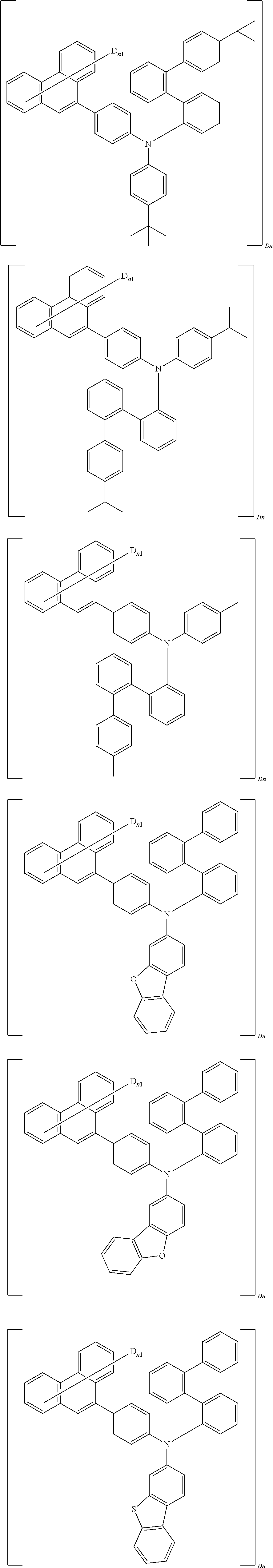

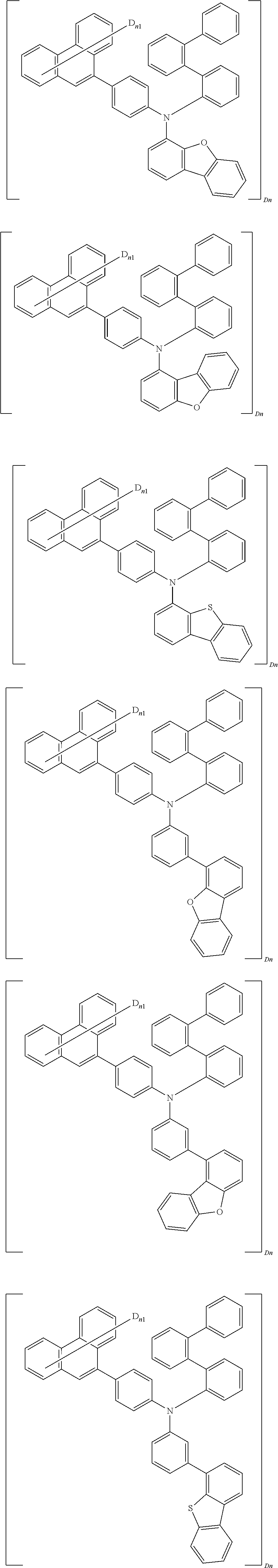

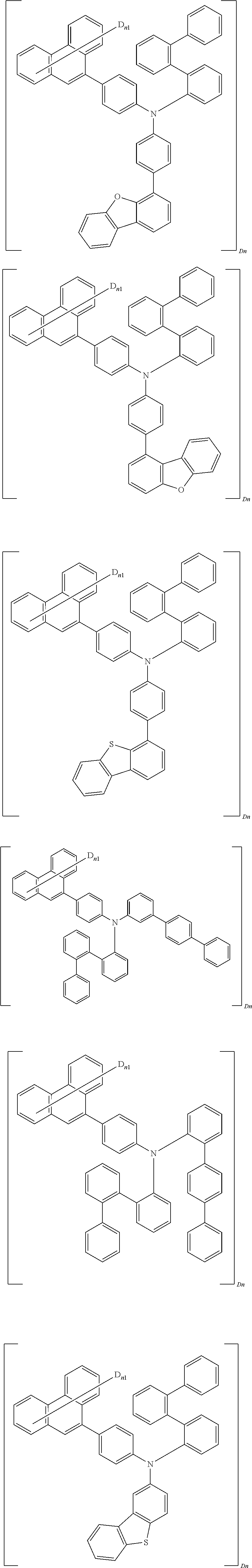

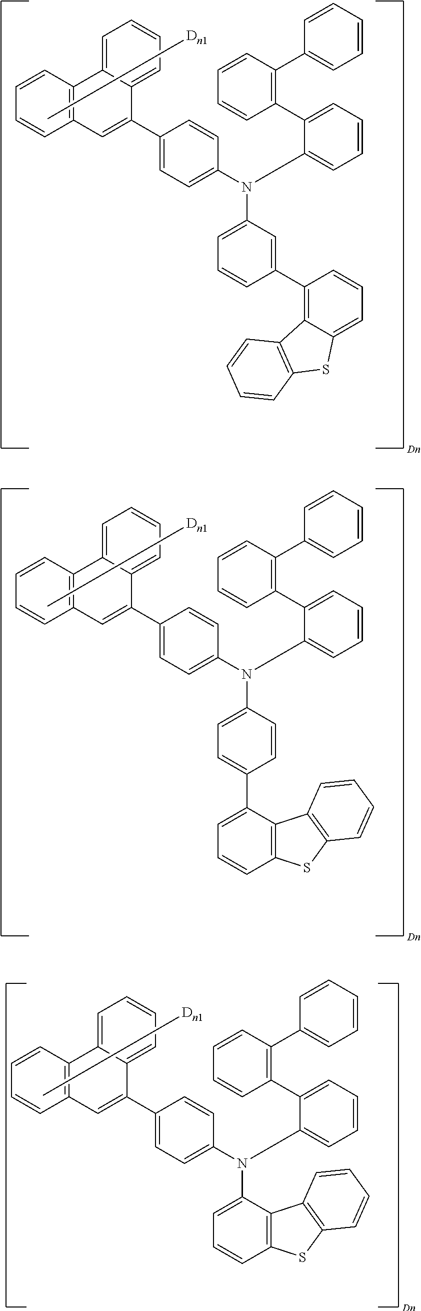

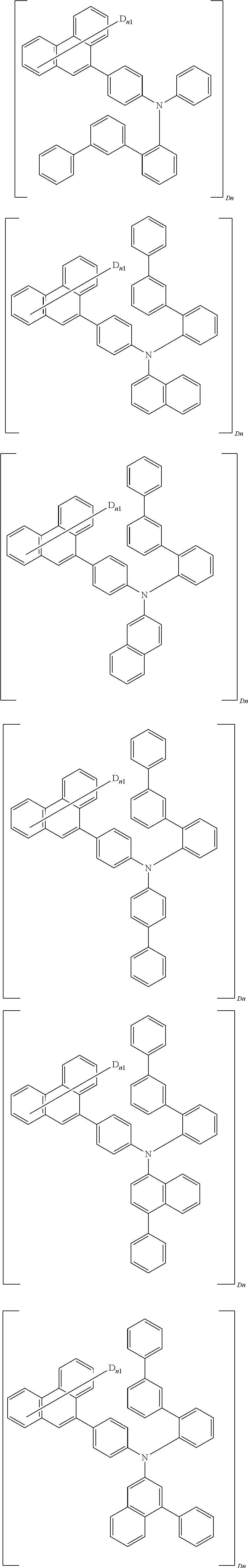

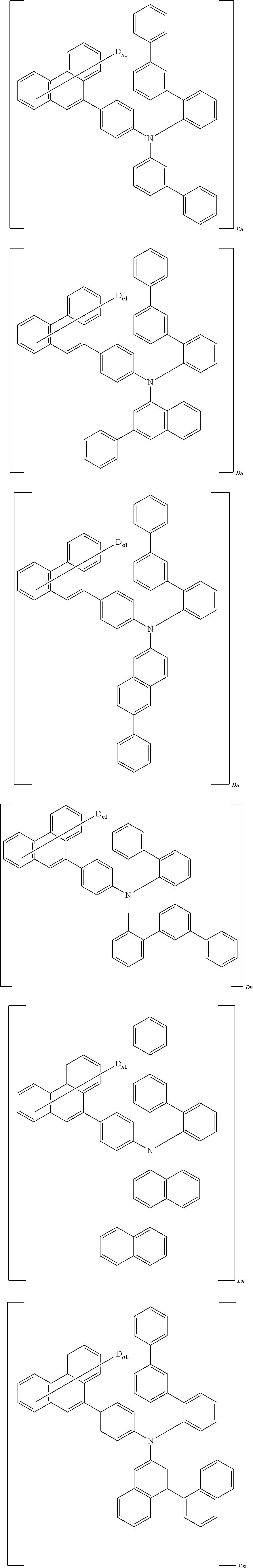

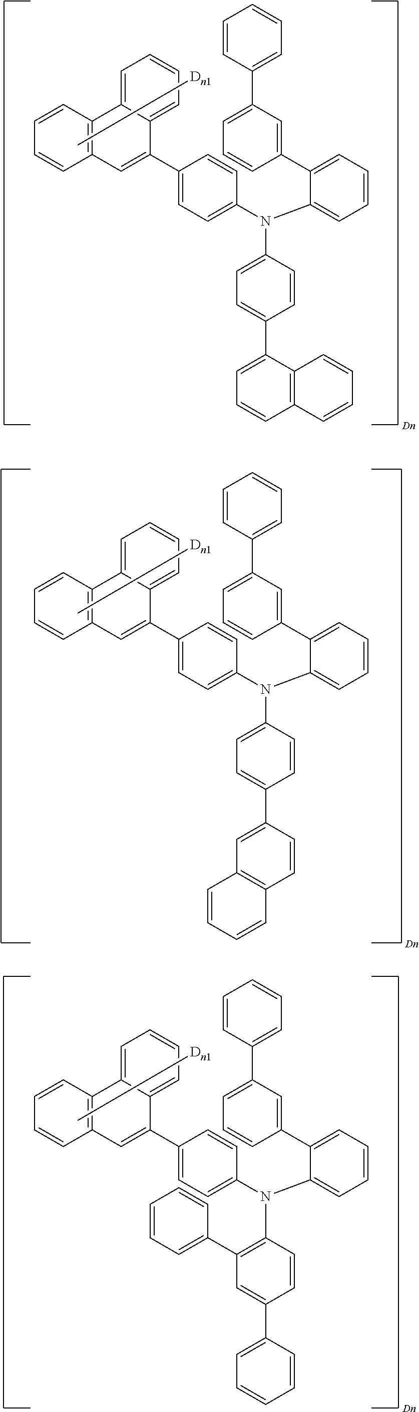

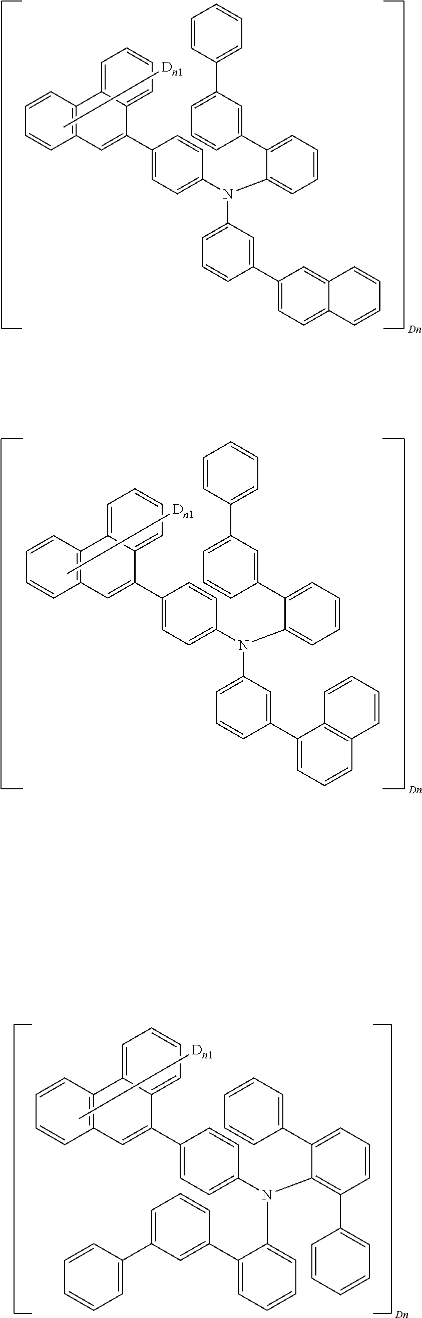

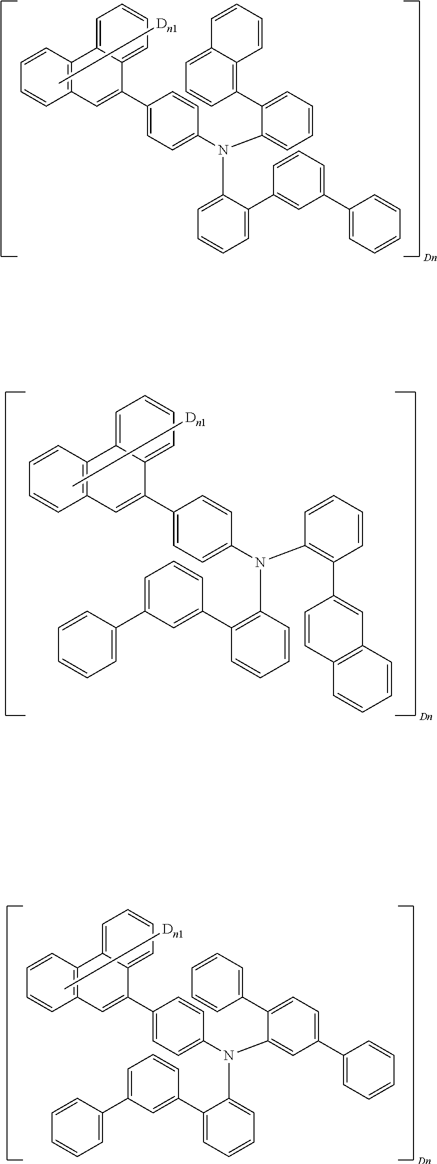

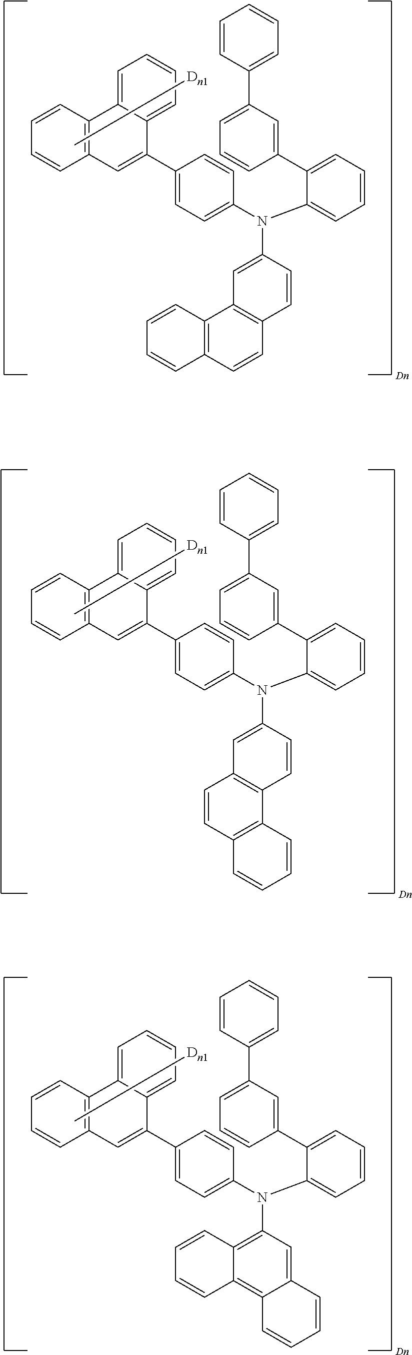

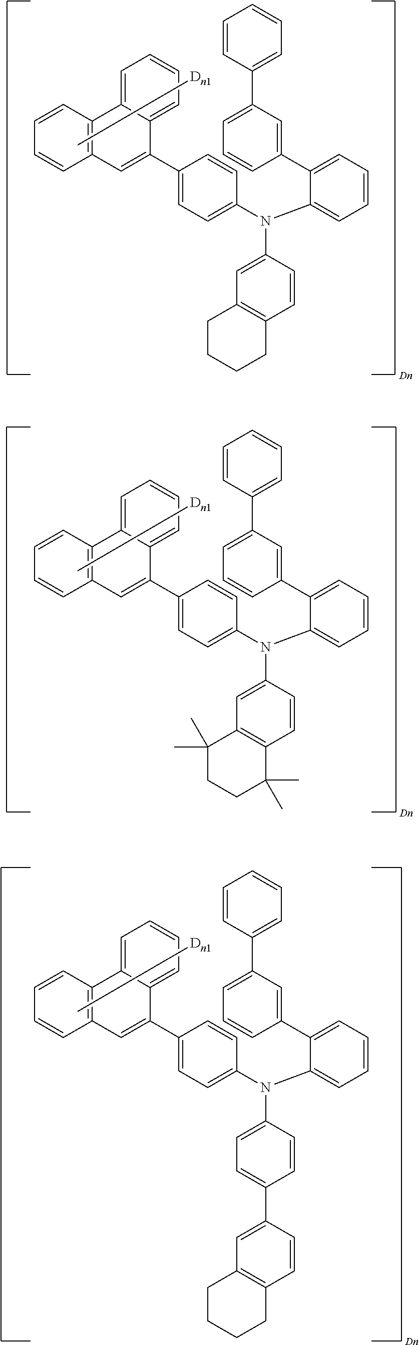

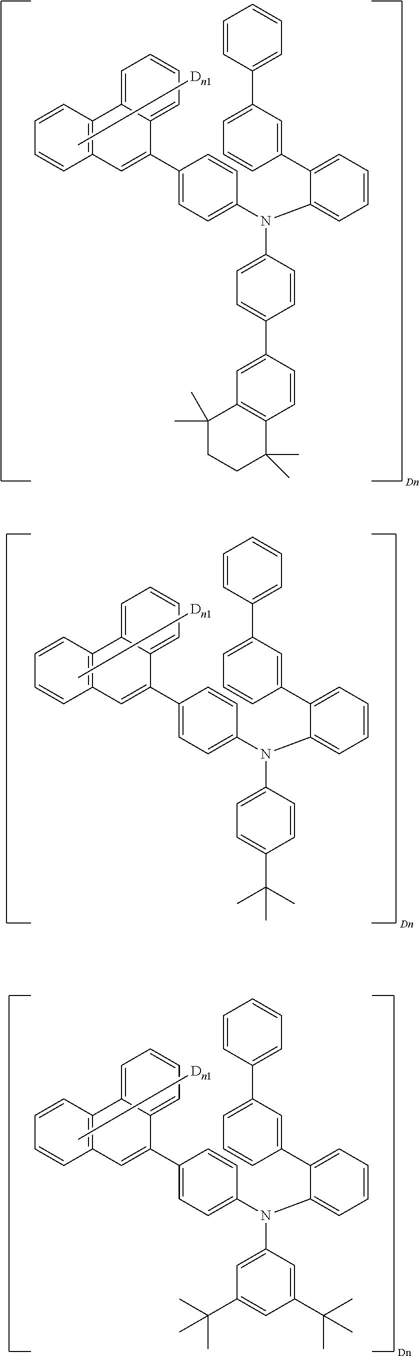

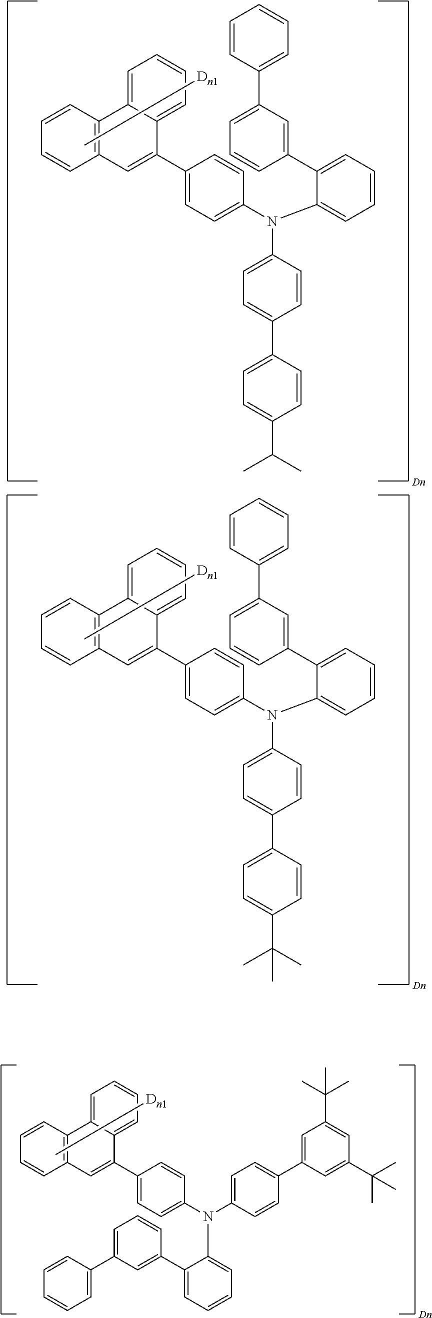

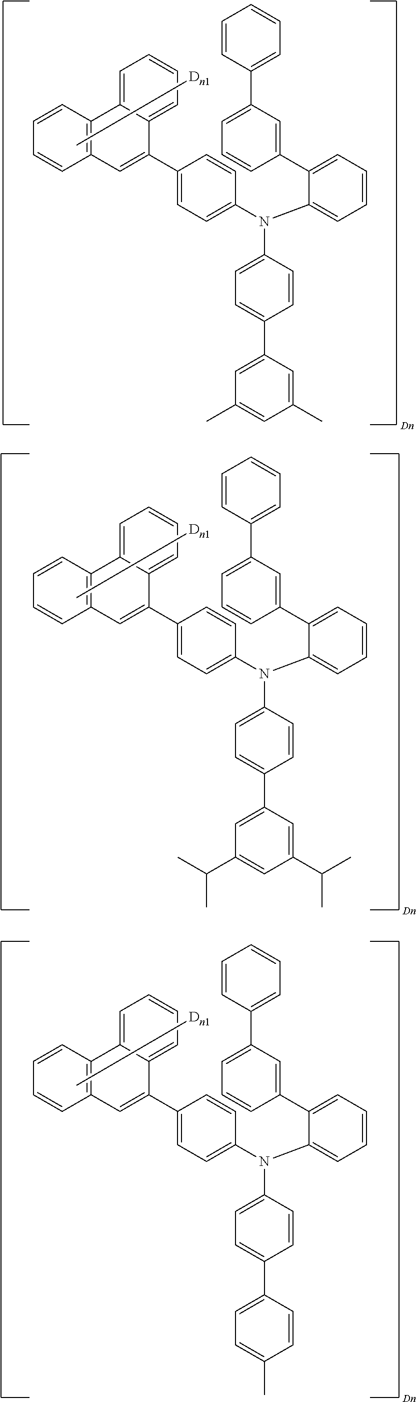

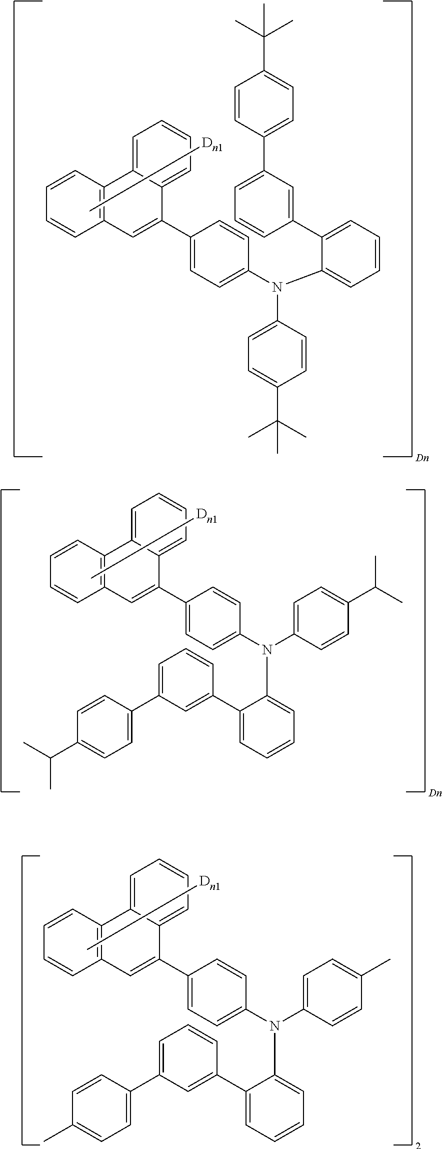

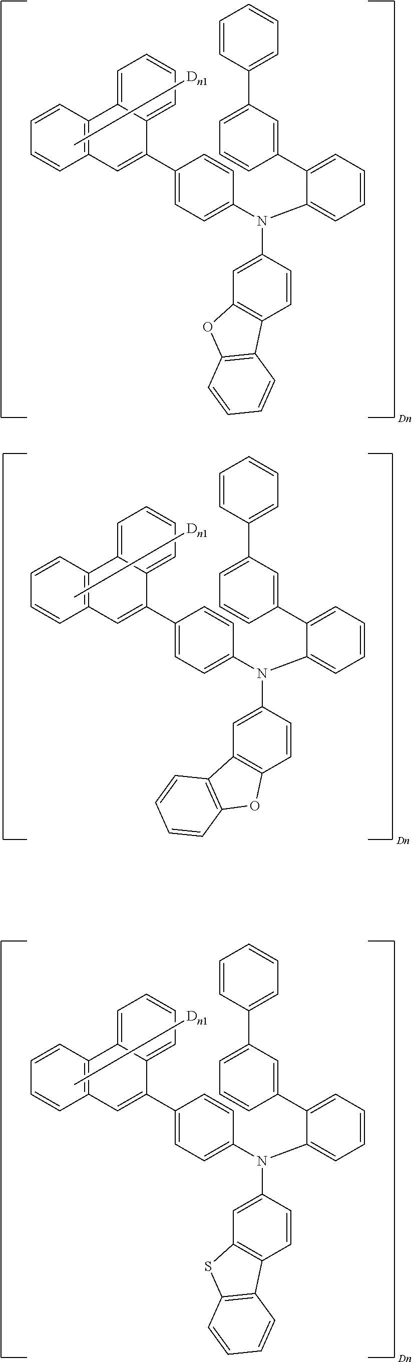

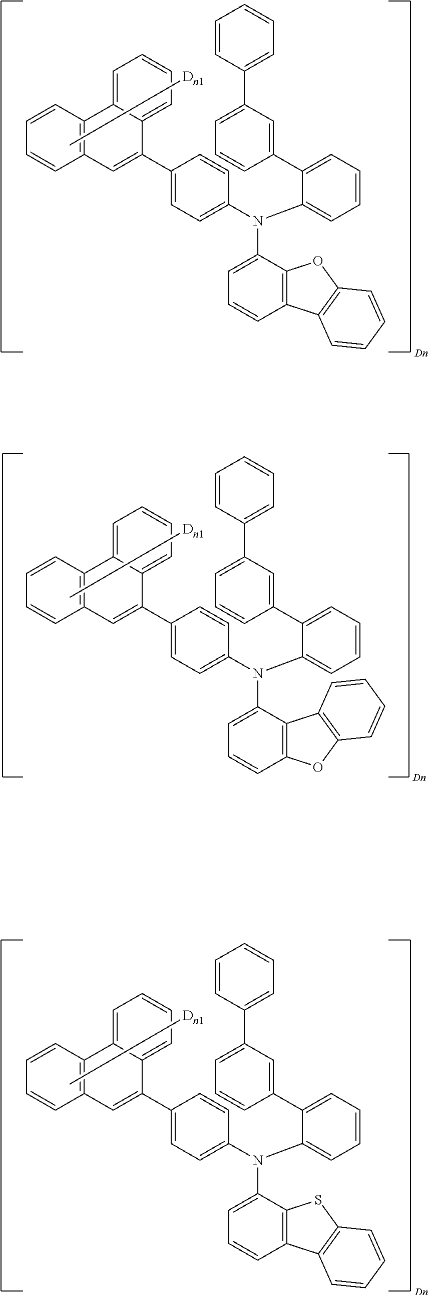

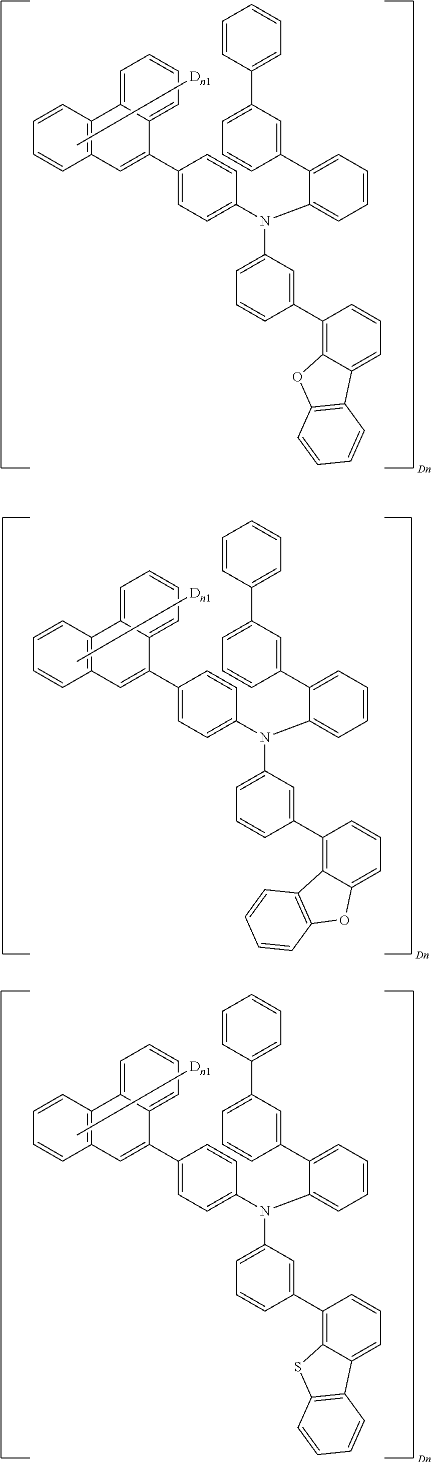

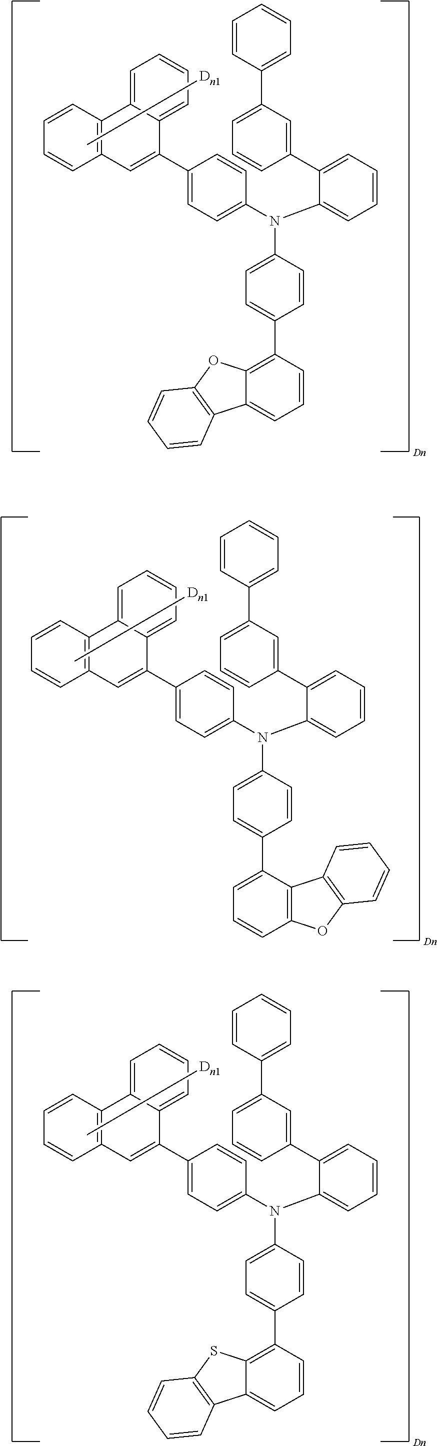

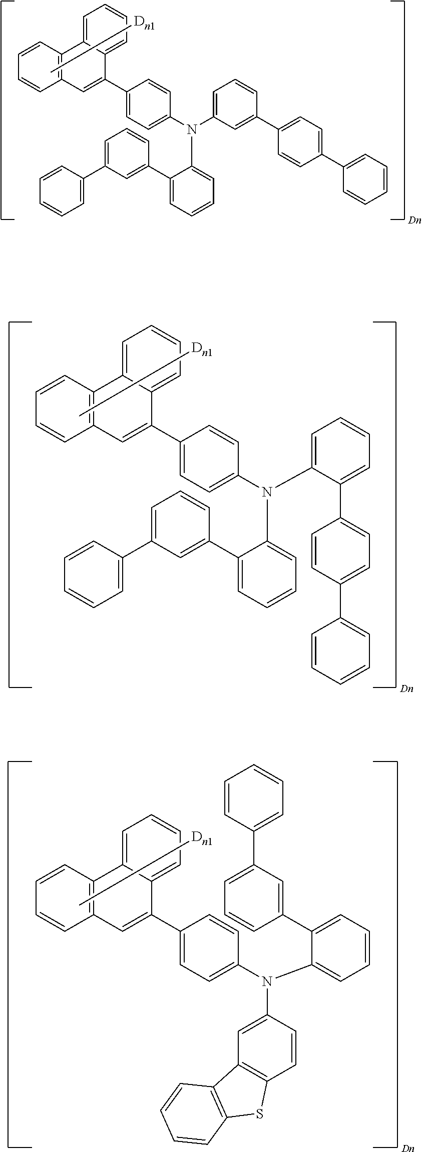

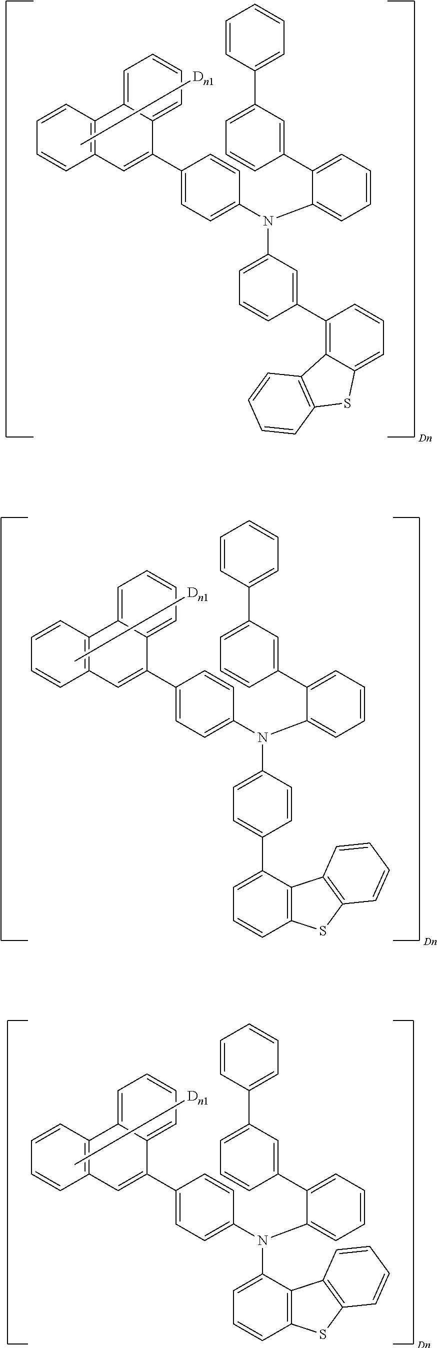

in the above group, D is deuterium,
n1 is an integer of 1 to 9,
n is the total number of deuterium substituted in the compound,
each of the above compounds has a deuterium substitution rate of 50% or more.

The n may be 16 or more, 17 or more, or 18 or more, and 50 or less, 45 or less, 40 or less, 38 or less, 36 or less, 34 or less, 32 or less, 30 or less, 28 or less, 26 or less, 24 or less, 23 or less, 22 or less, 21 or less, or 20 or less.

When the compound represented by Chemical Formula 1 is a compound represented by Chemical Formula 1-D as an example, the compound can be prepared by a preparation method as shown in the following Reaction Scheme 1, and other remaining compounds can also be prepared in a similar manner.

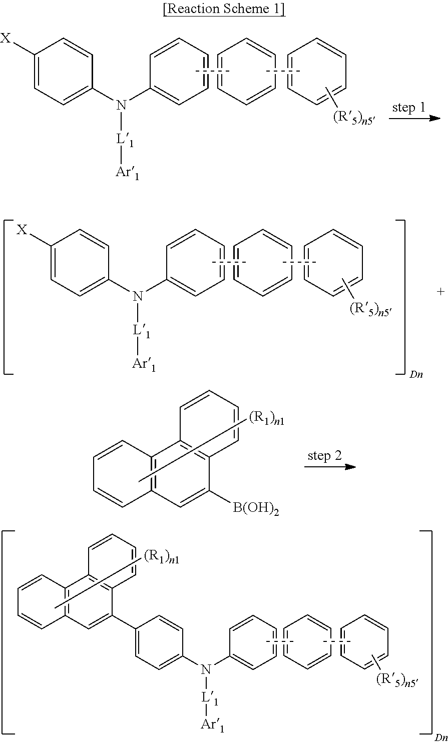

in Reaction Scheme 1, Dn, L′₁, Ar′₁, R₁, R′₅, n1 to n5′ are as defined in Chemical Formula 1-D, and X is halogen, preferably X is chloro or bromo.

In Reaction Scheme 1, the compound represented by Chemical Formula 1 is prepared by subjecting respective reactants substituted with deuterium to a Suzuki coupling reaction. Reaction Formula 1 proceeds by a deuterium substitution reaction (step 1) followed by a Suzuki coupling reaction (step 2). The deuterium substitution reaction is preferably carried out in the presence of D₂O, and the reactive group, catalyst, solvent, etc. for the deuterium substitution reaction can be changed to suit the desired product as known in the art. The Suzuki coupling reaction is preferably carried out in the presence of a palladium catalyst and a base, and a reactive group for the Suzuki coupling reaction can be modified as known in the art. The preparation method can be further embodied in Preparation Examples described hereinafter.

Further, according to the present disclosure, there is provided an organic light emitting device comprising a compound represented by Chemical Formula 1. In one example, the present disclosure provides an organic light emitting device comprising: a first electrode; a second electrode that is provided opposite to the first electrode; and one or more organic material layers that are provided between the first electrode and the second electrode, wherein at least one layer of the organic material layers includes the compound represented by Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, a hole transport auxiliary layer, a light emitting layer, a hole blocking layer, an electron transport layer, an electron injection layer and the like as the organic material layer. However, the structure of the organic light emitting device is not limited thereto, and it may include a smaller number of organic layers.

Further, the organic material layer may include a hole transport auxiliary layer or a light emitting layer, wherein the hole transport auxiliary layer or the organic material layer may include the compound represented by Chemical Formula 1.

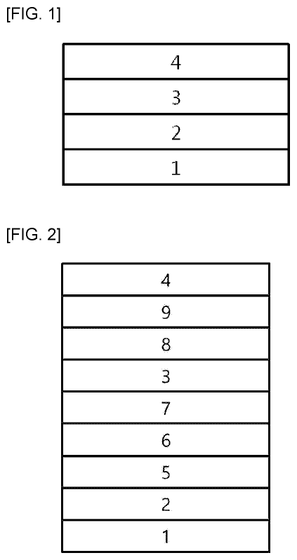

Further, the organic light emitting device according to the present disclosure may be a normal type organic light emitting device in which an anode, one or more organic material layers, and a cathode are sequentially stacked on a substrate. Further, the organic light emitting device according to the present disclosure may be an inverted type organic light emitting device in which a cathode, one or more organic material layers, and an anode are sequentially stacked on a substrate. For example, the structure of the organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, a hole transport auxiliary layer 7, a light emitting layer 3, a hole blocking layer 8, an electron transport and injection layer 9, and cathode 4. In such a structure, the compound represented by Chemical Formula 1 may be included in the hole transport auxiliary layer or the light emitting layer.

The organic light emitting device according to the present disclosure may be manufactured by materials and methods known in the art, except that at least one of the organic material layers includes the compound represented by Chemical Formula 1. Further, when the organic light emitting device includes a plurality of organic material layers, the organic material layers may be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Wherein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The hole transport auxiliary layer is a layer located between the hole transport layer and the light emitting layer. By including the hole transport auxiliary layer, holes are transported more smoothly from the hole transport layer to the light emitting layer, and excitons are confined in the light emitting layer to prevent light emission leakage, so that the organic light emitting device has excellent light emission efficiency. Preferably, the compound represented by Chemical Formula 1 may be used as a hole transport auxiliary material.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex(Alq₃); a carbazole-based compound; a dimerized styryl compound; BAlq; a 10-hydroxybenzoquinoline-metal compound; a benzoxazole, benzthiazole and benzimidazole-based compound; a poly(p-phenylenevinylene) (PPV)-based polymer; a spiro compound; polyfluorene, rubrene, and the like, but are not limited thereto.

The light emitting layer may include a host material and a dopant material. The host material includes a fused aromatic ring derivative, a heterocycle-containing compound, or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto. Preferably, the compound represented by Chemical Formula 1 may be used as a host material.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer that may receive the electrons from the electron injection layer and transport the electrons to the light emitting layer, and an electron transport material is suitably a material which may receive well injection of electrons from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, in the present disclosure, the “electron transport and injection layer” is a layer that performs both the roles of the electron injection layer and the electron transport layer, and the materials that perform the roles of each layer may be used alone or in combination, but are not limited thereto.

The organic light emitting device according to the present disclosure may be a bottom emission type device, a top emission type device, or a double side emission type device, and in particular, it may be a bottom emission type light emitting device that requires relatively high luminous efficiency.

In addition, the compound represented by Chemical Formula 1 may be included in an organic solar cell or an organic transistor in addition to an organic light emitting device.

Below, embodiments are described in more detail to assist in the understanding of the present disclosure. However, the following Examples are for illustrative purposes only, and are not intended to limit the scope of the present disclosure.

### Preparation Example 1

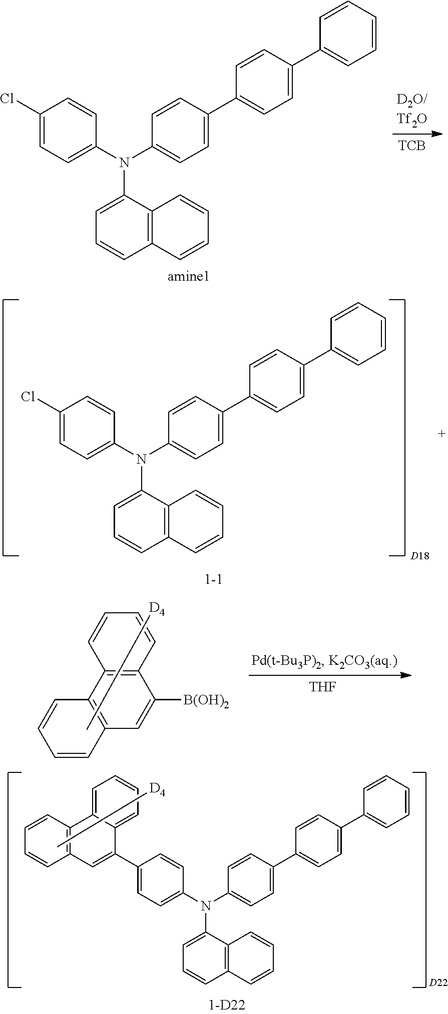

Compound amine1 (10 g, 20.7 mmol) was added to 200 ml of 1,2,4-trichlorobenzene, and the mixture was stirred at room temperature. In another vessel, I, deuterium oxide (15 g, 746.9 mmol) was added to trifluoromethanesulfonic anhydride (52.7 g, 186.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 1-1. (Yield: 48%, MS: [M+H]+=501).

Compound 1-1 (15 g, 30 mmol) and phenanthren-9-ylboronic acid_D4 (7.1 g, 31.5 mmol) were added to 300 ml of THE, and the mixture was stirred and refluxed. Then, potassium carbonate (12.4 g, 90 mmol) was dissolved in 100 ml of water and added thereto, and the mixture was sufficiently stirred and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.7 g of Compound 1-D22. (Yield: 76%, MS: [M+H]⁺=647)

### Preparation Example 2

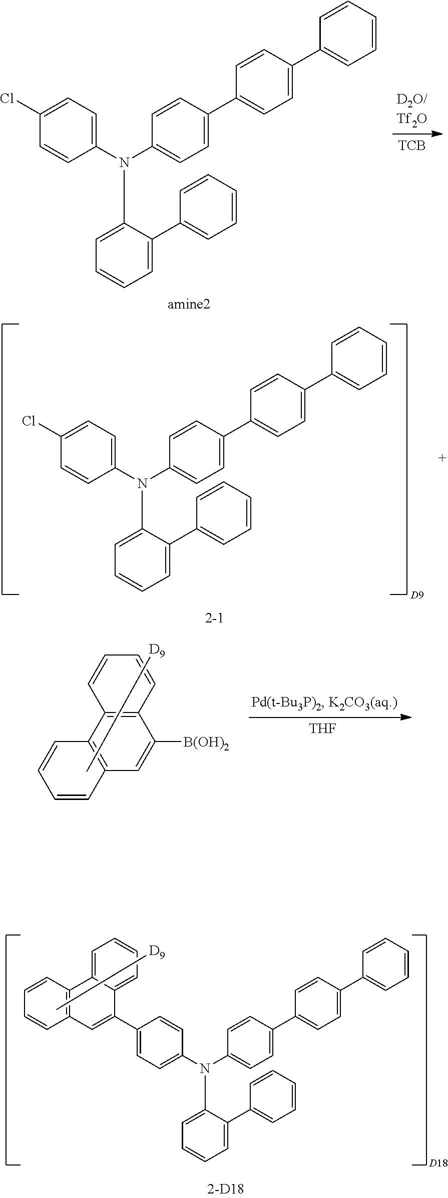

Compound amine2 (10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (33.3 g, 118.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.1 g of Compound 2-1. (Yield: 50%, MS: [M+H]+=518).

Compound 2-1 (15 g, 29 mmol) and phenanthren-9-ylboronic acid_D9 (7 g, 30.5 mmol) were added to 300 ml of THF, and the mixture was stirred and refluxed. Then, potassium carbonate (12 g, 87 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.5 g of Compound 2-D18. (Yield: 75%, MS: [M+H]+=669).

### Preparation Example 3

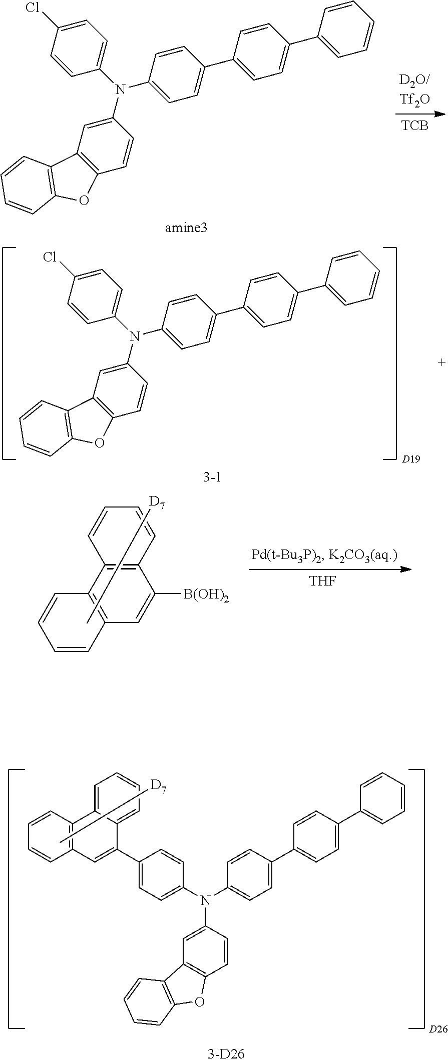

Compound amine3 (10 g, 19.2 mmol) was added to 200 ml of 1,2,4-trichlorobenzene, and the mixture was stirred at room temperature. In another vessel, deuterium oxide (13.8 g, 689.6 mmol) was added to trifluoromethanesulfonic anhydride (48.6 g, 172.4 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 3-1. (Yield: 48%, MS: [M+H]+=542).

Compound 3-1 (15 g, 27.7 mmol) and phenanthren-9-ylboronic acid_D7 (6.7 g, 29.1 mmol) were added to 300 ml of THF, and the mixture was stirred and refluxed. Then, potassium carbonate (11.5 g, 83.2 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.5 g of Compound 3-D26. (Yield: 60%, MS: [M+H]+=691).

### Preparation Example 4

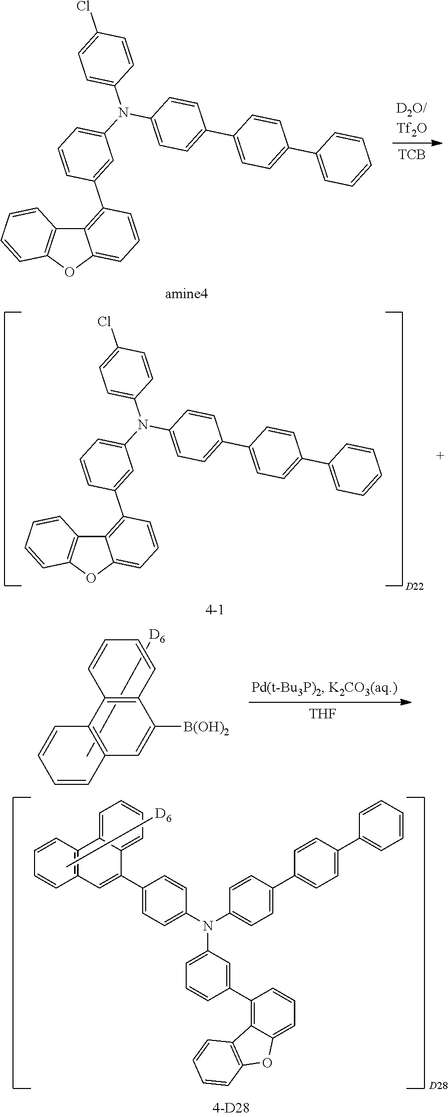

Compound amine4 (10 g, 16.7 mmol) was added to 200 ml of 1,2,4-trichlorobenzene, and the mixture was stirred at room temperature. In another vessel, deuterium oxide (12.1 g, 601.9 mmol) was added to trifluoromethanesulfonic anhydride (42.5 g, 150.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.3 g of Compound 4-1. (Yield: 41%, MS: [M+H]+=621).

Compound 4-1 (15 g, 24.2 mmol) and phenanthren-9-ylboronic acid_D6 (5.8 g, 25.4 mmol) were added to 300 ml of THF, and the mixture was stirred and refluxed. Then, potassium carbonate (10 g, 72.6 mmol) was dissolved in 100 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.9 g of Compound 4-D28. (Yield: 75%, MS: [M+H]+=769).

### Preparation Example 5

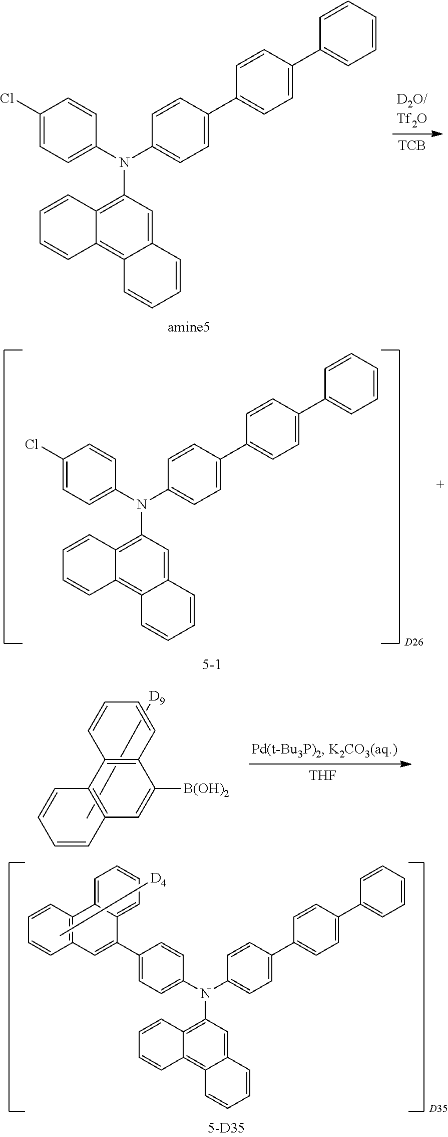

Compound amine5 (10 g, 18.8 mmol) was added to 200 ml of 1,2,4-trichlorobenzene, and the mixture was stirred at room temperature. In another vessel, deuterium oxide (13.6 g, 676.6 mmol) was added to trifluoromethanesulfonic anhydride (63.6 g, 225.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 15 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.2 g of Compound 5-1. (Yield: 50%, MS: [M+H]+=559).

Compound 5-1 (15 g, 26.9 mmol) and phenanthren-9-ylboronic acid_D9 (6.5 g, 28.2 mmol) were added to 300 ml of THF, and the mixture was stirred and refluxed. Then, potassium carbonate (11.1 g, 80.6 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.1 g of Compound 5-D35. (Yield: 74%, MS: [M+H]+=710).

### Preparation Example 6

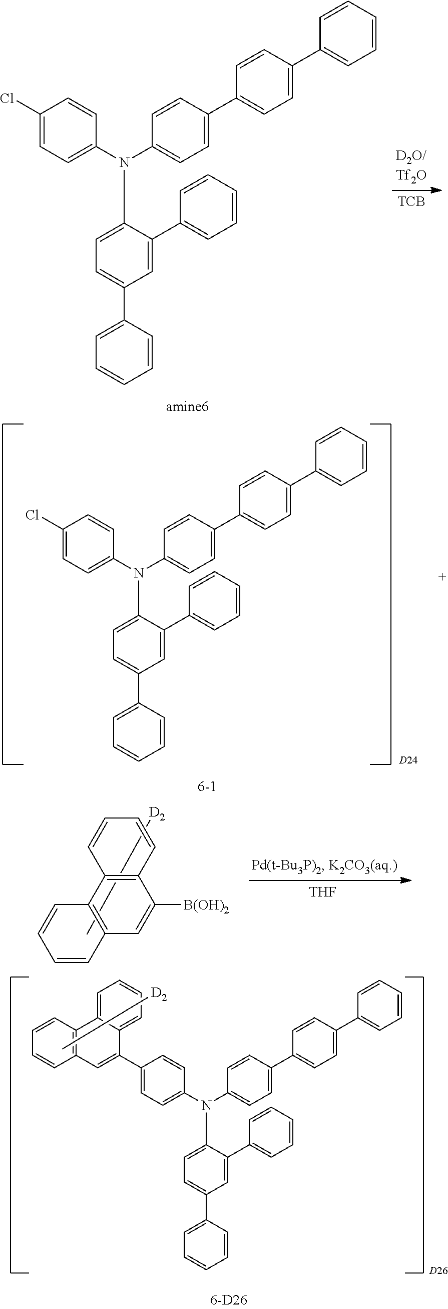

Compound amine6 (10 g, 17.1 mmol) was added to 200 ml of 1,2,4-trichlorobenzene, and the mixture was stirred at room temperature. In another vessel, I, deuterium oxide (12.3 g, 616.3 mmol) was added to trifluoromethanesulfonic anhydride (43.5 g, 154.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.7 g of Compound 6-1. (Yield: 45%, MS: [M+H]+=609).

Compound 6-1 (15 g, 24.7 mmol) and phenanthren-9-ylboronic acid_D2 (5.8 g, 25.9 mmol) were added to 300 ml of THF, and the mixture was stirred and refluxed. Then, potassium carbonate (10.2 g, 74 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.9 g of Compound 6-D26. (Yield: 75%, MS: [M+H]+=753).

### Preparation Example 7

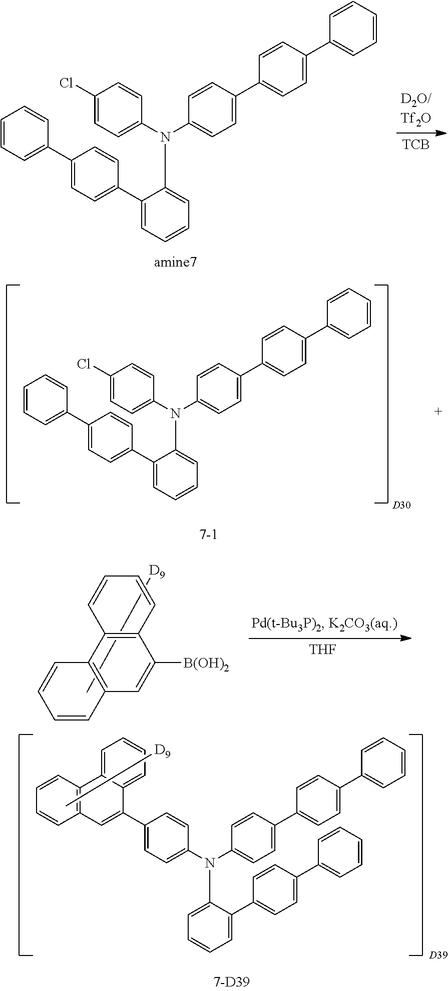

Compound amine7 (10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (58 g, 205.4 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 15 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.8 g of Compound 7-1. (Yield: 46%, MS: [M+H]+=615).

Compound 7-1 (15 g, 24.4 mmol) and phenanthren-9-ylboronic acid_D9 (5.9 g, 25.6 mmol) were added to 300 ml of THF, and the mixture was stirred and refluxed. Then, potassium carbonate (10.1 g, 73.3 mmol) was dissolved in 100 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.4 g of Compound 7-D39. (Yield: 61%, MS: [M+H]+=766).

### Preparation Example 8

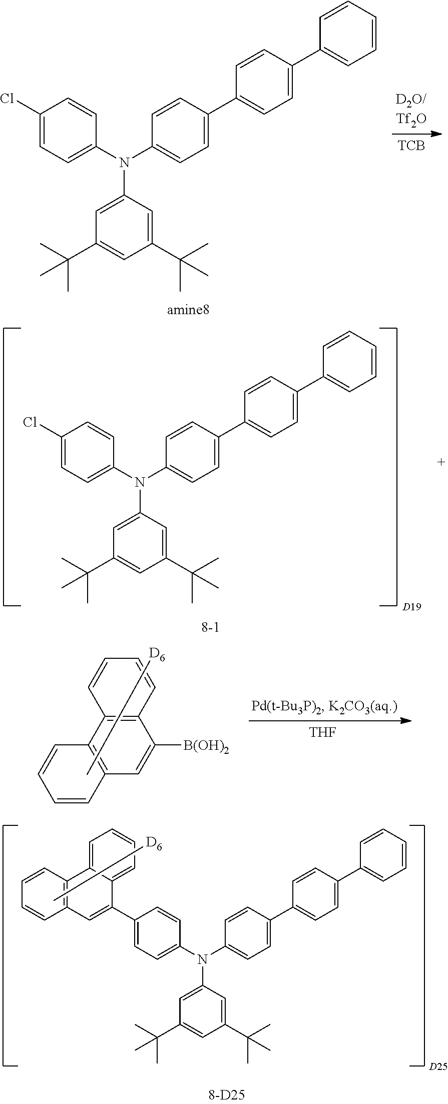

Compound amine8 (10 g, 18.4 mmol) was added to 200 ml of 1,2,4-trichlorobenzene, and the mixture was stirred at room temperature. In another vessel, deuterium oxide (13.3 g, 661.5 mmol) was added to trifluoromethanesulfonic anhydride (31.1 g, 110.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.8 g of Compound 8-1. (Yield: 47%, MS: [M+H]+=561).

Compound 8-1(15 g, 26.8 mmol) and phenanthren-9-ylboronic acid_D6 (6.4 g, 28.1 mmol) were added to 300 ml of THF, and the mixture was stirred and refluxed. Then, potassium carbonate (11.1 g, 80.3 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.1 g of Compound 8-D25. (Yield: 69%, MS: [M+H]+=709).

### Preparation Example 9

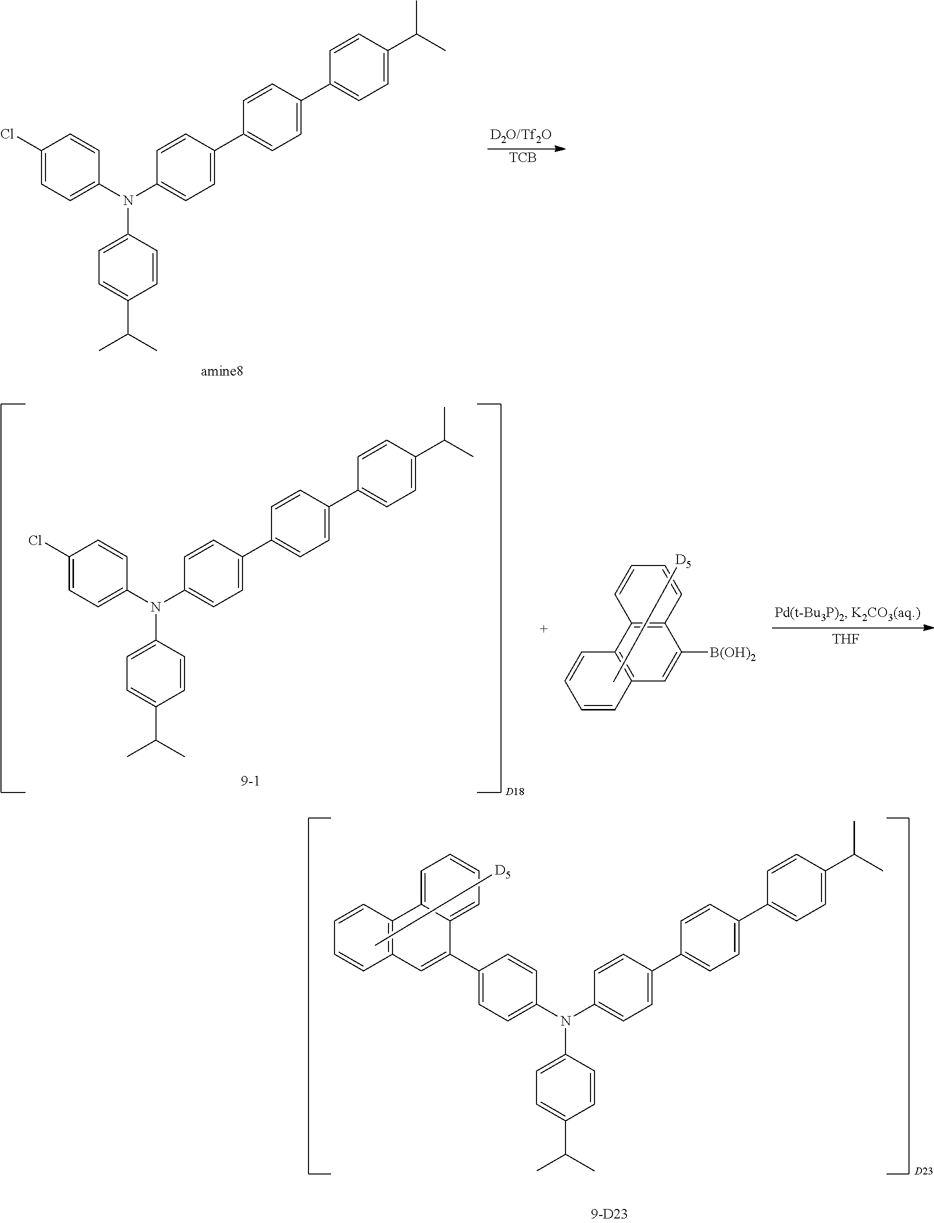

Compound amine9(10 g, 19.4 mmol) was added to 200 ml of 1,2,4-trichlorobenzene, and the mixture was stirred at room temperature. In another vessel, deuterium oxide (14 g, 697.5 mmol) was added to trifluoromethanesulfonic anhydride (32.8 g, 116.2 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.4 g of Compound 9-1. (Yield: 43%, MS: [M+H]+=531).

Compound 9-1(15 g, 28.3 mmol) and phenanthren-9-ylboronic acid_D5 (6.7 g, 29.7 mmol) were added to 300 ml of THF, and the mixture was stirred and refluxed. Then, potassium carbonate (11.7 g, 84.9 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 15.3 g of Compound 9-D23. (Yield: 80%, MS: [M+H]+=678).

### Preparation Example 10

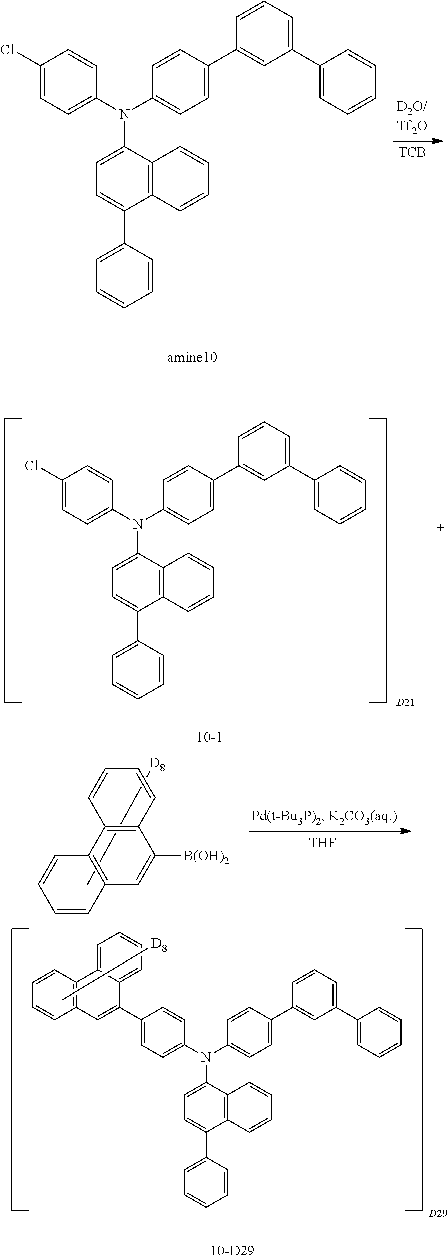

Compound amine10(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (45.5 g, 161.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.4 g of Compound 10-1. (Yield: 42%, MS: [M+H]+=580).

Compound 10-1(15 g, 25.9 mmol) and phenanthren-9-ylboronic acid_D8(6.3 g, 27.2 mmol) were added to 300 ml of THF, and the mixture was stirred and refluxed. Then, potassium carbonate (10.7 g, 77.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.6 g of Compound 10-D29. (Yield: 67%, MS: [M+H]+=730).

### Preparation Example 11

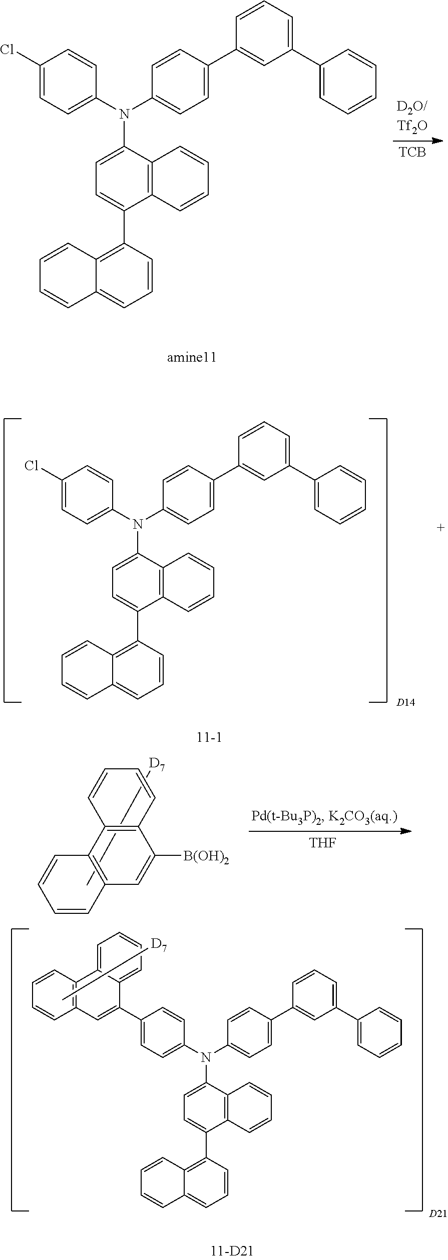

Compound amine11(10 g, 16.4 mmol) was added to 200 ml of 1,2,4-trichlorobenzene, and the mixture was stirred at room temperature. In another vessel, deuterium oxide (11.9 g, 591.9 mmol) was added to trifluoromethanesulfonic anhydride (27.8 g, 98.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.9 g of Compound 11-1. (Yield: 48%, MS: [M+H]+=623).

Compound 11-1(15 g, 24.1 mmol) and phenanthren-9-ylboronic acid_D7(5.8 g, 25.3 mmol) were added to 300 ml of THF, and the mixture was stirred and refluxed. Then, potassium carbonate (10 g, 72.3 mmol) was dissolved in 100 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.5 g of Compound 11-D21. (Yield: 62%, MS: [M+H]+=772).

### Preparation Example 12

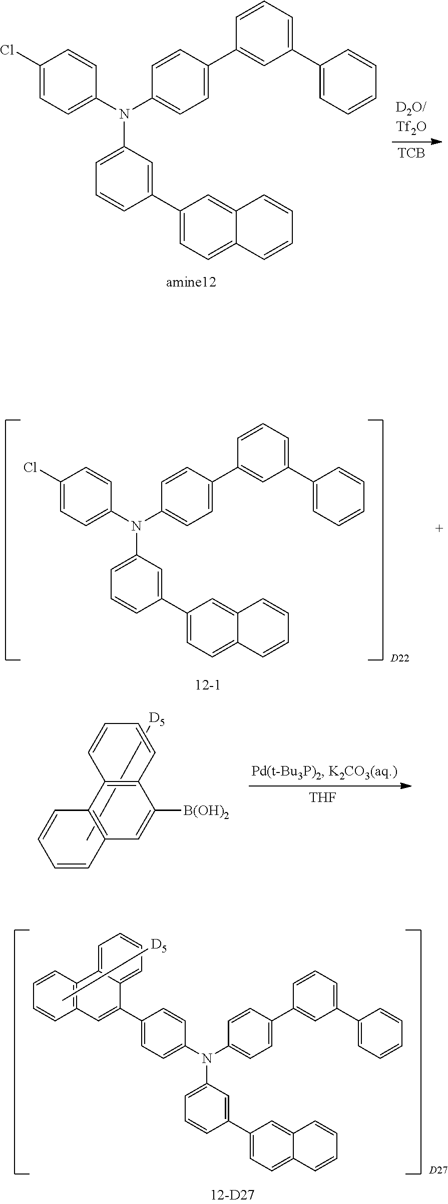

Compound amine12(10 g, 17.9 mmol) was added to 200 ml of 1,2,4-trichlorobenzene, and the mixture was stirred at room temperature. In another vessel, deuterium oxide to (12.9 g, 645 mmol) was added trifluoromethanesulfonic anhydride (45.5 g, 161.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.1 g of Compound 12-1. (Yield: 49%, MS: [M+H]+=581).

Compound 12-1(15 g, 25.9 mmol) and phenanthren-9-ylboronic acid_D5(6.2 g, 27.1 mmol) were added to 300 ml of THF, and the mixture was stirred and refluxed. Then, potassium carbonate (10.7 g, 77.6 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.5 g of Compound 12-D27. (Yield: 61%, MS: [M+H]+=728).

### Preparation Example 13

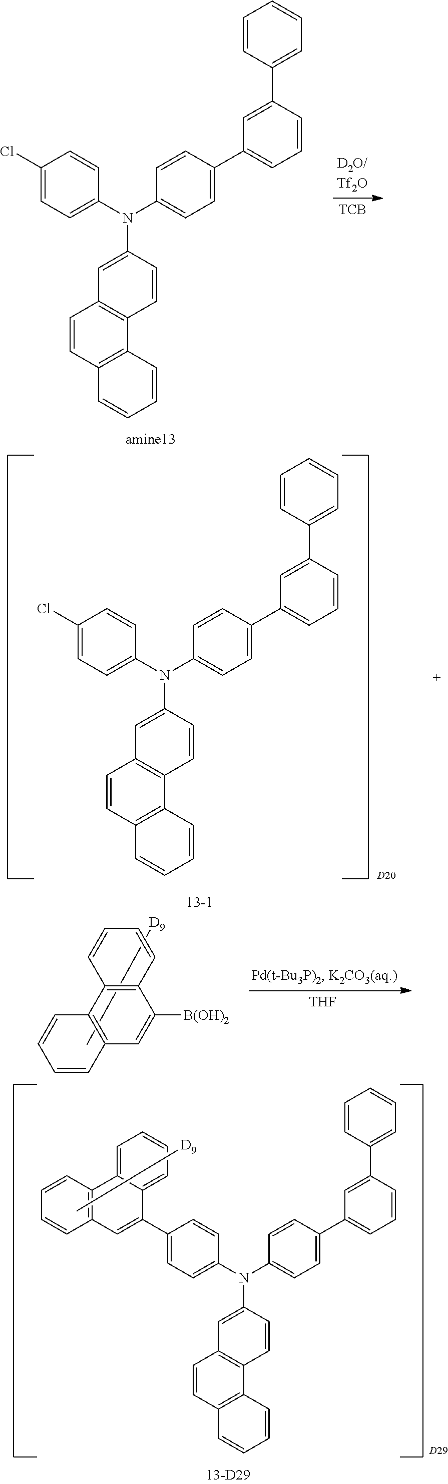

Compound amine13(10 g, 18.8 mmol) was added to 200 ml of 1,2,4-trichlorobenzene, and the mixture was stirred at room temperature. In another vessel, deuterium oxide (13.6 g, 676.6 mmol) was added to trifluoromethanesulfonic anhydride (47.7 g, 169.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.1 g of Compound 13-1. (Yield: 49%, MS: [M+H]+=553).

Compound 13-1(15 g, 27.2 mmol) and phenanthren-9-ylboronic acid_D9(6.6 g, 28.5 mmol) were added to 300 ml of THF, and the mixture was stirred and refluxed. Then, potassium carbonate (11.3 g, 81.5 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 15.1 g of Compound 13-D29. (Yield: 79%, MS: [M+H]+=704).

### Preparation Example 14

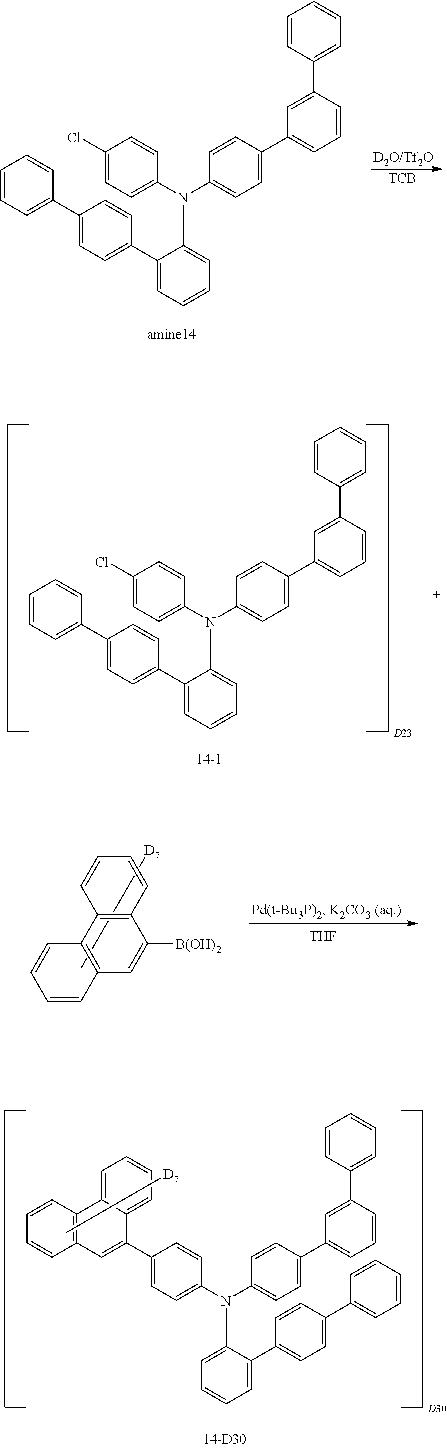

Compound amine14(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (43.5 g, 154.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.5 g of Compound 14-1. (Yield: 43%, MS: [M+H]+=608).

Compound 14-1(15 g, 24.7 mmol) and phenanthren-9-ylboronic acid_D7(5.9 g, 25.9 mmol) were added to 300 ml of THF, and the mixture was stirred and refluxed. Then, potassium carbonate (10.2 g, 74.1 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12 g of Compound 14-D30. (Yield: 64%, MS: [M+H]+=757).

### Preparation Example 15

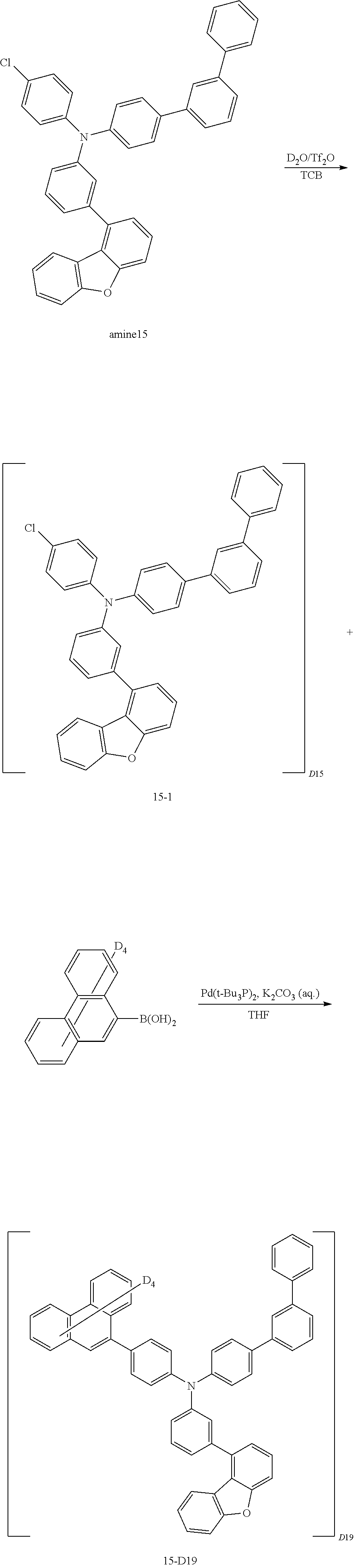

Compound amine15(10 g, 16.7 mmol) was added to 200 ml of 1,2,4-trichlorobenzene, and the mixture was stirred at room temperature. In another vessel, deuterium oxide (12.1 g, 601.9 mmol) was added to trifluoromethanesulfonic anhydride (28.3 g, 100.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.4 g of Compound 15-1. (Yield: 43%, MS: [M+H]+=614).

Compound 15-1(15 g, 24.5 mmol) and phenanthren-9-ylboronic acid_D4(5.8 g, 25.7 mmol) were added to 300 ml of THF, and the mixture was stirred and refluxed. Then, potassium carbonate (10.1 g, 73.4 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.7 g of Compound 15-D19. (Yield: 74%, MS: [M+H]+=760).

### Preparation Example 16

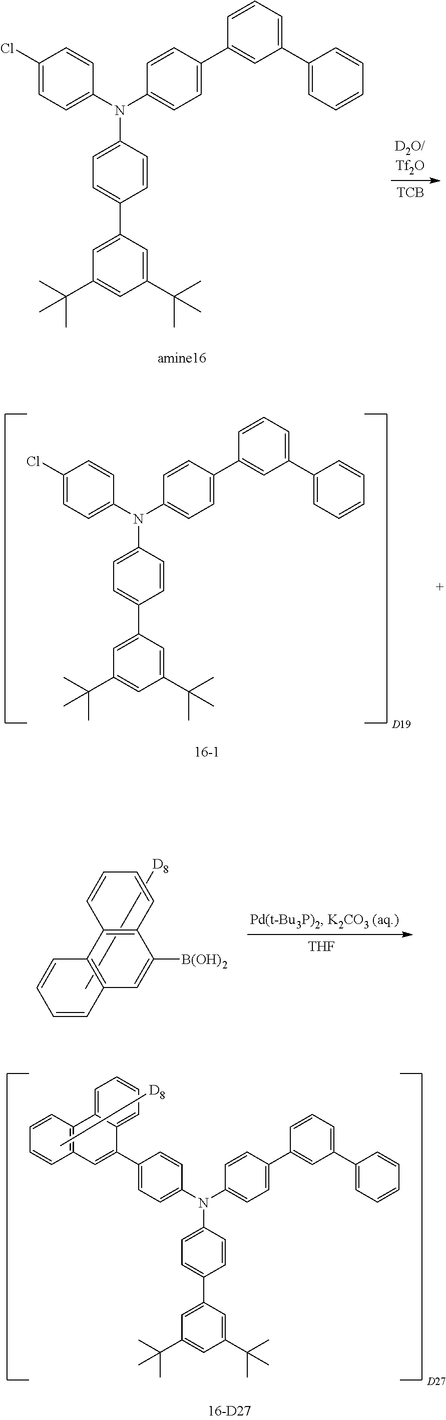

Compound amine16(10 g, 16.1 mmol) was added to 200 ml of 1,2,4-trichlorobenzene, and the mixture was stirred at room temperature. In another vessel, deuterium oxide (11.6 g, 580.4 mmol) was added to trifluoromethanesulfonic anhydride (27.3 g, 96.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 16-1. (Yield: 49%, MS: [M+H]+=638).

Compound 16-1(15 g, 23.5 mmol) and phenanthren-9-ylboronic acid_D8(5.7 g, 24.7 mmol) were added to 300 ml of THF, and the mixture was stirred and refluxed. Then, potassium carbonate (9.8 g, 70.6 mmol) was dissolved in 100 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.5 g of Compound 16-D27. (Yield: 62%, MS: [M+H]+=788).

### Preparation Example 17

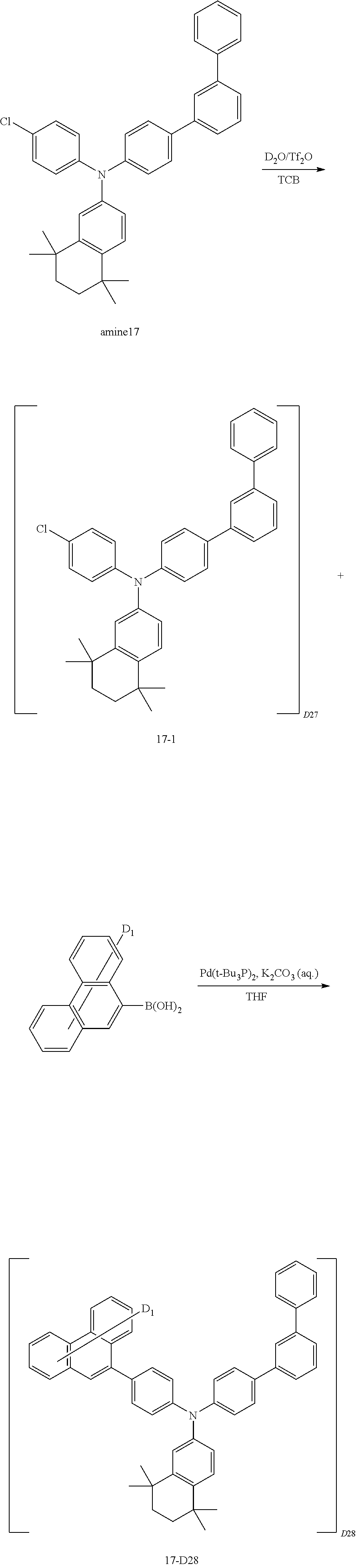

Compound amine17(10 g, 18.4 mmol) was added to 200 ml of 1,2,4-trichlorobenzene, and the mixture was stirred at room temperature. In another vessel, deuterium oxide (13.3 g, 664 mmol) was added to trifluoromethanesulfonic anhydride (46.8 g, 166 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.1 g of Compound 17-1. (Yield: 49%, MS: [M+H]+=570).

Compound 17-1(15 g, 26.4 mmol) and phenanthren-9-ylboronic acid_D1(6.2 g, 27.7 mmol) were added to 300 ml of THF, and the mixture was stirred and refluxed. Then, potassium carbonate (10.9 g, 79.1 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.1 g of Compound 17-D28. (Yield: 75%, MS: [M+H]+=713).

### Preparation Example 18

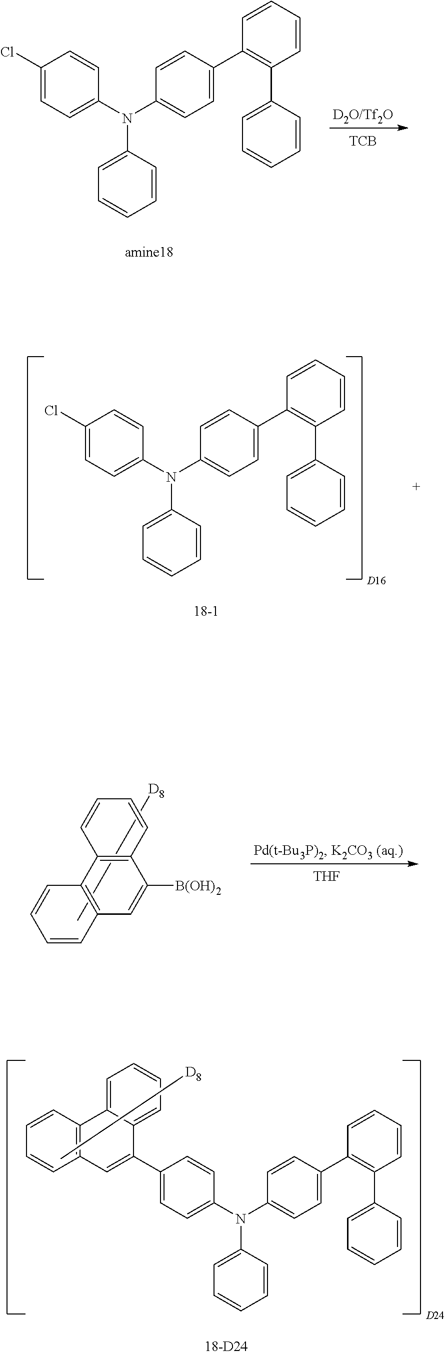

Compound amine18(10 g, 23.2 mmol) was added to 200 ml of 1,2,4-trichlorobenzene, and the mixture was stirred at room temperature. In another vessel, deuterium oxide (16.7 g, 833.4 mmol) was added to trifluoromethanesulfonic anhydride (58.8 g, 208.4 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.3 g of Compound 18-1. (Yield: 41%, MS: [M+H]+=449).

Compound 18-1(15 g, 33.5 mmol) and phenanthren-9-ylboronic acid_D8(8.1 g, 35.2 mmol) were added to 300 ml of THF, and the mixture was stirred and refluxed. Then, potassium carbonate (13.9 g, 100.5 mmol) was dissolved in 100 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 15.8 g of Compound 18-D24. (Yield: 79%, MS: [M+H]+=599).

### Preparation Example 19

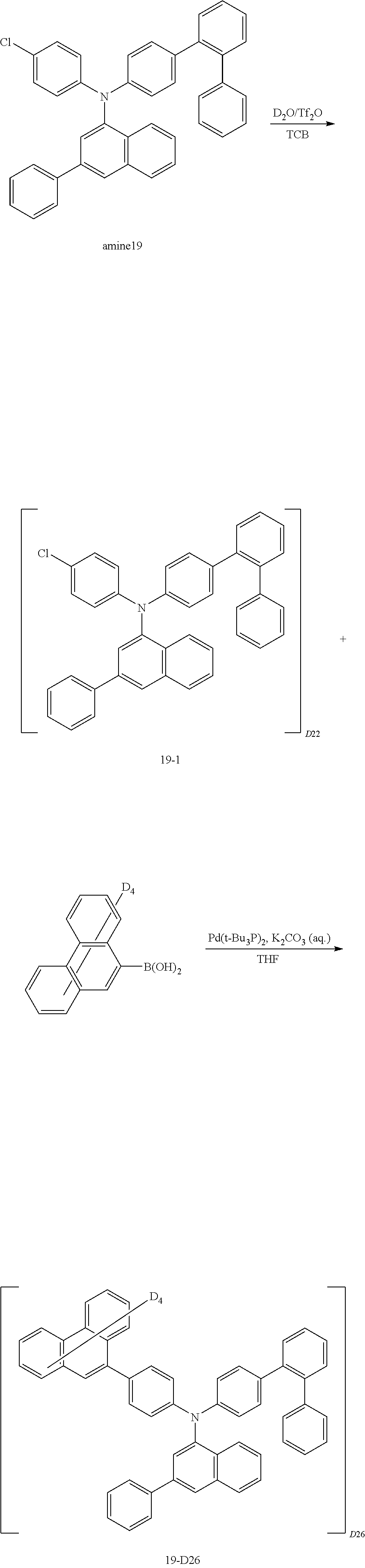

Compound amine19(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (45.5 g, 161.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.3 g of Compound 19-1. (Yield: 41%, MS: [M+H]+=581).

Compound 19-1(15 g, 25.9 mmol) and phenanthren-9-ylboronic acid_D4(6.1 g, 27.1 mmol) were added to 300 ml of THF, and the mixture was stirred and refluxed. Then, potassium carbonate (10.7 g, 77.6 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.2 g of Compound 19-D26. (Yield: 65%, MS: [M+H]+=727).

### Preparation Example 20

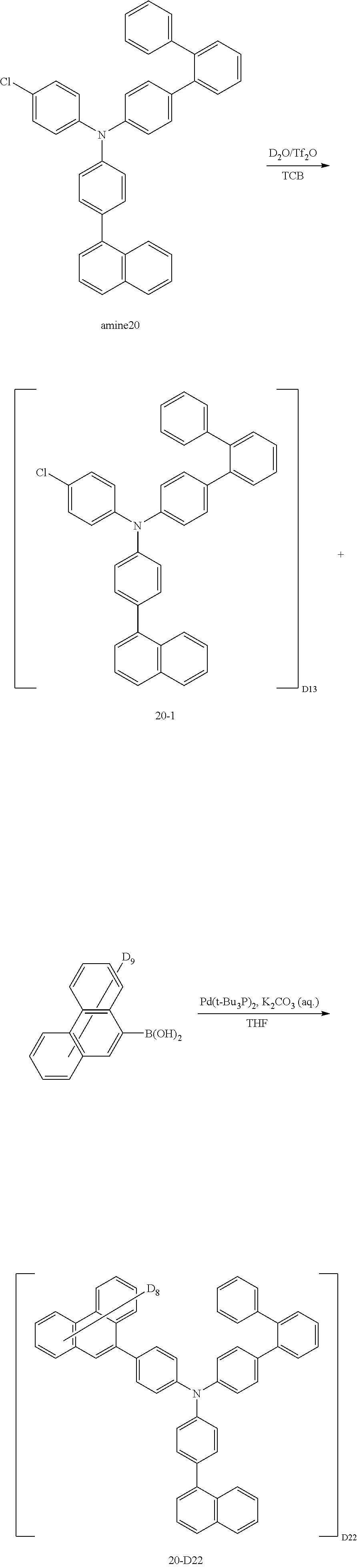

Compound amine20(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (30.3 g, 107.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.9 g of Compound 20-1. (Yield: 48%, MS: [M+H]+=572).

Compound 20-1(15 g, 26.3 mmol) and phenanthren-9-ylboronic acid_D9(6.4 g, 27.6 mmol) were added to 300 ml of THF, and the mixture was stirred and refluxed. Then, potassium carbonate (10.9 g, 78.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.7 g of Compound 20-D22. (Yield: 72%, MS: [M+H]+=723).

### Preparation Example 21

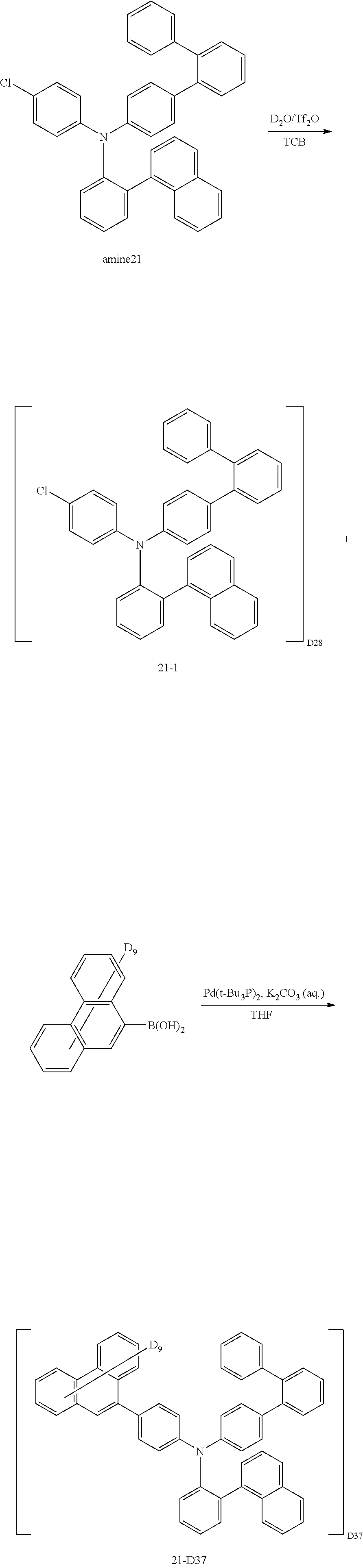

Compound amine21(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (60.7 g, 215 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 15 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.3 g of Compound 21-1. (Yield: 50%, MS: [M+H]+=587).

Compound 21-1(15 g, 25.6 mmol) and phenanthren-9-ylboronic acid_D9(6.2 g, 26.9 mmol) were added to 300 ml of THF, and the mixture was stirred and refluxed. Then, potassium carbonate (10.6 g, 76.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.2 g of Compound 21-D37. (Yield: 70%, MS: [M+H]+=738).

### Preparation Example 22

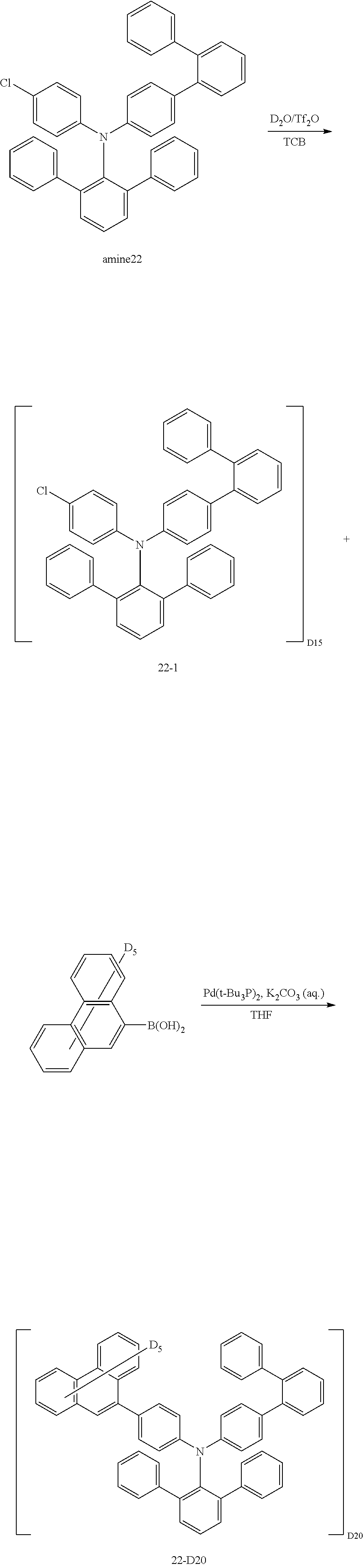

Compound amine22(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (29 g, 102.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.2 g of Compound 22-1. (Yield: 41%, MS: [M+H]+=600).

Compound 22-1(15 g, 25 mmol) and phenanthren-9-ylboronic acid_D5(6 g, 26.3 mmol) were added to 300 ml of THF, and the mixture was stirred and refluxed. Then, potassium carbonate (10.4 g, 75.1 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.3 g of Compound 22-D20. (Yield: 71%, MS: [M+H]+=747).

### Preparation Example 23

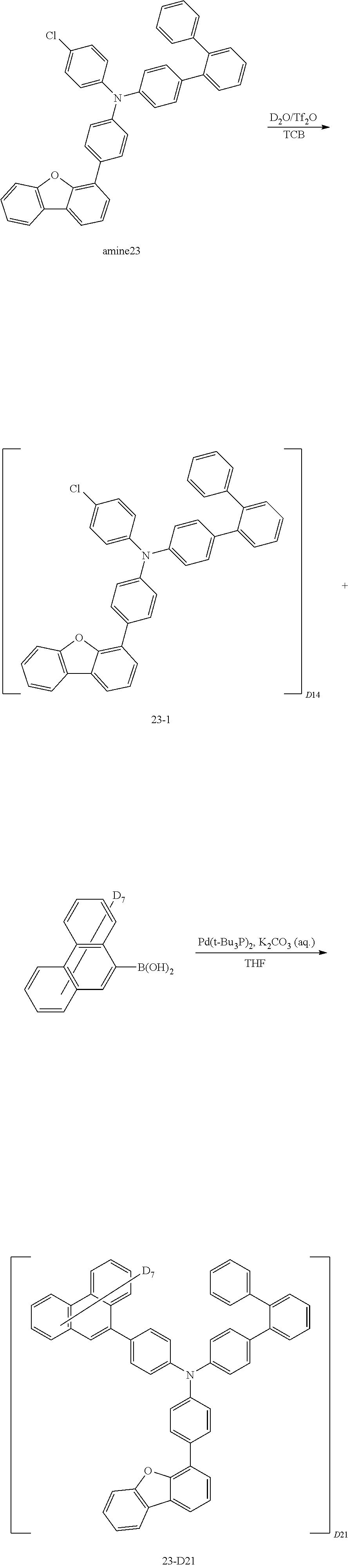

Compound amine23(10 g, 16.7 mmol) was added to 200 ml of 1,2,4-trichlorobenzene, and the mixture was stirred at room temperature. In another vessel, deuterium oxide (12.1 g, 601.9 mmol) was added to trifluoromethanesulfonic anhydride (28.3 g, 100.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.1 g of Compound 23-1. (Yield: 50%, MS: [M+H]+=613).

Compound 23-1(15 g, 24.5 mmol) and phenanthren-9-ylboronic acid_D7(5.9 g, 25.7 mmol) were added to 300 ml of THF, and the mixture was stirred and refluxed. Then, potassium carbonate (10.2 g, 73.5 mmol) was dissolved in 100 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.5 g of Compound 23-D21. (Yield: 67%, MS: [M+H]+=762).

### Preparation Example 24

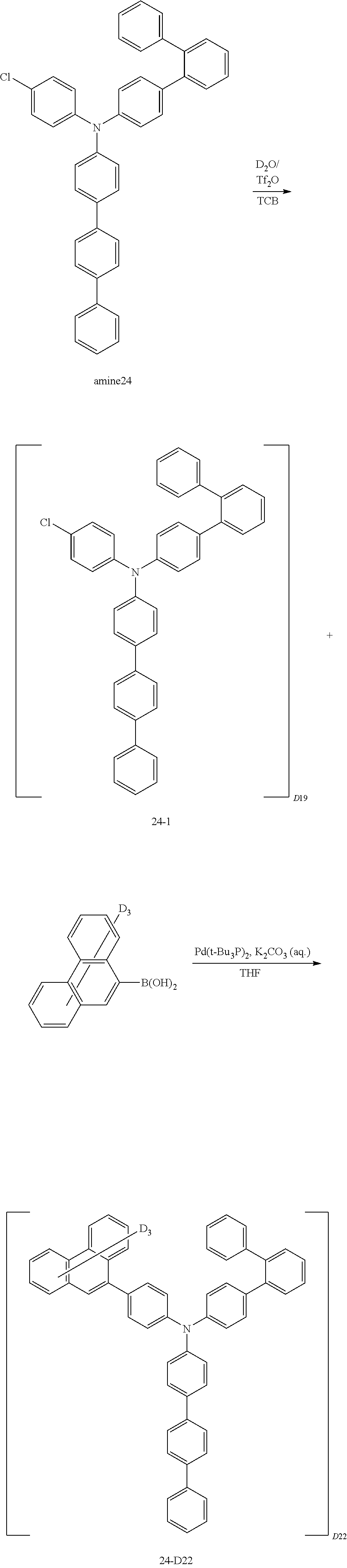

Compound amine24(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (43.5 g, 154.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 24-1. (Yield: 48%, MS: [M+H]+=604).

Compound 24-1(15 g, 24.9 mmol) and phenanthren-9-ylboronic acid_D3(5.9 g, 26.1 mmol) were added to 300 ml of THF, and the mixture was stirred and refluxed. Then, potassium carbonate (10.3 g, 74.6 mmol) was dissolved in 100 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.3 g of Compound 24-D22. (Yield: 66%, MS: [M+H]+=749).

### Preparation Example 25

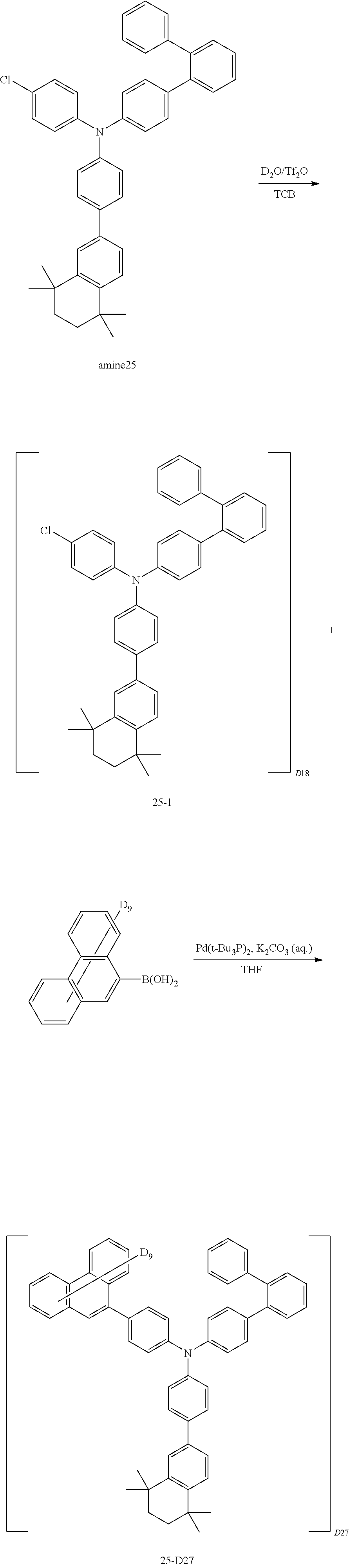

Compound amine25(10 g, 16.2 mmol) was added to 200 ml of 1,2,4-trichlorobenzene, and the mixture was stirred at room temperature. In another vessel, deuterium oxide (11.7 g, 582.3 mmol) was added to trifluoromethanesulfonic anhydride (27.4 g, 97 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 25-1. (Yield: 49%, MS: [M+H]+=637).

Compound 25-1(15 g, 23.6 mmol) and phenanthren-9-ylboronic acid_D9(5.7 g, 24.8 mmol) were added to 300 ml of THF, and the mixture was stirred and refluxed. Then, potassium carbonate (9.8 g, 70.7 mmol) was dissolved in 100 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.1 g of Compound 25-D27. (Yield: 65%, MS: [M+H]+=788).

### Preparation Example 26

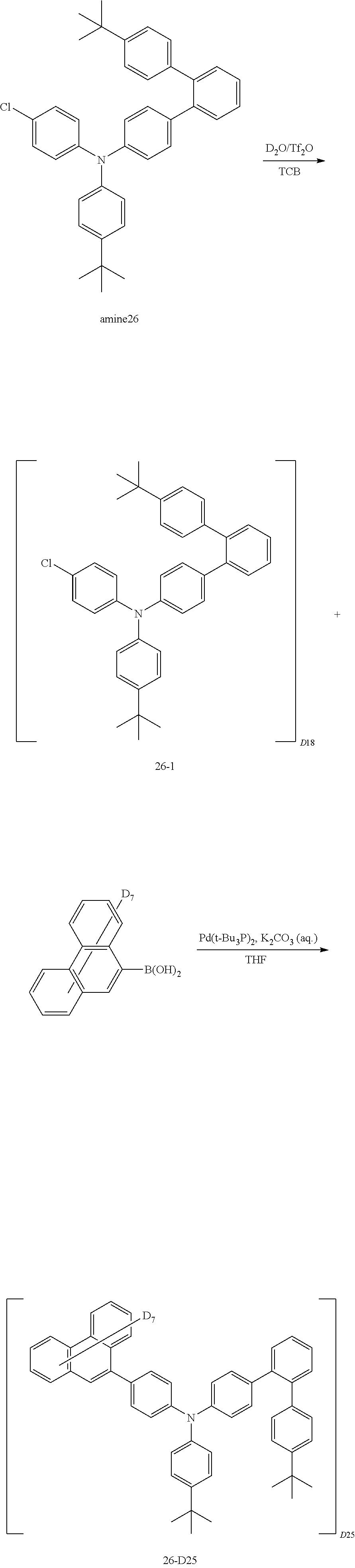

Compound amine26(10 g, 18.4 mmol) was added to 200 ml of 1,2,4-trichlorobenzene, and the mixture was stirred at room temperature. In another vessel, deuterium oxide (13.3 g, 661.5 mmol) was added to trifluoromethanesulfonic anhydride (31.1 g, 110.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.4 g of Compound 26-1. (Yield: 43%, MS: [M+H]+=563).

Compound 26-1(15 g, 26.7 mmol) and phenanthren-9-ylboronic acid_D7(6.4 g, 28 mmol) were added to 300 ml of THF, and the mixture was stirred and refluxed. Then, potassium carbonate (11.1 g, 80 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12 g of Compound 26-D25. (Yield: 63%, MS: [M+H]+=712).

### Preparation Example 27

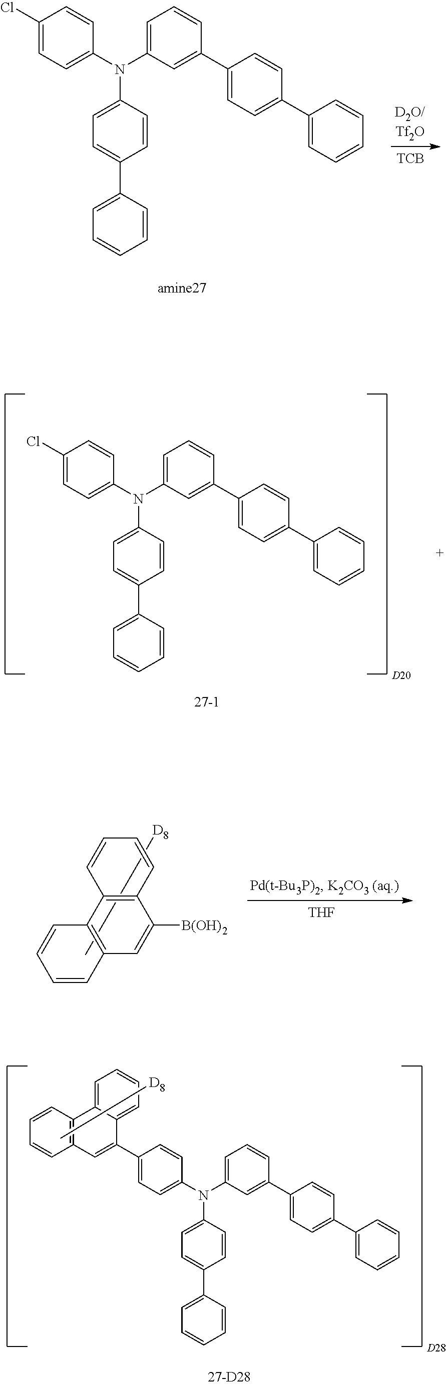

Compound amine27(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (50 g, 177.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 27-1. (Yield: 48%, MS: [M+H]+=529).

Compound 27-1(15 g, 28.4 mmol) and phenanthren-9-ylboronic acid_D8(6.9 g, 29.8 mmol) were added to 300 ml of THF, and the mixture was stirred and refluxed. Then, potassium carbonate (11.8 g, 85.2 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.1 g of Compound 27-D28. (Yield: 63%, MS: [M+H]+=679).

### Preparation Example 28

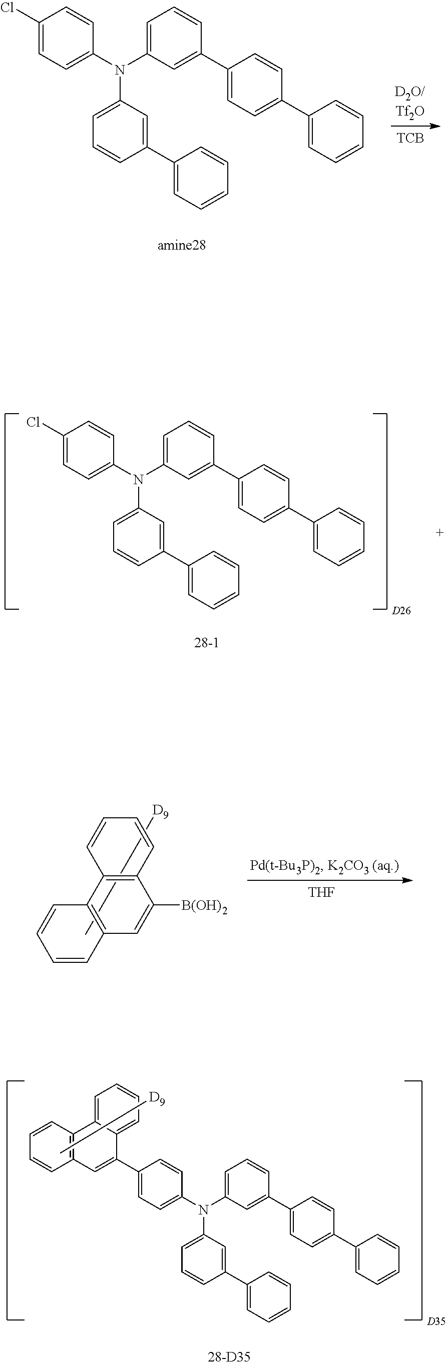

Compound amine28(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (66.6 g, 236.2 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 15 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.2 g of Compound 28-1. (Yield: 49%, MS: [M+H]+=535).

Compound 28-1(15 g, 28.1 mmol) and phenanthren-9-ylboronic acid_D9(6.8 g, 29.5 mmol) were added to 300 ml of THF, and the mixture was stirred and refluxed. Then, potassium carbonate (11.6 g, 84.3 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.5 g of Compound 28-D35. (Yield: 65%, MS: [M+H]+=686).

### Preparation Example 29

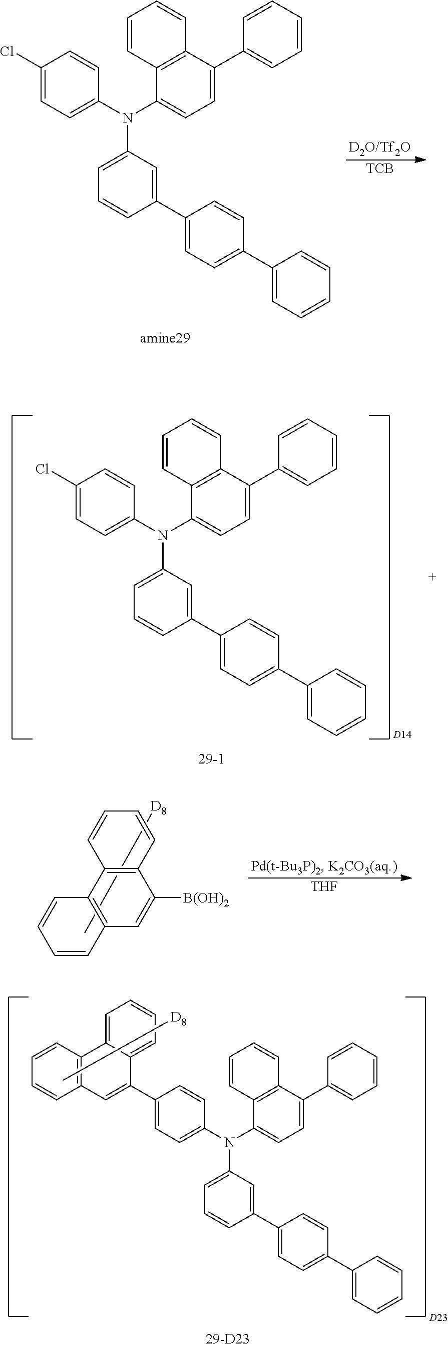

Compound amine29(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (30.3 g, 107.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.2 g of Compound 29-1. (Yield: 41%, MS: [M+H]+=573).

Compound 29-1(15 g, 26.2 mmol) and phenanthren-9-ylboronic acid_D9(6.4 g, 27.5 mmol) were added to 300 ml of THE, and the mixture was stirred and refluxed. Then, potassium carbonate (10.9 g, 78.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.4 g of Compound 29-D23. (Yield: 60%, MS: [M+H]+=724).

### Preparation Example 30

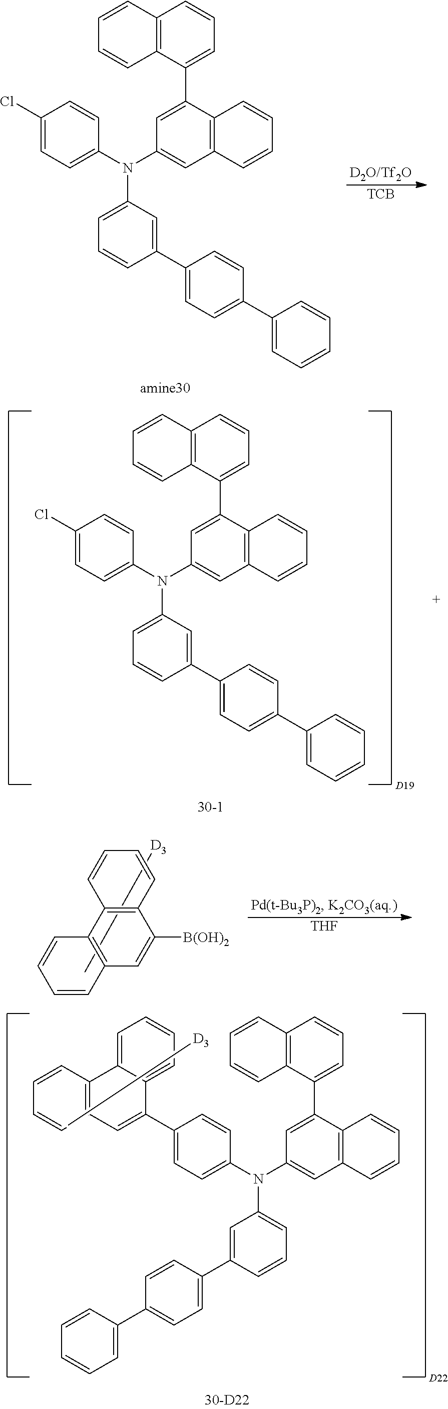

Compound amine30(10 g, 16.4 mmol) was added to 200 ml of 1,2,4-trichlorobenzene, and the mixture was stirred at room temperature. In another vessel, deuterium oxide (11.9 g, 591.9 mmol) was added to trifluoromethanesulfonic anhydride (41.8 g, 148 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.2 g of Compound 30-1. (Yield: 50%, MS: [M+H]+=628).

Compound 30-1(15 g, 23.9 mmol) and phenanthren-9-ylboronic acid_D3(5.7 g, 25.1 mmol) were added to 300 ml of THF, and the mixture was stirred and refluxed. Then, potassium carbonate (9.9 g, 71.7 mmol) was dissolved in 100 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.3 g of Compound 30-D22. (Yield: 61%, MS: [M+H]+=773).

### Preparation Example 31

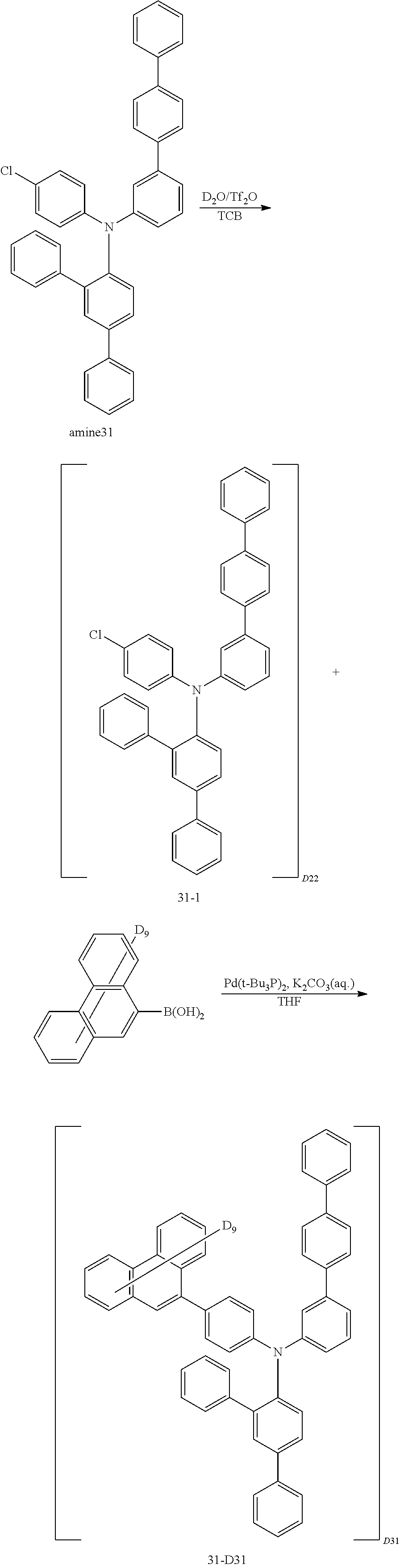

Compound amine31(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (43.5 g, 154.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.8 g of Compound 31-1. (Yield: 46%, MS: [M+H]+=607).

Compound 31-1(15 g, 24.7 mmol) and phenanthren-9-ylboronic acid_D9(6 g, 26 mmol) were added to 300 ml of THF, and the mixture was stirred and refluxed. Then, potassium carbonate (10.3 g, 74.2 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.6 g of Compound 31-D31. (Yield: 78%, MS: [M+H]+=758).

### Preparation Example 32

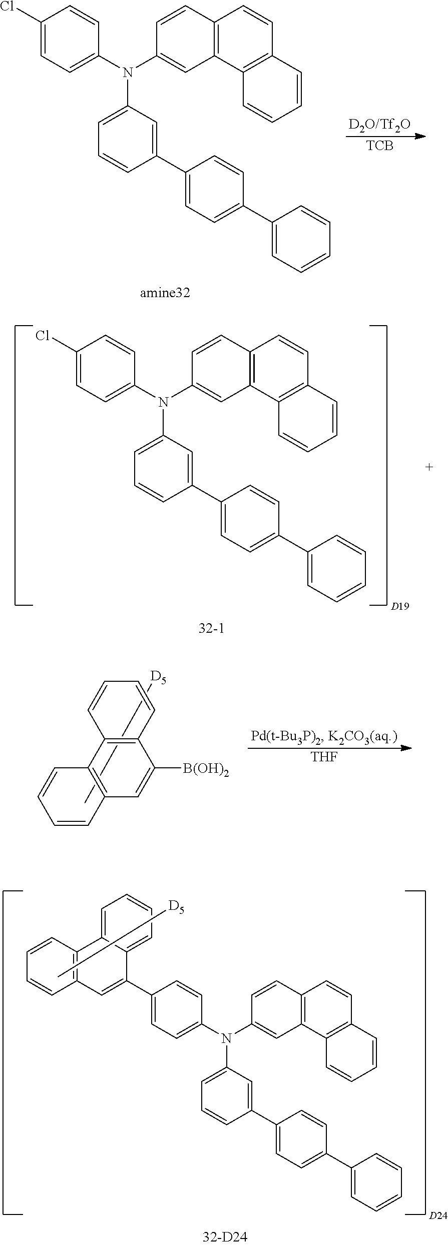

Compound amine32(10 g, 18.8 mmol) was added to 200 ml of 1,2,4-trichlorobenzene, and the mixture was stirred at room temperature. In another vessel, deuterium oxide (13.6 g, 676.6 mmol) was added to trifluoromethanesulfonic anhydride (47.7 g, 169.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.3 g of Compound 32-1. (Yield: 42%, MS: [M+H]+=552).

Compound 32-1(15 g, 27.2 mmol) and phenanthren-9-ylboronic acid_D5(6.5 g, 28.6 mmol) were added to 300 ml of THF, and the mixture was stirred and refluxed. Then, potassium carbonate (11.3 g, 81.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.5 g of Compound 32-D24. (Yield: 71%, MS: [M+H]+=699).

### Preparation Example 33

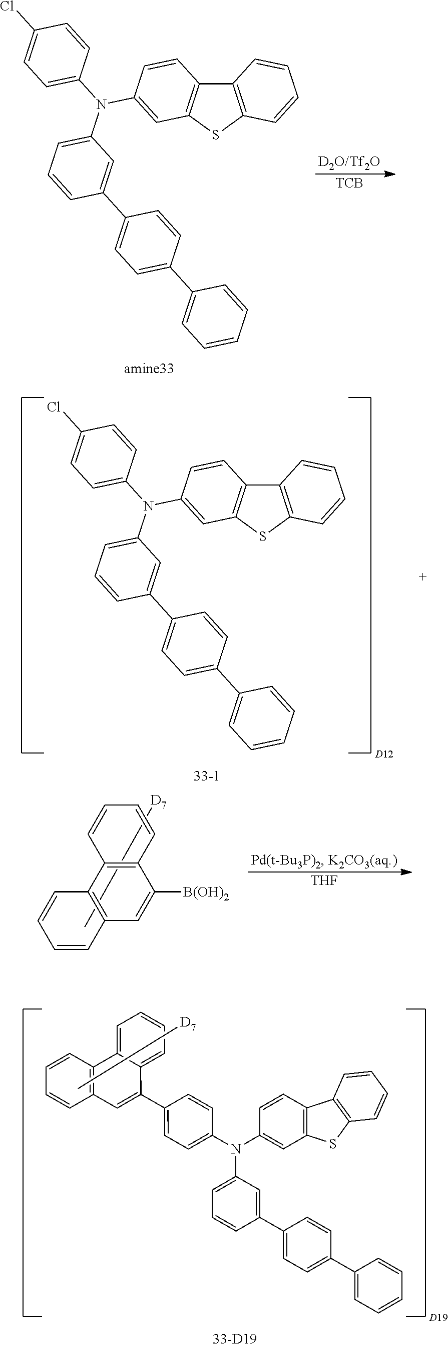

Compound amine33(10 g, 18.6 mmol) was added to 200 ml of 1,2,4-trichlorobenzene, and the mixture was stirred at room temperature. In another vessel, deuterium oxide (13.4 g, 669 mmol) was added to trifluoromethanesulfonic anhydride (31.5 g, 111.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.5 g of Compound 33-1. (Yield: 44%, MS: [M+H]+=551).

Compound 33-1(15 g, 27.3 mmol) and phenanthren-9-ylboronic acid_D7(6.6 g, 28.6 mmol) were added to 300 ml of THF, and the mixture was stirred and refluxed. Then, potassium carbonate (11.3 g, 81.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.3 g of Compound 33-D19. (Yield: 75%, MS: [M+H]+=700).

### Preparation Example 34

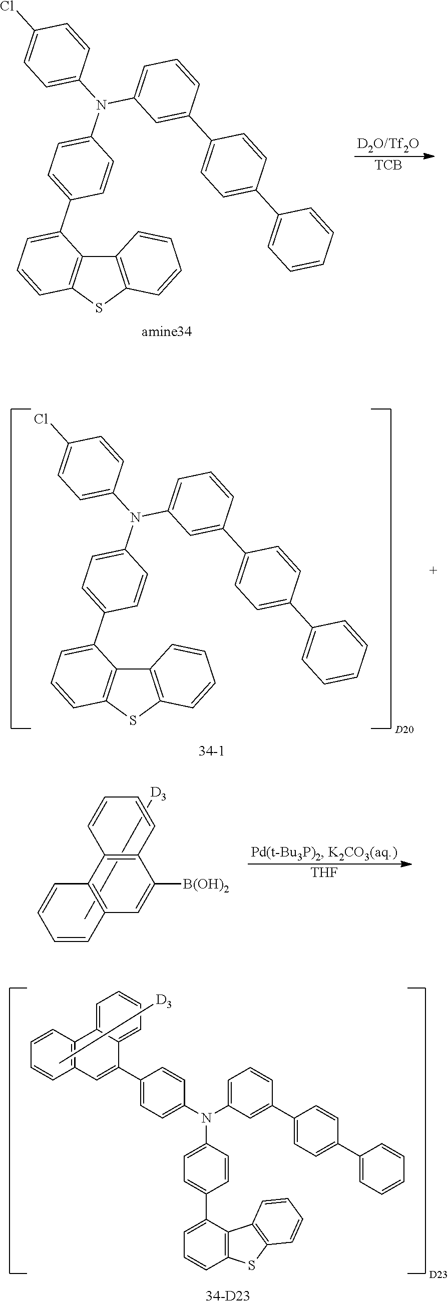

Compound amine34(10 g, 16.3 mmol) was added to 200 ml of 1,2,4-trichlorobenzene, and the mixture was stirred at room temperature. In another vessel, deuterium oxide (11.7 g, 586.1 mmol) was added to trifluoromethanesulfonic anhydride (41.3 g, 146.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.5 g of Compound 34-1. (Yield: 44%, MS: [M+H]+=635).

Compound 34-1(15 g, 23.7 mmol) and phenanthren-9-ylboronic acid_D3(5.6 g, 24.8 mmol) were added to 300 ml of THF, and the mixture was stirred and refluxed. Then, potassium carbonate (9.8 g, 71 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.7 g of Compound 34-D23. (Yield: 69%, MS: [M+H]+=780).

### Preparation Example 35

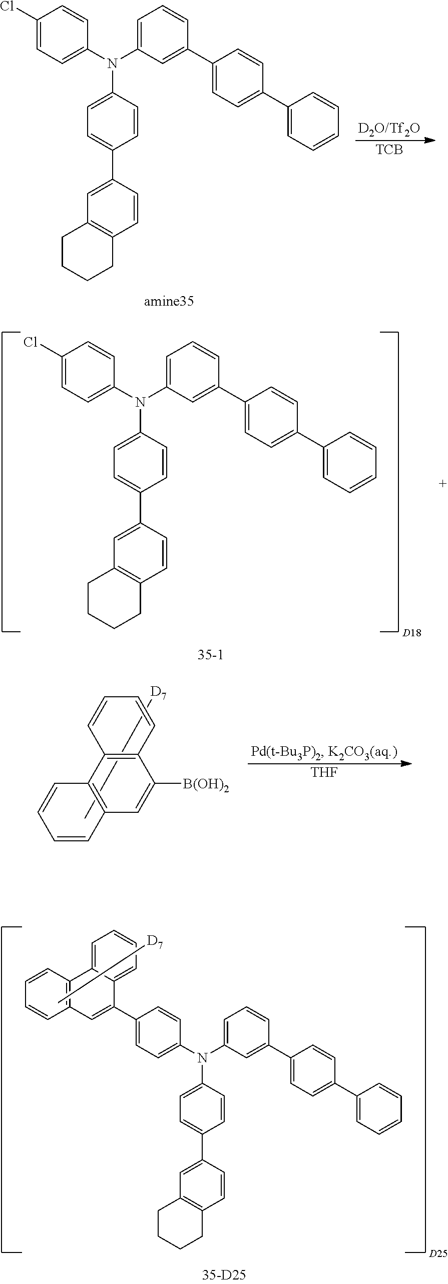

Compound amine35(10 g, 17.8 mmol) was added to 200 ml of 1,2,4-trichlorobenzene, and the mixture was stirred at room temperature. In another vessel, deuterium oxide (12.8 g, 640.4 mmol) was added to trifluoromethanesulfonic anhydride (45.2 g, 160.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.7 g of Compound 35-1. (Yield: 46%, MS: [M+H]+=581).

Compound 35-1(15 g, 25.9 mmol) and phenanthren-9-ylboronic acid_D7(6.2 g, 27.1 mmol) were added to 300 ml of THF, and the mixture was stirred and refluxed. Then, potassium carbonate (10.7 g, 77.6 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.3 g of Compound 35-D25. (Yield: 60%, MS: [M+H]+=730).

### Preparation Example 36

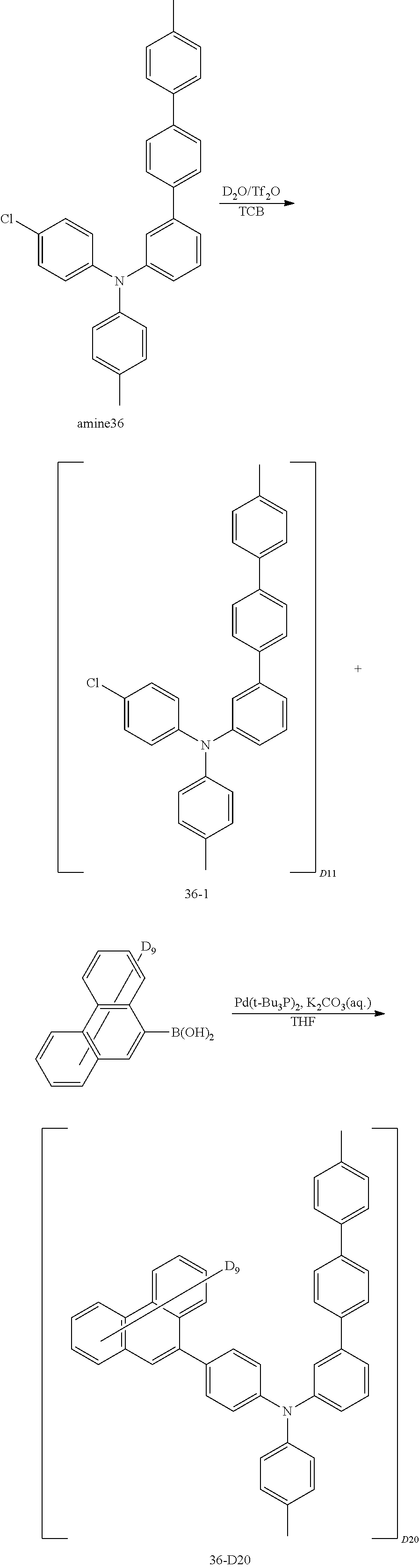

Compound amine36(10 g, 21.7 mmol) was added to 200 ml of 1,2,4-trichlorobenzene, and the mixture was stirred at room temperature. In another vessel, deuterium oxide (15.7 g, 782.6 mmol) was added to trifluoromethanesulfonic anhydride (36.8 g, 130.4 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.1 g of Compound 36-1. (Yield: 40%, MS: [M+H]+=472).

Compound 36-1(15 g, 31.8 mmol) and phenanthren-9-ylboronic acid_D9(7.7 g, 33.4 mmol) were added to 300 ml of THF, and the mixture was stirred and refluxed. Then, potassium carbonate (13.2 g, 95.5 mmol) was dissolved in 100 ml of water and added thereto, and the mixture was sufficiently stirred and then bis(tri-tert-butylphosphine)palladium(0) (0.2 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.5 g of Compound 36-D20. (Yield: 63%, MS: [M+H]+=623).

### Preparation Example 37

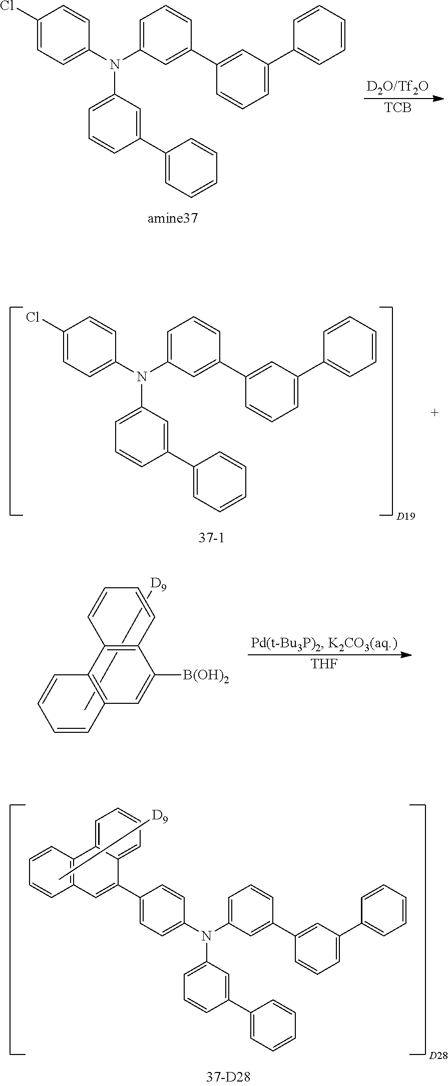

Compound amine37(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (50 g, 177.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 37-1. (Yield: 48%, MS: [M+H]+=528).

Compound 37-1(15 g, 28.5 mmol) and phenanthren-9-ylboronic acid_D9(6.9 g, 29.9 mmol) were added to 300 ml of THF, and the mixture was stirred and refluxed. Then, potassium carbonate (11.8 g, 85.4 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.2 g of Compound 37-D28. (Yield: 74%, MS: [M+H]+=677).

### Preparation Example 38

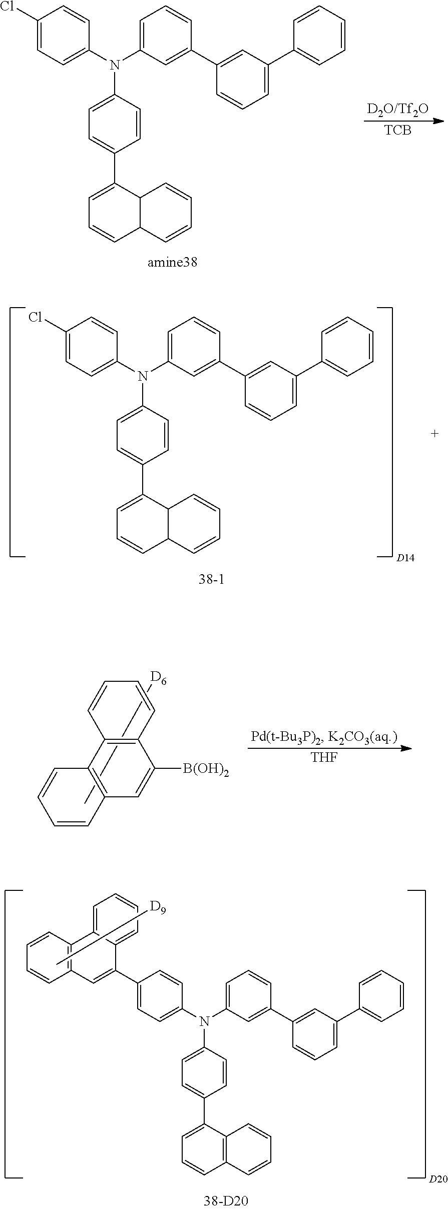

Compound amine38(10 g, 17.9 mmol) was added to 200 ml of 1,2,4-trichlorobenzene, and the mixture was stirred at room temperature. In another vessel, deuterium oxide (12.9 g, 642.7 mmol) was added to trifluoromethanesulfonic anhydride (30.2 g, 107.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 38-1. (Yield: 49%, MS: [M+H]+=575).

Compound 38-1(15 g, 26.1 mmol) and phenanthren-9-ylboronic acid_D6(6.3 g, 27.4 mmol) were added to 300 ml of THF, and the mixture was stirred and refluxed. Then, potassium carbonate (10.8 g, 78.4 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.4 g of Compound 38-D20. (Yield: 66%, MS: [M+H]+=721).

### Preparation Example 39

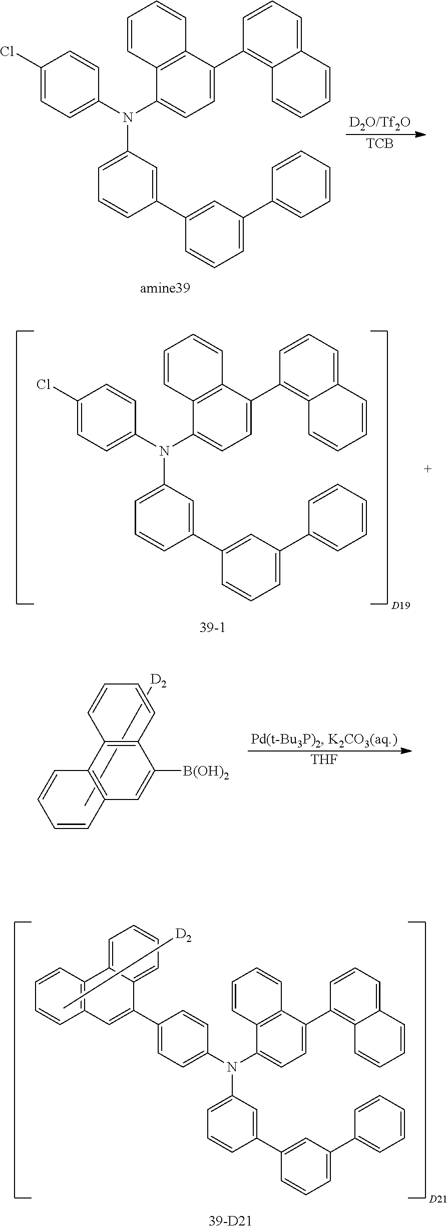

Compound amine39(10 g, 16.4 mmol) was added to 200 ml of 1,2,4-trichlorobenzene, and the mixture was stirred at room temperature. In another vessel, deuterium oxide (11.9 g, 591.9 mmol) was added to trifluoromethanesulfonic anhydride (41.8 g, 148 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.9 g of Compound 39-1. (Yield: 48%, MS: [M+H]+=628).

Compound 39-1(15 g, 23.9 mmol) and phenanthren-9-ylboronic acid_D2(5.6 g, 25.1 mmol) were added to 300 ml of THF, and the mixture was stirred and refluxed. Then, potassium carbonate (9.9 g, 71.7 mmol) was dissolved in 100 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.2 g of Compound 39-D21. (Yield: 66%, MS: [M+H]+=772).

### Preparation Example 40

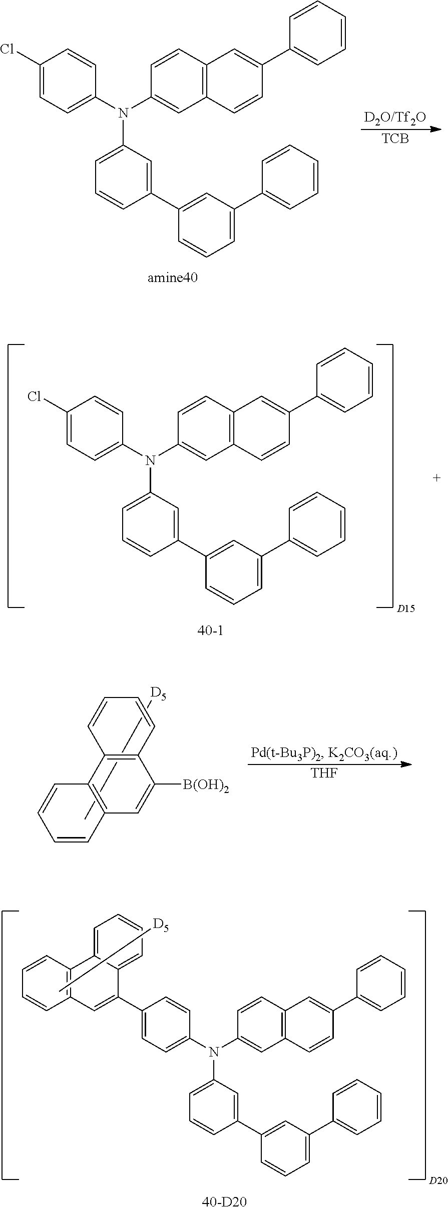

Compound amine40(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (30.3 g, 107.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 40-1. (Yield: 49%, MS: [M+H]+=574).

Compound 40-1(15 g, 26.2 mmol) and phenanthren-9-ylboronic acid_D5(6.2 g, 27.5 mmol) were added to 300 ml of THF, and the mixture was stirred and refluxed. Then, potassium carbonate (10.9 g, 78.5 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.4 g of Compound 40-D20. (Yield: 66%, MS: [M+H]+=721).

### Preparation Example 41

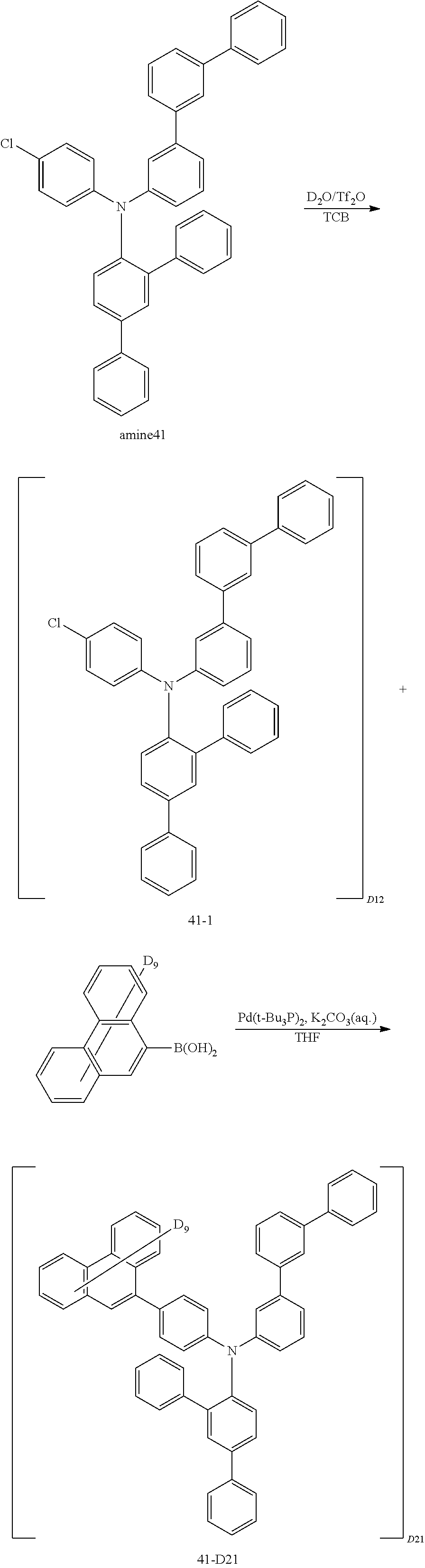

Compound amine41(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (29 g, 102.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.1 g of Compound 41-1. (Yield: 40%, MS: [M+H]+=597).

Compound 41-1(15 g, 25.2 mmol) and phenanthren-9-ylboronic acid_D9(6.1 g, 26.4 mmol) were added to 300 ml of THE, and the mixture was stirred and refluxed. Then, potassium carbonate (10.4 g, 75.5 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.9 g of Compound 41-D21. (Yield: 74%, MS: [M+H]+=748).

### Preparation Example 42

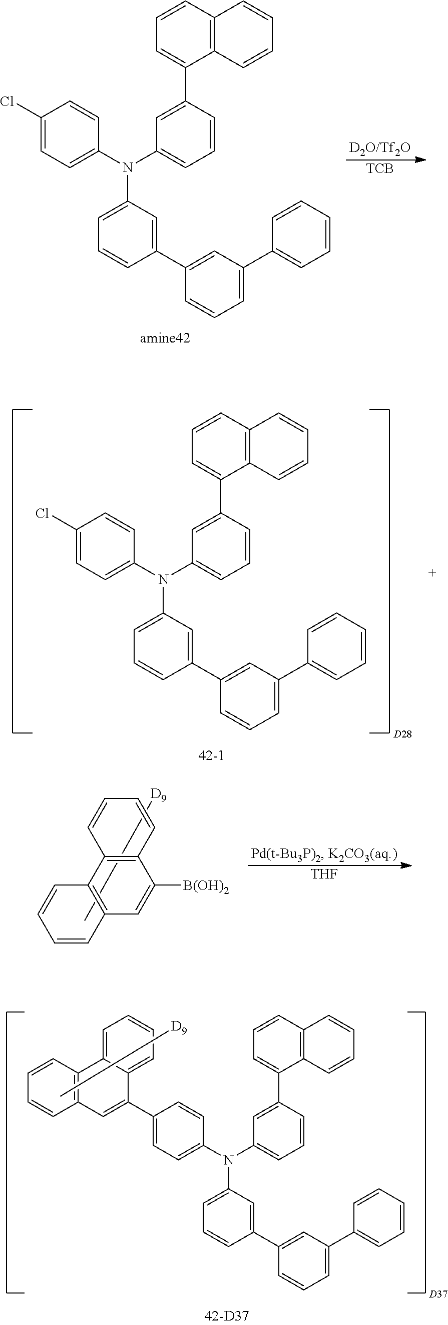

Compound amine42(10 g, 17.9 mmol) was added to 200 ml of 1,2,4-trichlorobenzene, and the mixture was stirred at room temperature. In another vessel, deuterium (12.9 oxide g, 645 mmol) was added to trifluoromethanesulfonic anhydride (60.7 g, 215 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 15 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.6 g of Compound 42-1. (Yield: 44%, MS: [M+H]+=587).

Compound 42-1(15 g, 25.6 mmol) and phenanthren-9-ylboronic acid_D9(6.2 g, 26.9 mmol) were added to 300 ml of THF, and the mixture was stirred and refluxed. Then, potassium carbonate (10.6 g, 76.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.6 g of Compound 42-D37. (Yield: 72%, MS: [M+H]+=738).

### Preparation Example 43

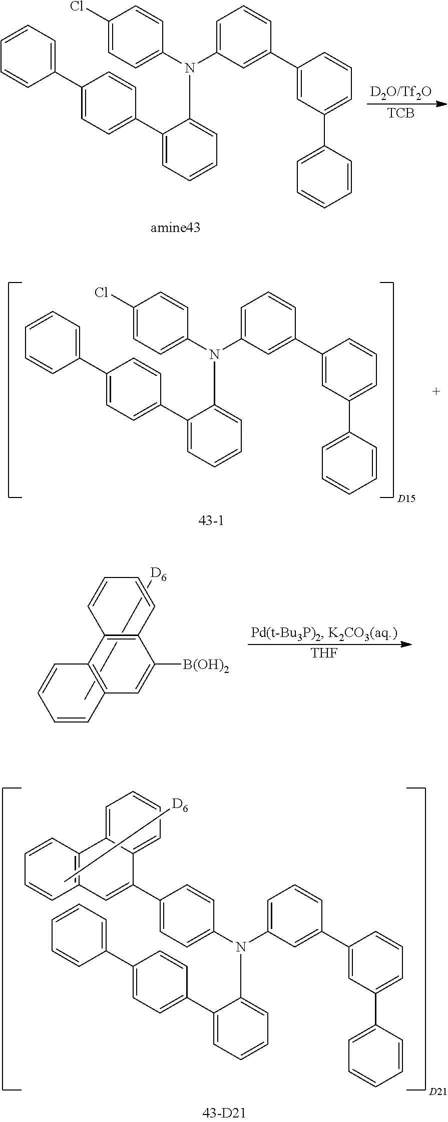

Compound amine43(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (29 g, 102.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.2 g of Compound 43-1. (Yield: 41%, MS: [M+H]+=600).

Compound 43-1(15 g, 25 mmol) and phenanthren-9-ylboronic acid_D6(6 g, 26.3 mmol) were added to 300 ml of THF, and the mixture was stirred and refluxed. Then, potassium carbonate (10.4 g, 75.1 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.5 g of Compound 43-D21. (Yield: 72%, MS: [M+H]+=748).

### Preparation Example 44

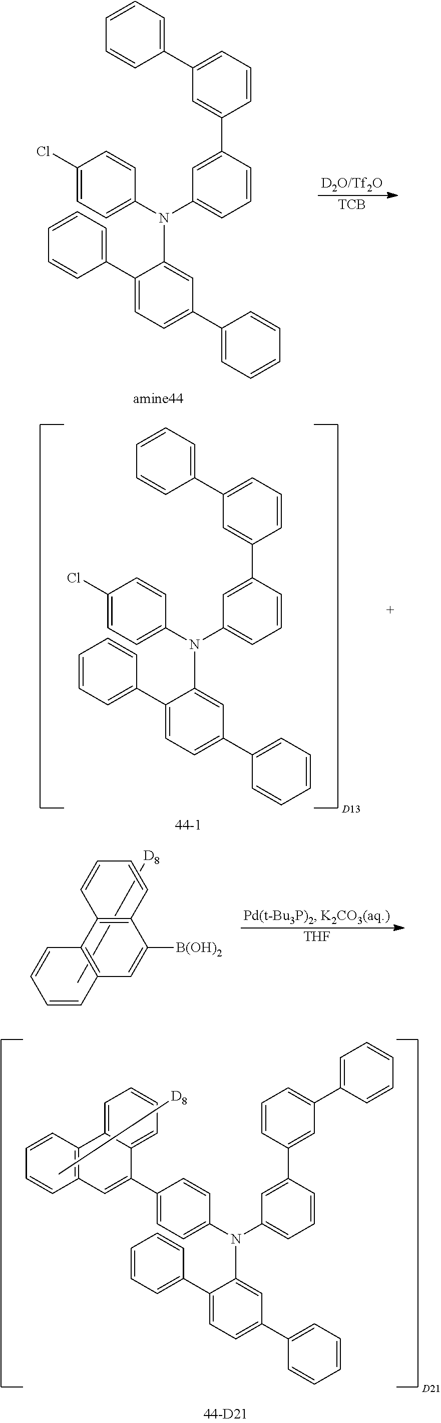

Compound amine44(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (29 g, 102.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.6 g of Compound 44-1. (Yield: 45%, MS: [M+H]+=598).

Compound 44-1(15 g, 25.1 mmol) and phenanthren-9-ylboronic acid_D8(6.1 g, 26.4 mmol) were added to 300 ml of THE, and the mixture was stirred and refluxed. Then, potassium carbonate (10.4 g, 75.4 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.3 g of Compound 44-D21. (Yield: 76%, MS: [M+H]+=748).

### Preparation Example 45

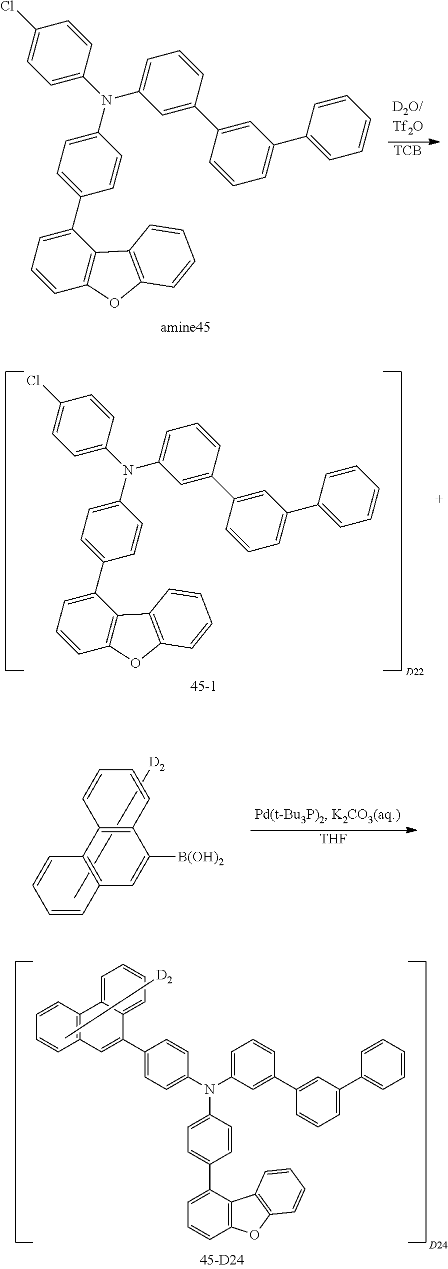

Compound amine45(10 g, 16.7 mmol) was added to 200 ml of 1,2,4-trichlorobenzene, and the mixture was stirred at room temperature. In another vessel, deuterium oxide (12.1 g, 601.9 mmol) was added to trifluoromethanesulfonic anhydride (42.5 g, 150.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.1 g of Compound 45-1. (Yield: 49%, MS: [M+H]+=621).

Compound 45-1(15 g, 24.2 mmol) and phenanthren-9-ylboronic acid_D2(5.7 g, 25.4 mmol) were added to 300 ml of THF, and the mixture was stirred and refluxed. Then, potassium carbonate (10 g, 72.6 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.2 g of Compound 45-D24. (Yield: 66%, MS: [M+H]+=765).

### Preparation Example 46

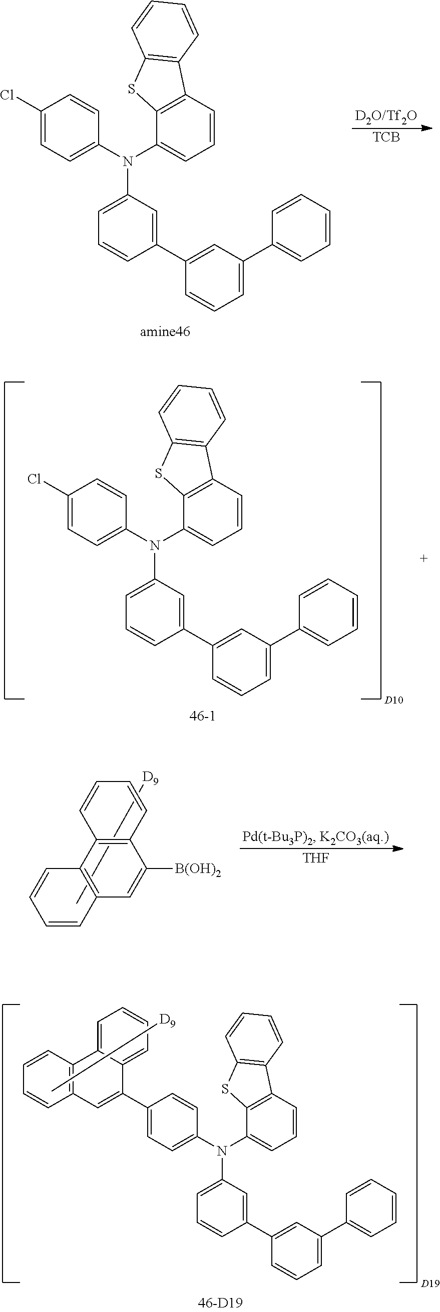

Compound amine46(10 g, 18.6 mmol) was added to 200 ml of 1,2,4-trichlorobenzene, and the mixture was stirred at room temperature. In another vessel, deuterium to oxide (13.4 g, 669 mmol) was added trifluoromethanesulfonic anhydride (31.5 g, 111.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 46-1. (Yield: 49%, MS: [M+H]+=549).

Compound 46-1(15 g, 27.4 mmol) and phenanthren-9-ylboronic acid_D9(6.6 g, 28.7 mmol) were added to 300 ml of THF, and the mixture was stirred and refluxed. Then, potassium carbonate (11.3 g, 82.1 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14 g of Compound 46-D19. (Yield: 73%, MS: [M+H]+=700).

### Preparation Example 47

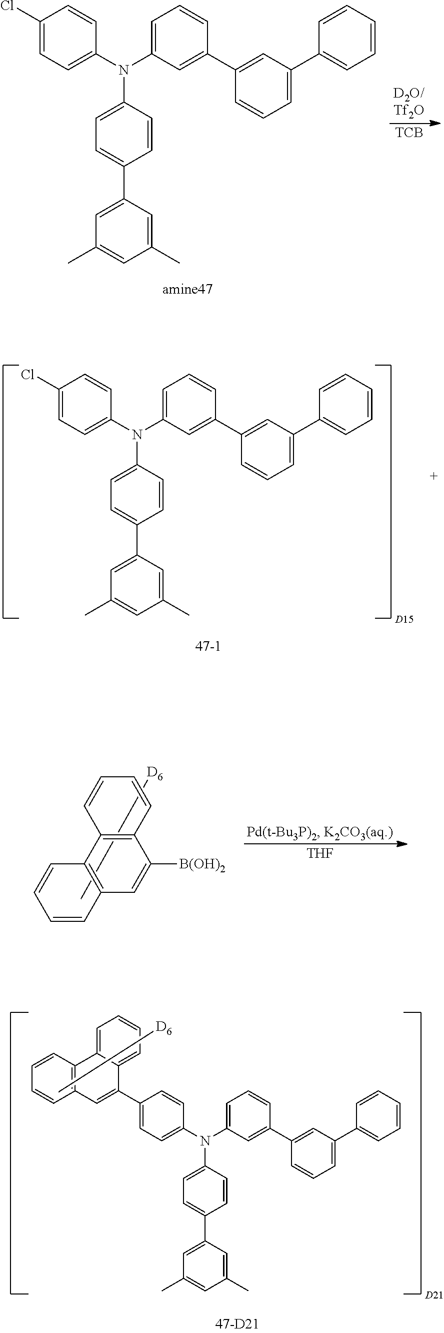

Compound amine47(10 g, 18.7 mmol) was added to 200 ml of 1,2,4-trichlorobenzene, and the mixture was stirred at room temperature. In another vessel, deuterium oxide (13.4 g, 671.5 mmol) was added t trifluoromethanesulfonic anhydride (31.6 g, 111.9 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.3 g of Compound 47-1. (Yield: 42%, MS: [M+H]+=552).

Compound 47-1(15 g, 27.2 mmol) and phenanthren-9-ylboronic acid_D6(6.5 g, 28.6 mmol) were added to 300 ml of THF, and the mixture was stirred and refluxed. Then, potassium carbonate (11.3 g, 81.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.3 g of Compound 47-D21. (Yield: 75%, MS: [M+H]+=700).

### Preparation Example 48

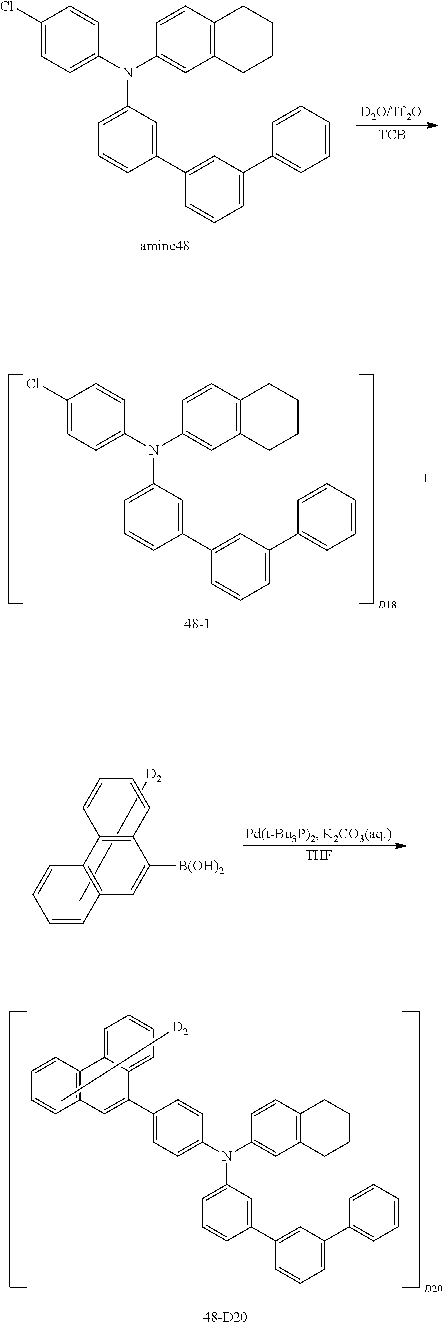

Compound amine48(10 g, 20.6 mmol) was added to 200 ml of 1,2,4-trichlorobenzene, and the mixture was stirred at room temperature. In another vessel, deuterium oxide (14.8 g, 740.6 mmol) was added to trifluoromethanesulfonic anhydride (52.2 g, 185.2 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.4 g of Compound 48-1. (Yield: 42%, MS: [M+H]+=505).

Compound 48-1(15 g, 29.8 mmol) and phenanthren-9-ylboronic acid_D2(7 g, 31.2 mmol) were added to 300 ml of THF, and the mixture was stirred and refluxed. Then, potassium carbonate (12.3 g, 89.3 mmol) was dissolved in 100 ml of water and added thereto, and the mixture was sufficiently stirred and then bis(tri-tert-butylphosphine)palladium(0) (0.2 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.6 g of Compound 48-D20. (Yield: 60%, MS: [M+H]+=649).

### Preparation Example 49

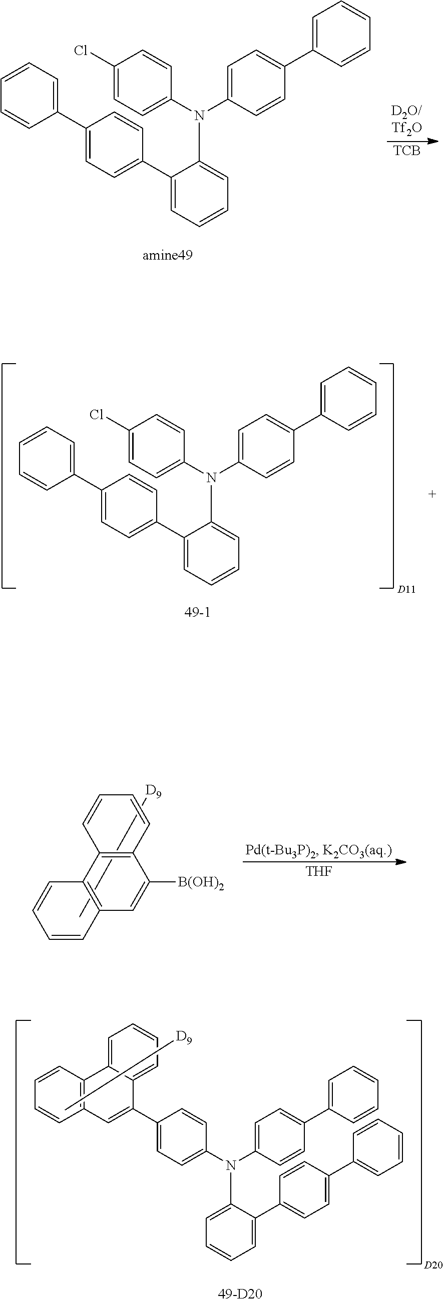

Compound amine49(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (33.3 g, 118.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.2 g of Compound 49-1. (Yield: 41%, MS: [M+H]+=520).

Compound 49-1(15 g, 28.9 mmol) and phenanthren-9-ylboronic acid_D9(7 g, 30.3 mmol) were added to 300 ml of THF, and the mixture was stirred and refluxed. Then, potassium carbonate (12 g, 86.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.2 g of Compound 49-D20. (Yield: 68%, MS: [M+H]+=671).

### Preparation Example 50

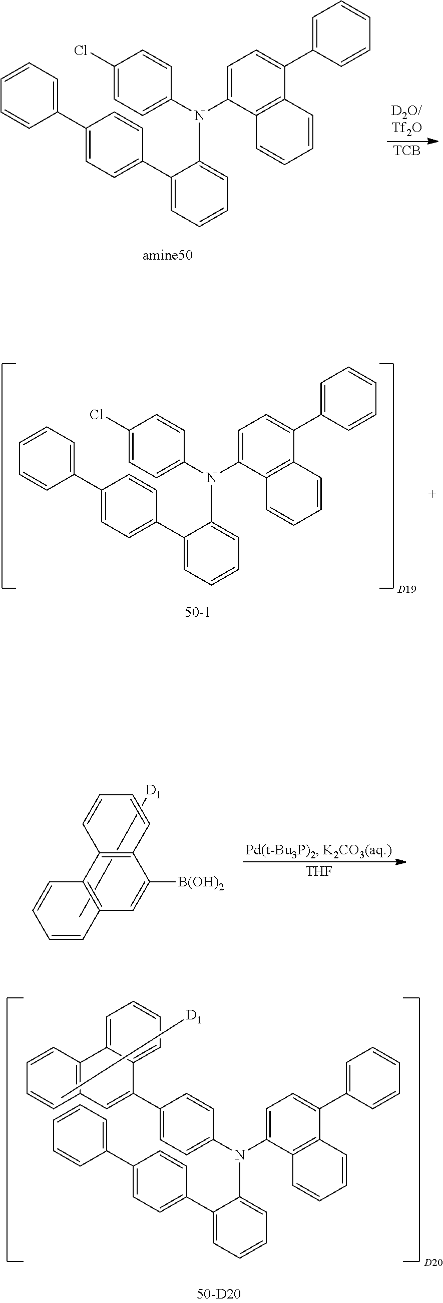

Compound amine50(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (45.5 g, 161.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.2 g of Compound 50-1. (Yield: 50%, MS: [M+H]+=578).

Compound 50-1(15 g, 26 mmol) and phenanthren-9-ylboronic acid_D1(6.1 g, 27.3 mmol) were added to 300 ml of THE, and the mixture was stirred and refluxed. Then, potassium carbonate (10.8 g, 78 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 15 g of Compound 50-D20. (Yield: 80%, MS: [M+H]+=721).

### Preparation Example 51

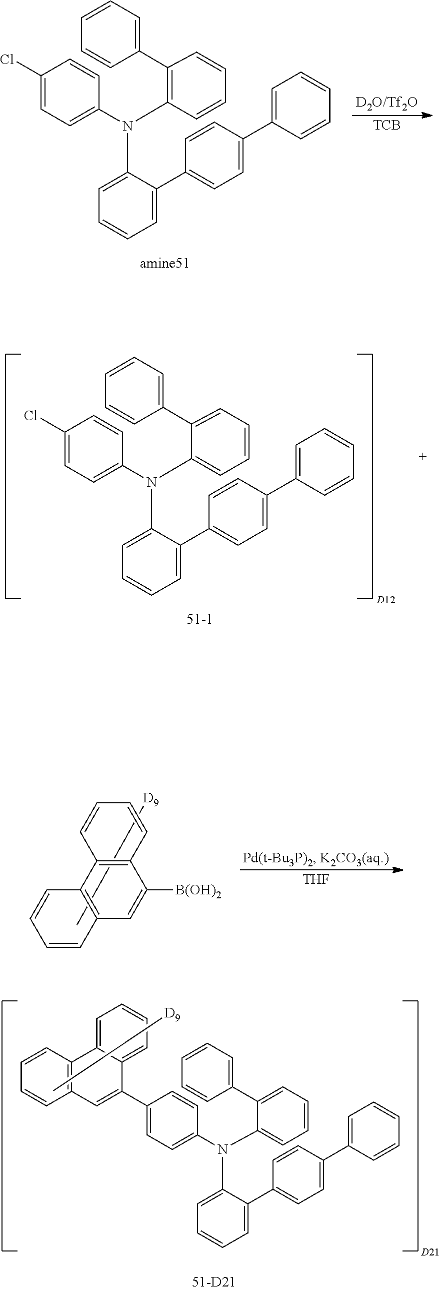

Compound amine51(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (33.3 g, 118.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.5 g of Compound 51-1. (Yield: 44%, MS: [M+H]+=521).

Compound 51-1(15 g, 28.8 mmol) and phenanthren-9-ylboronic acid_D9(7 g, 30.3 mmol) were added to 300 ml of THE, and the mixture was stirred and refluxed. Then, potassium carbonate (12 g, 86.5 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 15.5 g of Compound 51-D21. (Yield: 80%, MS: [M+H]+=672).

### Preparation Example 52

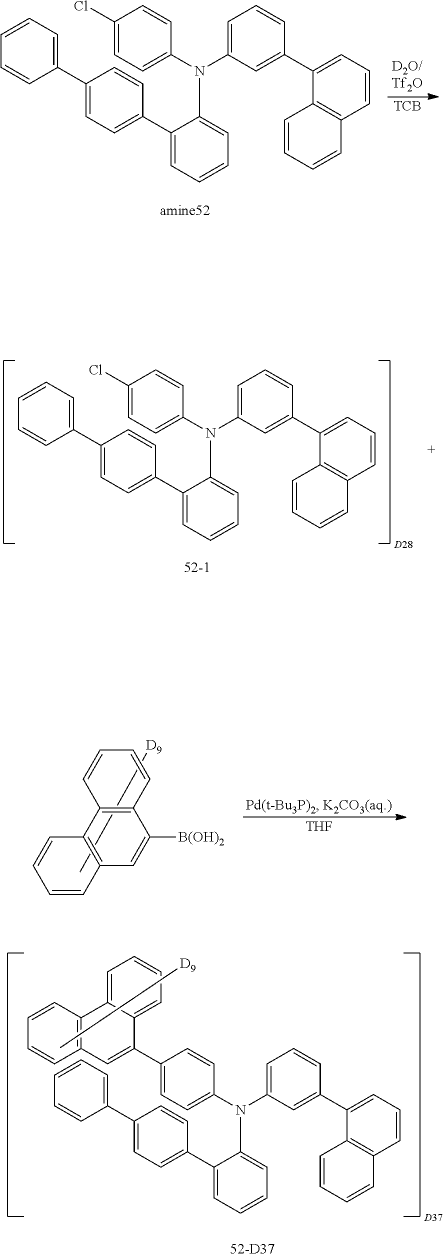

Compound amine52(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (60.7 g, 215 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 15 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.3 g of Compound 52-1. (Yield: 50%, MS: [M+H]+=587).

Compound 52-1(15 g, 25.6 mmol) and phenanthren-9-ylboronic acid_D9(6.2 g, 26.9 mmol) were added to 300 ml of THF, and the mixture was stirred and refluxed. Then, potassium carbonate (10.6 g, 76.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.9 g of Compound 52-D37. (Yield: 79%, MS: [M+H]+=738).

### Preparation Example 53

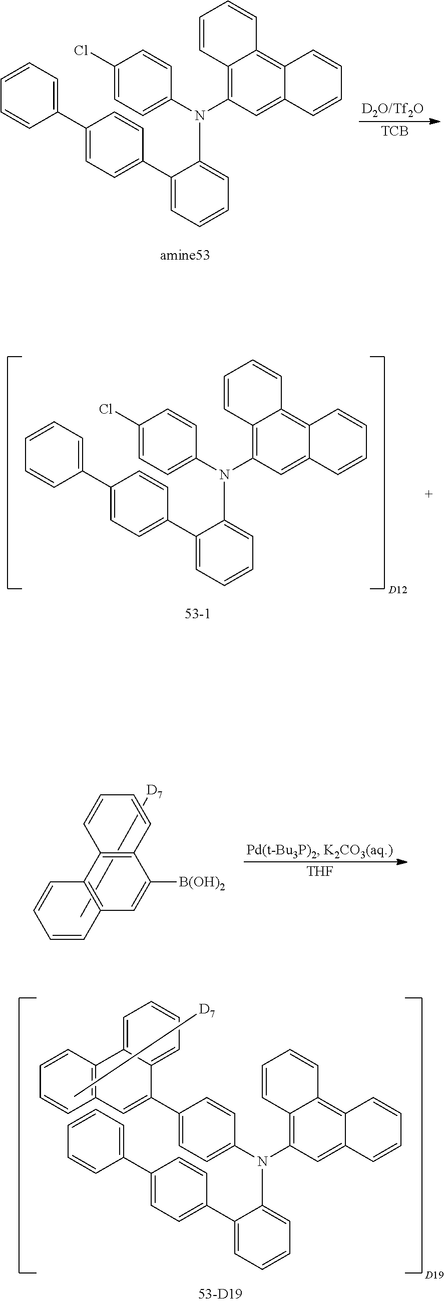

Compound amine53(10 g, 18.8 mmol) was added to 200 ml of 1,2,4-trichlorobenzene, and the mixture was stirred at room temperature. In another vessel, deuterium oxide (13.6 g, 676.6 mmol) was added to trifluoromethanesulfonic anhydride (31.8 g, 112.8 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.8 g of Compound 53-1. (Yield: 47%, MS: [M+H]+=545).

Compound 53-1(15 g, 27.6 mmol) and phenanthren-9-ylboronic acid_D7(6.6 g, 28.9 mmol) were added to 300 ml of THF, and the mixture was stirred and refluxed. Then, potassium carbonate (11.4 g, 82.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.1 g of Compound 53-D19. (Yield: 74%, MS: [M+H]+=694).

### Preparation Example 54

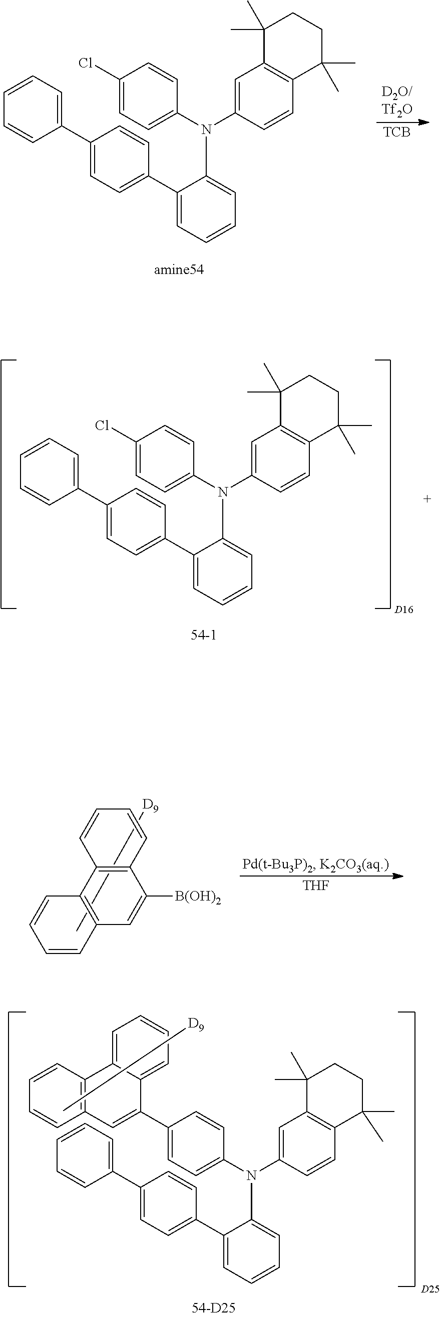

Compound amine54(10 g, 18.4 mmol) was added to 200 ml of 1,2,4-trichlorobenzene, and the mixture was stirred at room temperature. In another vessel, deuterium oxide (13.3 g, 664 mmol) was added to trifluoromethanesulfonic anhydride (31.2 g, 110.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.9 g of Compound 54-1. (Yield: 48%, MS: [M+H]+=559).

Compound 54-1(15 g, 26.9 mmol) and phenanthren-9-ylboronic acid_D9(6.5 g, 28.2 mmol) were added to 300 ml of THF, and the mixture was stirred and refluxed. Then, potassium carbonate (11.1 g, 80.6 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.1 g of Compound 54-D25. (Yield: 74%, MS: [M+H]+=710).

### Preparation Example 55

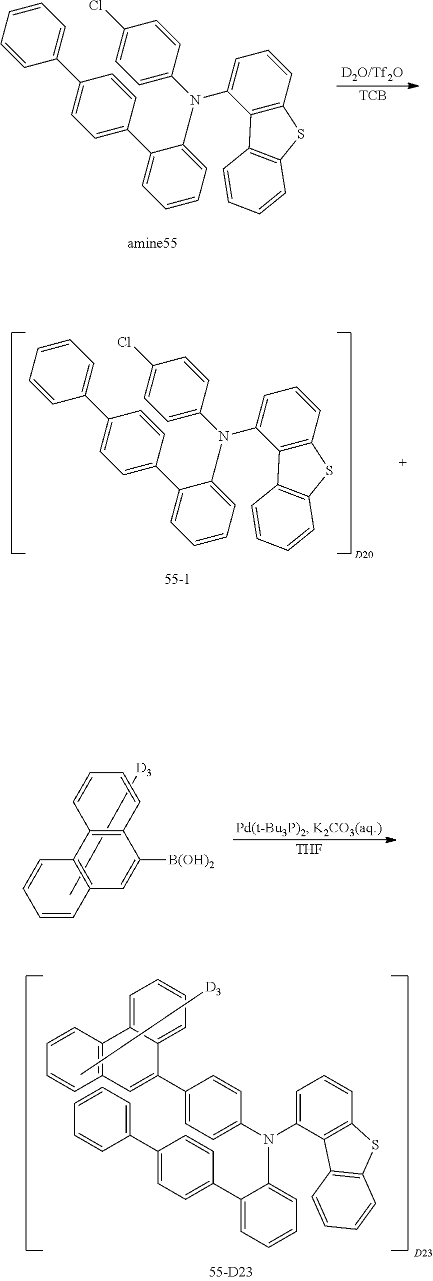

Compound amine55(10 g, 18.6 mmol) was added to 200 ml of 1,2,4-trichlorobenzene, and the mixture was stirred at room temperature. In another vessel, deuterium oxide (13.4 g, 669 mmol) was added to trifluoromethanesulfonic anhydride (47.2 g, 167.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.9 g of Compound 55-1. (Yield: 47%, MS: [M+H]+=559).

Compound 55-1(15 g, 26.9 mmol) and phenanthren-9-ylboronic acid_D3(6.4 g, 28.2 mmol) were added to 300 ml of THF, and the mixture was stirred and refluxed. Then, potassium carbonate (11.1 g, 80.6 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.3 g of Compound 55-D23. (Yield: 60%, MS: [M+H]+=704).

### Preparation Example 56

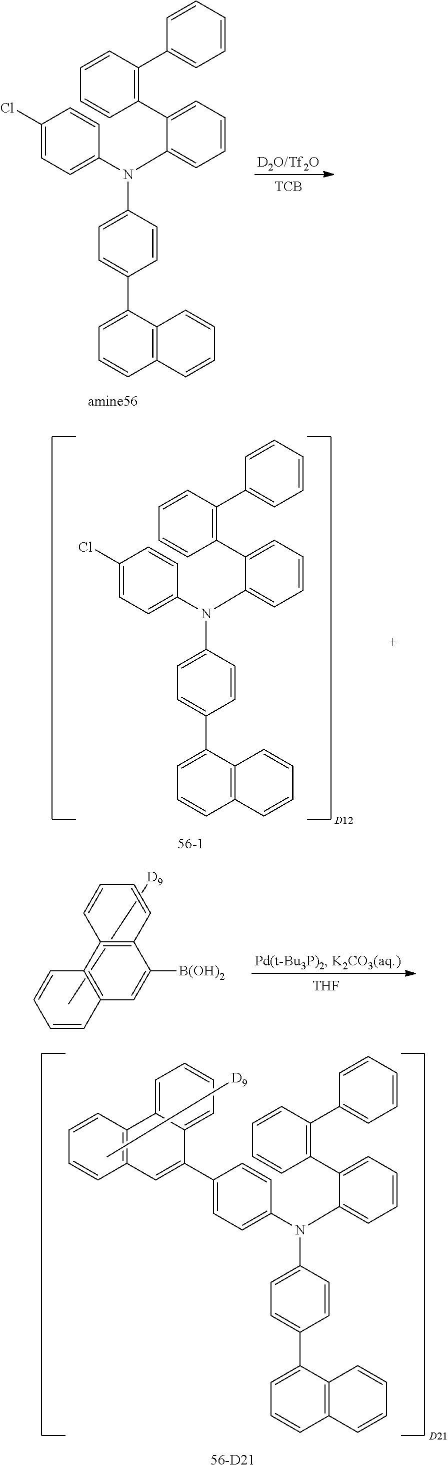

Compound amine56(10 g, 17.9 mmol) was added to 200 ml of 1,2,4-trichlorobenzene, and the mixture was stirred at room temperature. In another vessel, deuterium (12.9 oxide g, 645 mmol) was added to trifluoromethanesulfonic anhydride (30.3 g, 107.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.1 g of Compound 56-1. (Yield: 40%, MS: [M+H]+=571).

Compound 56-1(15 g, 26.3 mmol) and phenanthren-9-ylboronic acid_D9(6.4 g, 27.6 mmol) were added to 300 ml of THF, and the mixture was stirred and refluxed. Then, potassium carbonate (10.9 g, 78.9 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.4 g of Compound 56-D21. (Yield: 60%, MS: [M+H]+=722).

### Preparation Example 57

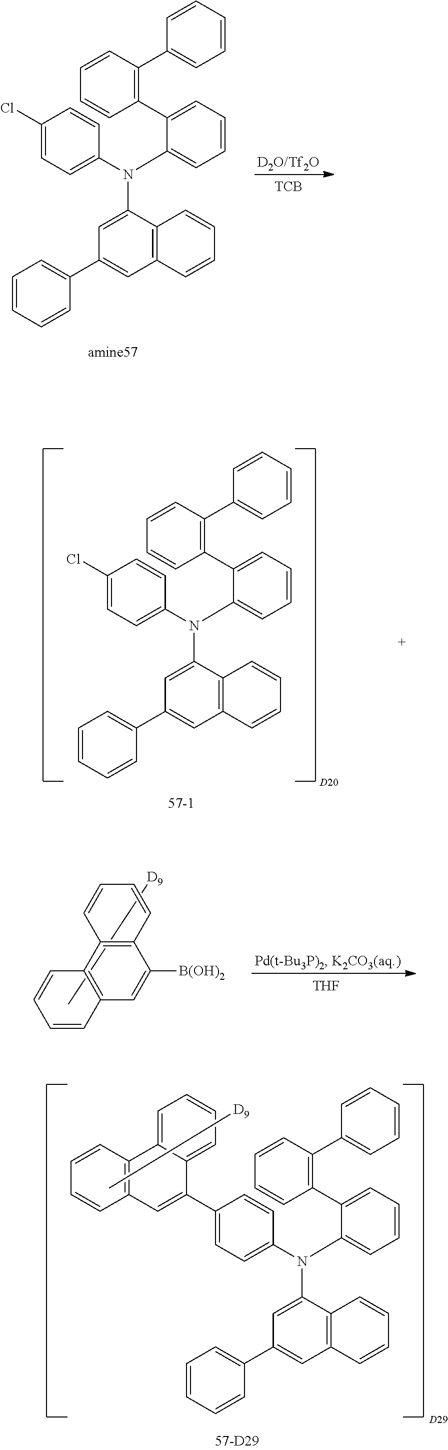

Compound amine57(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (45.5 g, 161.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.6 g of Compound 57-1. (Yield: 44%, MS: [M+H]+=579).

Compound 57-1(15 g, 25.9 mmol) and phenanthren-9-ylboronic acid_D9(6.3 g, 27.2 mmol) were added to 300 ml of THF, and the mixture was stirred and refluxed. Then, potassium carbonate (10.8 g, 77.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.7 g of Compound 57-D29. (Yield: 62%, MS: [M+H]+=730).

### Preparation Example 58

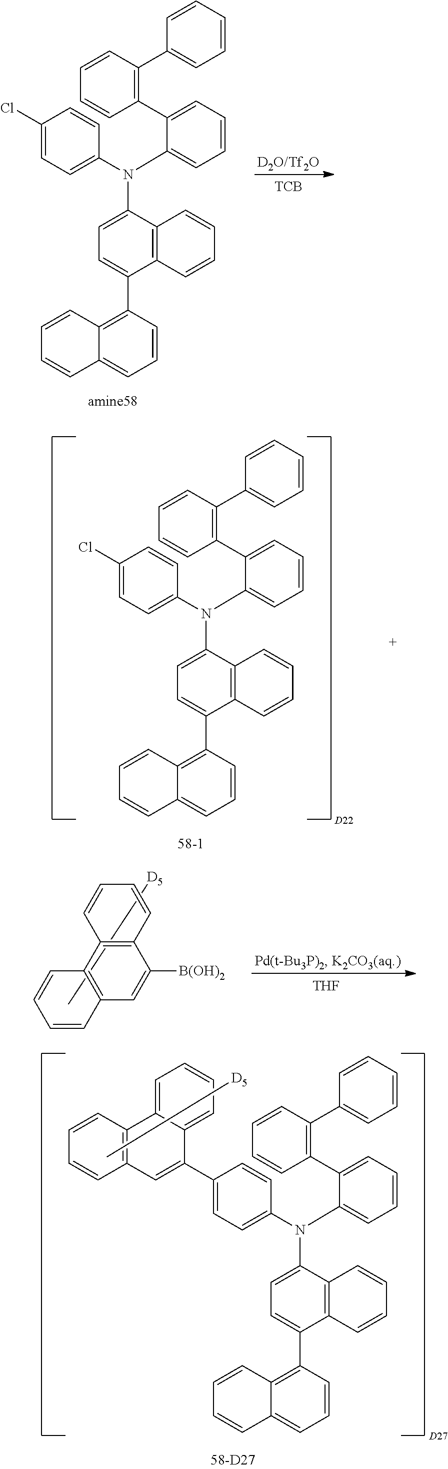

Compound amine58(10 g, 16.4 mmol) was added to 200 ml of 1,2,4-trichlorobenzene, and the mixture was stirred at room temperature. In another vessel, deuterium oxide (11.9 g, 591.9 mmol) was added to trifluoromethanesulfonic anhydride (41.8 g, 148 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.1 g of Compound 58-1. (Yield: 40%, MS: [M+H]+=631).

Compound 58-1(15 g, 23.8 mmol) and phenanthren-9-ylboronic acid_D5(5.7 g, 25 mmol) were added to 300 ml of THE, and the mixture was stirred and refluxed. Then, potassium carbonate (9.9 g, 71.4 mmol) was dissolved in 100 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.2 g of Compound 58-D27. (Yield: 77%, MS: [M+H]+=778).

### Preparation Example 59

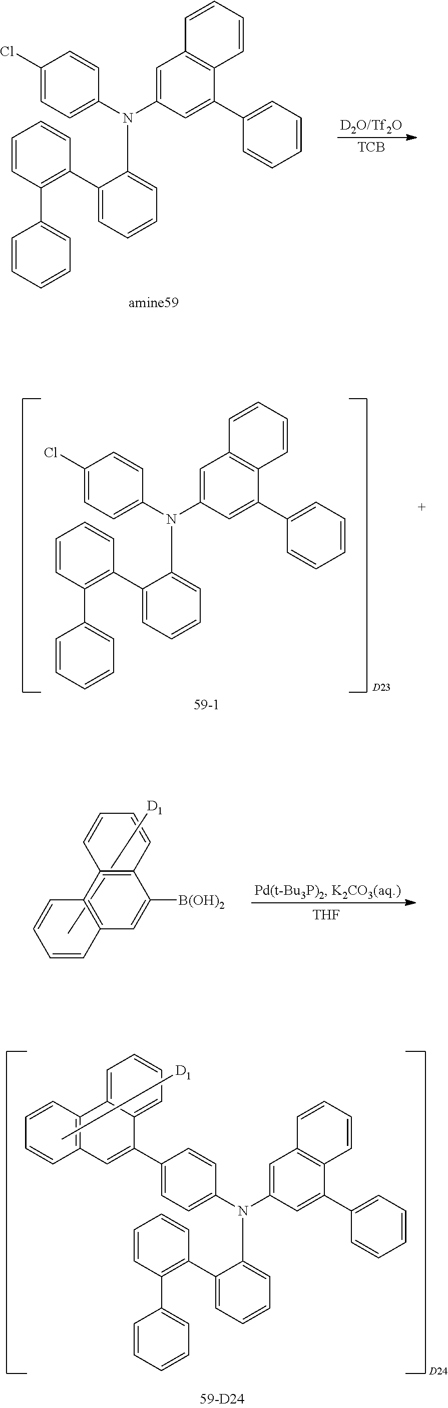

Compound amine59(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (45.5 g, 161.3 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.3 g of Compound 59-1. (Yield: 41%, MS: [M+H]+=582).

Compound 59-1(15 g, 25.8 mmol) and phenanthren-9-ylboronic acid_D1(6 g, 27.1 mmol) were added to 300 ml of THF, and the mixture was stirred and refluxed. Then, potassium carbonate (10.7 g, 77.4 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.2 g of Compound 59-D24. (Yield: 60%, MS: [M+H]+=725).

### Preparation Example 60

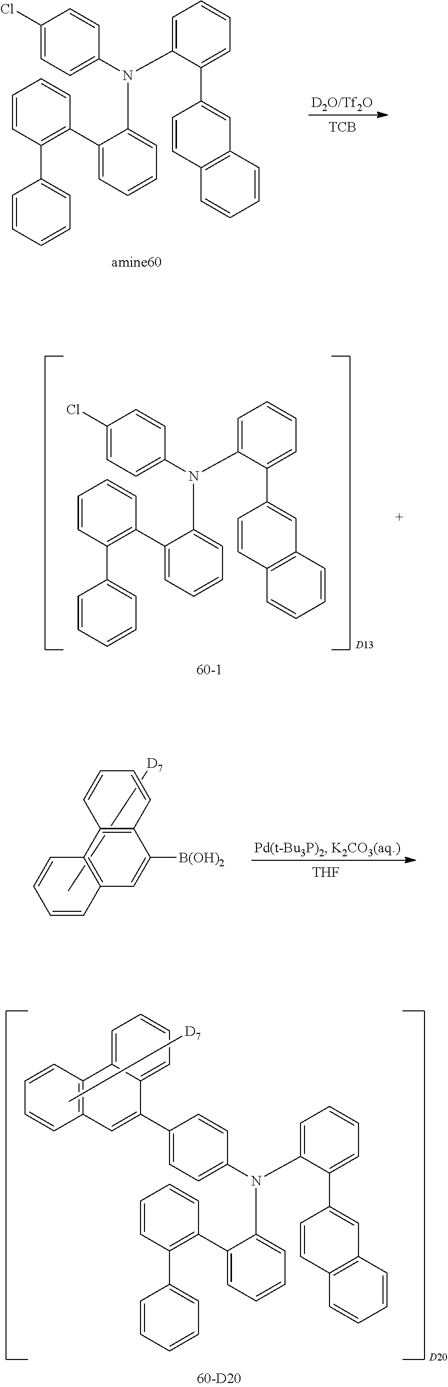

Compound amine60(10 g, 17.9 mmol) was added to 200 ml of 1,2,4-trichlorobenzene, and the mixture was stirred at room temperature. In another vessel, deuterium oxide (12.9 g, 645 mmol) was added to trifluoromethanesulfonic anhydride (30.3 g, 107.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.7 g of Compound 60-1. (Yield: 46%, MS: [M+H]+=572).

Compound 60-1(15 g, 26.3 mmol) and phenanthren-9-ylboronic acid_D7(6.3 g, 27.6 mmol) were added to 300 ml of THF, and the mixture was stirred and refluxed. Then, potassium carbonate (10.9 g, 78.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.2 g of Compound 60-D20. (Yield: 75%, MS: [M+H]+=721).

### Preparation Example 61

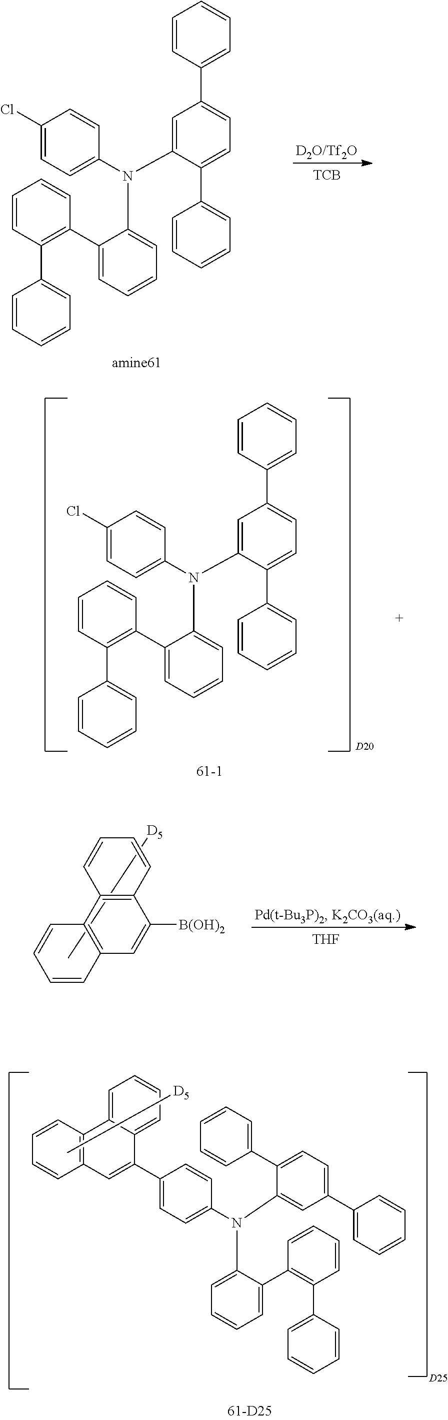

Compound amine61(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (43.5 g, 154.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.2 g of Compound 61-1. (Yield: 41%, MS: [M+H]+=605).

Compound 61-1(15 g, 24.8 mmol) and phenanthren-9-ylboronic acid_D5(5.9 g, 26.1 mmol) were added to 300 ml of THF, and the mixture was stirred and refluxed. Then, potassium carbonate (10.3 g, 74.5 mmol) was dissolved in 100 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 11.6 g of Compound 61-D25. (Yield: 62%, MS: [M+H]+=752).

### Preparation Example 62

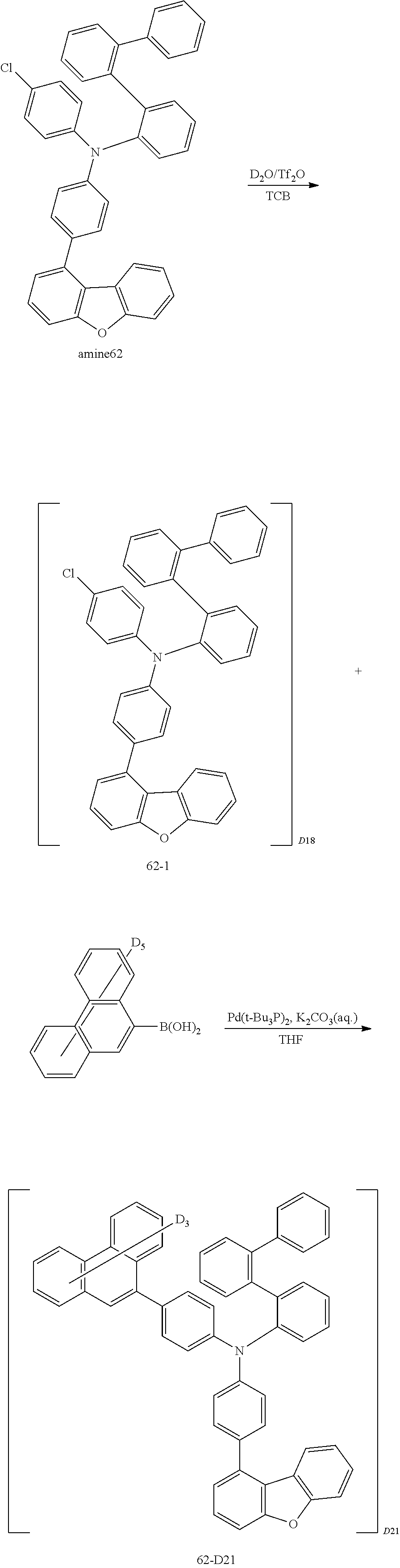

Compound amine62(10 g, 16.7 mmol) was added to 200 ml of 1,2,4-trichlorobenzene, and the mixture was stirred at room temperature. In another vessel, deuterium oxide (12.1 g, 601.9 mmol) was added to trifluoromethanesulfonic anhydride (42.5 g, 150.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.6 g of Compound 62-1. (Yield: 45%, MS: [M+H]+=617).

Compound 62-1(15 g, 24.3 mmol) and phenanthren-9-ylboronic acid_D3(5.8 g, 25.6 mmol) were added to 300 ml of THF, and the mixture was stirred and refluxed. Then, potassium carbonate (10.1 g, 73 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.2 g of Compound 62-D21. (Yield: 71%, MS: [M+H]+=762).

### Preparation Example 63

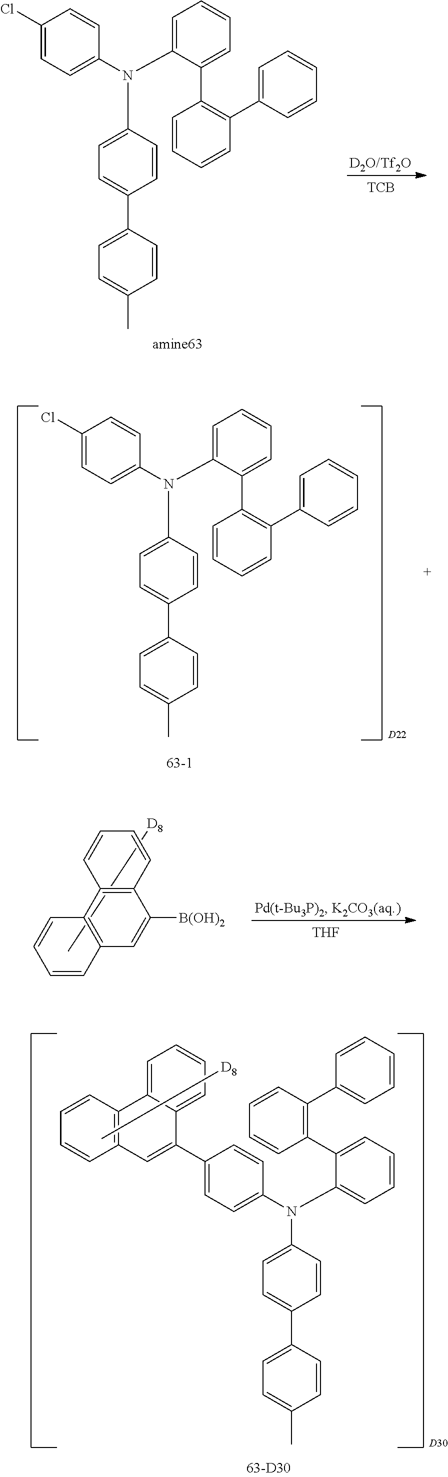

Compound amine63(10 g, 19.2 mmol) was added to 200 ml of 1,2,4-trichlorobenzene, and the mixture was stirred at room temperature. In another vessel, deuterium oxide (13.8 g, 689.5 mmol) was added to trifluoromethanesulfonic anhydride (48.6 g, 172.4 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.8 g of Compound 63-1. (Yield: 46%, MS: [M+H]+=545).

Compound 63-1(15 g, 27.6 mmol) and phenanthren-9-ylboronic acid_D8(6.7 g, 28.9 mmol) were added to 300 ml of THF, and the mixture was stirred and refluxed. Then, potassium carbonate (11.4 g, 82.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.2 g of Compound 63-D30. (Yield: 69%, MS: [M+H]+=695).

### Preparation Example 64

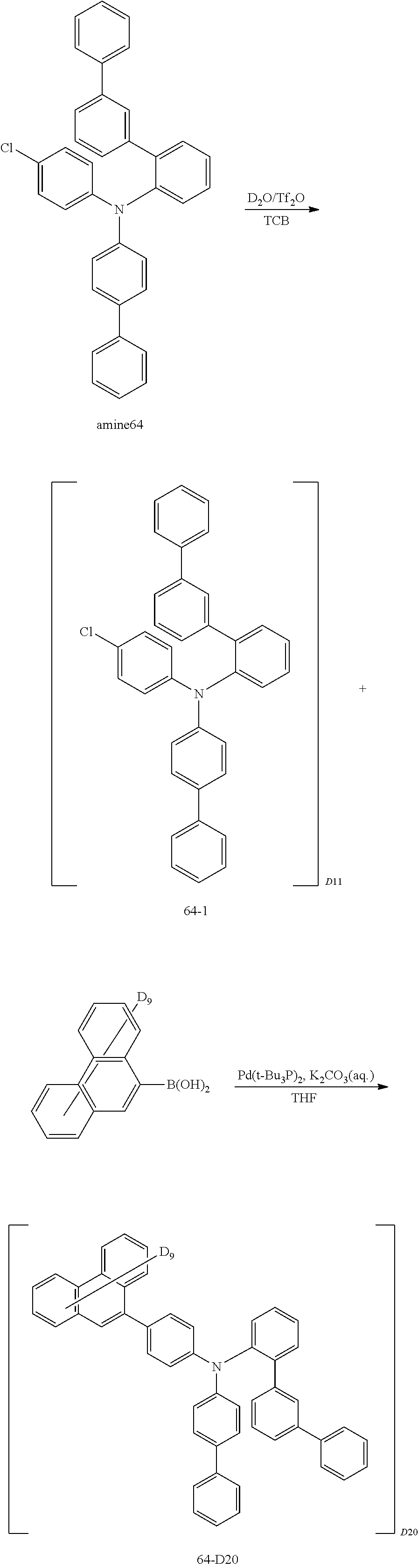

Compound amine64(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (33.3 g, 118.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.8 g of Compound 64-1. (Yield: 47%, MS: [M+H]+=520).

Compound 64-1(15 g, 28.9 mmol) and phenanthren-9-ylboronic acid_D9(7 g, 30.3 mmol) were added to 300 ml of THE, and the mixture was stirred and refluxed. Then, potassium carbonate (12 g, 86.7 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.1 g of Compound 64-D20. (Yield: 73%, MS: [M+H]+=671).

### Preparation Example 65

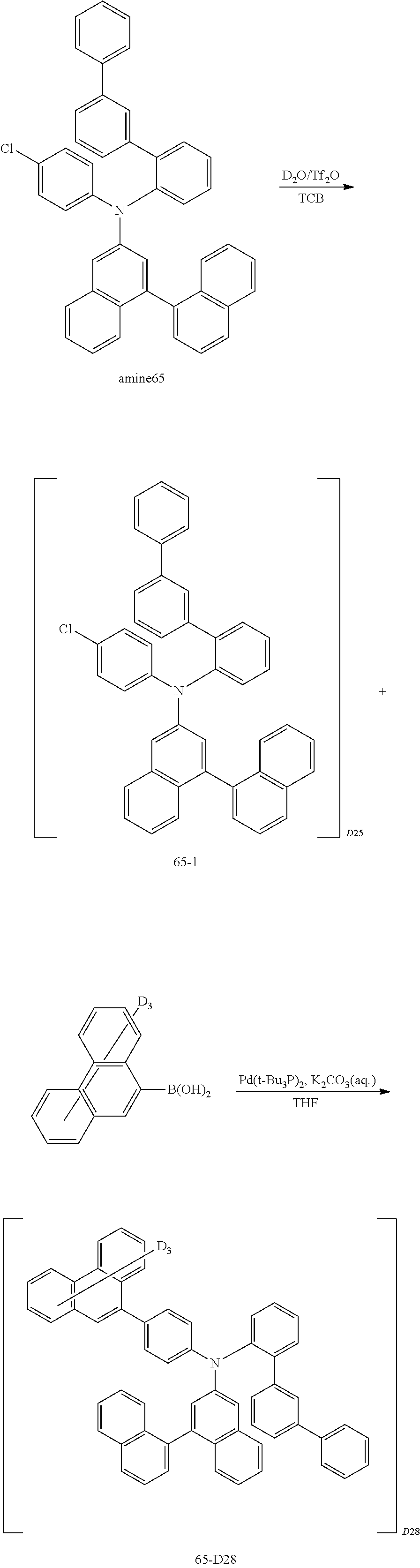

Compound amine65(10 g, 16.4 mmol) was added to 200 ml of 1,2,4-trichlorobenzene, and the mixture was stirred at room temperature. In another vessel, deuterium oxide (11.9 g, 591.9 mmol) was added to trifluoromethanesulfonic anhydride (41.8 g, 148 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.4 g of Compound 65-1. (Yield: 42%, MS: [M+H]+=634).

Compound 65-1(15 g, 23.7 mmol) and phenanthren-9-ylboronic acid_D3(5.6 g, 24.9 mmol) were added to 300 ml of THF, and the mixture was stirred and refluxed. Then, potassium carbonate (9.8 g, 71.1 mmol) was dissolved in 100 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.6 g of Compound 65-D28. (Yield: 79%, MS: [M+H]+=779).

### Preparation Example 66

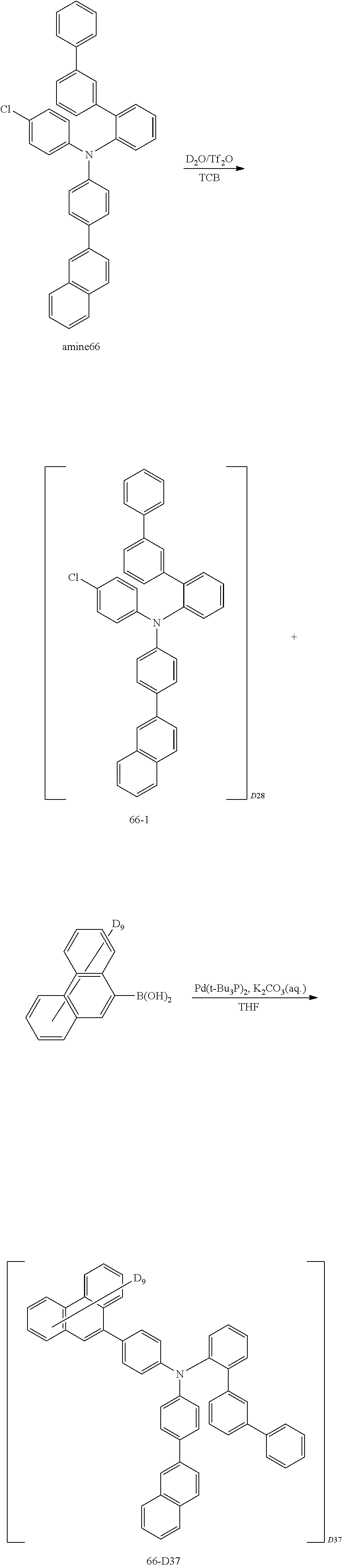

Compound amine66(10 g, 17.9 mmol) was added to 200 ml of 1,2,4-trichlorobenzene, and the mixture was stirred at room temperature. In another vessel, deuterium oxide (12.9 to 9 g, 645 mmol) was added trifluoromethanesulfonic anhydride (60.7 g, 215 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 15 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.5 g of Compound 66-1. (Yield: 43%, MS: [M+H]+=587).

Compound 66-1(15 g, 25.6 mmol) and phenanthren-9-ylboronic acid_D9(6.2 g, 26.9 mmol) were added to 300 ml of THF, and the mixture was stirred and refluxed. Then, potassium carbonate (10.6 g, 76.8 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.2 g of Compound 66-D37. (Yield: 70%, MS: [M+H]+=738).

### Preparation Example 67

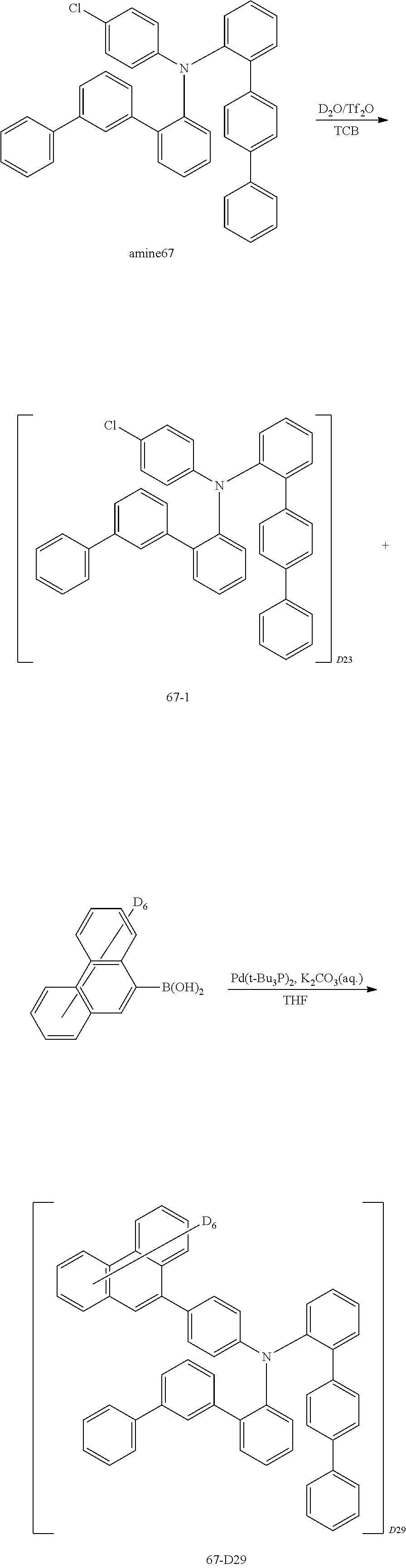

Compound amine67(10 g, 17.1 mmol) was added to 200 ml of 1,2,4-trichlorobenzene, and the mixture was stirred at room temperature. In another vessel, deuterium oxide (12.3 g, 616.3 mmol) was added to trifluoromethanesulfonic anhydride (43.5 g, 154.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.7 g of Compound 67-1. (Yield: 45%, MS: [M+H]+=608).

Compound 67-1(15 g, 24.7 mmol) and phenanthren-9-ylboronic acid_D6(5.9 g, 25.9 mmol) were added to 300 ml of THF, and the mixture was stirred and refluxed. Then, potassium carbonate (10.2 g, 74.1 mmol) was dissolved in 100 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12.9 g of Compound 67-D29. (Yield: 69%, MS: [M+H]+=756).

### Preparation Example 68

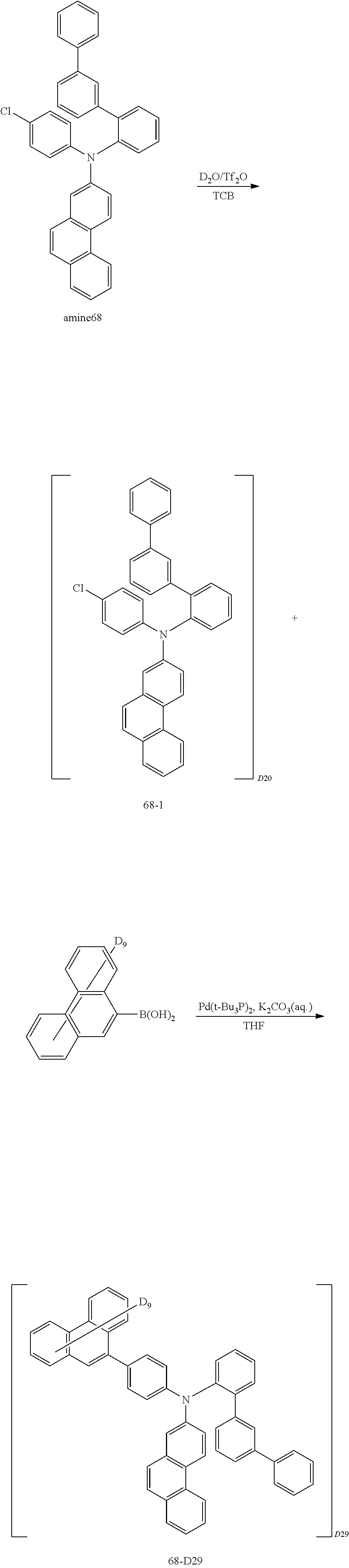

Compound amine68(10 g, 18.8 mmol) was added to 200 ml of 1,2,4-trichlorobenzene, and the mixture was stirred at room temperature. In another vessel, deuterium oxide (13.6 g, 676.6 mmol) was added to trifluoromethanesulfonic anhydride (47.7 g, 169.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.5 g of Compound 68-1. (Yield: 43%, MS: [M+H]+=553).

Compound 68-1(15 g, 27.2 mmol) and phenanthren-9-ylboronic acid_D9(6.6 g, 28.5 mmol) were added to 300 ml of THF, and the mixture was stirred and refluxed. Then, potassium carbonate (11.3 g, 81.5 mmol) was dissolved in 100 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 13.2 g of Compound 68-D29. (Yield: 69%, MS: [M+H]+=704).

### Preparation Example 69

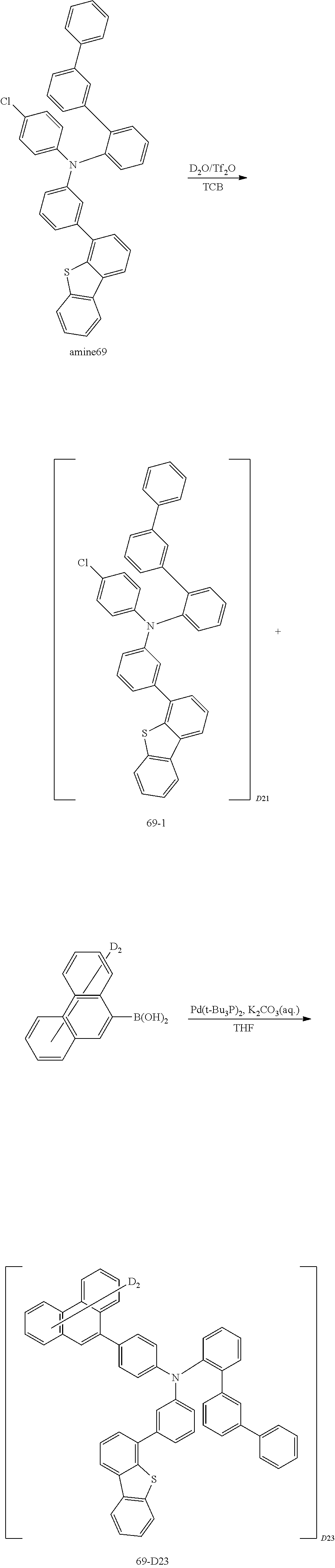

Compound amine69(10 g, 16.3 mmol) was added to 200 ml of 1,2,4-trichlorobenzene, and the mixture was stirred at room temperature. In another vessel, deuterium oxide (11.7 g, 586.1 mmol) was added to trifluoromethanesulfonic anhydride (41.3 g, 146.5 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.3 g of Compound 69-1. (Yield: 42%, MS: [M+H]+=636).

Compound 69-1(15 g, 23.6 mmol) and phenanthren-9-ylboronic acid_D2(5.6 g, 24.8 mmol) were added to 300 ml of THF, and the mixture was stirred and refluxed. Then, potassium carbonate (9.8 g, 70.8 mmol) was dissolved in 100 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 12 g of Compound 69-D23. (Yield: 65%, MS: [M+H]+=780).

### Preparation Example 70

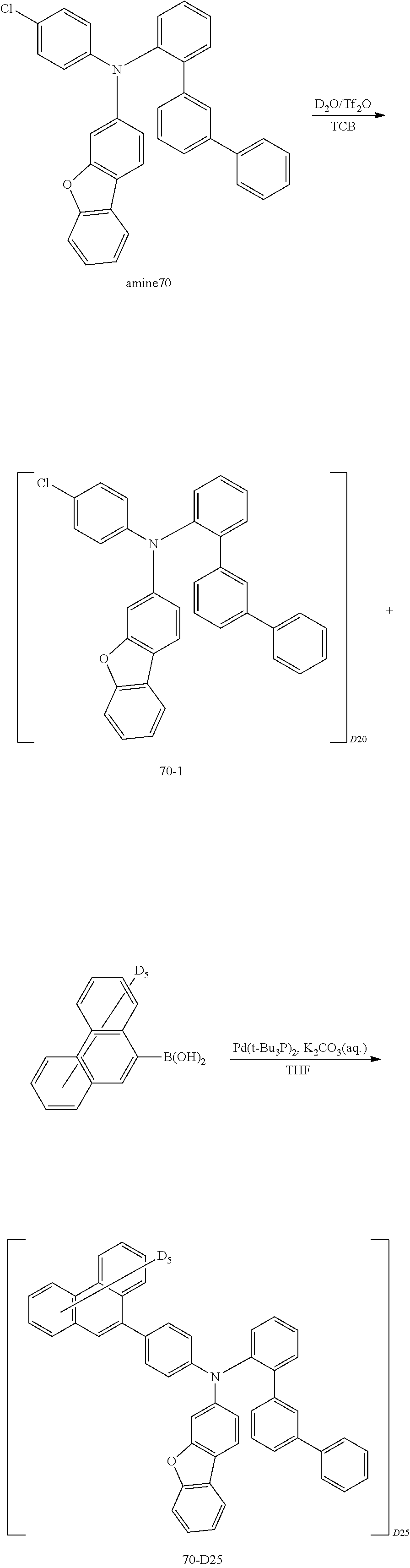

Compound amine70(10 g, 19.2 mmol) was added to 200 ml of 1,2,4-trichlorobenzene, and the mixture was stirred at room temperature. In another vessel, deuterium oxide (13.8 g, 689.6 mmol) was added to trifluoromethanesulfonic anhydride (48.6 g, 172.4 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.1 g of Compound 70-1. (Yield: 40%, MS: [M+H]+=541).

Compound 70-1(15 g, 27.7 mmol) and phenanthren-9-ylboronic acid_D5(6.6 g, 29.1 mmol) were added to 300 ml of THF, and the mixture was stirred and refluxed. Then, potassium carbonate (11.5 g, 83 mmol) was dissolved in 100 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.1 g of Compound 70-D25. (Yield: 74%, MS: [M+H]+=690).

### Preparation Example 71

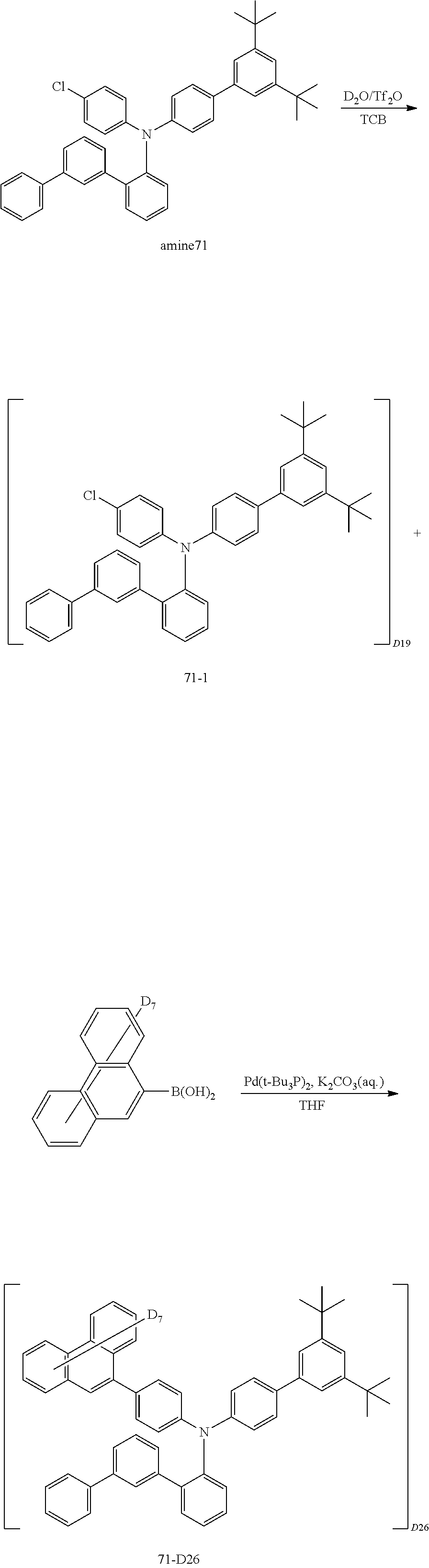

Compound amine71(10 g, 16.1 mmol) was added to 200 ml of 1,2,4-trichlorobenzene, and the mixture was stirred at room temperature. In another vessel, deuterium oxide (11.6 g, 580.4 mmol) was added to trifluoromethanesulfonic anhydride (27.3 g, 96.7 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.8 g of Compound 71-1. (Yield: 47%, MS: [M+H]+=640).

Compound 71-1(15 g, 23.5 mmol) and phenanthren-9-ylboronic acid_D7(5.6 g, 24.6 mmol) were added to 300 ml of THF, and the mixture was stirred and refluxed. Then, potassium carbonate (9.7 g, 70.4 mmol) was dissolved in 100 ml of water and added thereto, and the mixture was sufficiently stirred and then bis(tri-tert-butylphosphine)palladium(0) (0.1 g, 0.2 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.2 g of Compound 71-D26. (Yield: 77%, MS: [M+H]+=789).

### Preparation Example 72

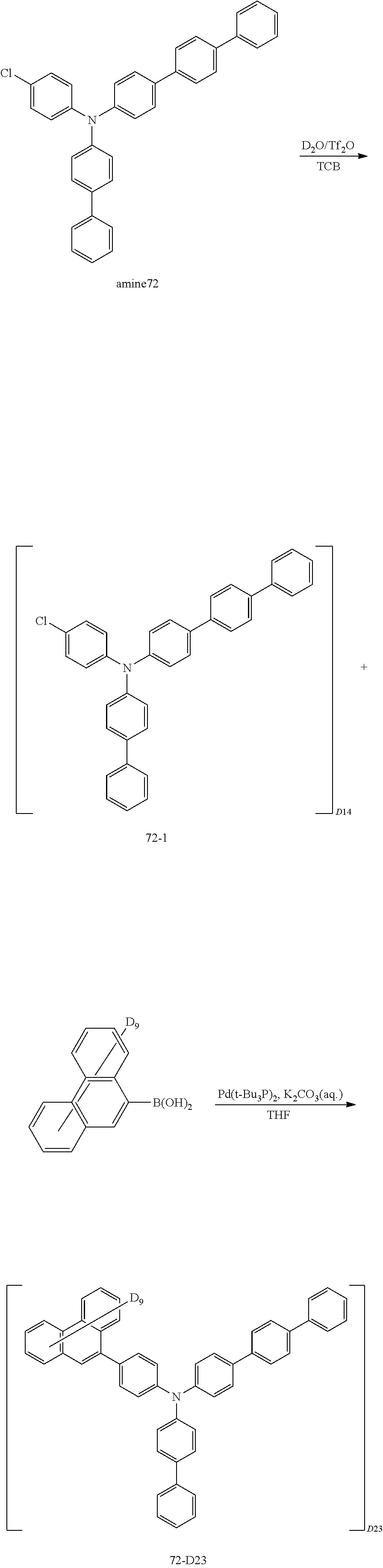

Compound amine72(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (33.3 g, 118.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 4.2 g of Compound 72-1. (Yield: 41%, MS: [M+H]+=523).

Compound 72-1(15 g, 28.7 mmol) and phenanthren-9-ylboronic acid_D9(7 g, 30.2 mmol) were added to 300 ml of THE, and the mixture was stirred and refluxed. Then, potassium carbonate (11.9 g, 86.2 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.9 g of Compound 72-D23. (Yield: 77%, MS: [M+H]+=674).

### Preparation Example 73

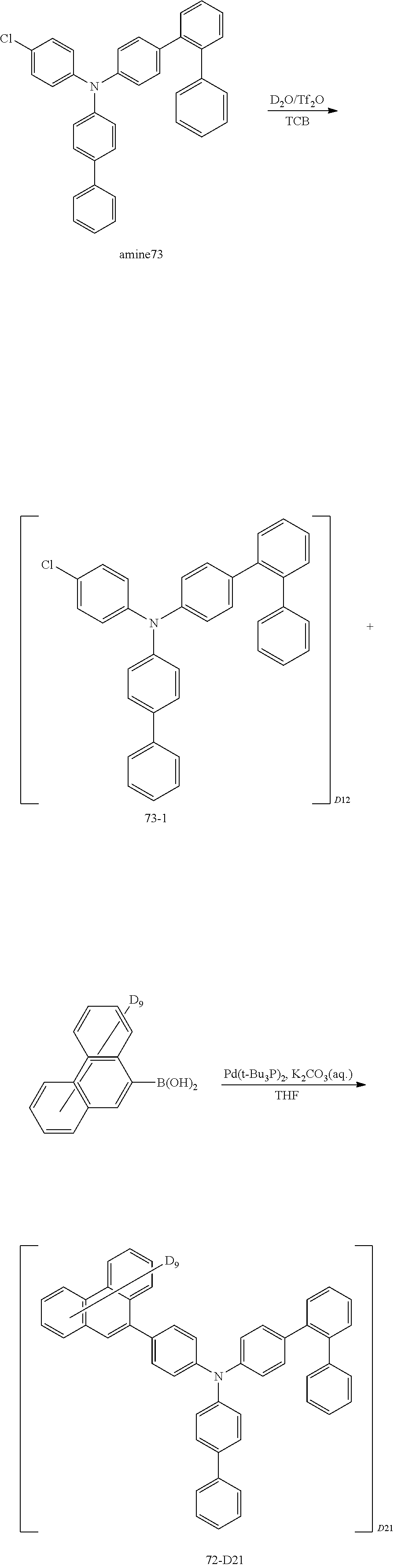

Compound amine73(10 g, 19.7 mmol) was added to 200 ml of 1,2,4-trichlorobenzene, and the mixture was stirred at room temperature. In another vessel, deuterium oxide (14.2 g, 708.6 mmol) was added to trifluoromethanesulfonic anhydride (33.3 g, 118.1 mmol) at 0 °C, and the mixture was stirred for 10 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140 °C and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5 g of Compound 73-1. (Yield: 49%, MS: [M+H]+=521).

Compound 73-1(15 g, 28.8 mmol) and phenanthren-9-ylboronic acid_D9(7 g, 30.3 mmol) were added to 300 ml of THF, and the mixture was stirred and refluxed. Then, potassium carbonate (12 g, 86.5 mmol) was dissolved in 100 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, the organic layer and the aqueous layer were separated, and the organic layer was distilled. This was again dissolved in chloroform, washed twice with water, and then the organic layer was separated, anhydrous magnesium sulfate was added thereto, stirred, then filtered, and the filtrate was distilled under reduced pressure. The concentrated compound was purified by a silica gel column chromatography to prepare 14.5 g of Compound 73-D21. (Yield: 75%, MS: [M+H]+=672).

### Example 1-1

A glass substrate on which ITO (indium tin oxide) was coated as a thin film to a thickness of 1,000 Å was put into distilled water in which a detergent was dissolved, and ultrasonically cleaned. A product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the ITO transparent electrode thus prepared, the following compound HI-1 was formed in a thickness of 1150 Å as a hole injection layer, but the following compound A-1was p-doped at a concentration of 1.5 wt. %. The following compound HT-1 was vacuum deposited on the hole injection layer to form a hole transport layer with a layer thickness of 800 Å. Then, the following compound 1-D22 was thermally vacuum deposited as the hole transport auxiliary layer to a layer thickness of 150 Å. Then, the following compound BH-1 and compound BD-1 were vacuum deposited in a weight ratio of 25:1 as a light emitting layer to a thickness of 250 Å. Next, the following compound HB-1 was vacuum deposited to a thickness of 50 Å as a hole blocking layer. Next, the following compound ET-1 and the following compound LiQ were thermally vacuum deposited at a weight ratio of 1:1 to a thickness of 310 Å as a layer that simultaneously performs electron transport and electron injection. Lithium fluoride(LiF) and aluminum were sequentially deposited to have a thickness of 12 Å and 1,000 Å, respectively, on the electron transport and injection layer, thereby forming a cathode, thereby manufacturing an organic light emitting device.

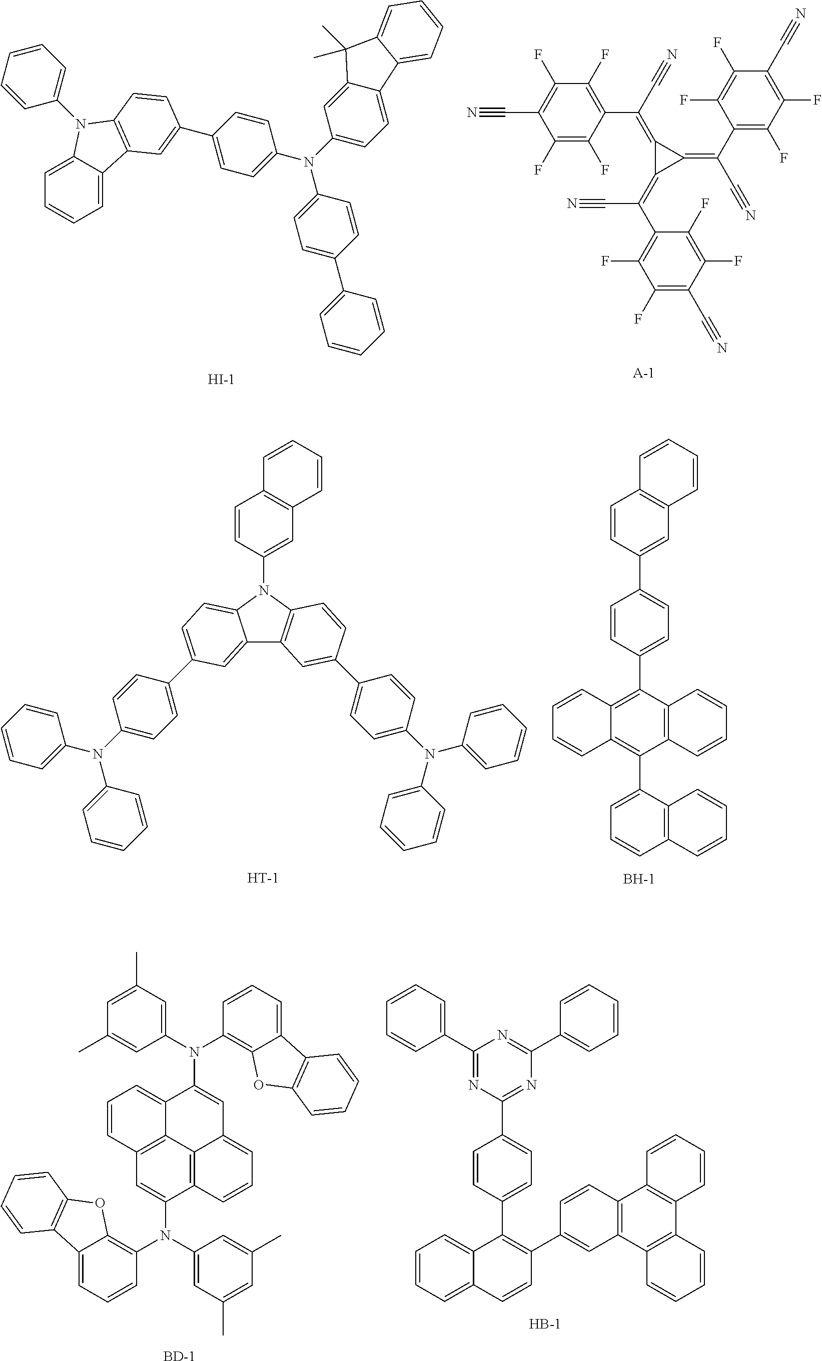

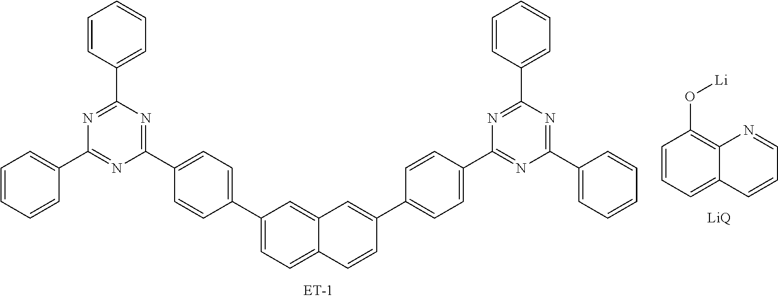

### Examples 1-2 to 1-73

The organic light emitting devices of Examples 1-2 to 1-73 were manufactured in the same manner as in Example 1-1, except that the compounds listed in Tables 1 and 2 below were used instead of Compound 1-D22.

### Comparative Examples 1-1 to 1-35

The organic light emitting devices of Comparative Examples 1-1 to 1-35 were manufactured in the same manner as in Example 1-1, except that the compounds listed in Table 3 below were used instead of Compound 1-D22. The compounds used in Comparative Examples 1-17 to 1-35 in Table 3 below are as follows.

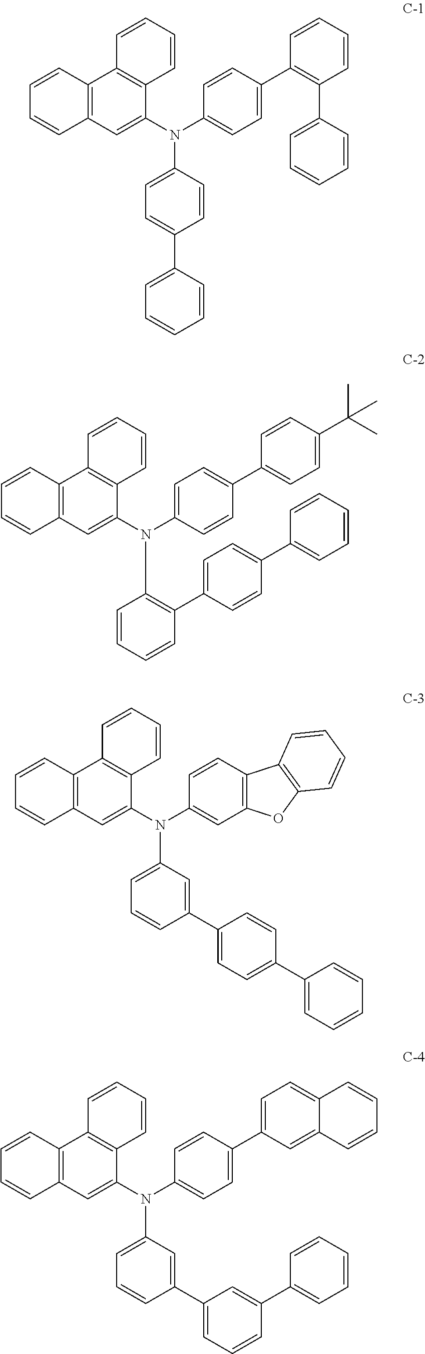 [SMILES: c1ccc(-c2ccc(N(c3ccc(-c4ccccc4-c4ccccc4)cc3)c3cc4ccccc4c4ccccc34)cc2)cc1 ; CC(C)(C)c1ccc(-c2ccc(N(c3ccccc3-c3ccc(-c4ccccc4)cc3)c3cc4ccccc4c4ccccc34)cc2)cc1 ; c1ccc(-c2ccc(-c3cccc(N(c4ccc5c(c4)oc4ccccc45)c4cc5ccccc5c5ccccc45)c3)cc2)cc1 ; c1ccc(-c2cccc(-c3cccc(N(c4ccc(-c5ccc6ccccc6c5)cc4)c4cc5ccccc5c5ccccc45)c3)c2)cc1]

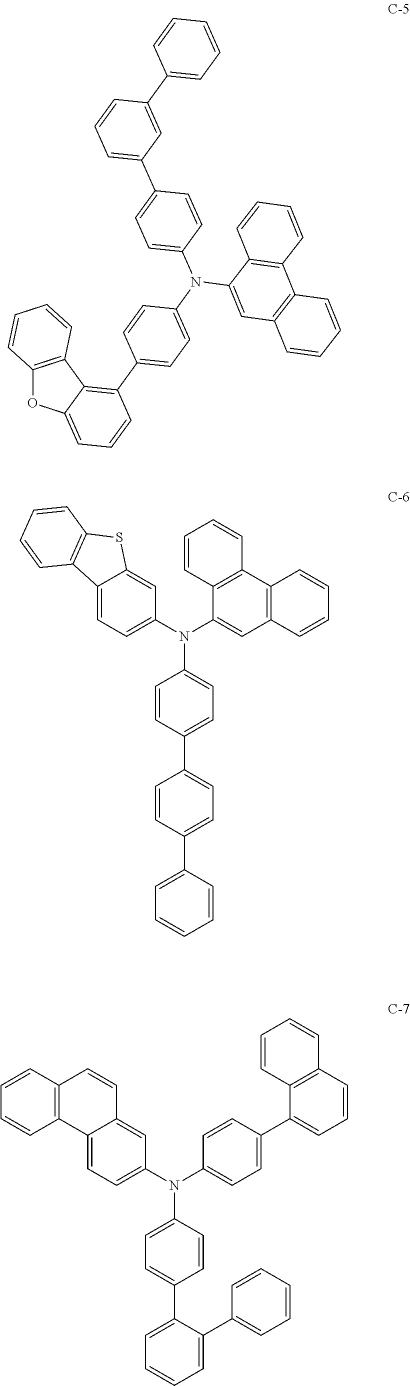 [SMILES: c1ccc(-c2cccc(-c3ccc(N(c4ccc(-c5cccc6oc7ccccc7c56)cc4)c4cc5ccccc5c5ccccc45)cc3)c2)cc1 ; c1ccc(-c2ccc(-c3ccc(N(c4ccc5c(c4)sc4ccccc45)c4cc5ccccc5c5ccccc45)cc3)cc2)cc1 ; c1ccc(-c2ccccc2-c2ccc(N(c3ccc(-c4cccc5ccccc45)cc3)c3ccc4c(ccc5ccccc54)c3)cc2)cc1]

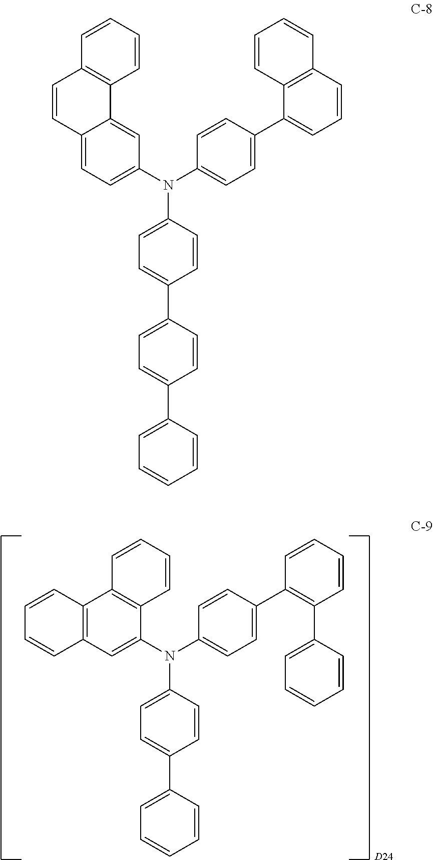

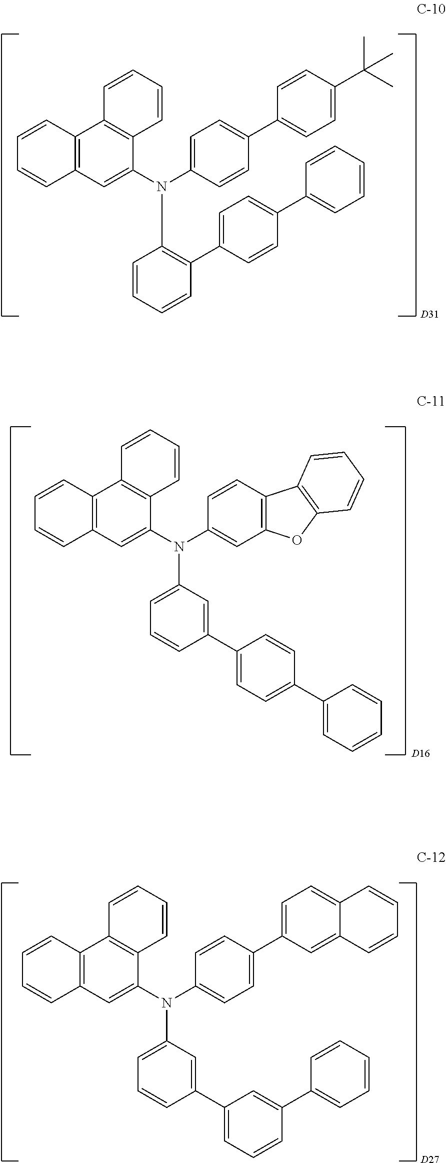

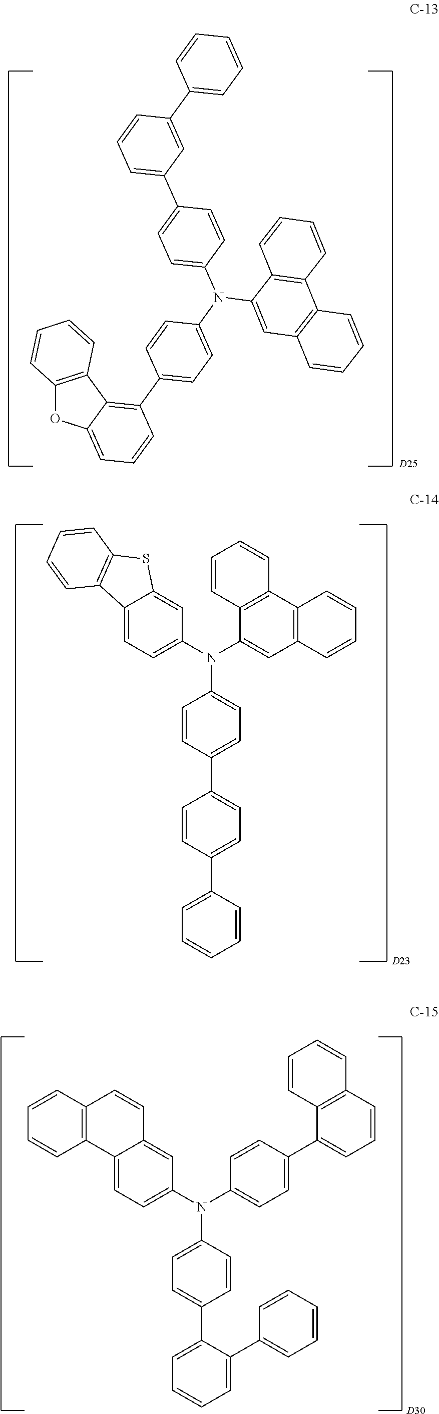

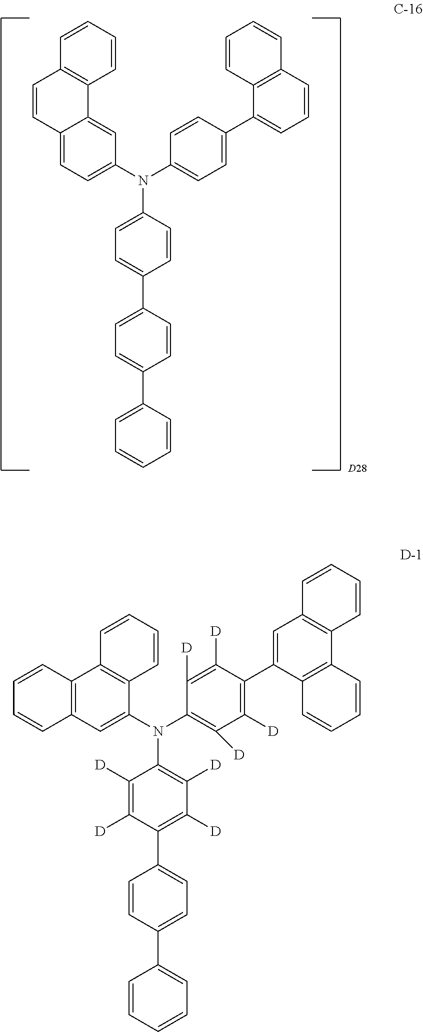

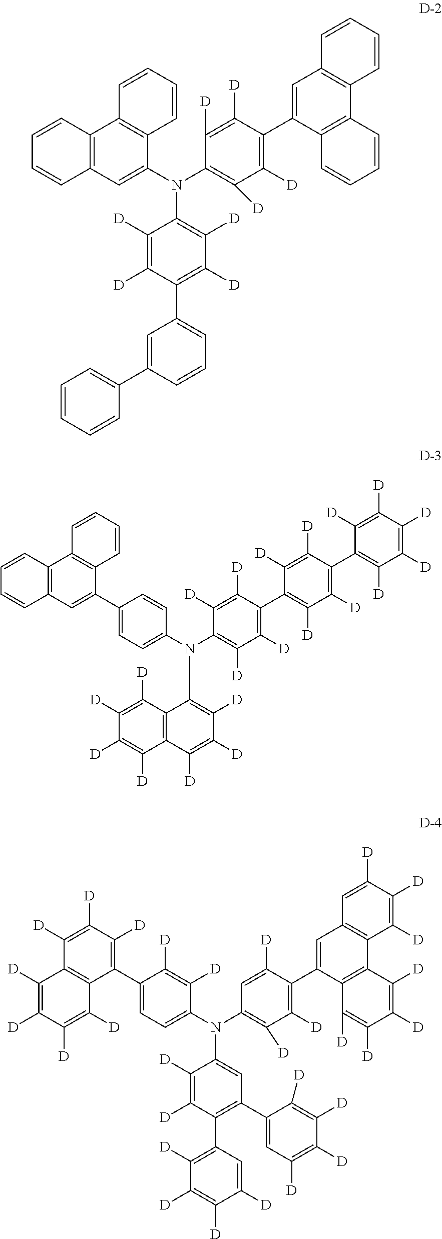

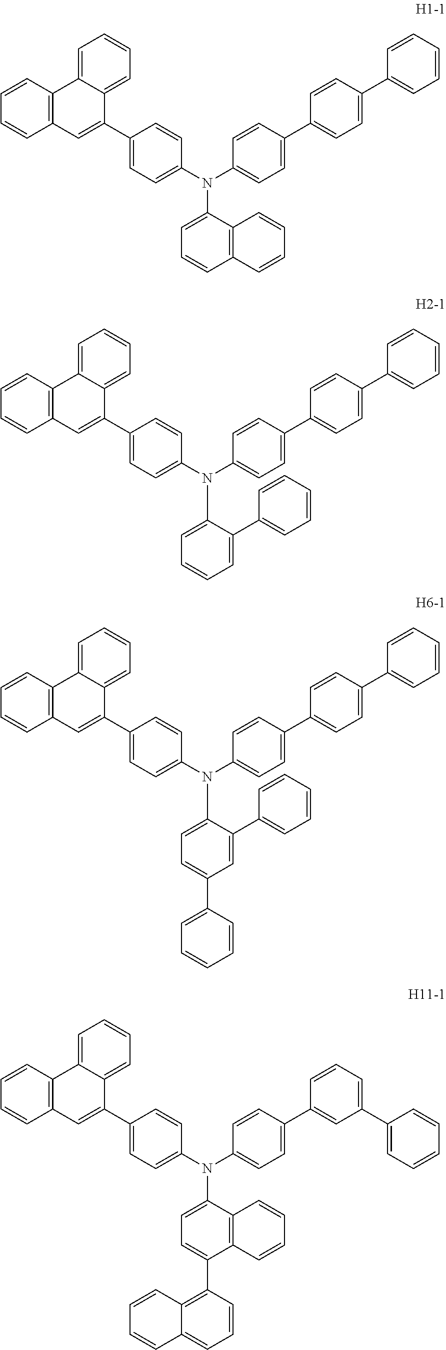 [SMILES: c1ccc(-c2ccc(-c3ccc(N(c4ccc(-c5cc6ccccc6c6ccccc56)cc4)c4cccc5ccccc45)cc3)cc2)cc1 ; c1ccc(-c2ccc(-c3ccc(N(c4ccc(-c5cc6ccccc6c6ccccc56)cc4)c4ccccc4-c4ccccc4)cc3)cc2)cc1 ; c1ccc(-c2ccc(-c3ccc(N(c4ccc(-c5cc6ccccc6c6ccccc56)cc4)c4ccc(-c5ccccc5)cc4-c4ccccc4)cc3)cc2)cc1 ; c1ccc(-c2cccc(-c3ccc(N(c4ccc(-c5cc6ccccc6c6ccccc56)cc4)c4ccc(-c5cccc6ccccc56)c5ccccc45)cc3)c2)cc1]

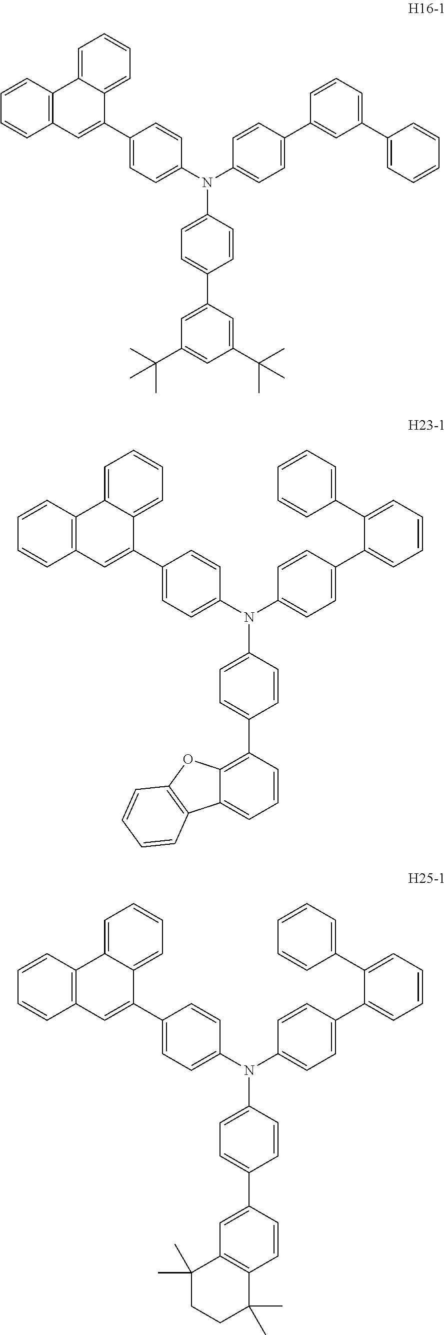 [SMILES: CC(C)(C)c1cc(-c2ccc(N(c3ccc(-c4cccc(-c5ccccc5)c4)cc3)c3ccc(-c4cc5ccccc5c5ccccc45)cc3)cc2)cc(C(C)(C)C)c1 ; c1ccc(-c2ccccc2-c2ccc(N(c3ccc(-c4cc5ccccc5c5ccccc45)cc3)c3ccc(-c4cccc5c4oc4ccccc45)cc3)cc2)cc1 ; CC1(C)CCC(C)(C)c2cc(-c3ccc(N(c4ccc(-c5ccccc5-c5ccccc5)cc4)c4ccc(-c5cc6ccccc6c6ccccc56)cc4)cc3)ccc21]

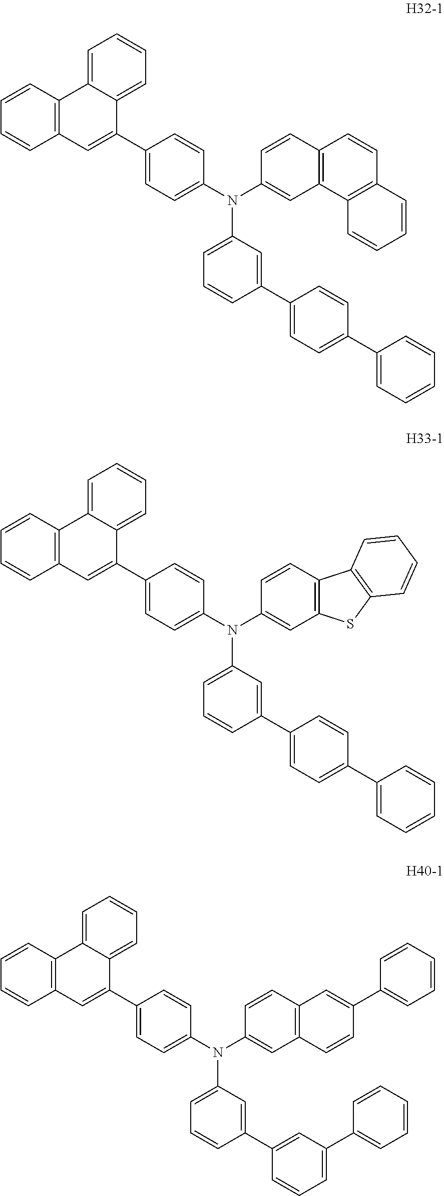 [SMILES: c1ccc(-c2ccc(-c3cccc(N(c4ccc(-c5cc6ccccc6c6ccccc56)cc4)c4ccc5ccc6ccccc6c5c4)c3)cc2)cc1 ; c1ccc(-c2ccc(-c3cccc(N(c4ccc(-c5cc6ccccc6c6ccccc56)cc4)c4ccc5c(c4)sc4ccccc45)c3)cc2)cc1 ; c1ccc(-c2cccc(-c3cccc(N(c4ccc(-c5cc6ccccc6c6ccccc56)cc4)c4ccc5cc(-c6ccccc6)ccc5c4)c3)c2)cc1]

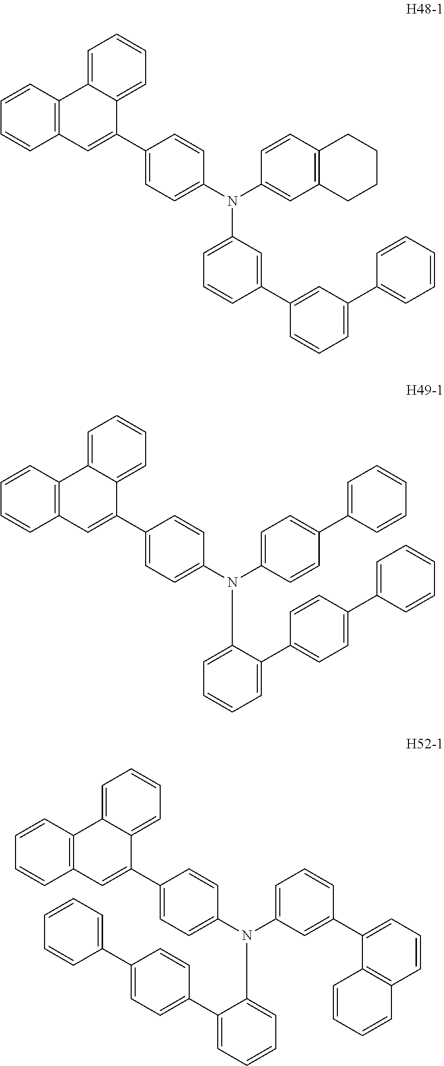 [SMILES: c1ccc(-c2cccc(-c3cccc(N(c4ccc(-c5cc6ccccc6c6ccccc56)cc4)c4ccc5c(c4)CCCC5)c3)c2)cc1 ; c1ccc(-c2ccc(-c3ccccc3N(c3ccc(-c4ccccc4)cc3)c3ccc(-c4cc5ccccc5c5ccccc45)cc3)cc2)cc1 ; c1ccc(-c2ccc(-c3ccccc3N(c3ccc(-c4cc5ccccc5c5ccccc45)cc3)c3cccc(-c4cccc5ccccc45)c3)cc2)cc1]

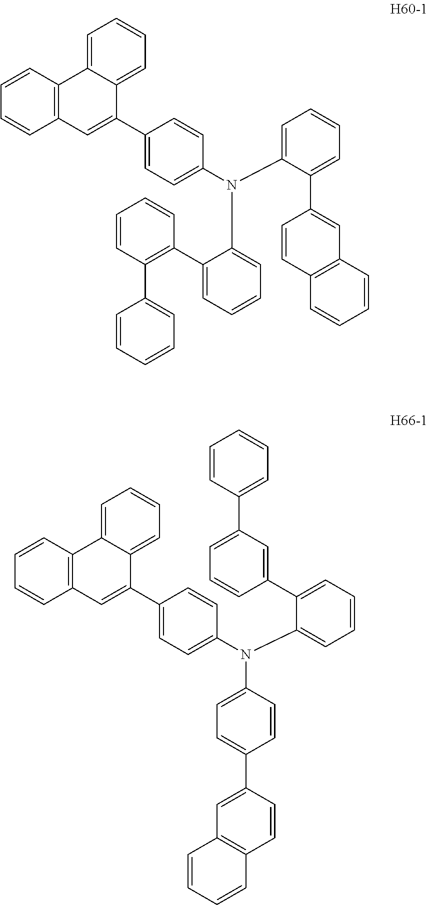 [SMILES: c1ccc(-c2ccccc2-c2ccccc2N(c2ccc(-c3cc4ccccc4c4ccccc34)cc2)c2ccccc2-c2ccc3ccccc3c2)cc1 ; c1ccc(-c2cccc(-c3ccccc3N(c3ccc(-c4ccc5ccccc5c4)cc3)c3ccc(-c4cc5ccccc5c5ccccc45)cc3)c2)cc1]

### Experimental Example 1

The voltage and efficiency were measured by applying a current (10 mA/cm²) to the organic light emitting devices manufactured in Examples 1-1 to 1-73 and Comparative Examples 1-1 to 1-35, and the results are shown in Tables 1 to 3 below. Lifetime T95 means the time required for the luminance to be reduced to 95% of the initial luminance (1000 nit).

TABLE 1

| | | | | | Lumi- |
|---|---|---|---|---|---|
| | | Volt- | Efficien- | Lifetime | nescent |
| | Compound | age(V) | cy(cd/A) | T95(hr) | color |
| Example | Compound 1- | 3.23 | 5.49 | 221 | Blue |
| 1-1 | D22 | | | | |
| Example | Compound 2- | 3.29 | 5.58 | 217 | Blue |
| 1-2 | D18 | | | | |
| Example | Compound 3- | 3.23 | 5.57 | 247 | Blue |
| 1-3 | D26 | | | | |
| Example | Compound 4- | 3.25 | 5.58 | 252 | Blue |
| 1-4 | D28 | | | | |
| Example | Compound 5- | 3.30 | 5.58 | 287 | Blue |
| 1-5 | D35 | | | | |
| Example | Compound 6- | 3.24 | 5.51 | 256 | Blue |
| 1-6 | D26 | | | | |
| Example | Compound 7- | 3.27 | 5.65 | 288 | Blue |
| 1-7 | D39 | | | | |
| Example | Compound 8- | 3.29 | 5.44 | 220 | Blue |
| 1-8 | D25 | | | | |
| Example | Compound 9- | 3.26 | 5.58 | 207 | Blue |
| 1-9 | D23 | | | | |
| Example | Compound | 3.37 | 5.36 | 25 | Blue |
| 1-10 | 10-D29 | | | | |
| Example | Compound | 3.41 | 5.25 | 212 | Blue |
| 1-11 | 11-D21 | | | | |
| Example | Compound | 3.35 | 5.22 | 247 | Blue |
| 1-12 | 12-D27 | | | | |
| Example | Compound | 3.34 | 5.28 | 256 | Blue |
| 1-13 | 13-D29 | | | | |
| Example | Compound | 3.38 | 5.32 | 247 | Blue |
| 1-14 | 14-D30 | | | | |
| Example | Compound | 3.35 | 5.34 | 213 | Blue |
| 1-15 | 15-D19 | | | | |
| Example | Compound | 3.40 | 5.31 | 205 | Blue |
| 1-16 | 16-D27 | | | | |
| Example | Compound | 3.41 | 5.28 | 254 | Blue |
| 1-17 | 17-D28 | | | | |
| Example | Compound | 3.40 | 5.30 | 247 | Blue |
| 1-18 | 18-D24 | | | | |
| Example | Compound | 3.36 | 5.18 | 256 | Blue |
| 1-19 | 19-D26 | | | | |
| Example | Compound | 3.39 | 5.24 | 23 | Blue |
| 1-20 | 20-D22 | | | | |
| Example | Compound | 3.36 | 5.33 | 289 | Blue |
| 1-21 | 21-D37 | | | | |
| Example | Compound | 3.36 | 5.16 | 222 | Blue |
| 1-22 | 22-D20 | | | | |
| Example | Compound | 3.38 | 5.21 | 221 | Blue |
| 1-23 | 23-D21 | | | | |
| Example | Compound | 3.41 | 5.16 | 245 | Blue |
| 1-24 | 24-D22 | | | | |
| Example | Compound | 3.34 | 5.26 | 216 | Blue |
| 1-25 | 25-D27 | | | | |
| Example | Compound | 3.38 | 5.14 | 205 | Blue |
| 1-26 | 26-D25 | | | | |
| Example | Compound | 3.48 | 4.94 | 257 | Blue |
| 1-27 | 27-D28 | | | | |
| Example | Compound | 3.52 | 5.02 | 278 | Blue |
| 1-28 | 28-D35 | | | | |
| Example | Compound | 3.50 | 4.99 | 213 | Blue |
| 1-29 | 29-D23 | | | | |
| Example | Compound | 3.48 | 5.12 | 254 | Blue |
| 1-30 | 30-D22 | | | | |
| Example | Compound | 3.43 | 5.09 | 242 | Blue |
| 1-31 | 31-D31 | | | | |
| Example | Compound | 3.43 | 4.93 | 253 | Blue |
| 1-32 | 32-D24 | | | | |
| Example | Compound | 3.47 | 4.96 | 221 | Blue |
| 1-33 | 33-D19 | | | | |
| Example | Compound | 3.47 | 5.03 | 246 | Blue |
| 1-34 | 34-D23 | | | | |
| Example | Compound | 3.50 | 5.05 | 258 | Blue |
| 1-35 | 35-D25 | | | | |
| Example | Compound | 3.51 | 5.04 | 222 | Blue |
| 1-36 | 36-D20 | | | | |
| Example | Compound | 3.55 | 4.90 | 242 | Blue |
| 1-37 | 37-D28 | | | | |
| Example | Compound | 3.54 | 4.85 | 215 | Blue |
| 1-38 | 38-D20 | | | | |
| Example | Compound | 3.59 | 4.72 | 258 | Blue |
| 1-39 | 39-D21 | | | | |
| Example | Compound | 3.58 | 4.78 | 219 | Blue |
| 1-40 | 40-D20 | | | | |

TABLE 2

| | | | | | Lumi- |
|---|---|---|---|---|---|
| | | Volt- | Efficien- | Lifetime | nescent |
| | Compound | age(V) | cy(cd/A) | T95(hr) | color |
| Example | Compound | 3.59 | 4.77 | 226 | Blue |
| 1-41 | 41-D21 | | | | |
| Example | Compound | 3.56 | 4.74 | 293 | Blue |
| 1-42 | 42-D37 | | | | |
| Example | Compound | 3.59 | 5.12 | 216 | Blue |
| 1-43 | 43-D21 | | | | |
| Example | Compound | 3.54 | 5.08 | 220 | Blue |
| 1-44 | 44-D21 | | | | |
| Example | Compound | 3.56 | 4.98 | 256 | Blue |
| 1-45 | 45-D24 | | | | |
| Example | Compound | 3.58 | 5.05 | 214 | Blue |
| 1-46 | 46-D19 | | | | |
| Example | Compound | 3.55 | 5.08 | 207 | Blue |
| 1-47 | 47-D21 | | | | |
| Example | Compound | 3.55 | 5.04 | 244 | Blue |
| 1-48 | 48-D20 | | | | |
| Example | Compound | 3.49 | 4.94 | 216 | Blue |
| 1-49 | 49-D20 | | | | |
| Example | Compound | 3.43 | 4.98 | 254 | Blue |
| 1-50 | 50-D20 | | | | |
| Example | Compound | 3.46 | 5.09 | 208 | Blue |
| 1-51 | 51-D21 | | | | |
| Example | Compound | 3.43 | 4.94 | 289 | Blue |
| 1-52 | 52-D37 | | | | |
| Example | Compound | 3.44 | 5.03 | 222 | Blue |
| 1-53 | 53-D19 | | | | |
| Example | Compound | 3.52 | 5.06 | 217 | Blue |
| 1-54 | 54-D25 | | | | |
| Example | Compound | 3.43 | 4.96 | 247 | Blue |
| 1-55 | 55-D23 | | | | |
| Example | Compound | 3.29 | 5.12 | 213 | Blue |
| 1-56 | 56-D21 | | | | |
| Example | Compound | 3.25 | 5.10 | 252 | Blue |
| 1-57 | 57-D29 | | | | |
| Example | Compound | 3.24 | 5.07 | 259 | Blue |
| 1-58 | 58-D27 | | | | |
| Example | Compound | 3.23 | 5.01 | 252 | Blue |
| 1-59 | 59-D24 | | | | |
| Example | Compound | 3.24 | 5.08 | 236 | Blue |
| 1-60 | 60-D20 | | | | |
| Example | Compound | 3.30 | 5.07 | 244 | Blue |
| 1-61 | 61-D25 | | | | |
| Example | Compound | 3.29 | 4.96 | 246 | Blue |
| 1-62 | 62-D21 | | | | |
| Example | Compound | 3.21 | 5.08 | 247 | Blue |
| 1-63 | 63-D30 | | | | |
| Example | Compound | 3.37 | 5.14 | 227 | Blue |
| 1-64 | 64-D20 | | | | |
| Example | Compound | 3.33 | 5.26 | 256 | Blue |
| 1-65 | 65-D28 | | | | |
| Example | Compound | 3.38 | 5.34 | 232 | Blue |
| 1-66 | 66-D37 | | | | |
| Example | Compound | 3.36 | 5.23 | 257 | Blue |
| 1-67 | 67-D29 | | | | |
| Example | Compound | 3.38 | 5.26 | 253 | Blue |
| 1-68 | 68-D29 | | | | |
| Example | Compound | 3.39 | 5.35 | 242 | Blue |
| 1-69 | 69-D23 | | | | |
| Example | Compound | 3.39 | 5.20 | 257 | Blue |
| 1-70 | 70-D25 | | | | |
| Example | Compound | 3.39 | 5.34 | 209 | Blue |
| 1-71 | 71-D26 | | | | |
| Example | Compound | 3.39 | 5.34 | 231 | Blue |
| 1-72 | 72-D23 | | | | |
| Example | Compound | 3.39 | 5.34 | 214 | Blue |
| 1-73 | 73-D21 | | | | |

TABLE 3

| | | | | | Lumi- |
|---|---|---|---|---|---|
| | | Volt- | Efficien- | Lifetime | nescent |
| | Compound | age(V) | cy(cd/A) | T95(hr) | color |
| Comparative | Compound | 3.72 | 4.65 | 135 | Blue |
| Example 1-1 | C-1 | | | | |
| Comparative | Compound | 3.92 | 4.42 | 141 | Blue |
| Example 1-2 | C-2 | | | | |
| Comparative | Compound | 3.88 | 4.55 | 133 | Blue |
| Example 1-3 | C-3 | | | | |
| Comparative | Compound | 3.74 | 4.67 | 128 | Blue |
| Example 1-4 | C-4 | | | | |
| Comparative | Compound | 3.78 | 4.63 | 134 | Blue |
| Example 1-5 | C-5 | | | | |
| Comparative | Compound | 3.74 | 4.54 | 137 | Blue |
| Example 1-6 | C-6 | | | | |
| Comparative | Compound | 3.88 | 4.48 | 131 | Blue |
| Example 1-7 | C-7 | | | | |
| Comparative | Compound | 3.83 | 4.62 | 125 | Blue |
| Example 1-8 | C-8 | | | | |
| Comparative | Compound | 3.70 | 4.67 | 157 | Blue |
| Example 1-9 | C-9 | | | | |
| Comparative | Compound | 3.94 | 4.45 | 157 | Blue |
| Example 1-10 | C-10 | | | | |
| Comparative | Compound | 3.86 | 4.53 | 155 | Blue |
| Example 1-11 | C-11 | | | | |
| Comparative | Compound | 3.71 | 4.69 | 149 | Blue |
| Example 1-12 | C-12 | | | | |
| Comparative | Compound | 3.75 | 4.66 | 152 | Blue |
| Example 1-13 | C-13 | | | | |
| Comparative | Compound | 3.72 | 4.52 | 158 | Blue |
| Example 1-14 | C-14 | | | | |
| Comparative | Compound | 3.89 | 4.50 | 153 | Blue |
| Example 1-15 | C-15 | | | | |
| Comparative | Compound | 3.80 | 4.65 | 152 | Blue |
| Example 1-16 | C-16 | | | | |
| Comparative | Compound | 3.21 | 5.51 | 170 | Blue |
| Example 1-17 | D-1 | | | | |
| Comparative | Compound | 3.28 | 5.60 | 165 | Blue |
| Example 1-18 | D-2 | | | | |
| Comparative | Compound | 3.26 | 5.50 | 184 | Blue |
| Example 1-19 | D-3 | | | | |
| Comparative | Compound | 3.43 | 5.22 | 191 | Blue |
| Example 1-20 | D-4 | | | | |
| Comparative | Compound | 3.21 | 5.51 | 178 | Blue |
| Example 1-21 | H1-1 | | | | |
| Comparative | Compound | 3.28 | 5.60 | 173 | Blue |
| Example 1-22 | H2-1 | | | | |
| Comparative | Compound | 3.26 | 5.50 | 186 | Blue |
| Example 1-23 | H6-1 | | | | |
| Comparative | Compound | 3.43 | 5.22 | 176 | Blue |
| Example 1-24 | H11-1 | | | | |
| Comparative | Compound | 3.38 | 5.33 | 180 | Blue |
| Example 1-25 | H16-1 | | | | |
| Comparative | Compound | 3.37 | 5.22 | 192 | Blue |
| Example 1-26 | H23-1 | | | | |
| Comparative | Compound | 3.36 | 5.25 | 188 | Blue |
| Example 1-27 | H25-1 | | | | |
| Comparative | Compound | 3.45 | 4.92 | 165 | Blue |
| Example 1-28 | H32-1 | | | | |
| Comparative | Compound | 3.45 | 4.97 | 192 | Blue |
| Example 1-29 | H33-1 | | | | |
| Comparative | Compound | 3.59 | 4.76 | 188 | Blue |
| Example 1-30 | H40-1 | | | | |
| Comparative | Compound | 3.57 | 5.02 | 183 | Blue |
| Example 1-31 | H48-1 | | | | |
| Comparative | Compound | 3.50 | 4.92 | 177 | Blue |
| Example 1-32 | H49-1 | | | | |
| Comparative | Compound | 3.46 | 4.92 | 183 | Blue |
| Example 1-33 | H52-1 | | | | |
| Comparative | Compound | 3.21 | 5.10 | 194 | Blue |
| Example 1-34 | H60-1 | | | | |
| Comparative | Compound | 3.36 | 5.36 | 188 | Blue |
| Example 1-35 | H66-1 | | | | |

As shown in Table 1 and Table 2, the compound of the present disclosure was used as a hole transport auxiliary layer in the blue light emitting layer. Compared with Comparative Examples in Table 3, it was confirmed that the organic light emitting device using the compound of the present disclosure showed improvement effects in terms of driving voltage, efficiency, and lifetime. In addition, when the lifetime characteristics of the compound of the present disclosure were confirmed before and after deuterium substitution, it was confirmed that the lifetime was increased due to the deuterium substitution effect. It is considered that the deuterium substitution of the compound contributes to the stability of the molecule, thereby exhibiting the effect of improving the OLED lifetime characteristics.

### Example 2-1

In Example 1-1, instead of the compound of the present disclosure as a hole transport auxiliary layer, the following compound EB-1 was thermally vacuum deposited to a thickness of 150 Å, instead of Compound BH-1 and Compound BD-1 as the light emitting layer, the following compound RH-1 as the first host, the compound 1-D22 of the present invention as the second host, and the following compound Dp-7 as the dopant were used in a weight ratio of 49:49:2, and vacuum deposited to a thickness of 400 Å to manufacture a red light emitting device.

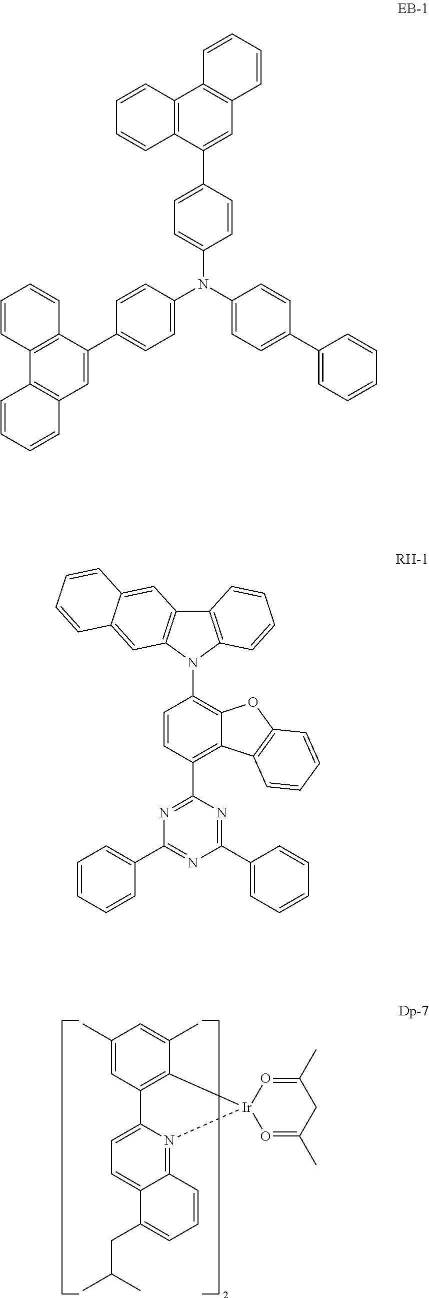

### Examples 2-2 to 2-73

The organic light emitting devices of Examples 2-2 to 2-73 were manufactured in the same manner as Example 2-1, except that the compounds shown in Tables 4 and 5 below were used instead of Compound 1-D22 used in Example 2-1.

### Comparative Examples 2-1 to 2-31

The organic light emitting devices of Comparative Examples 2-1 to 2-31 were manufactured in the same manner as Example 2-1, except that the following compounds C-1 to C-16 and the comparative example compounds listed in Table 6 were used instead of Compound 1-D22.

### Experimental Example 2

The voltage and efficiency were measured (based on 15 mA/cm²) by applying a current to the organic light emitting devices manufactured in Examples 2-1 to 2-73 and Comparative Examples 2-1 to 2-31, and the results are shown in Tables 4 to 6 below. Lifetime T95 means the time required for the luminance to be reduced to 95% of the initial luminance (6,000 nit).

TABLE 4

| | | | | | Lifetime | |
|---|---|---|---|---|---|---|
| | | | Voltage | Efficiency | T95 | Luminescent |
| | First host | Second host | (V) | (cd/A) | (hr) | color |
| Example | Compound | Compound | 3.32 | 26.82 | 334 | Red |
| 2-1 | RH-1 | 1-D22 | | | | |
| Example | | Compound | 3.38 | 25.17 | 286 | Red |
| 2-2 | | 2-D18 | | | | |
| Example | | Compound | 3.38 | 26.89 | 321 | Red |
| 2-3 | | 3-D26 | | | | |
| Example | | Compound | 3.41 | 25.26 | 331 | Red |
| 2-4 | | 4-D28 | | | | |
| Example | | Compound | 3.36 | 26.72 | 370 | Red |
| 2-5 | | 5-D35 | | | | |
| Example | | Compound | 3.41 | 25.41 | 333 | Red |
| 2-6 | | 6-D26 | | | | |
| Example | | Compound | 3.38 | 25.55 | 356 | Red |
| 2-7 | | 7-D39 | | | | |
| Example | | Compound | 3.41 | 25.45 | 292 | Red |
| 2-8 | | 8-D25 | | | | |
| Example | | Compound | 3.35 | 25.17 | 281 | Red |
| 2-9 | | 9-D23 | | | | |
| Example | | Compound | 3.36 | 23.54 | 334 | Red |
| 2-10 | | 10-D29 | | | | |
| Example | | Compound | 3.37 | 24.06 | 303 | Red |
| 2-11 | | 11-D21 | | | | |
| Example | | Compound | 3.42 | 24.08 | 329 | Red |
| 2-12 | | 12-D27 | | | | |
| Example | | Compound | 3.33 | 23.67 | 325 | Red |
| 2-13 | | 13-D29 | | | | |
| Example | | Compound | 3.34 | 24.08 | 333 | Red |
| 2-14 | | 14-D30 | | | | |
| Example | | Compound | 3.33 | 23.92 | 286 | Red |
| 2-15 | | 15-D19 | | | | |
| Example | | Compound | 3.41 | 23.46 | 284 | Red |
| 2-16 | | 16-D27 | | | | |
| Example | | Compound | 3.37 | 23.53 | 320 | Red |
| 2-17 | | 17-D28 | | | | |
| Example | | Compound | 3.41 | 25.15 | 317 | Red |
| 2-18 | | 18-D24 | | | | |
| Example | | Compound | 3.45 | 26.36 | 323 | Red |
| 2-19 | | 19-D26 | | | | |
| Example | | Compound | 3.45 | 26.73 | 302 | Red |
| 2-20 | | 20-D22 | | | | |
| Example | | Compound | 3.44 | 25.74 | 366 | Red |
| 2-21 | | 21-D37 | | | | |
| Example | | Compound | 3.51 | 25.62 | 290 | Red |
| 2-22 | | 22-D20 | | | | |
| Example | | Compound | 3.48 | 25.17 | 285 | Red |
| 2-23 | | 23-D21 | | | | |
| Example | | Compound | 3.47 | 25.23 | 334 | Red |
| 2-24 | | 24-D22 | | | | |
| Example | | Compound | 3.50 | 26.07 | 288 | Red |
| 2-25 | | 25-D27 | | | | |
| Example | | Compound | 3.46 | 25.27 | 298 | Red |
| 2-26 | | 26-D25 | | | | |
| Example | | Compound | 3.40 | 23.93 | 329 | Red |
| 2-27 | | 27-D28 | | | | |
| Example | | Compound | 3.31 | 24.71 | 370 | Red |
| 2-28 | | 28-D35 | | | | |
| Example | | Compound | 3.36 | 23.84 | 283 | Red |
| 2-29 | | 29-D23 | | | | |
| Example | | Compound | 3.40 | 24.53 | 332 | Red |
| 2-30 | | 30-D22 | | | | |
| Example | | Compound | 3.35 | 24.60 | 333 | Red |
| 2-31 | | 31-D31 | | | | |
| Example | | Compound | 3.32 | 24.59 | 332 | Red |
| 2-32 | | 32-D24 | | | | |
| Example | | Compound | 3.40 | 23.71 | 300 | Red |
| 2-33 | | 33-D19 | | | | |
| Example | | Compound | 3.38 | 24.60 | 330 | Red |
| 2-34 | | 34-D23 | | | | |
| Example | | Compound | 3.38 | 24.42 | 317 | Red |
| 2-35 | | 35-D25 | | | | |
| Example | | Compound | 3.53 | 22.60 | 300 | Red |
| 2-36 | | 36-D20 | | | | |
| Example | | Compound | 3.69 | 21.65 | 316 | Red |
| 2-37 | | 37-D28 | | | | |
| Example | | Compound | 3.62 | 21.89 | 299 | Red |
| 2-38 | | 38-D20 | | | | |
| Example | | Compound | 3.61 | 21.75 | 329 | Red |
| 2-39 | | 39-D21 | | | | |
| Example | | Compound | 3.68 | 21.81 | 282 | Red |
| 2-40 | | 40-D20 | | | | |

TABLE 5

| | | | | | Lifetime | |
|---|---|---|---|---|---|---|
| | | | Voltage | Efficiency | T95 | Luminescent |
| | First host | Second host | (V) | (cd/A) | (hr) | color |
| Example | Compound | Compound | 3.66 | 22.04 | 302 | Red |
| 2-41 | RH-1 | 41-D21 | | | | |
| Example | | Compound | 3.69 | 21.91 | 355 | Red |
| 2-42 | | 42-D37 | | | | |
| Example | | Compound | 3.69 | 22.02 | 284 | Red |
| 2-43 | | 43-D21 | | | | |
| Example | | Compound | 3.72 | 22.33 | 286 | Red |
| 2-44 | | 44-D21 | | | | |
| Example | | Compound | 3.72 | 22.10 | 321 | Red |
| 2-45 | | 45-D24 | | | | |
| Example | | Compound | 3.61 | 22.10 | 288 | Red |
| 2-46 | | 46-D19 | | | | |
| Example | | Compound | 3.65 | 21.47 | 297 | Red |
| 2-47 | | 47-D21 | | | | |
| Example | | Compound | 3.64 | 21.62 | 324 | Red |
| 2-48 | | 48-D20 | | | | |
| Example | | Compound | 3.52 | 22.84 | 284 | Red |
| 2-49 | | 49-D20 | | | | |
| Example | | Compound | 3.59 | 22.67 | 328 | Red |
| 2-50 | | 50-D20 | | | | |
| Example | | Compound | 3.59 | 22.81 | 295 | Red |
| 2-51 | | 51-D21 | | | | |
| Example | | Compound | 3.57 | 23.05 | 368 | Red |
| 2-52 | | 52-D37 | | | | |
| Example | | Compound | 3.55 | 23.01 | 294 | Red |
| 2-53 | | 53-D19 | | | | |
| Example | | Compound | 3.61 | 23.02 | 300 | Red |
| 2-54 | | 54-D25 | | | | |
| Example | | Compound | 3.54 | 22.59 | 318 | Red |
| 2-55 | | 55-D23 | | | | |
| Example | | Compound | 3.35 | 23.02 | 283 | Red |
| 2-56 | | 56-D21 | | | | |
| Example | | Compound | 3.38 | 22.74 | 325 | Red |
| 2-57 | | 57-D29 | | | | |
| Example | | Compound | 3.38 | 23.02 | 322 | Red |
| 2-58 | | 58-D27 | | | | |
| Example | | Compound | 3.35 | 22.85 | 321 | Red |
| 2-59 | | 59-D24 | | | | |
| Example | | Compound | 3.40 | 22.78 | 294 | Red |
| 2-60 | | 60-D20 | | | | |
| Example | | Compound | 3.40 | 22.56 | 318 | Red |
| 2-61 | | 61-D25 | | | | |
| Example | | Compound | 3.32 | 22.45 | 331 | Red |
| 2-62 | | 62-D21 | | | | |
| Example | | Compound | 3.38 | 22.96 | 331 | Red |
| 2-63 | | 63-D30 | | | | |
| Example | | Compound | 3.31 | 22.70 | 295 | Red |
| 2-64 | | 64-D20 | | | | |
| Example | | Compound | 3.37 | 23.03 | 331 | Red |
| 2-65 | | 65-D28 | | | | |
| Example | | Compound | 3.40 | 22.46 | 366 | Red |
| 2-66 | | 66-D37 | | | | |
| Example | | Compound | 3.42 | 22.49 | 323 | Red |
| 2-67 | | 67-D29 | | | | |
| Example | | Compound | 3.33 | 22.93 | 331 | Red |
| 2-68 | | 68-D29 | | | | |
| Example | | Compound | 3.35 | 23.08 | 329 | Red |
| 2-69 | | 69-D23 | | | | |
| Example | | Compound | 3.38 | 22.65 | 320 | Red |
| 2-70 | | 70-D25 | | | | |
| Example | | Compound | 3.41 | 23.02 | 282 | Red |
| 2-71 | | 71-D26 | | | | |
| Example | | Compound | 3.38 | 22.65 | 305 | Red |
| 2-72 | | 72-D23 | | | | |
| Example | | Compound | 3.41 | 23.02 | 286 | Red |
| 2-73 | | 73-D21 | | | | |

TABLE 6

| | | | | | Lifetime | |
|---|---|---|---|---|---|---|
| | | Second | Voltage | Efficiency | T95 | Luminescent |
| | First host | host | (V) | (cd/A) | (hr) | color |
| Comparative | Compound | Compound | 3.81 | 19.85 | 183 | Red |
| Example 2-1 | RH-1 | C-1 | | | | |
| Comparative | | Compound | 3.93 | 18.36 | 177 | Red |
| Example 2-2 | | C-2 | | | | |
| Comparative | | Compound | 3.82 | 19.73 | 178 | Red |
| Example 2-3 | | C-3 | | | | |
| Comparative | | Compound | 3.79 | 20.96 | 172 | Red |
| Example 2-4 | | C-4 | | | | |
| Comparative | | Compound | 3.80 | 20.74 | 189 | Red |
| Example 2-5 | | C-5 | | | | |
| Comparative | | Compound | 3.88 | 18.61 | 179 | Red |
| Example 2-6 | | C-6 | | | | |
| Comparative | | Compound | 3.86 | 20.57 | 182 | Red |
| Example 2-7 | | C-7 | | | | |
| Comparative | | Compound | 3.84 | 19.95 | 188 | Red |
| Example 2-8 | | C-8 | | | | |
| Comparative | | Compound | 3.82 | 19.87 | 215 | Red |
| Example 2-9 | | C-9 | | | | |
| Comparative | | Compound | 3.91 | 18.33 | 203 | Red |
| Example 2-10 | | C-10 | | | | |
| Comparative | | Compound | 3.80 | 19.77 | 193 | Red |
| Example 2-11 | | C-11 | | | | |
| Comparative | | Compound | 3.76 | 20.92 | 205 | Red |
| Example 2-12 | | C-12 | | | | |
| Comparative | | Compound | 3.82 | 20.79 | 217 | Red |
| Example 2-13 | | C-13 | | | | |
| Comparative | | Compound | 3.86 | 18.64 | 204 | Red |
| Example 2-14 | | C-14 | | | | |
| Comparative | | Compound | 3.87 | 20.52 | 219 | Red |
| Example 2-15 | | C-15 | | | | |
| Comparative | | Compound | 3.82 | 19.98 | 207 | Red |
| Example 2-16 | | C-16 | | | | |
| Comparative | | Compound | 3.32 | 26.85 | 241 | Red |
| Example 2-17 | | H1-1 | | | | |
| Comparative | | Compound | 3.38 | 25.14 | 232 | Red |
| Example 2-18 | | H2-1 | | | | |
| Comparative | | Compound | 3.42 | 25.42 | 245 | Red |
| Example 2-19 | | H6-1 | | | | |
| Comparative | | Compound | 3.35 | 24.03 | 251 | Red |
| Example 2-20 | | H11-1 | | | | |
| Comparative | | Compound | 3.43 | 23.48 | 249 | Red |
| Example 2-21 | | H16-1 | | | | |
| Comparative | | Compound | 3.45 | 25.19 | 245 | Red |
| Example 2-22 | | H23-1 | | | | |
| Comparative | | Compound | 3.52 | 26.10 | 241 | Red |
| Example 2-23 | | H25-1 | | | | |
| Comparative | | Compound | 3.30 | 24.60 | 238 | Red |
| Example 2-24 | | H32-1 | | | | |
| Comparative | | Compound | 3.43 | 23.74 | 261 | Red |
| Example 2-25 | | H33-1 | | | | |
| Comparative | | Compound | 3.66 | 21.80 | 235 | Red |
| Example 2-26 | | H40-1 | | | | |
| Comparative | | Compound | 3.62 | 21.64 | 243 | Red |
| Example 2-27 | | H48-1 | | | | |
| Comparative | | Compound | 3.50 | 22.81 | 236 | Red |
| Example 2-28 | | H49-1 | | | | |
| Comparative | | Compound | 3.58 | 23.09 | 248 | Red |
| Example 2-29 | | H52-1 | | | | |
| Comparative | | Compound | 3.42 | 22.72 | 245 | Red |
| Example 2-30 | | H60-1 | | | | |
| Comparative | | Compound | 3.39 | 22.52 | 245 | Red |
| Example 2-31 | | H66-1 | | | | |

As shown in Tables 4 and 5, the compound of the present disclosure was used as the second host among the hosts of the red light emitting layer. Compared with Comparative Examples in Table 6, it was confirmed that the organic light emitting device using the compound of the present disclosure showed improvement effects in terms of driving voltage, efficiency, and lifetime. In addition, when the lifetime characteristics of the compound of the present disclosure were confirmed before and after deuterium substitution, it was confirmed that the lifetime was increased due to deuterium substitution effect. It is considered that deuterium substitution in the host compound in the red light emitting device contributes to the stability of the molecule, thereby exhibiting the effect of improving OLED lifetime characteristics.

[Description of Symbols]

| | |
|---|---|
| 1: substrate | 2: anode |
| 3: light emitting layer | 4: cathode |
| 5: hole injection layer | 6: hole transport layer |
| 7: hole transport auxiliary layer | 8: hole blocking layer |
| 9: electron transport and inject layer | |

1. A compound represented by the following Chemical Formula 1: 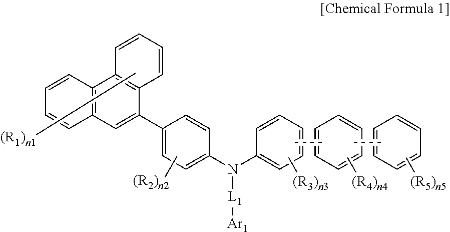
in Chemical Formula 1,
Ar₁ is a substituted or unsubstituted C₆₋₆₀ aryl; a substituted or unsubstituted dibenzofuranyl; or a substituted or unsubstituted dibenzothiophenyl,
L₁ is a single bond; a substituted or unsubstituted C₆₋₆₀ arylene; a substituted or unsubstituted dibenzofuranylene; or a substituted or unsubstituted dibenzothiophenylene,
R₁ is deuterium,
R₂ to R₄ are each independently hydrogen or deuterium,
R₅ is hydrogen, deuterium, or a substituted or unsubstituted C₁₋₆₀ alkyl,
n1 is an integer of 1 to 9,
n2 to n4 are each independently an integer of 1 to 4,
n5 is an integer of 1 to 5,
wherein the compound represented by Chemical Formula 1 has a deuterium substitution rate of 50% or more.

2. The compound as claimed in claim 1, wherein:
the Chemical Formula 1 is represented by the following Chemical Formulas 1-1 to 1-8: 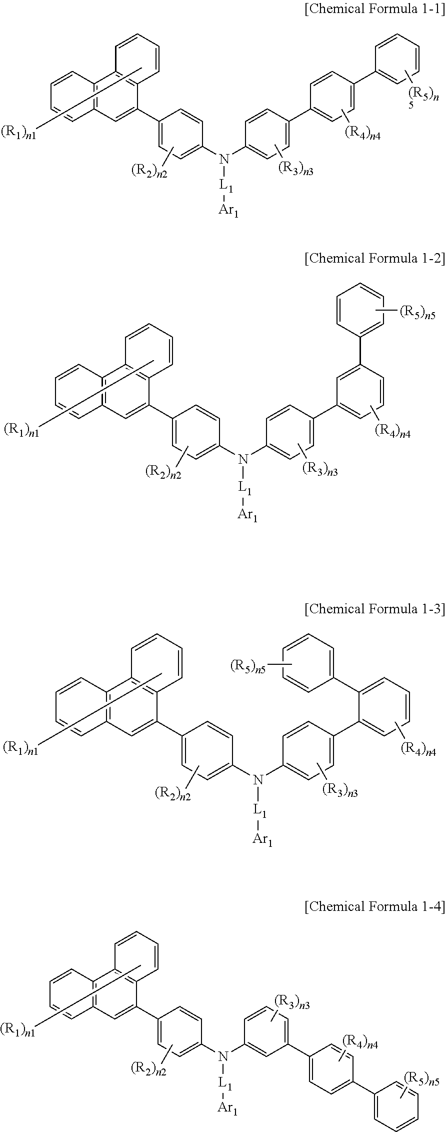 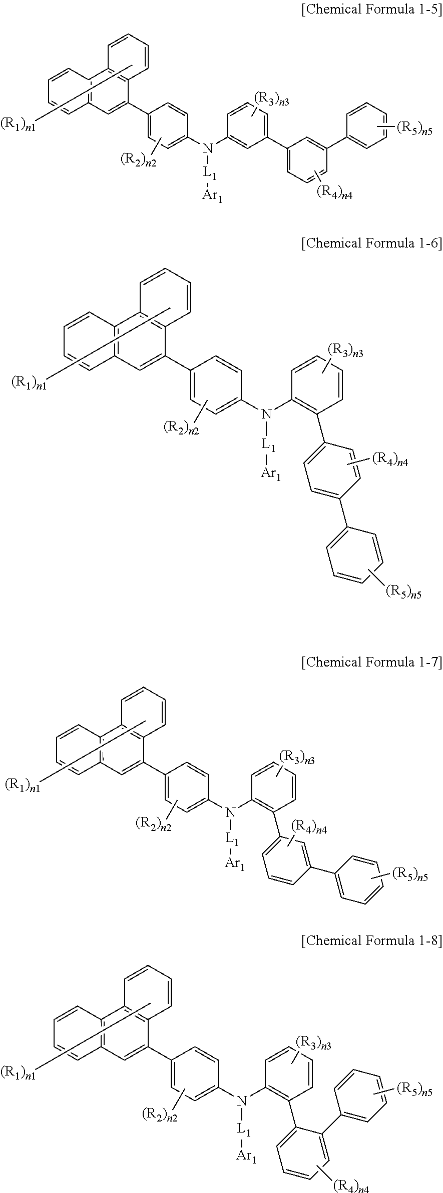
in Chemical Formulas 1-1 to 1-8,
Ar₁, L₁, R₁ to R₅ and n1 to n5 are as defined in claim 1.

3. The compound as claimed in claim 1, wherein:
Ar₁ is phenyl, biphenylyl, terphenylyl, naphthyl, phenanthrenyl, tetrahydronaphthyl, tetramethyltetrahydronaphthyl, phenyl substituted with one or two methyl groups, phenyl substituted with one or two isopropyl groups, phenyl substituted with one or two tertbutyl groups, dibenzofuranyl, or dibenzothiophenyl,
wherein the phenyl, biphenylyl, terphenylyl, naphthyl, phenanthrenyl, tetrahydronaphthyl, tetramethyltetrahydronaphthyl, phenyl substituted with one or two methyl groups, phenyl substituted with one or two isopropyl groups, phenyl substituted with one or two tertbutyl groups, dibenzofuranyl, or dibenzothiophenyl are each independently unsubstituted, or substituted with at least one deuterium.

4. The compound as claimed in claim 1, wherein:
L₁ is a single bond, phenylene, biphenyldiyl, naphthalenediyl, or phenyl naphthalenediyl,
wherein the phenylene, biphenyldiyl, naphthalenediyl, and phenyl naphthalenediyl are each independently unsubstituted, or substituted with at least one deuterium.

5. The compound as claimed in claim 1, wherein:
R₅ is hydrogen, deuterium, methyl, isopropyl, or tertbutyl,
wherein the methyl, isopropyl and tertbutyl are each independently unsubstituted, or substituted with at least one deuterium.

6. The compound as claimed in claim 1, wherein
the compound represented by Chemical Formula 1 is any one selected from the group consisting of the following compounds,
in the following group, D is deuterium,
n1 is an integer of 1 to 9,
n is the total number of deuterium substituted in the compound, and
each compound in the following group has a deuterium substitution rate of 50% or more:

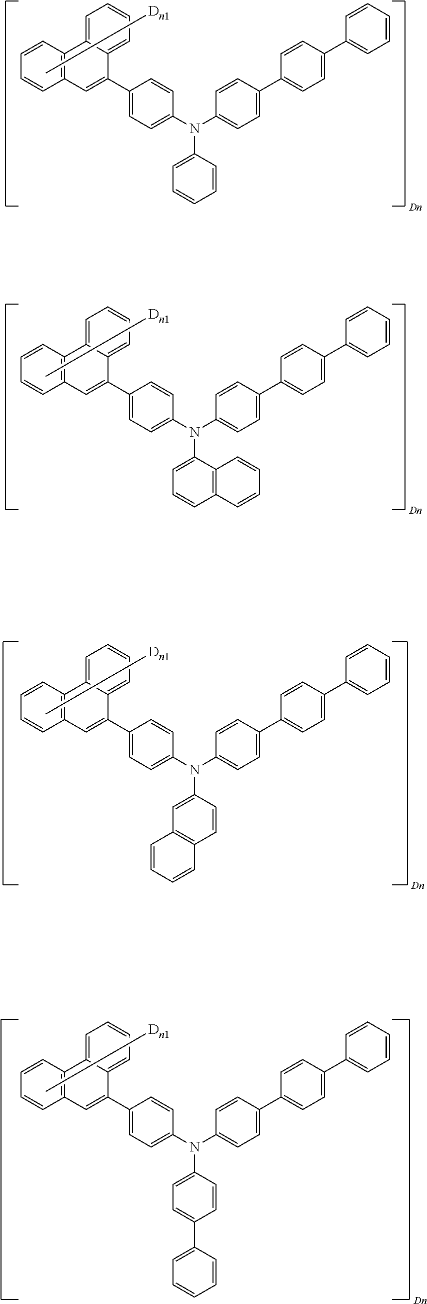

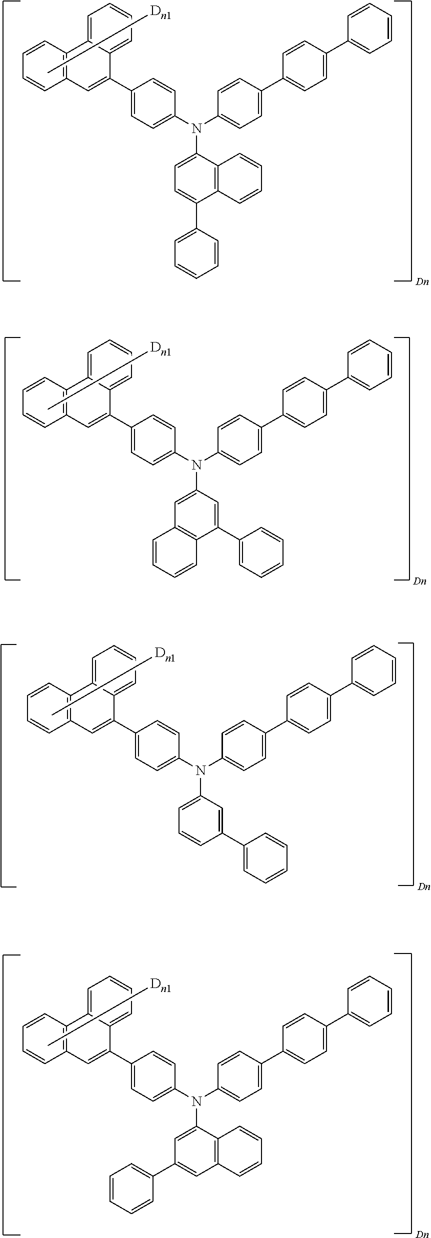

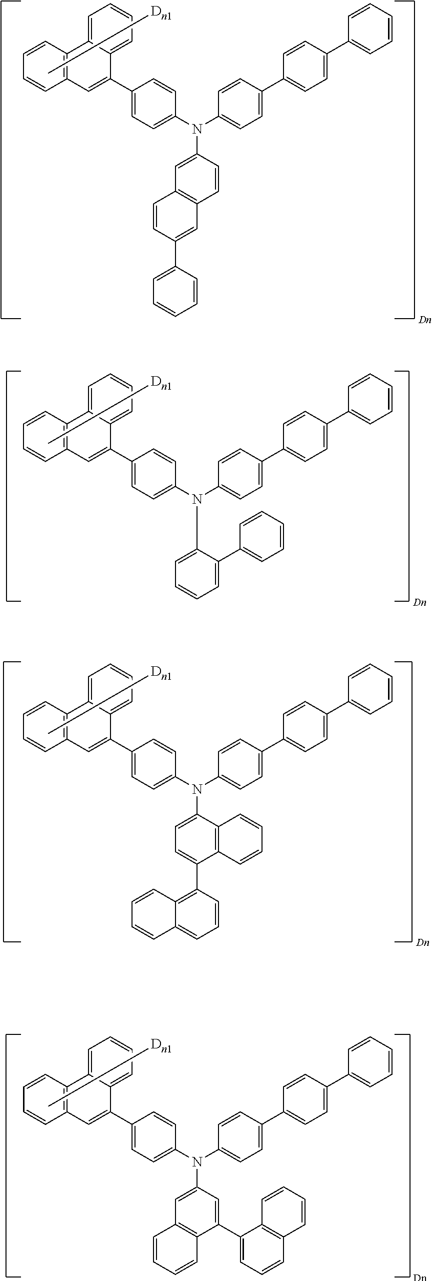

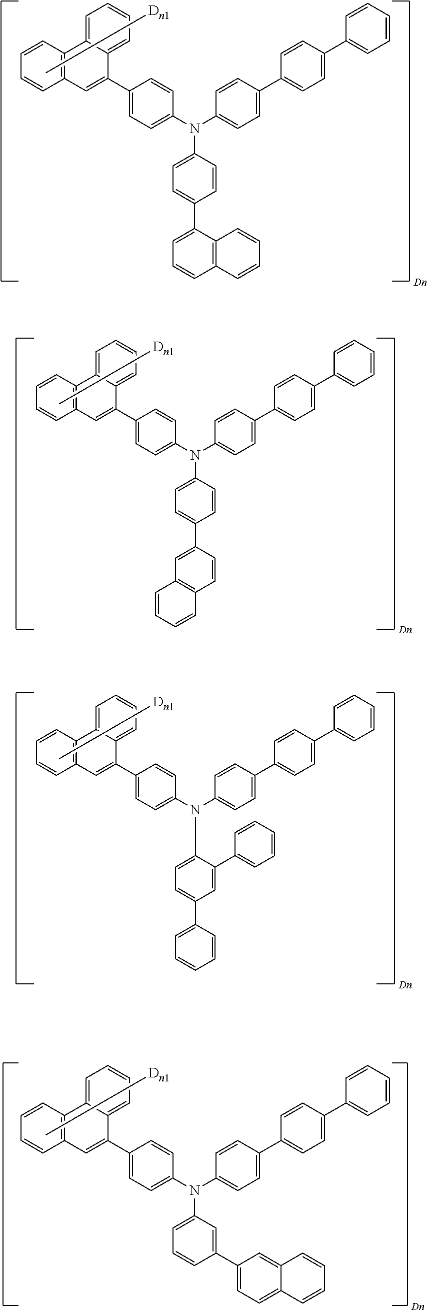

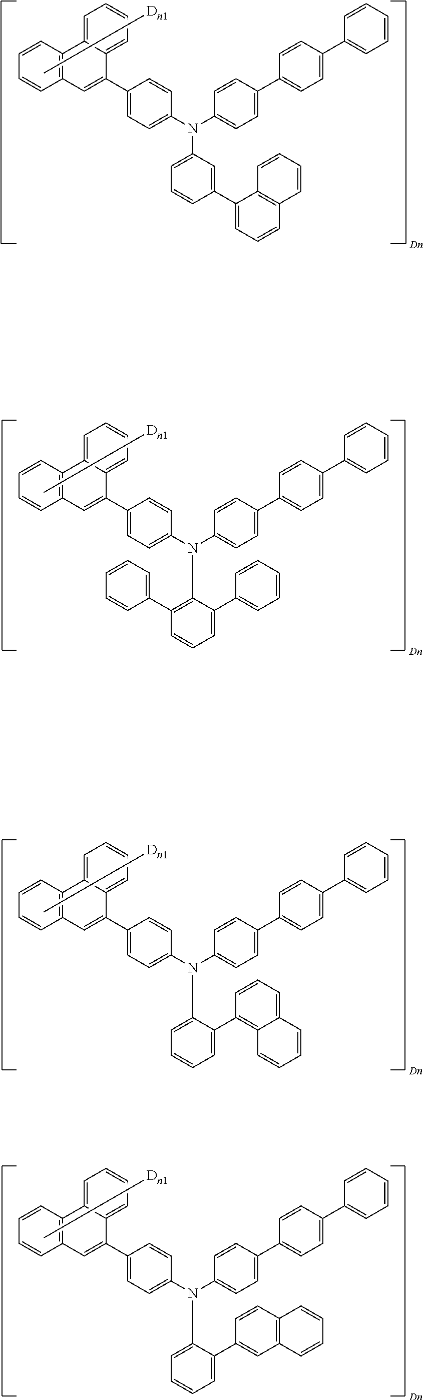

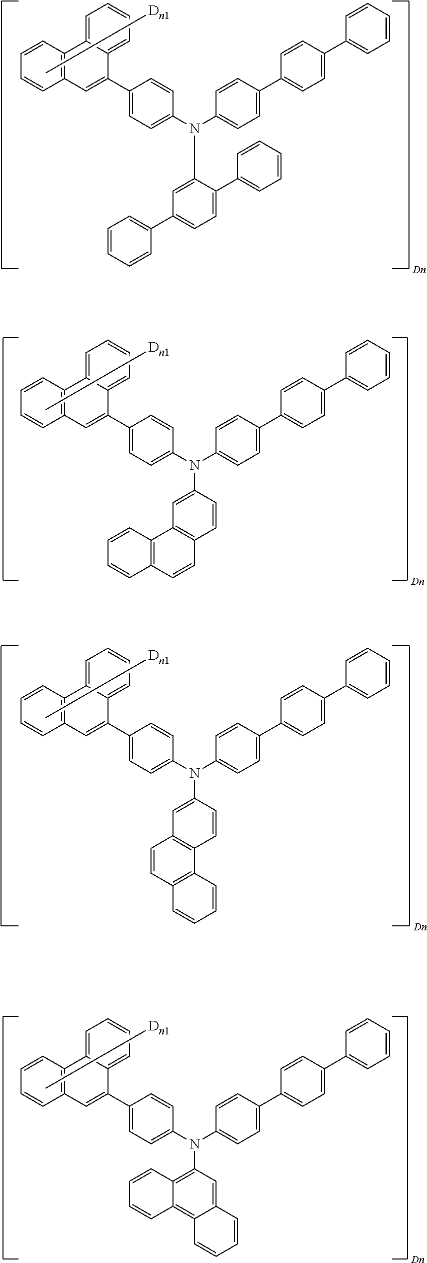

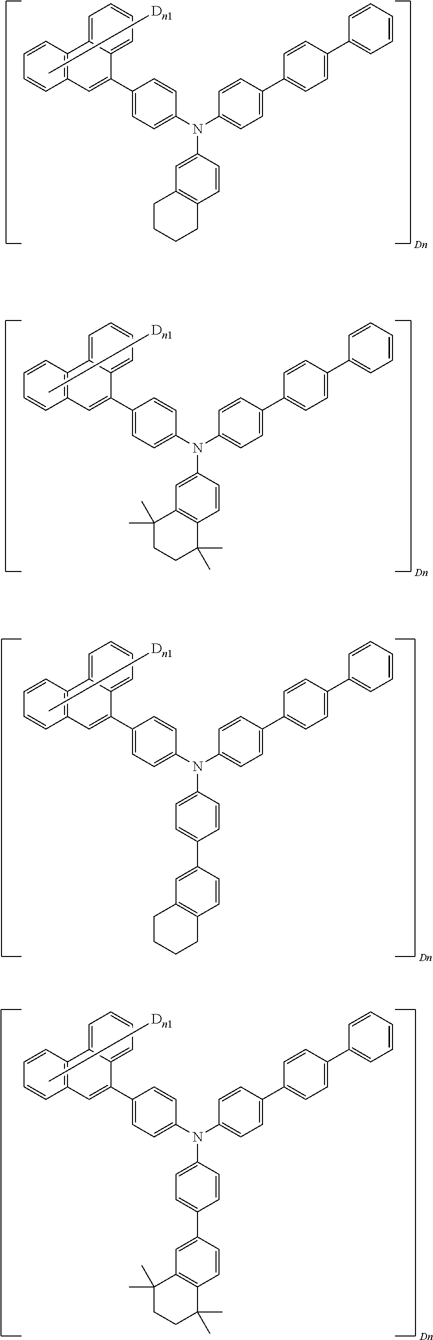

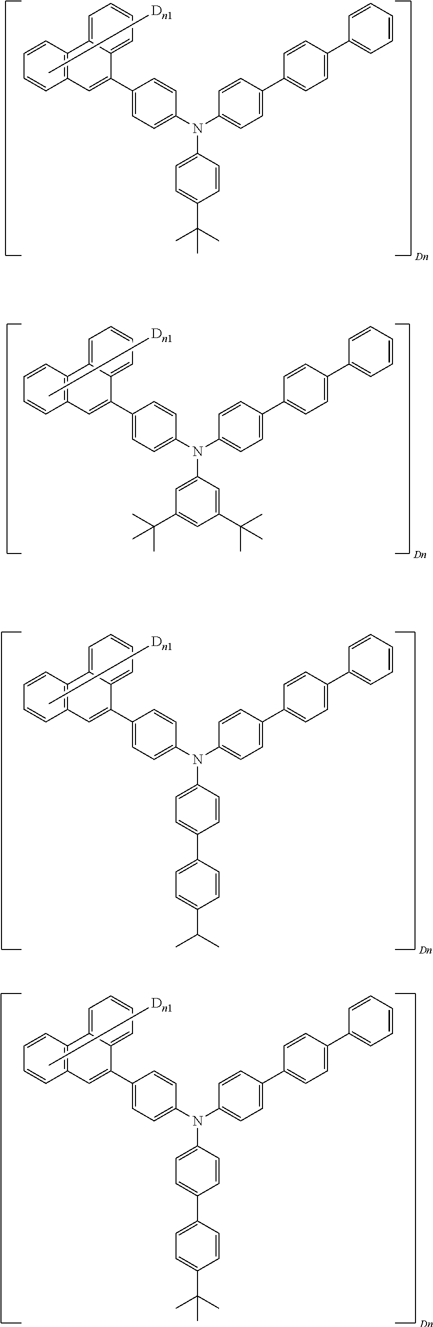

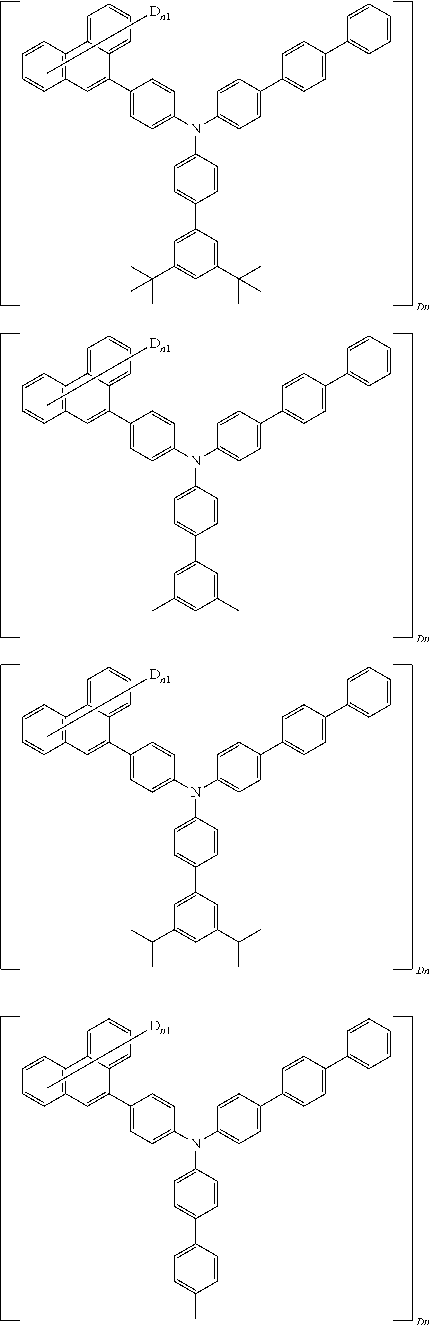

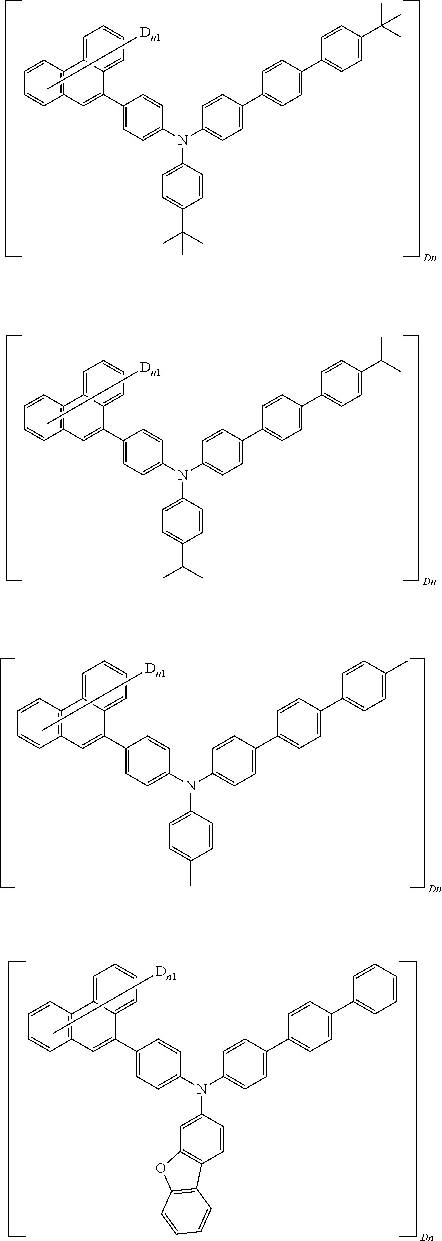

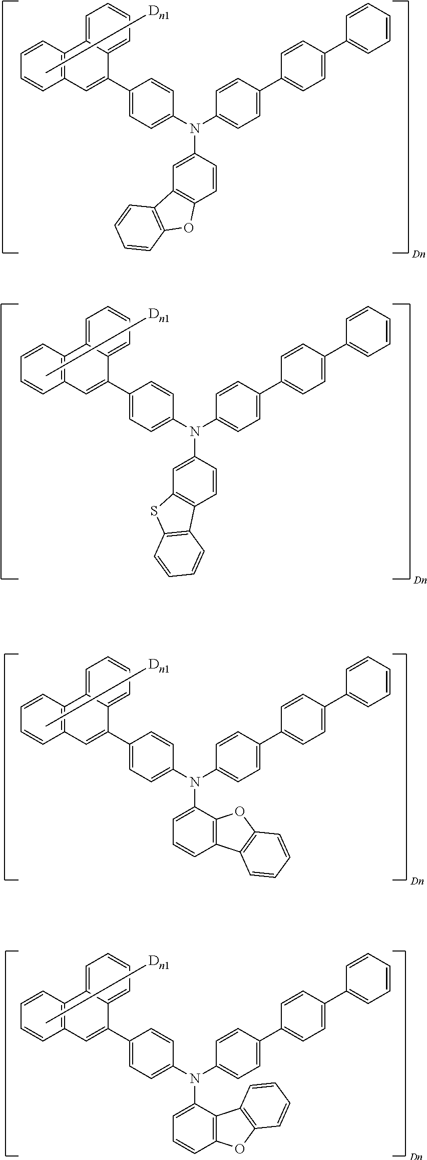

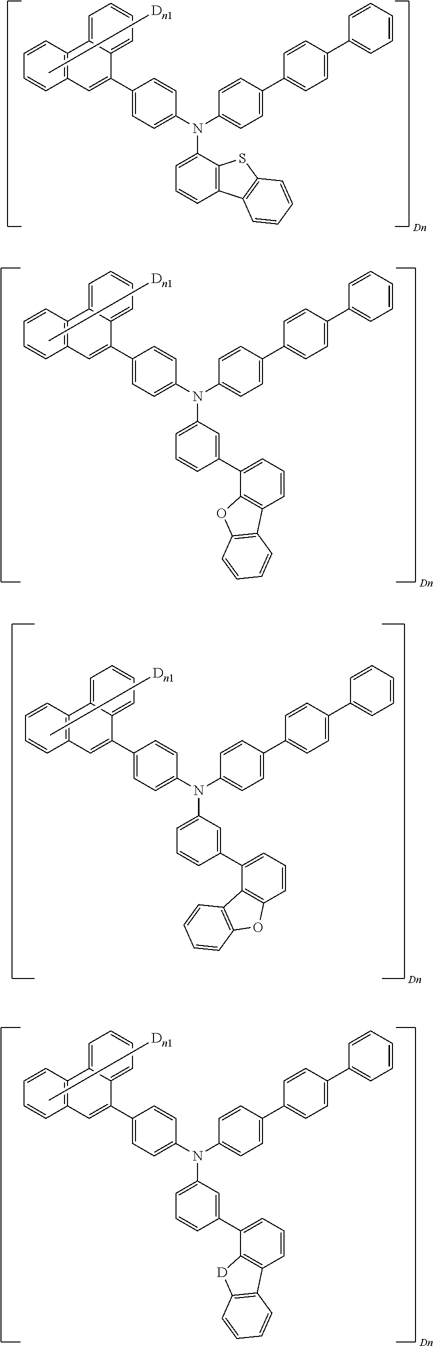

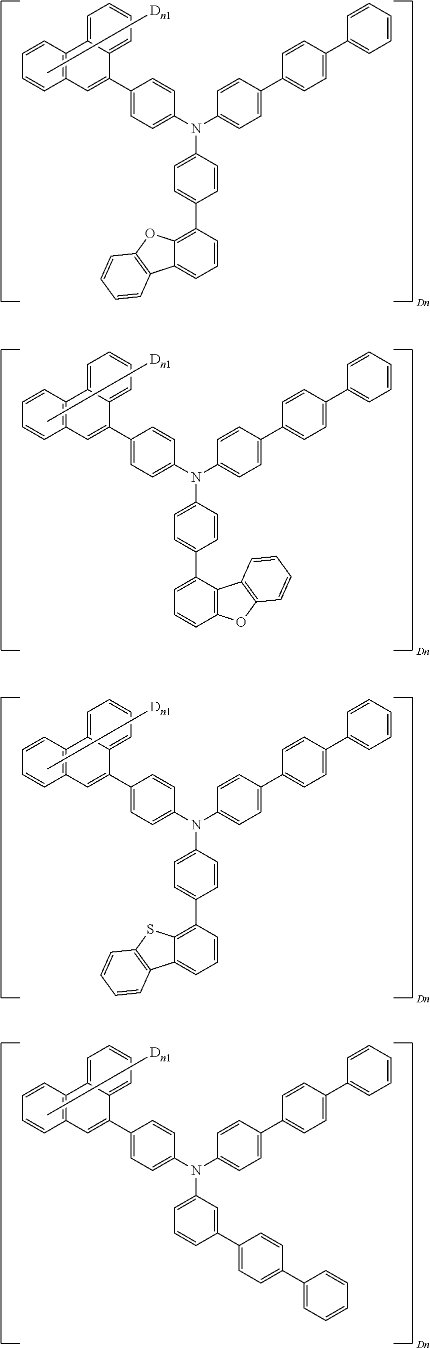

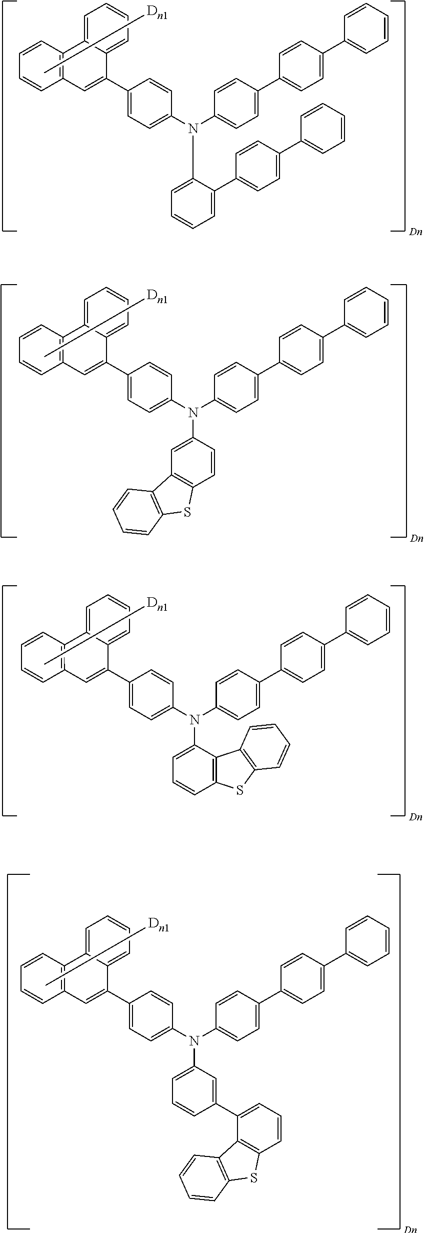

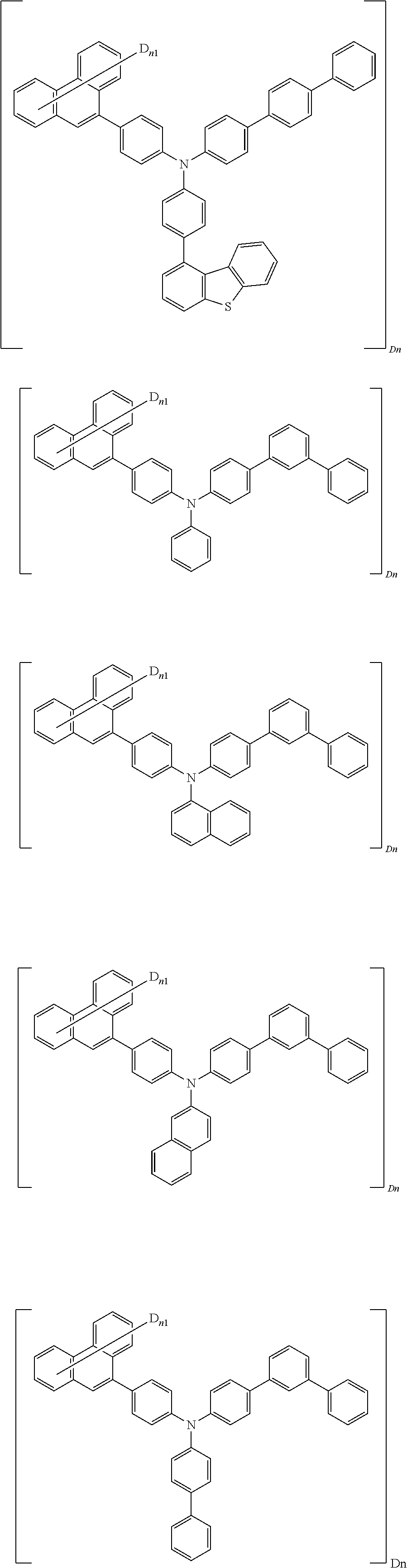

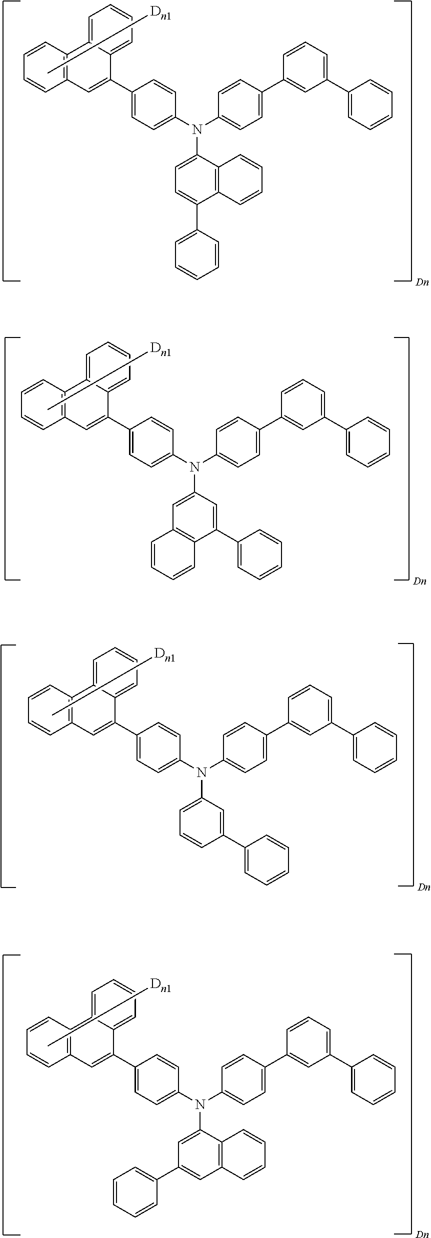

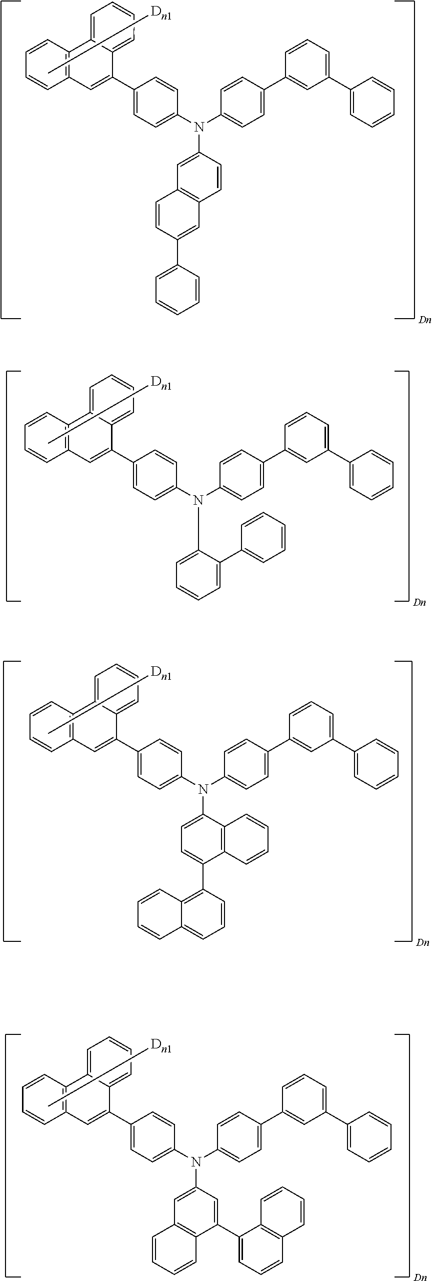

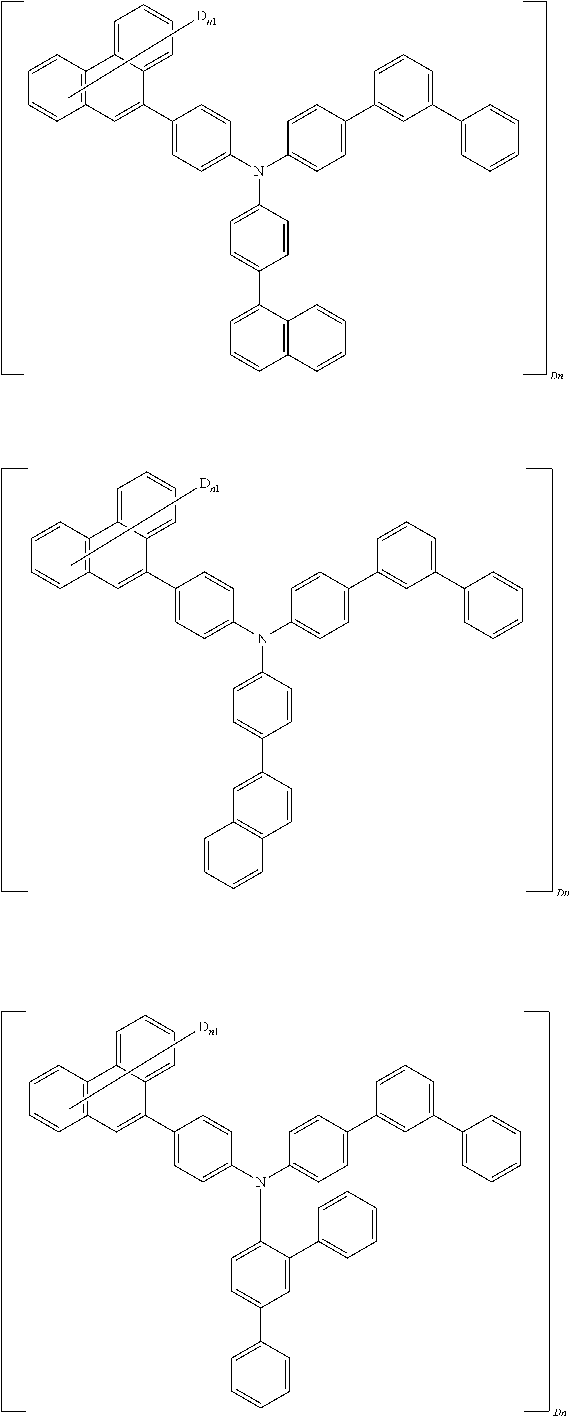

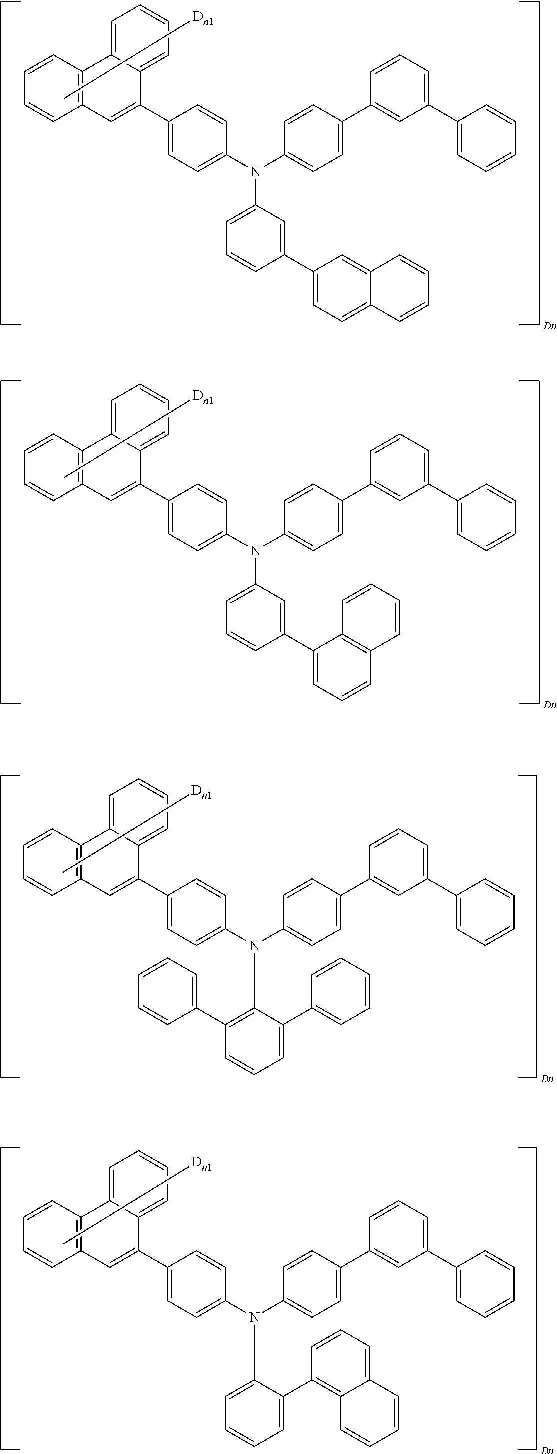

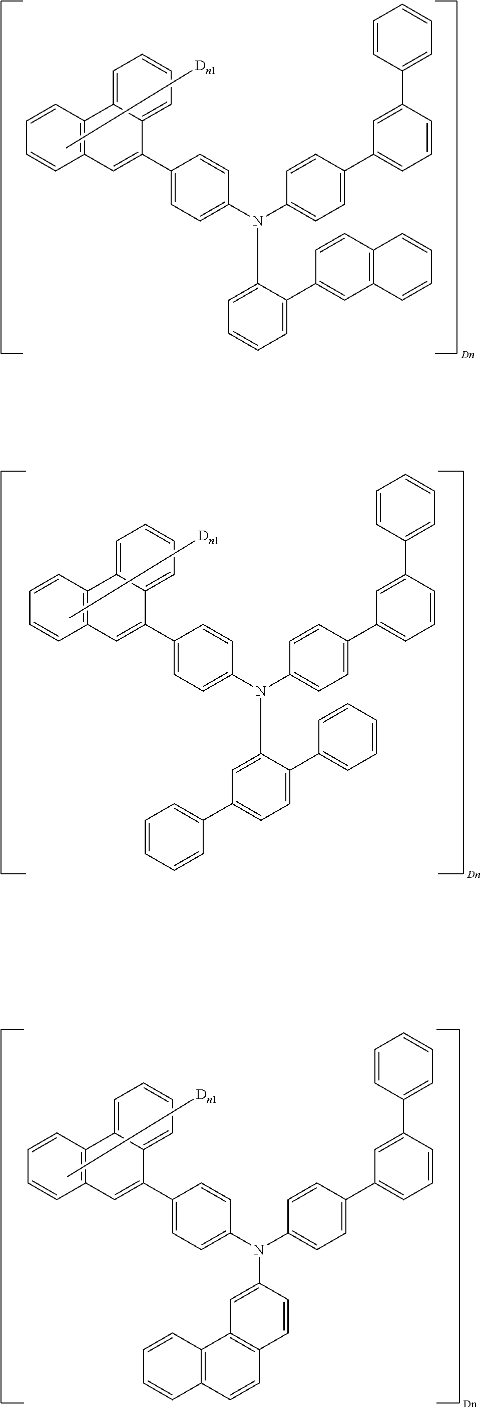

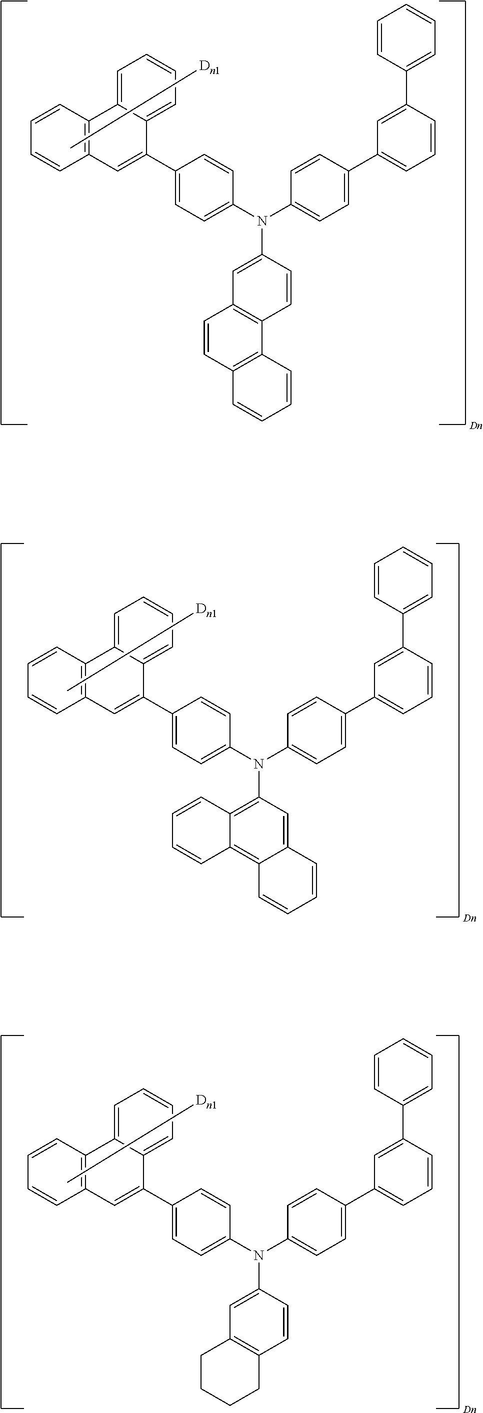

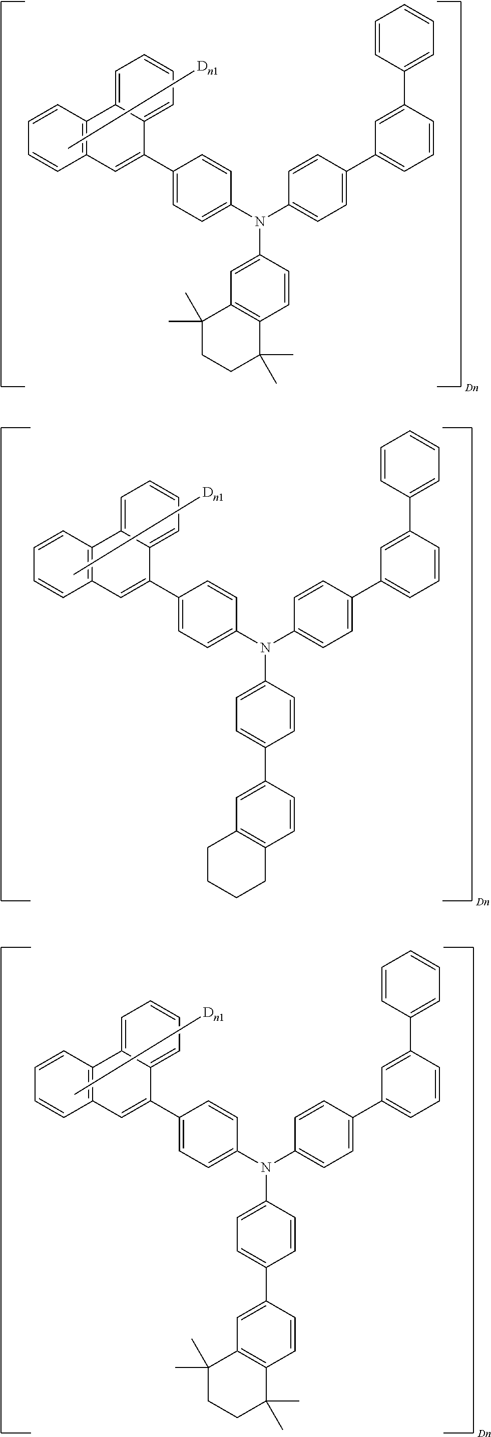

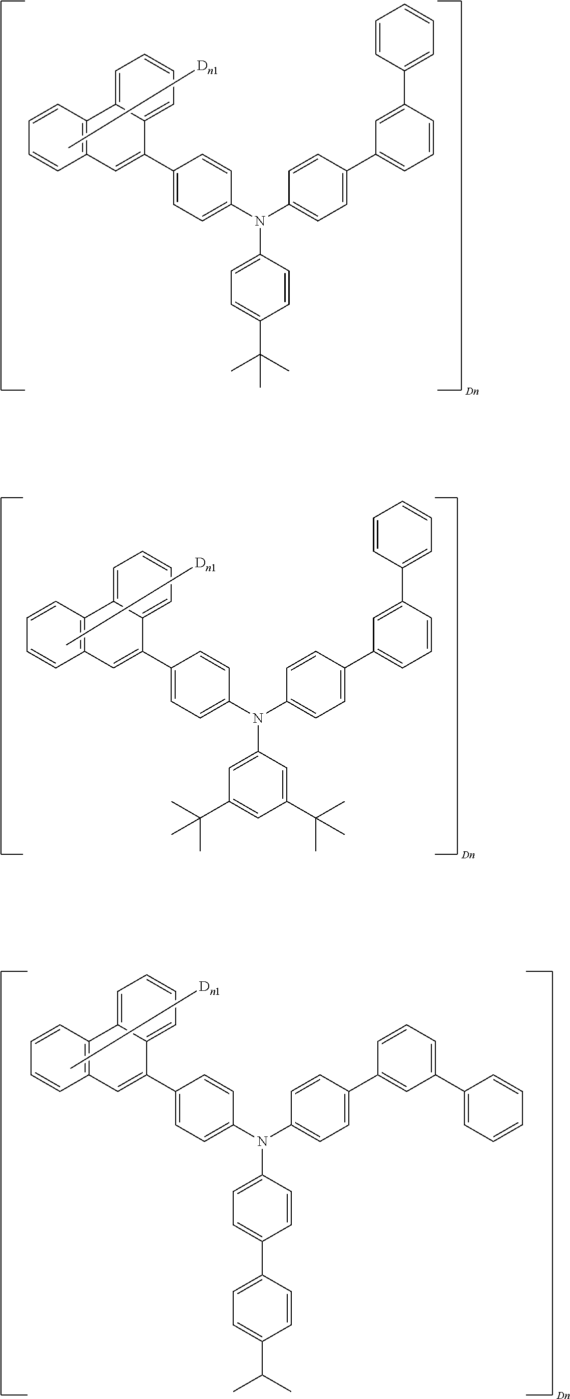

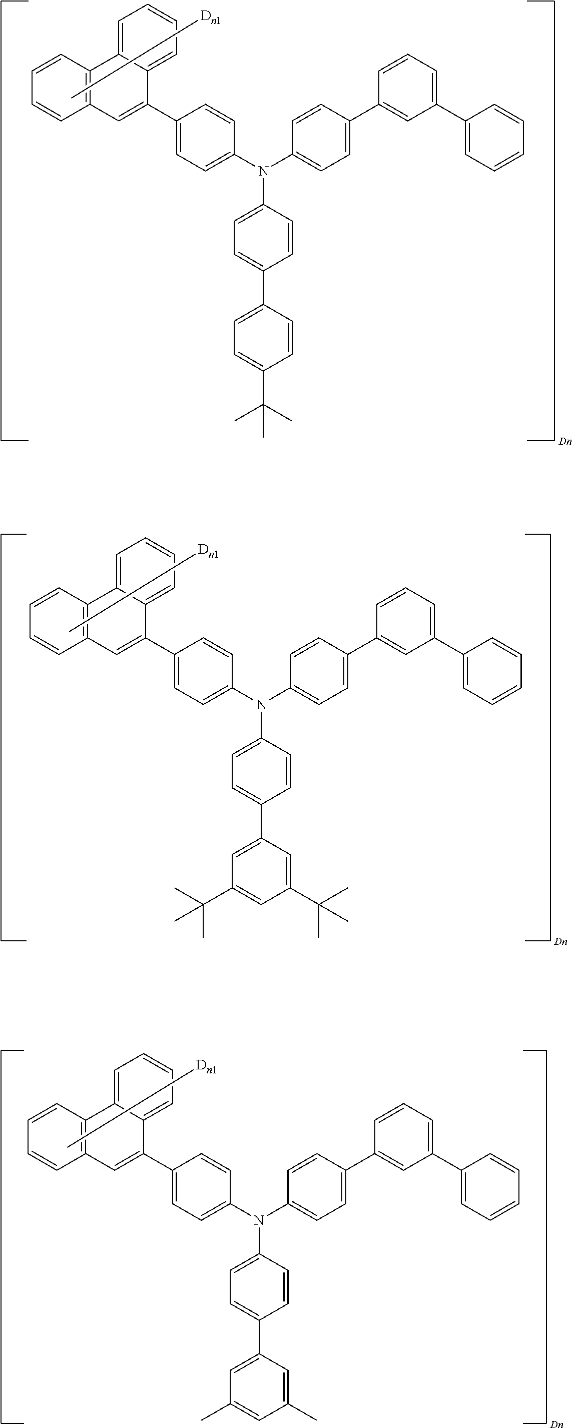

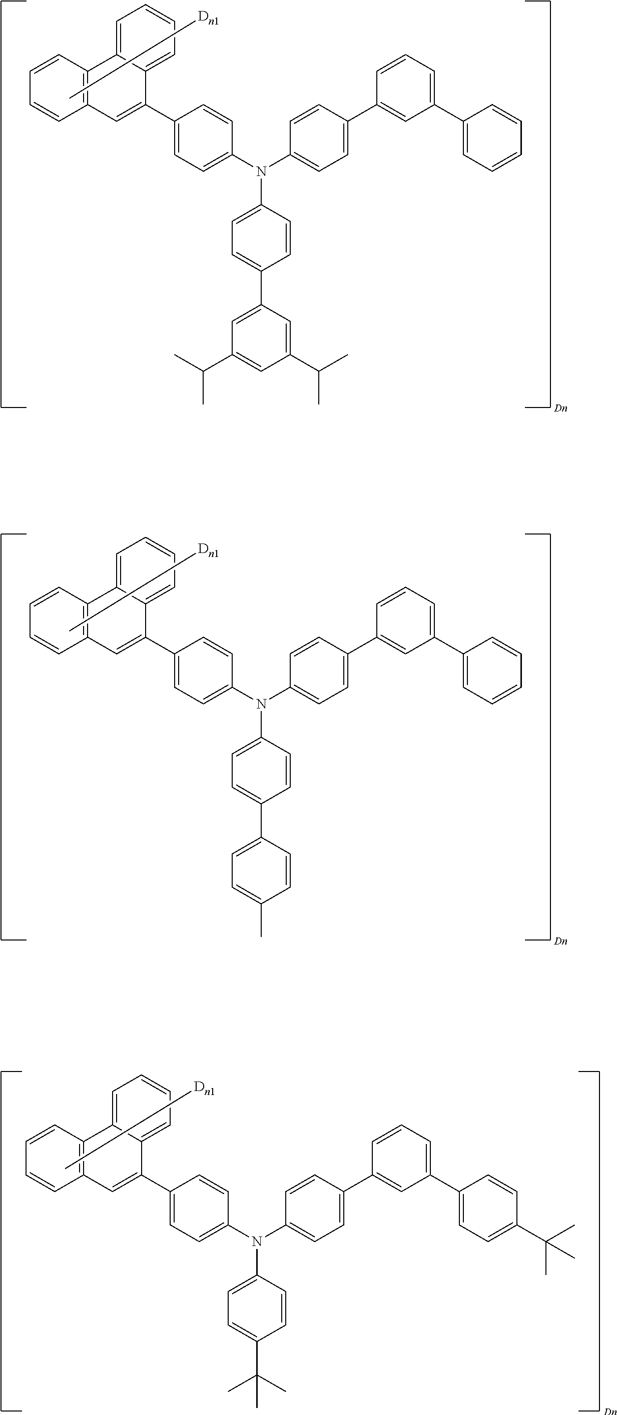

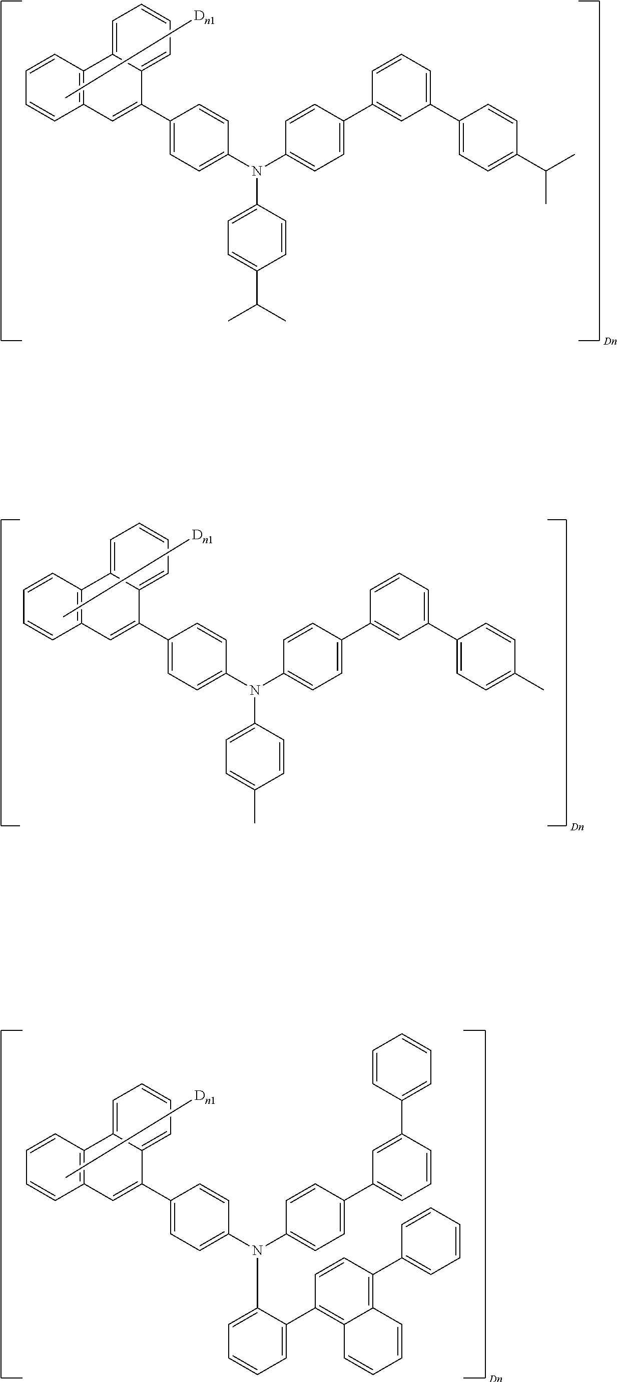

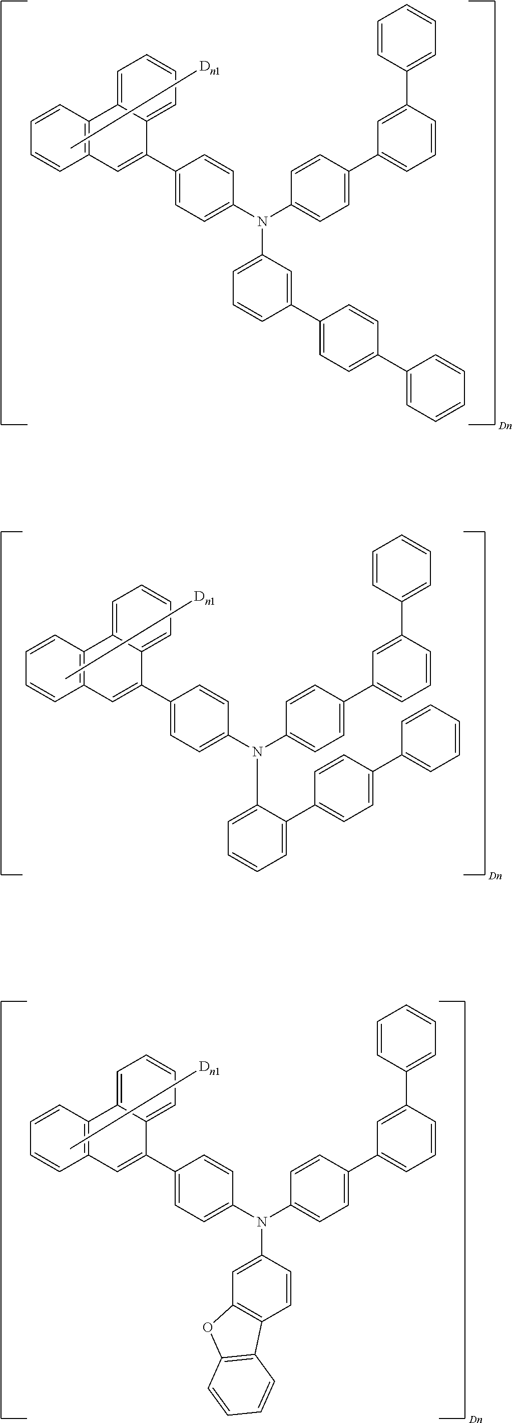

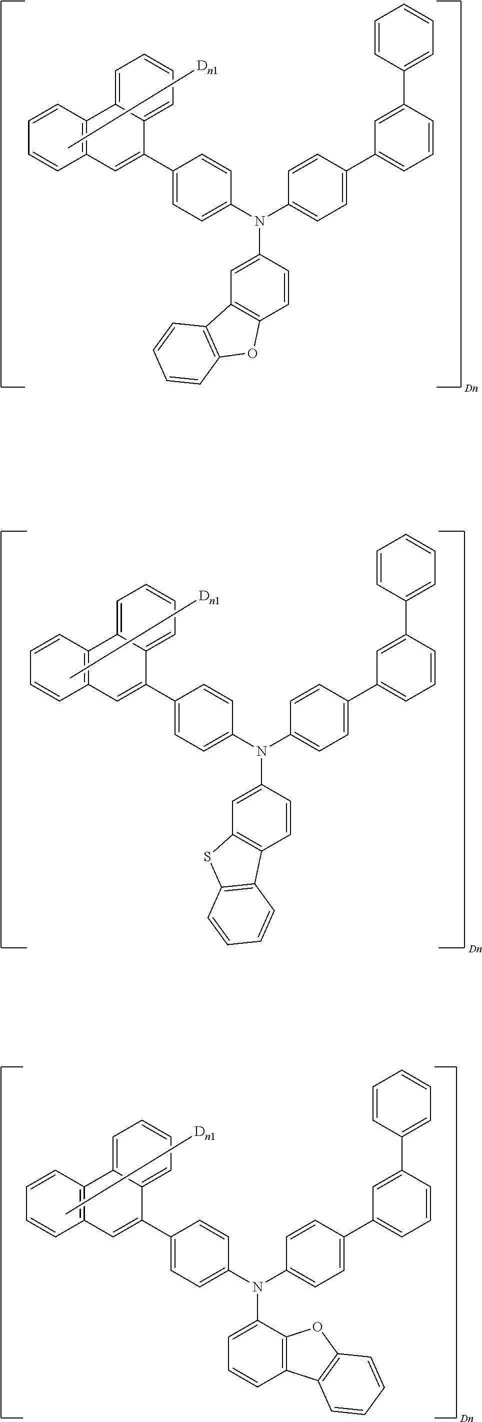

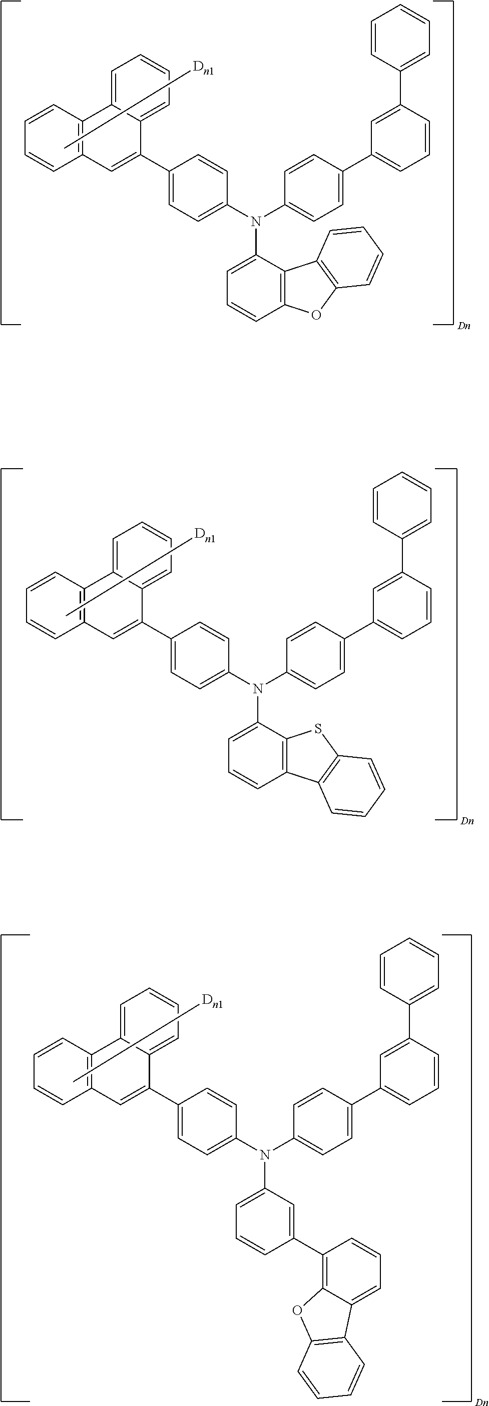

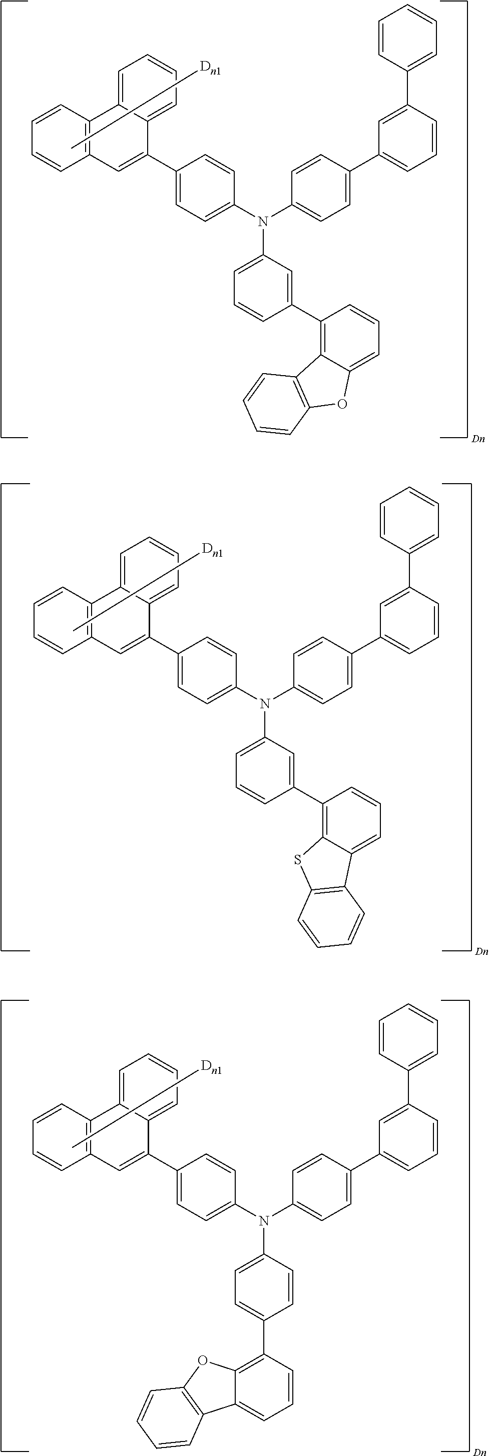

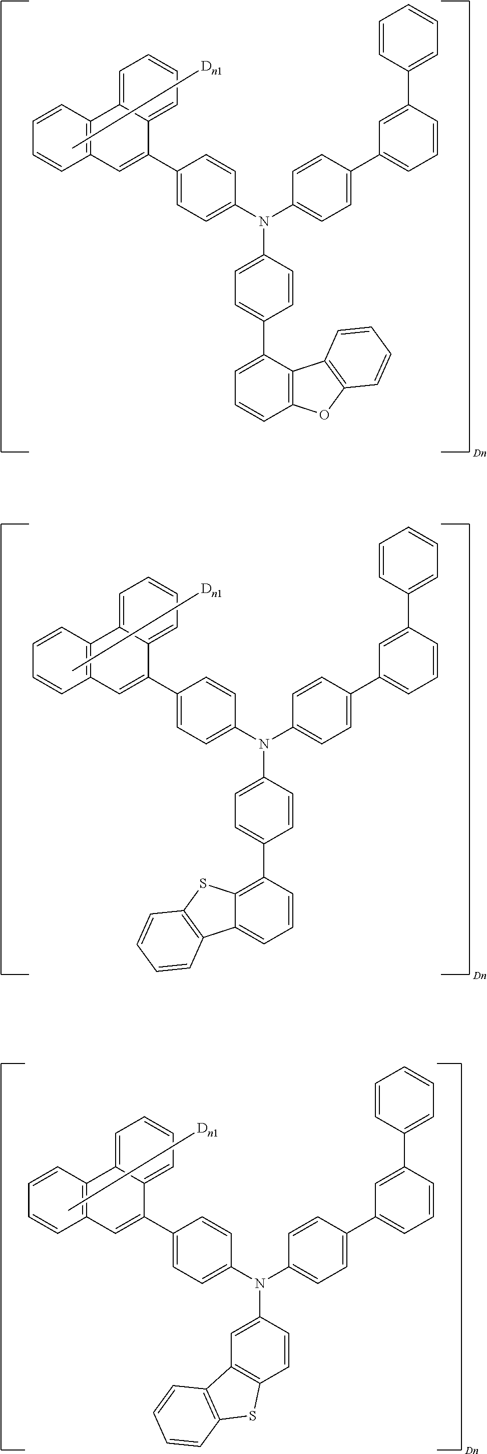

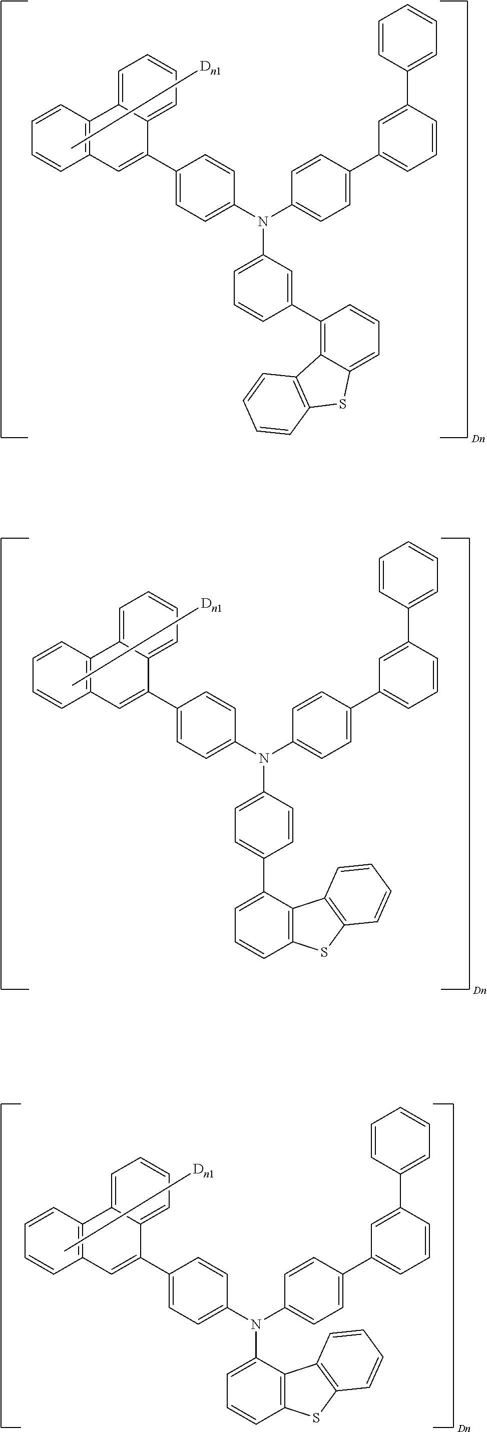

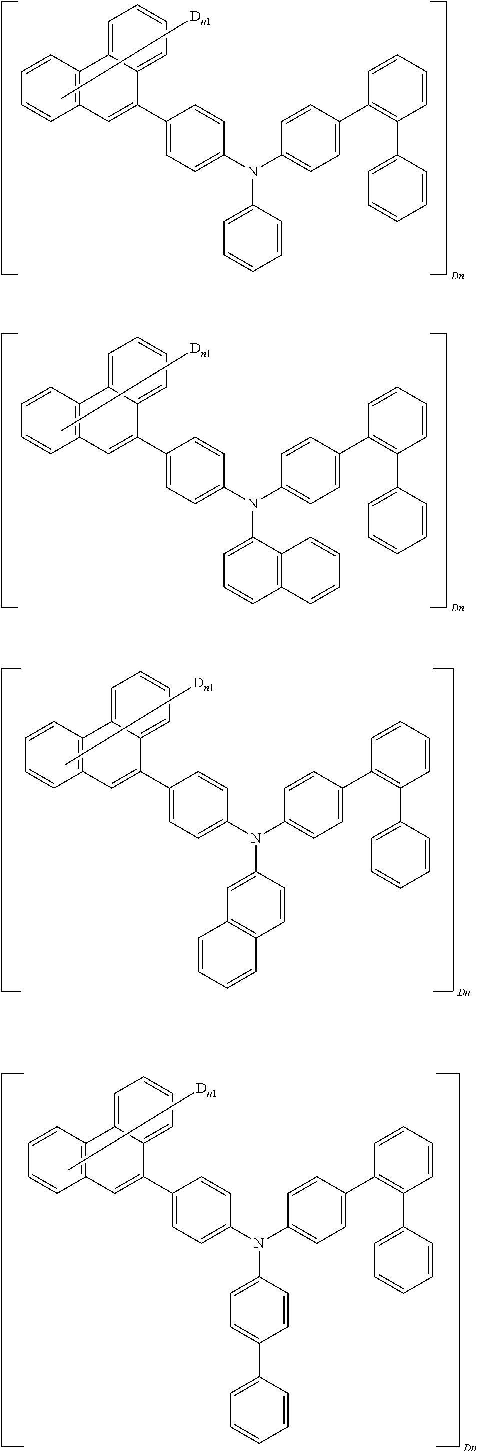

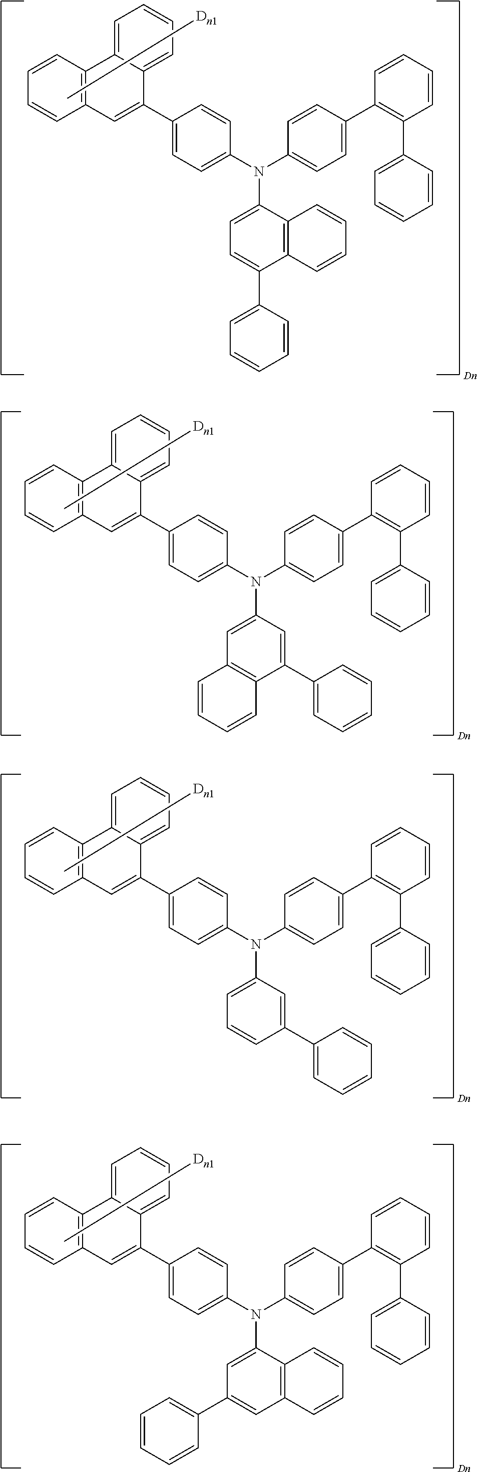

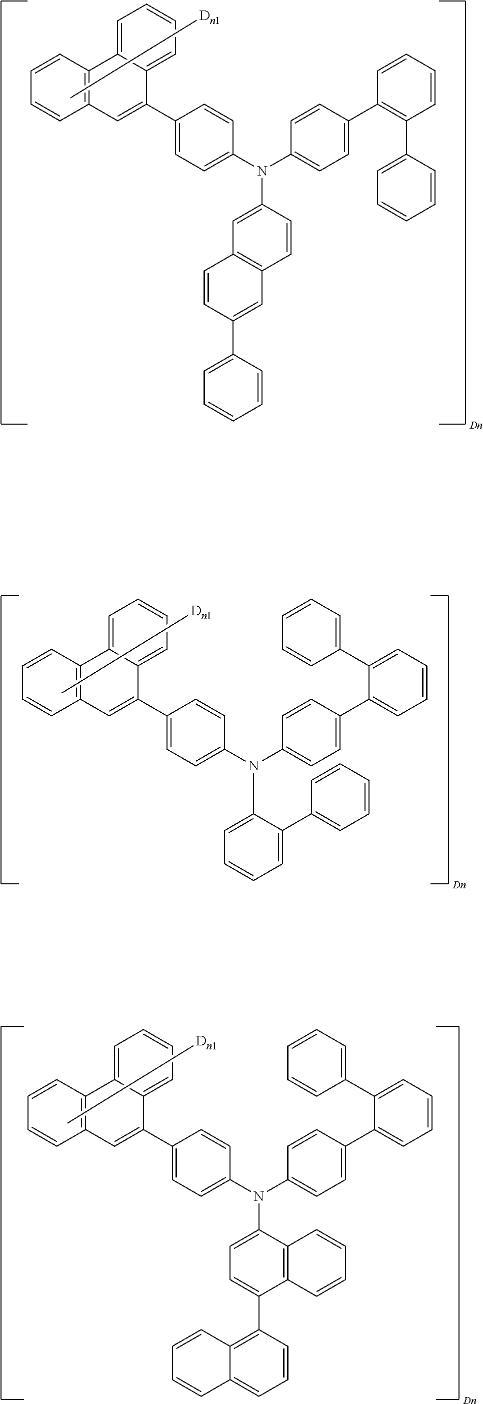

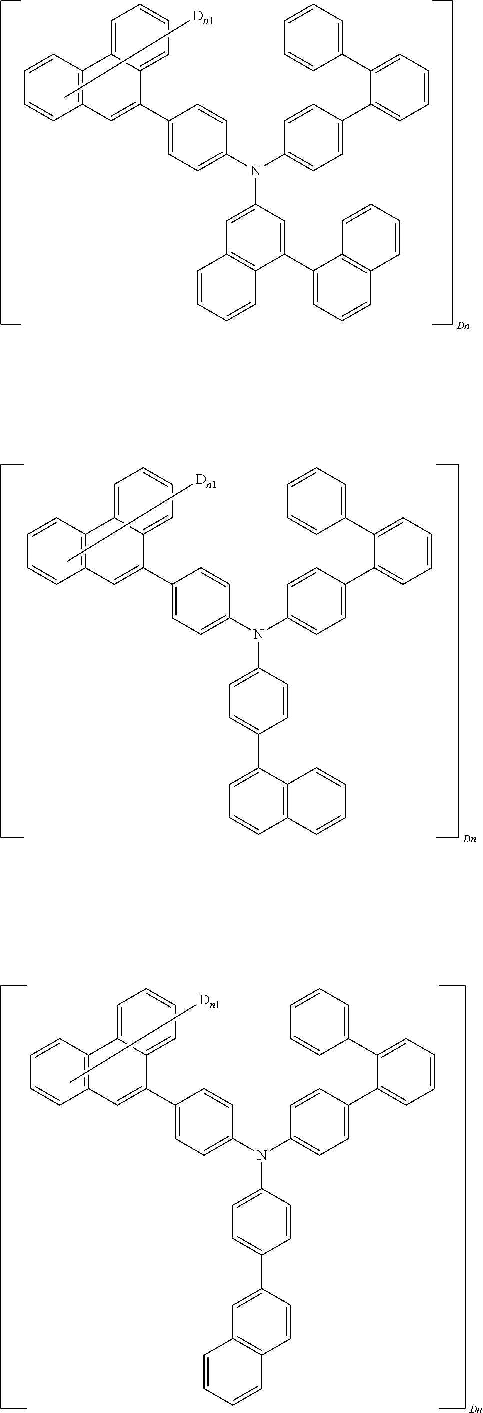

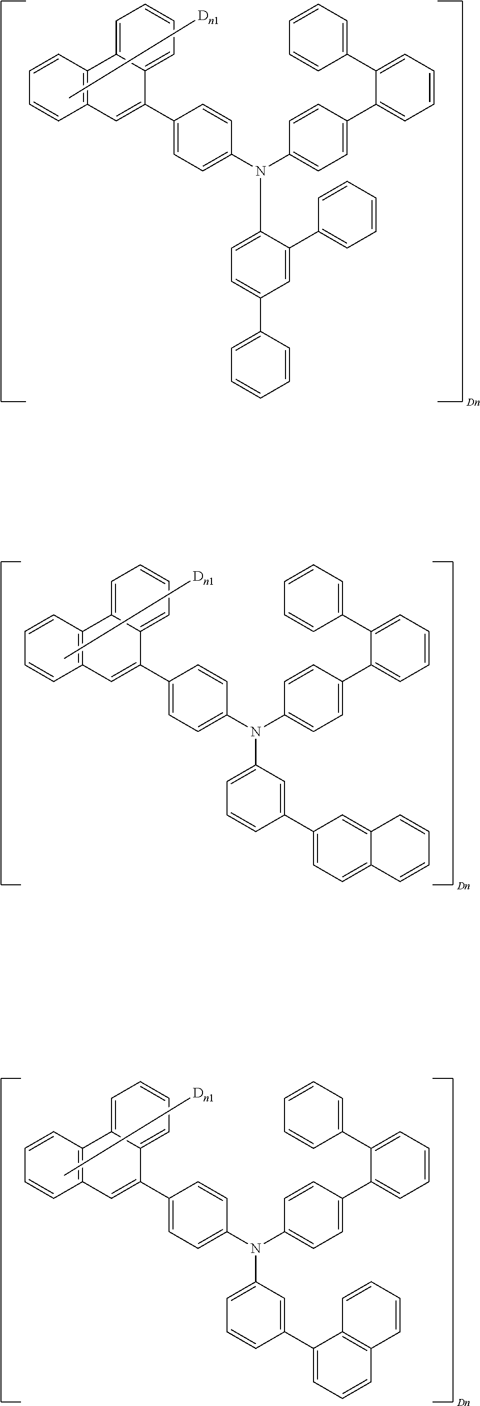

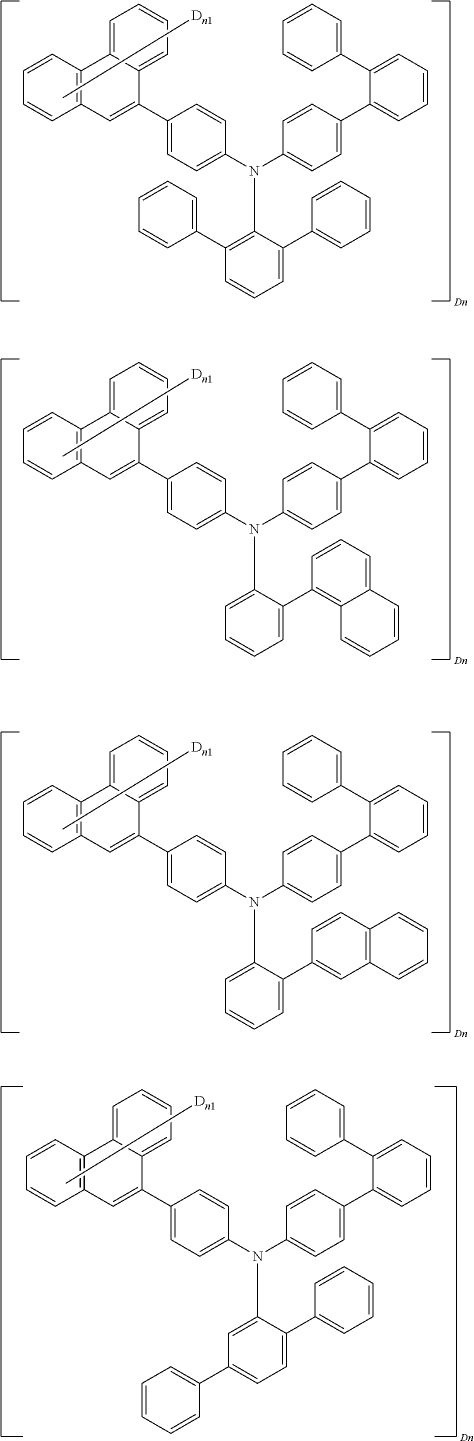

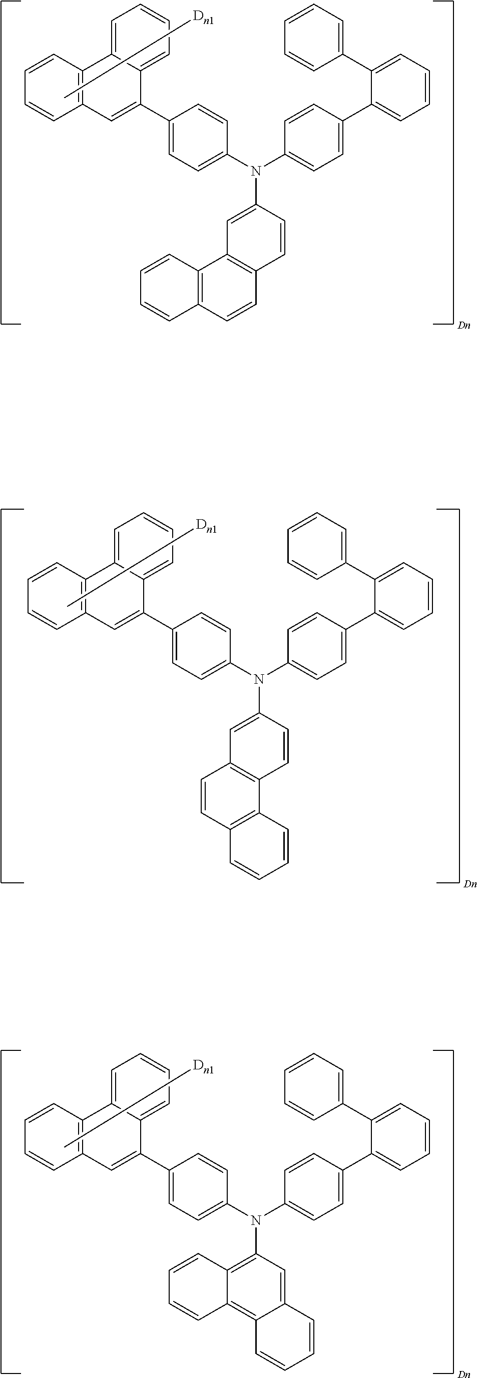

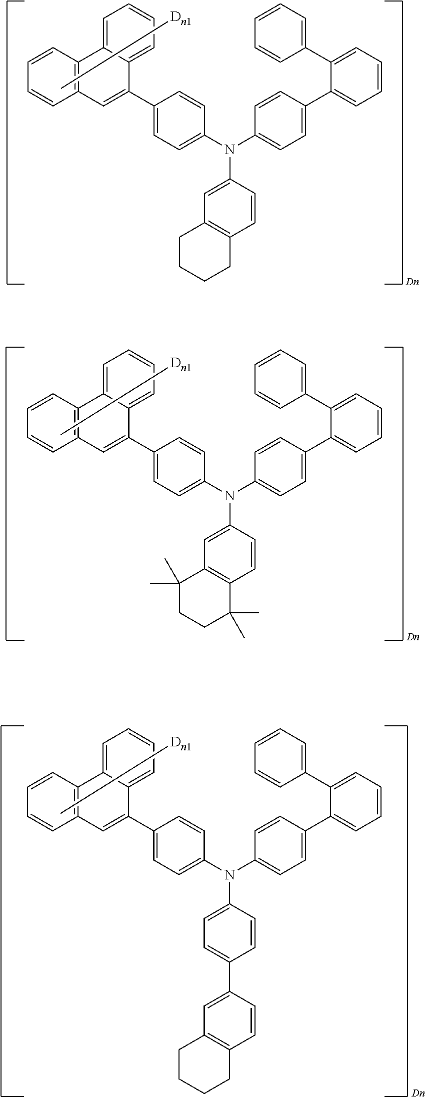

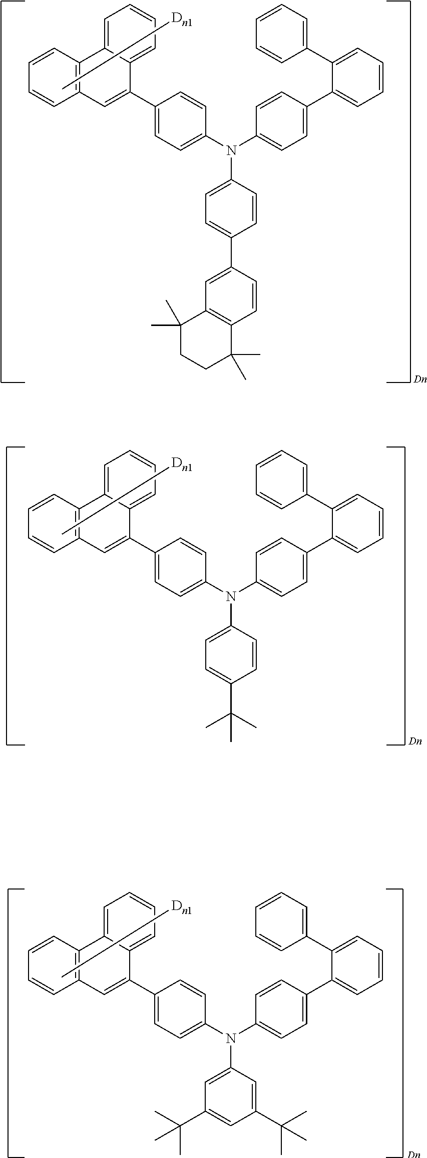

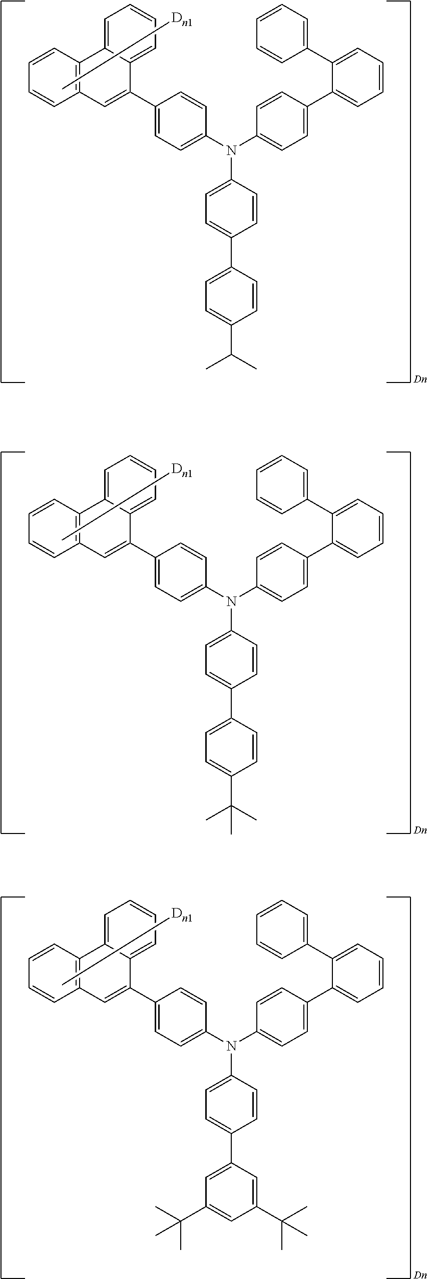

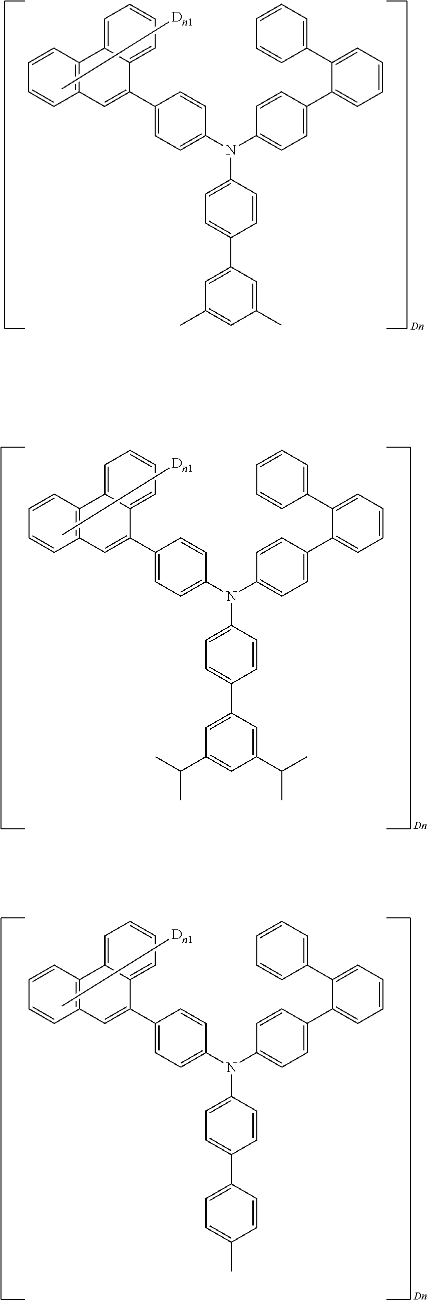

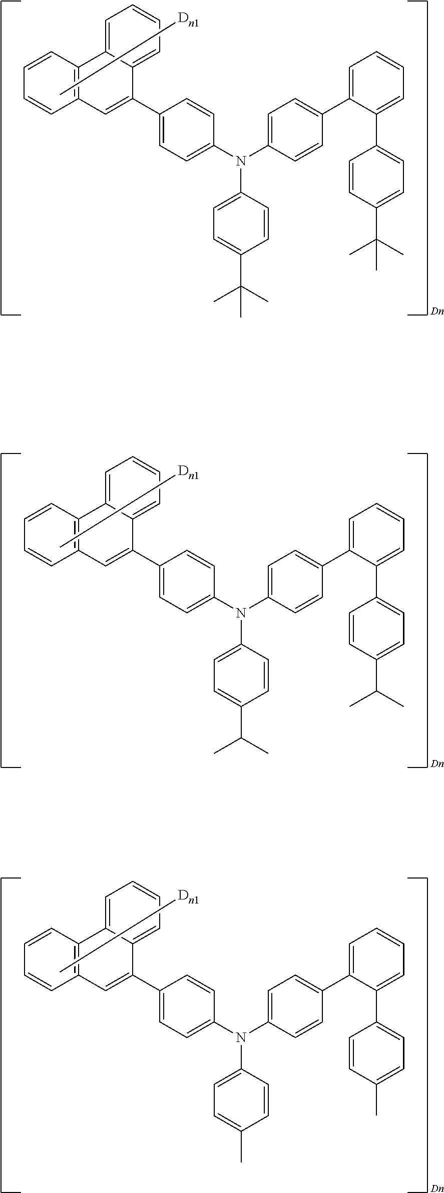

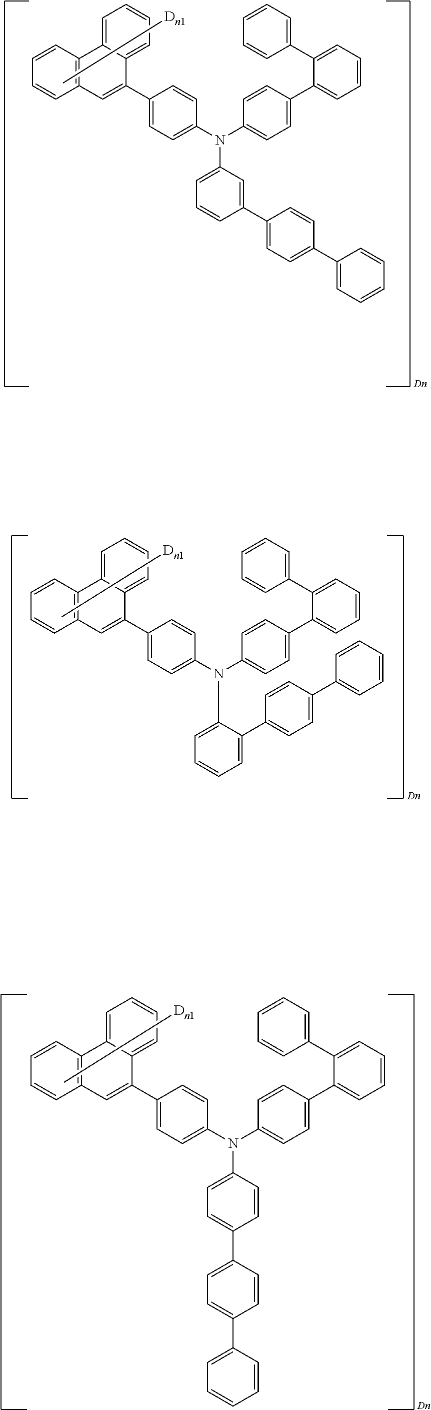

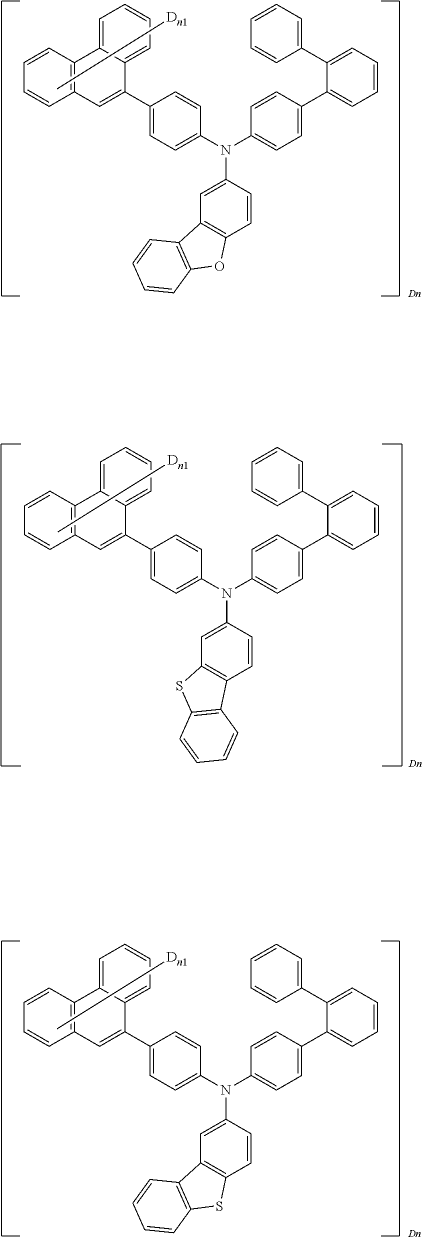

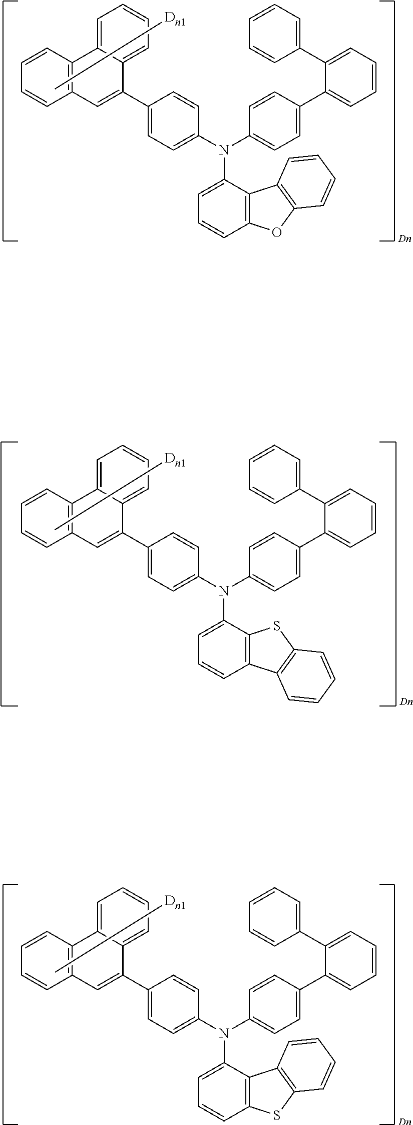

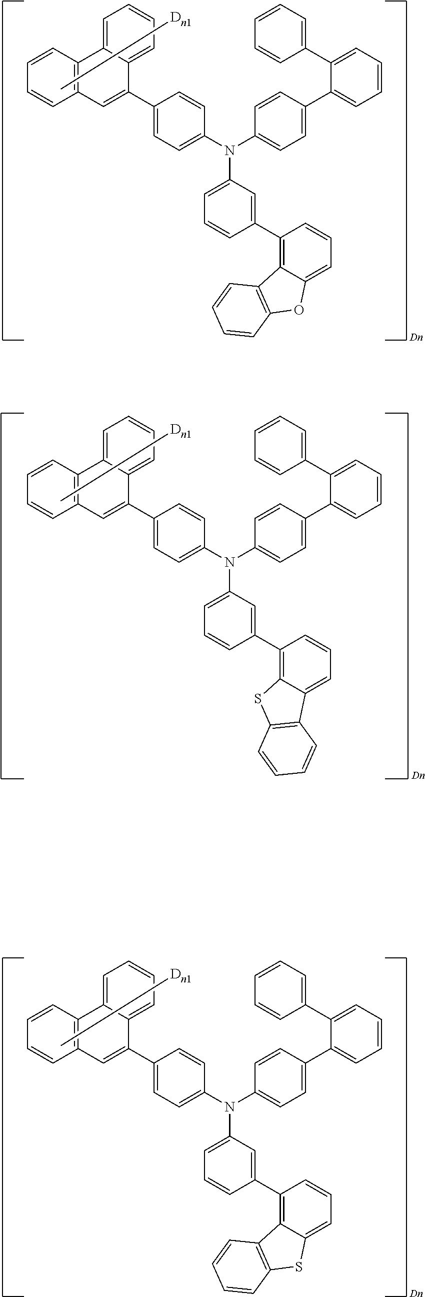

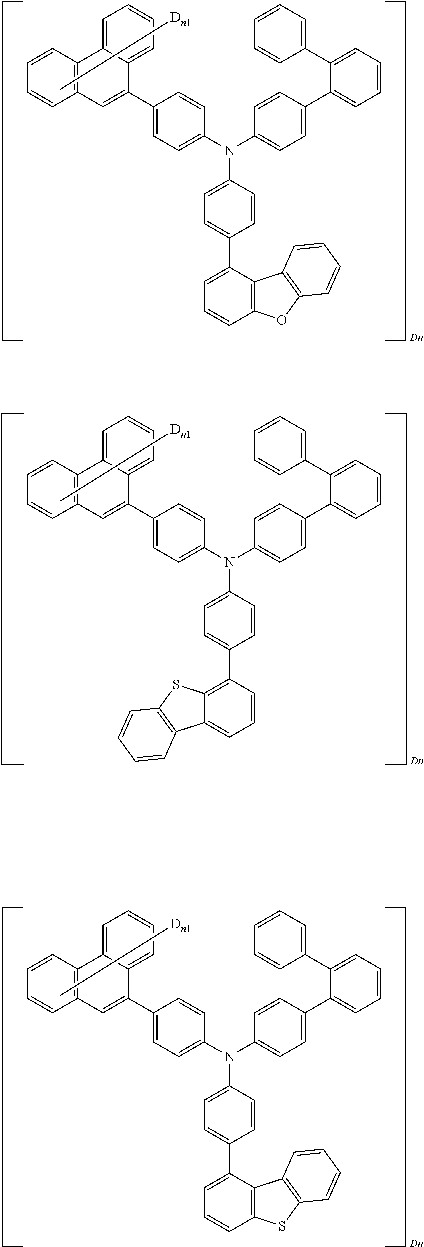

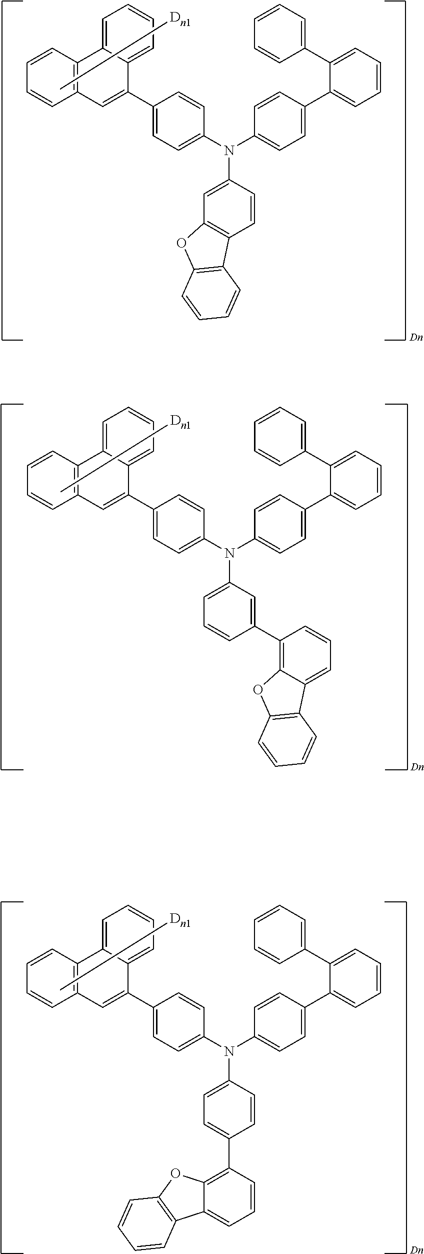

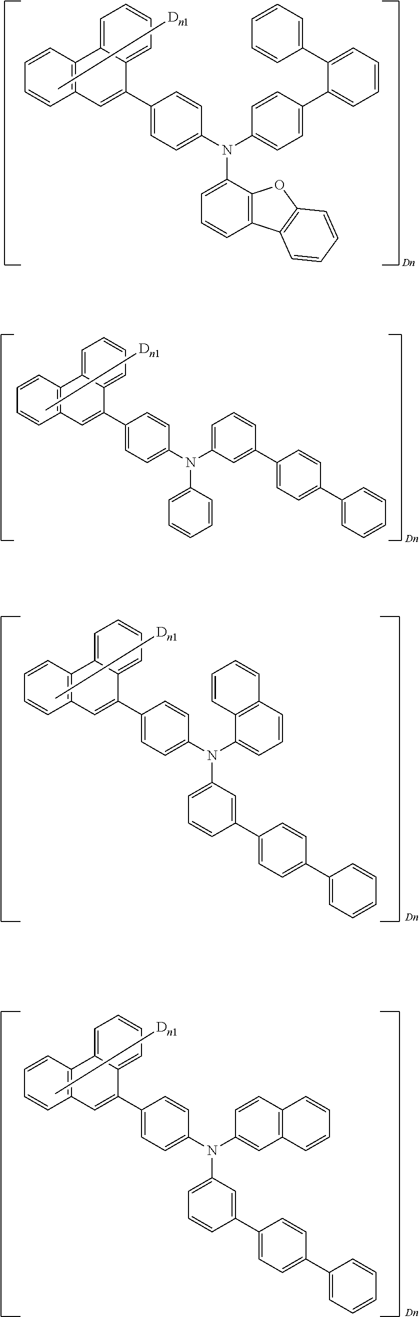

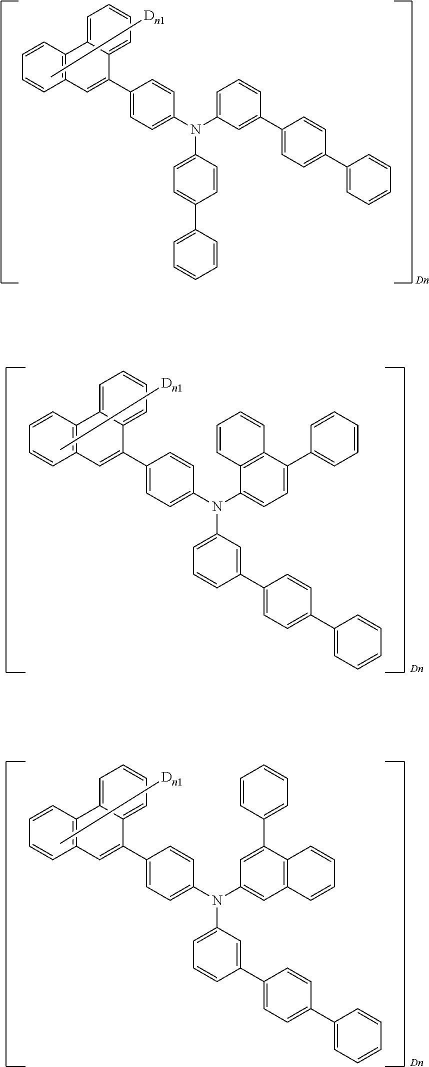

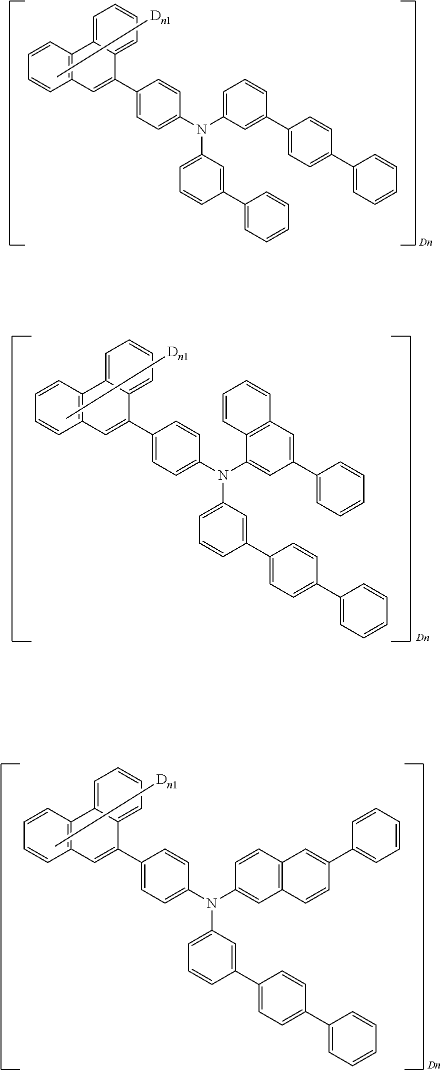

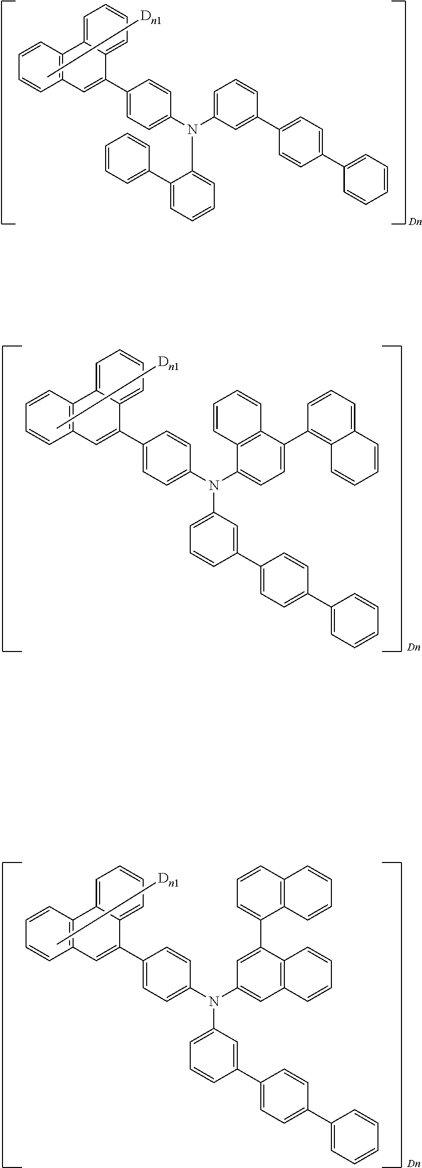

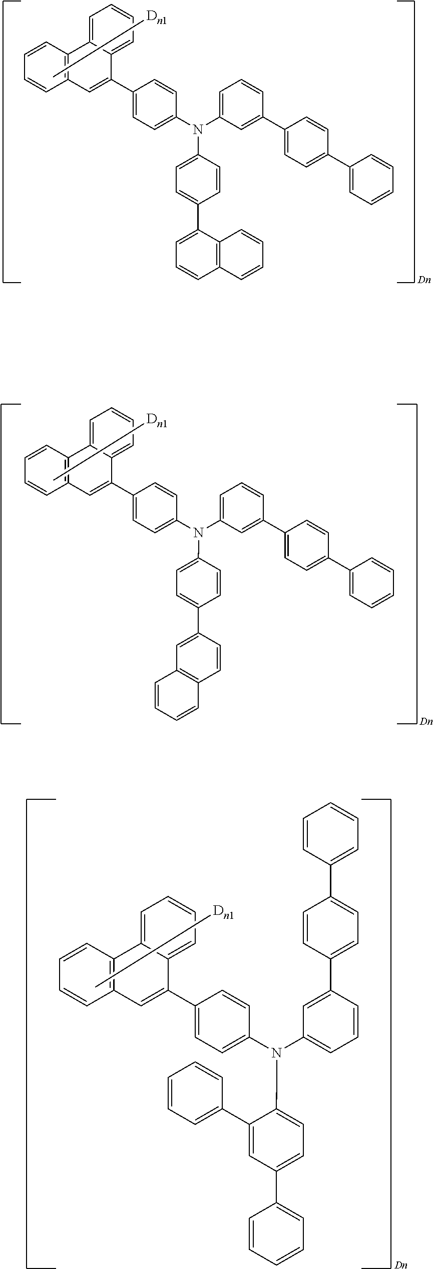

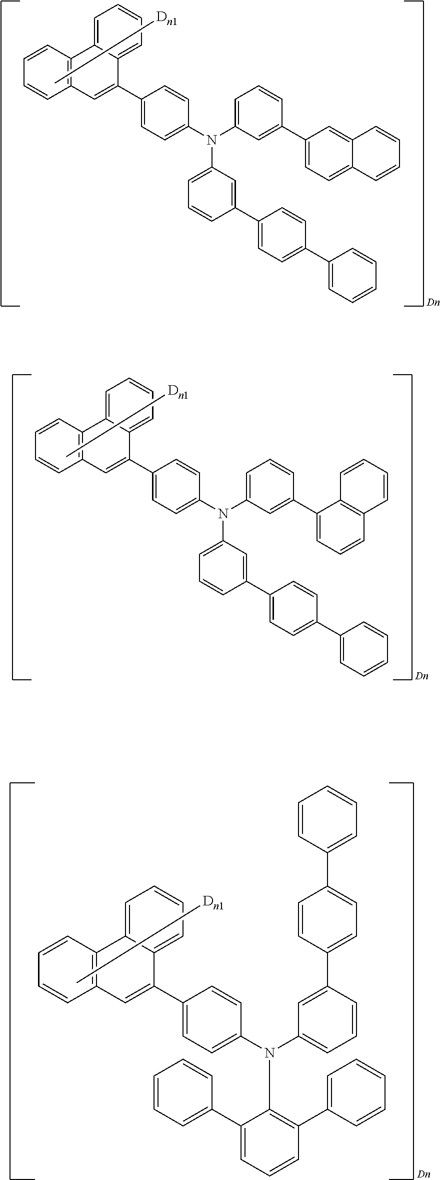

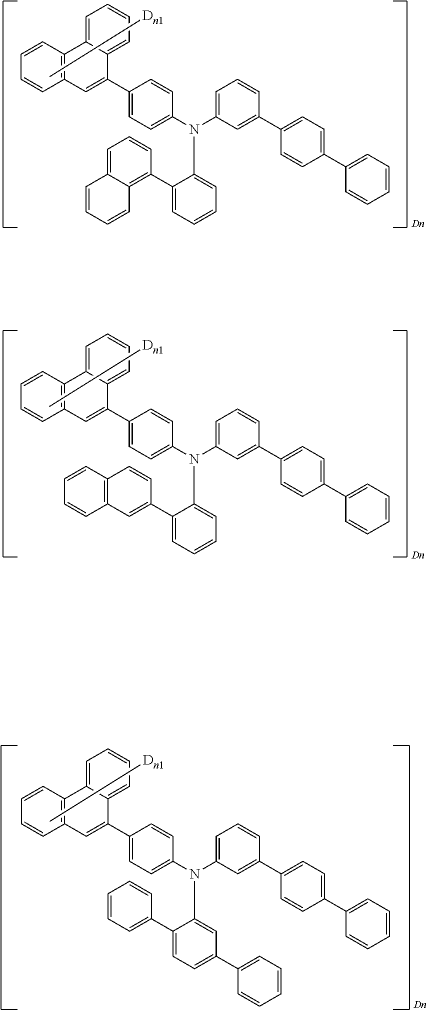

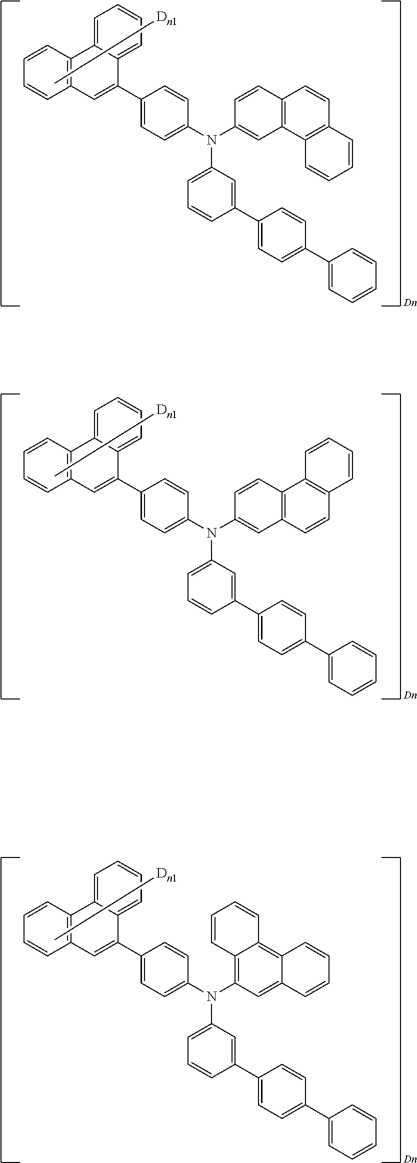

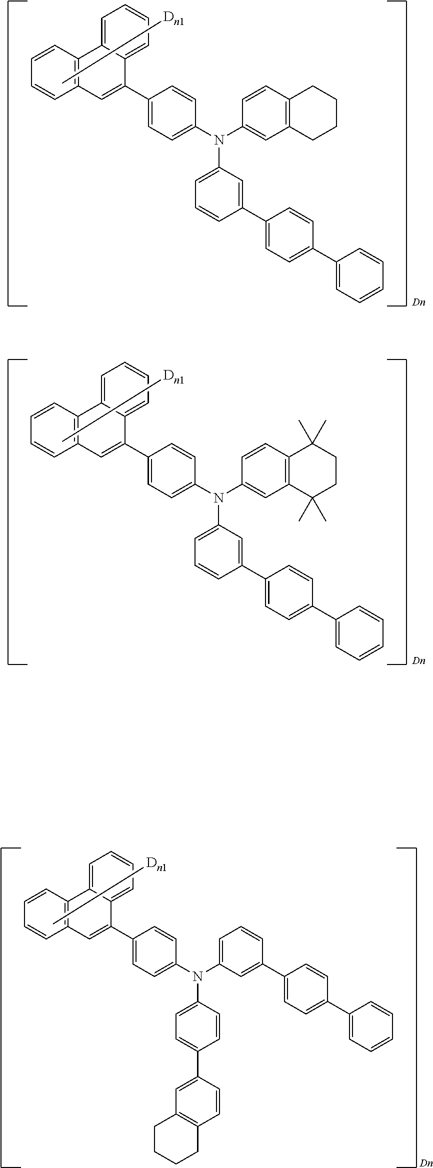

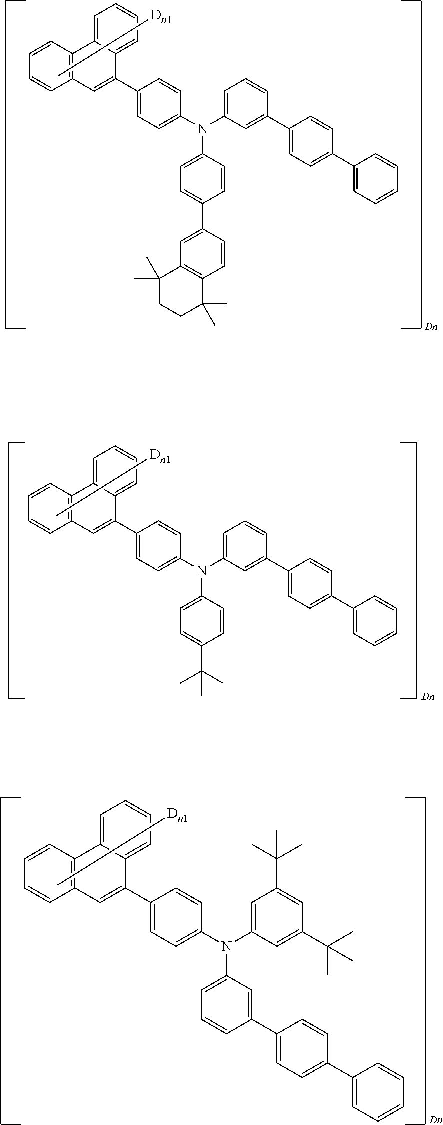

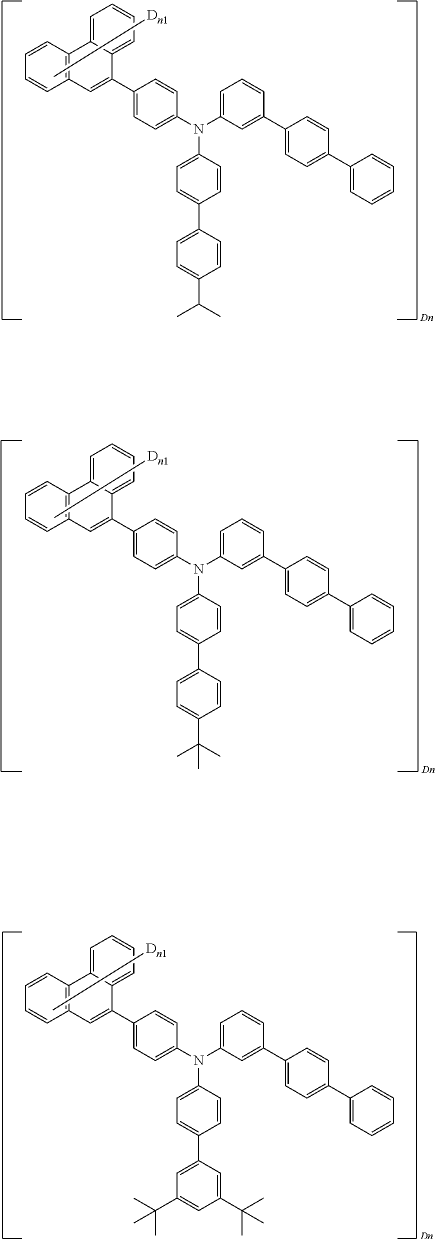

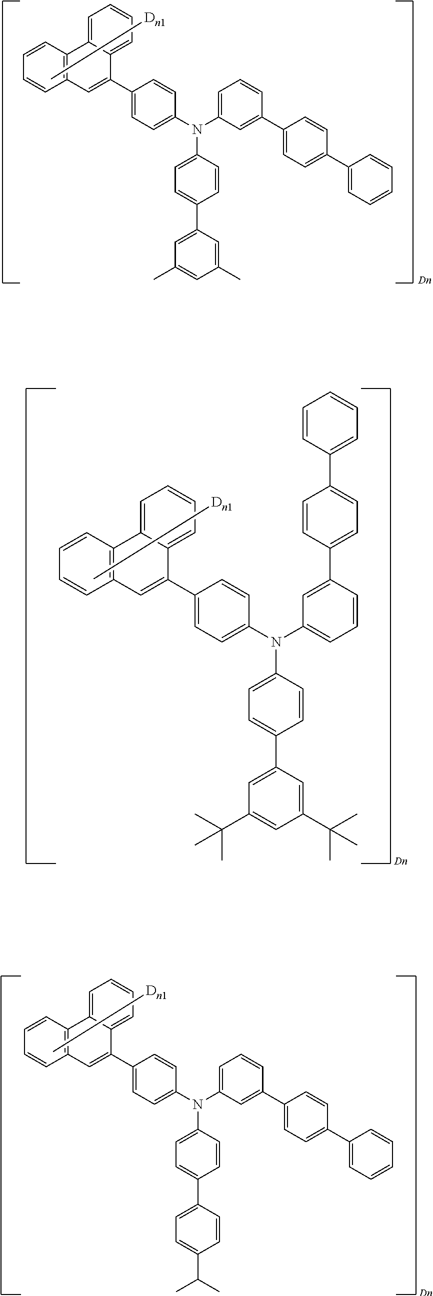

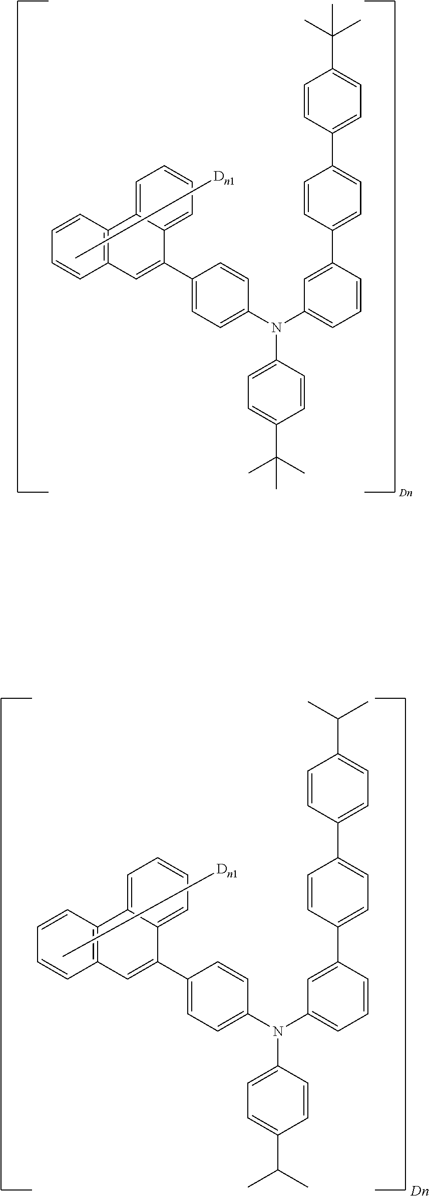

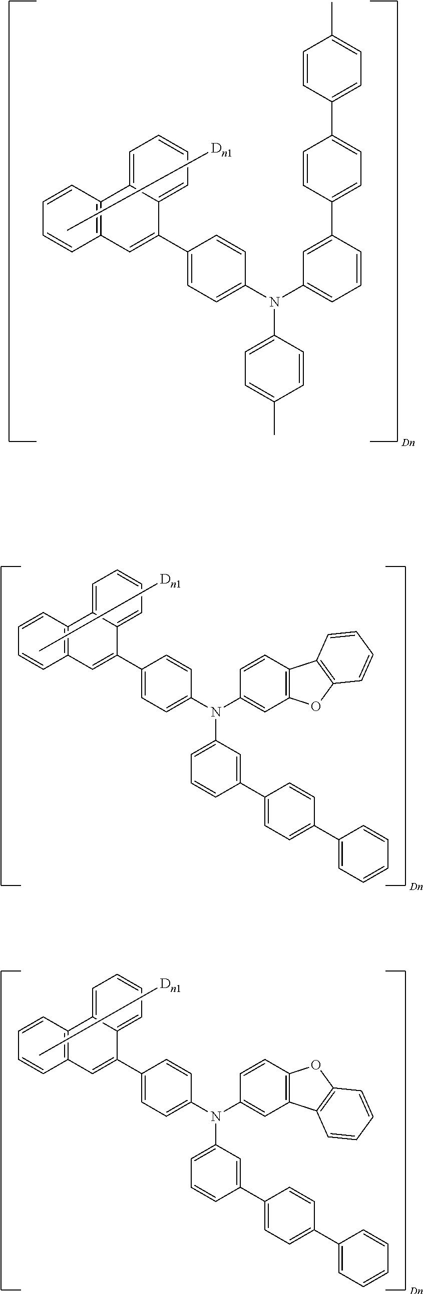

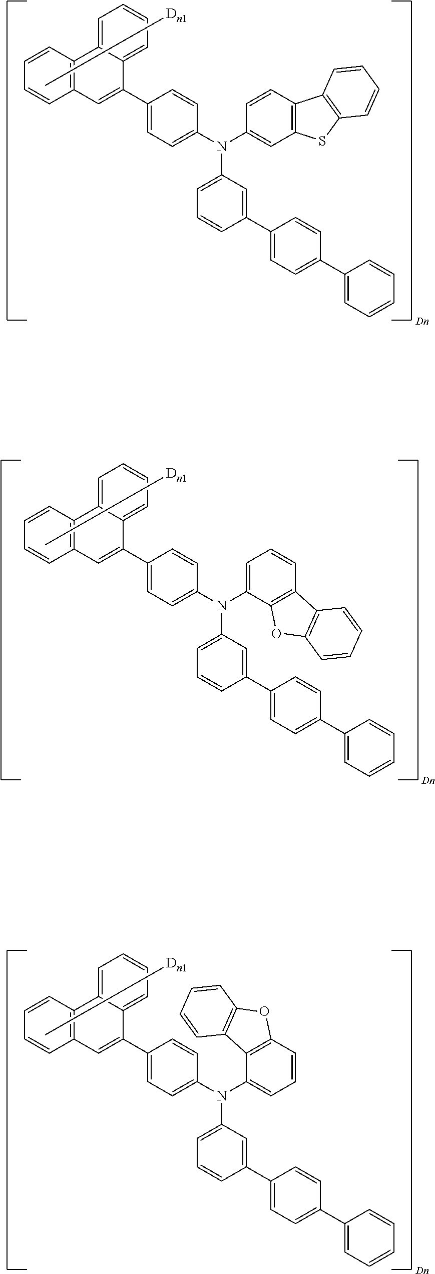

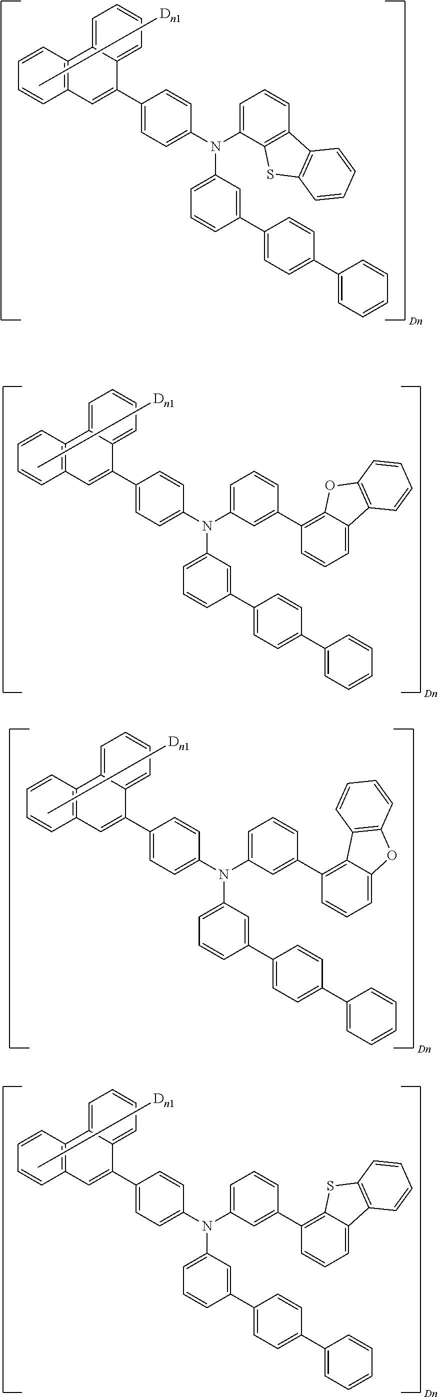

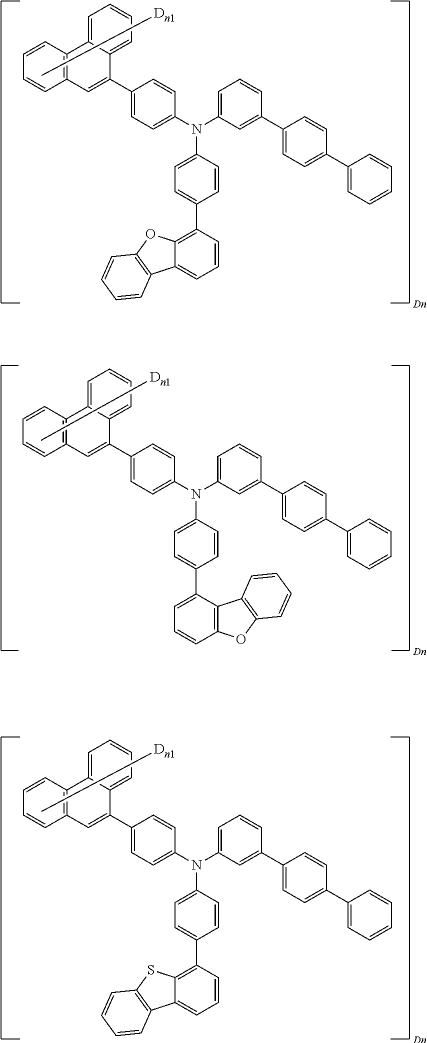

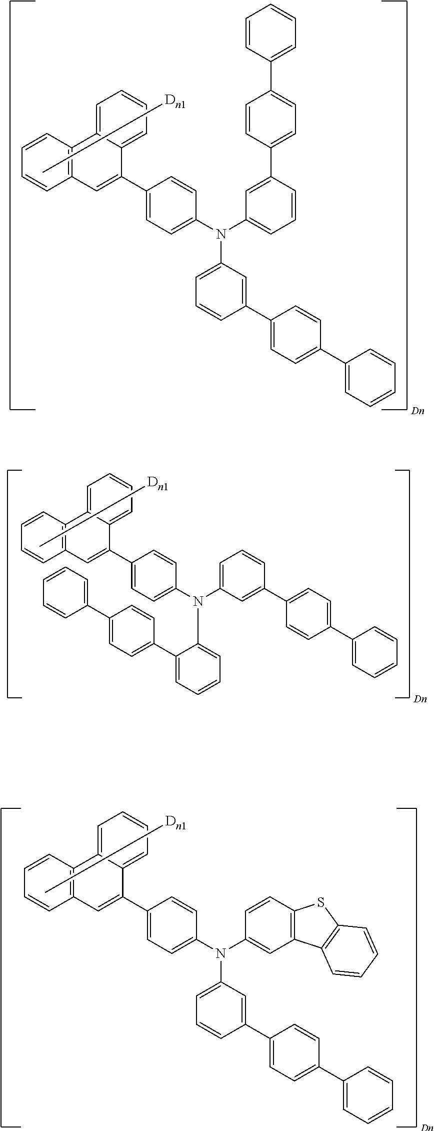

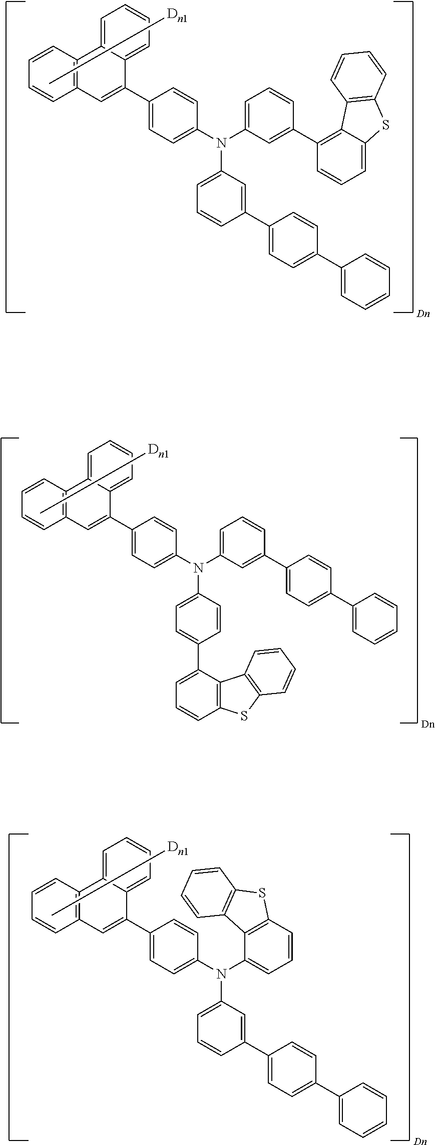

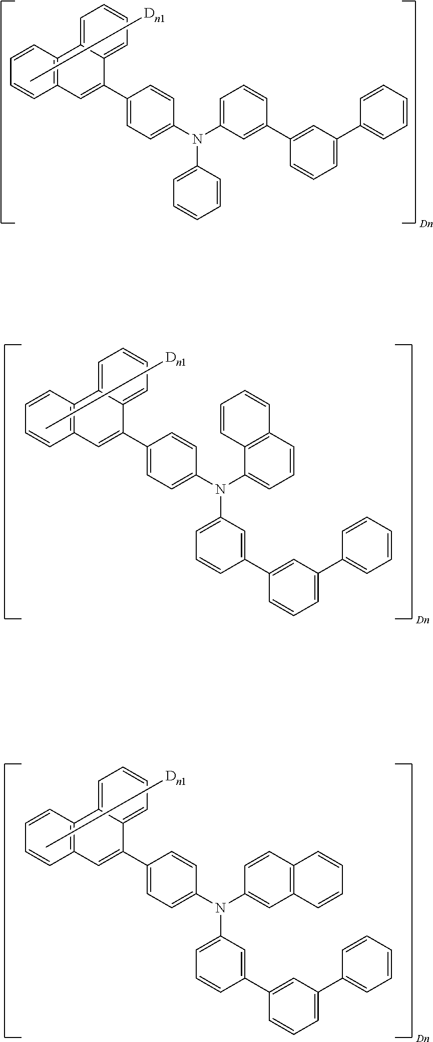

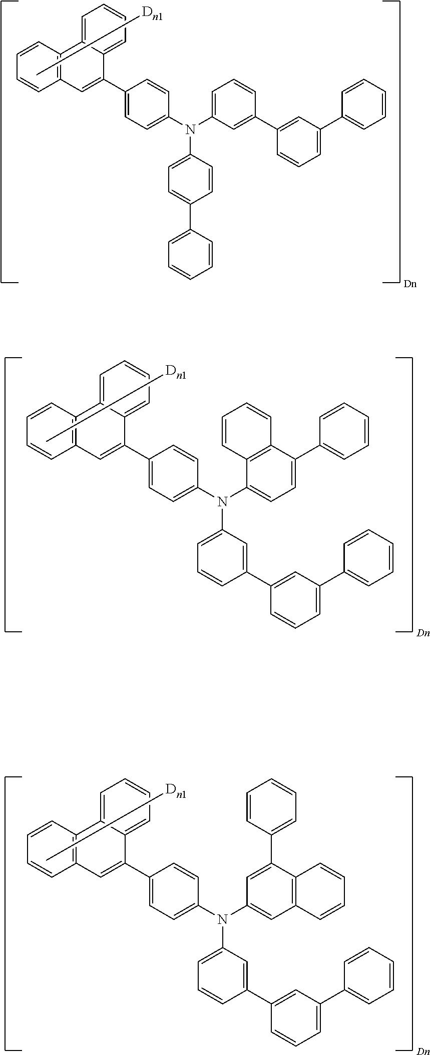

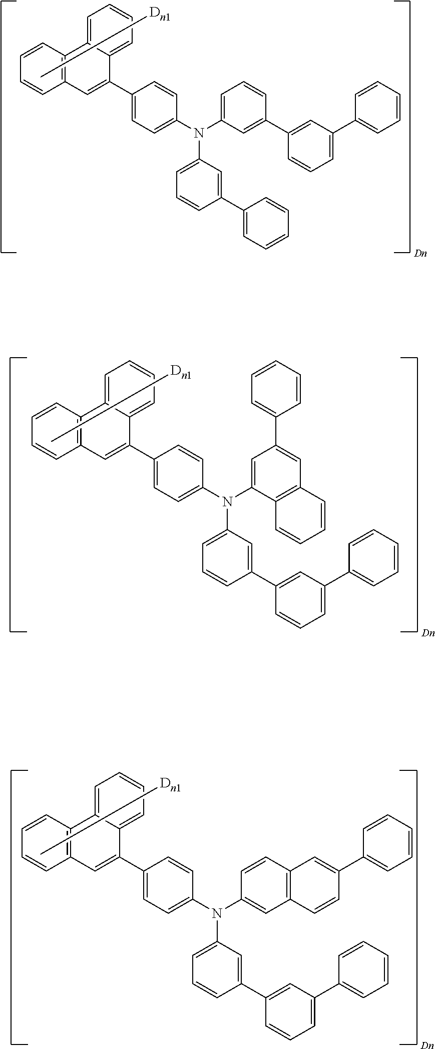

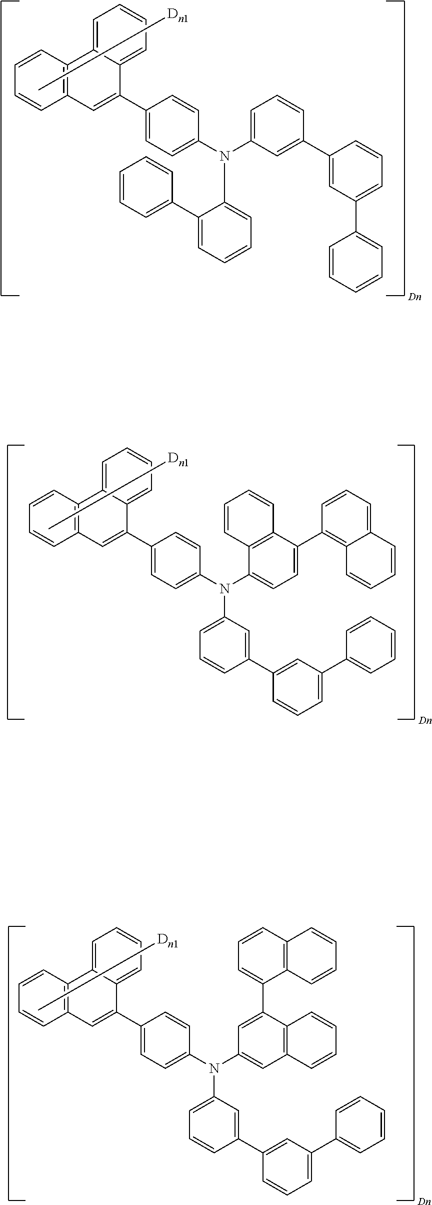

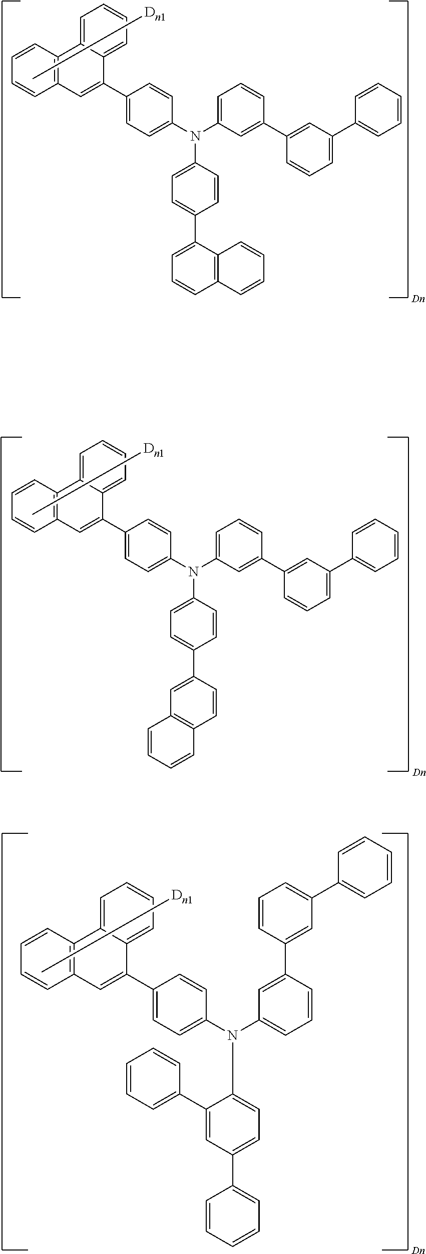

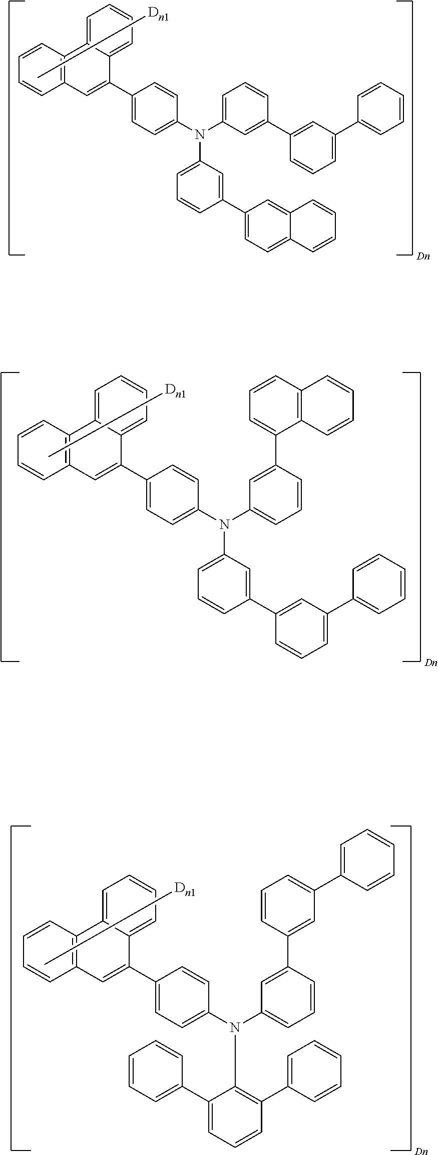

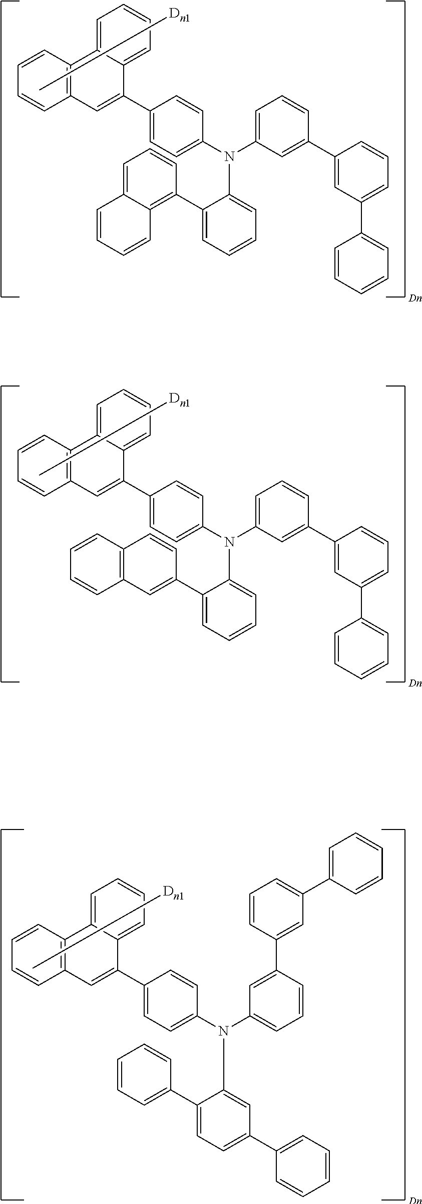

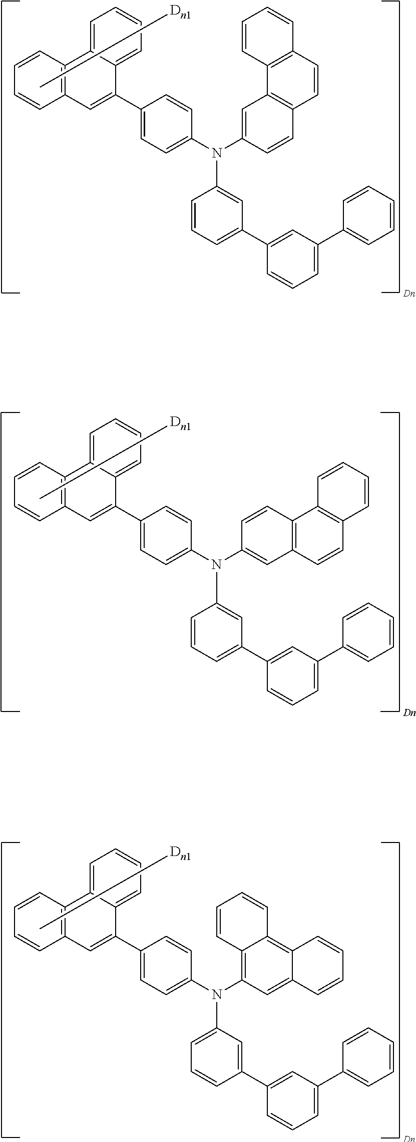

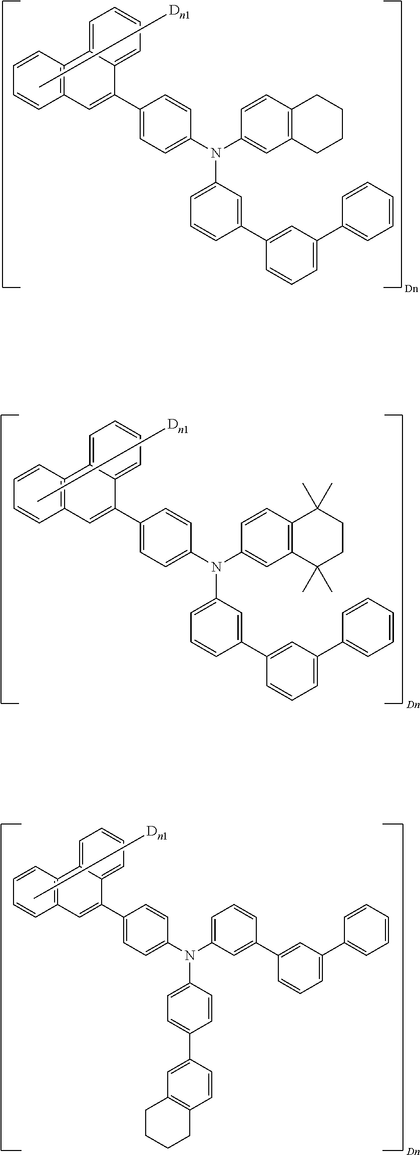

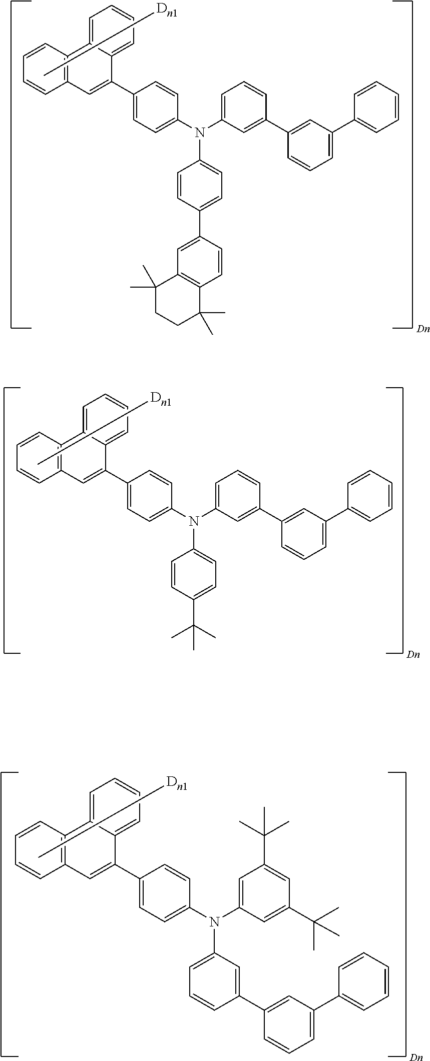

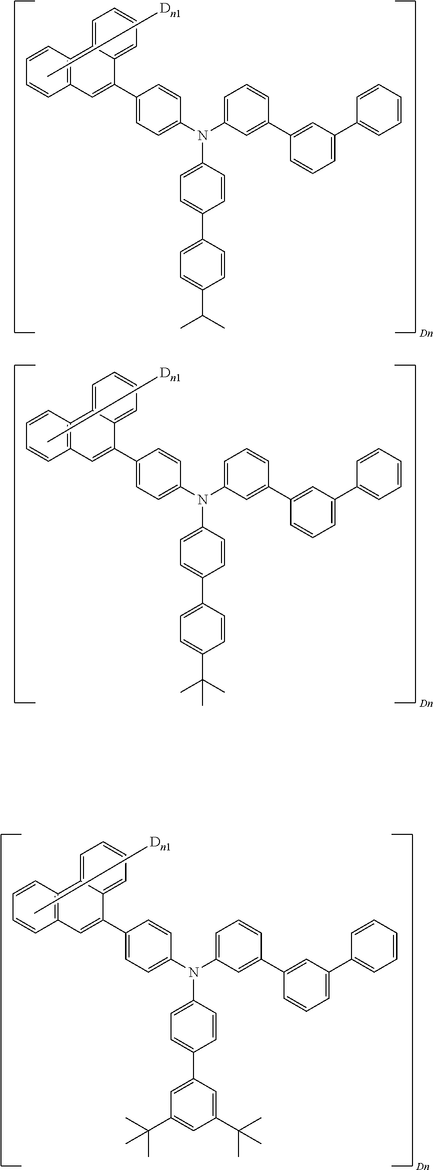

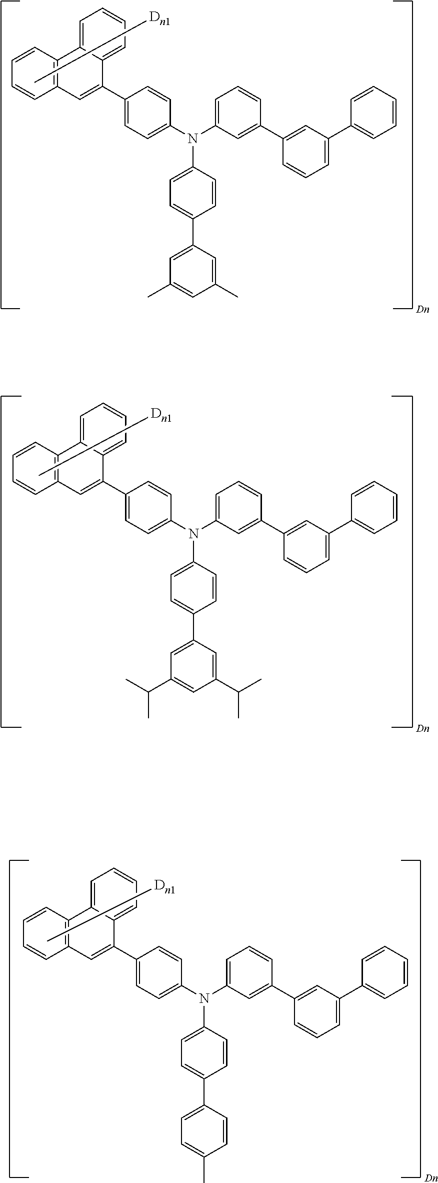

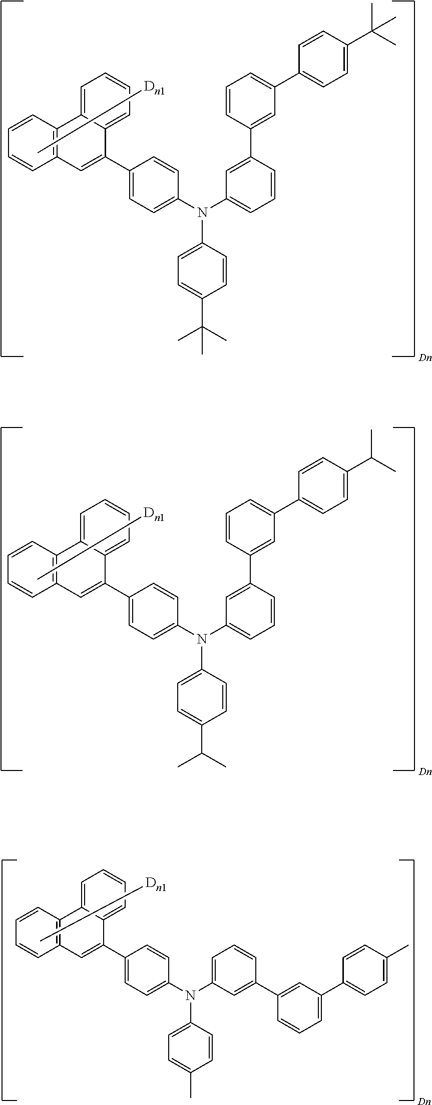

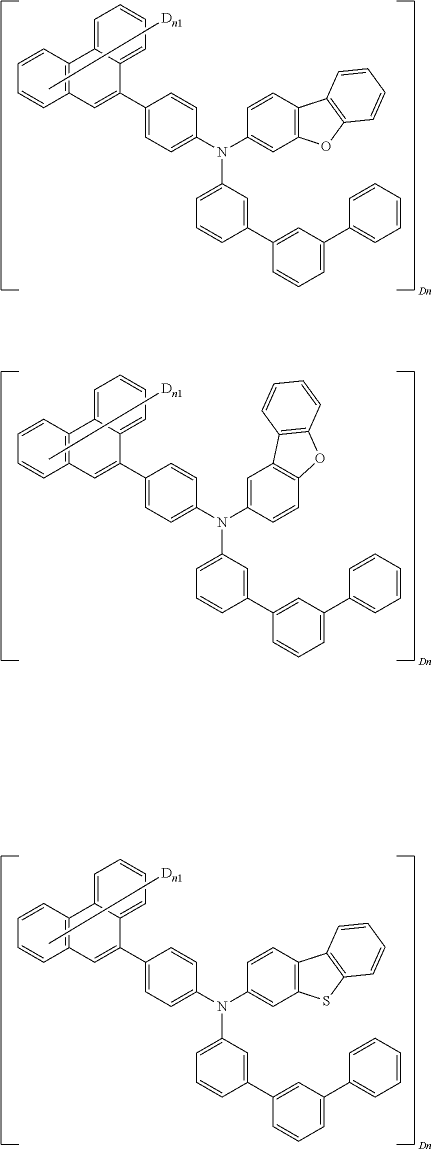

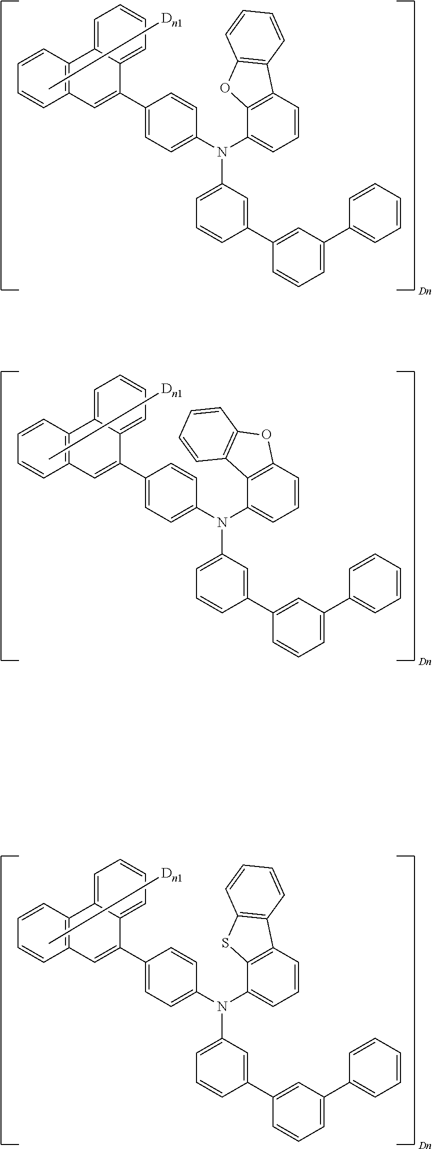

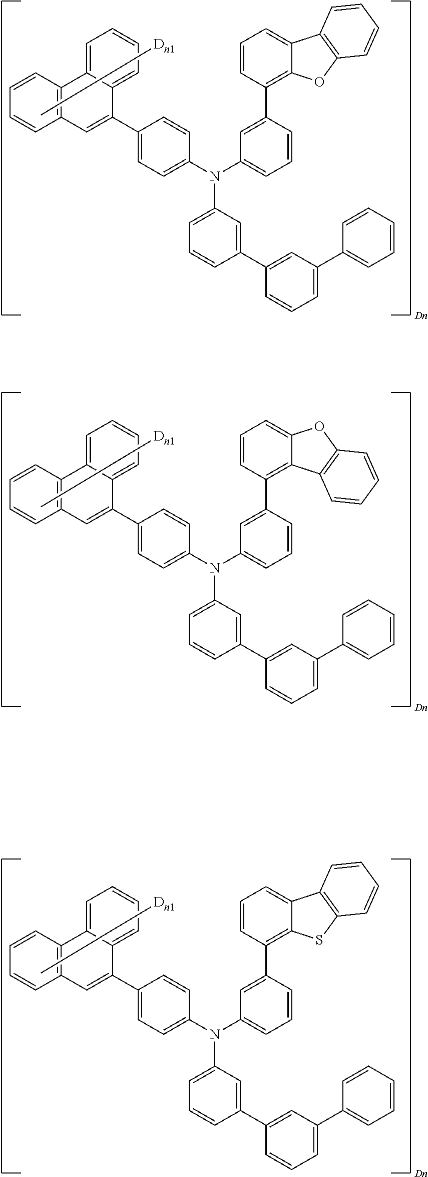

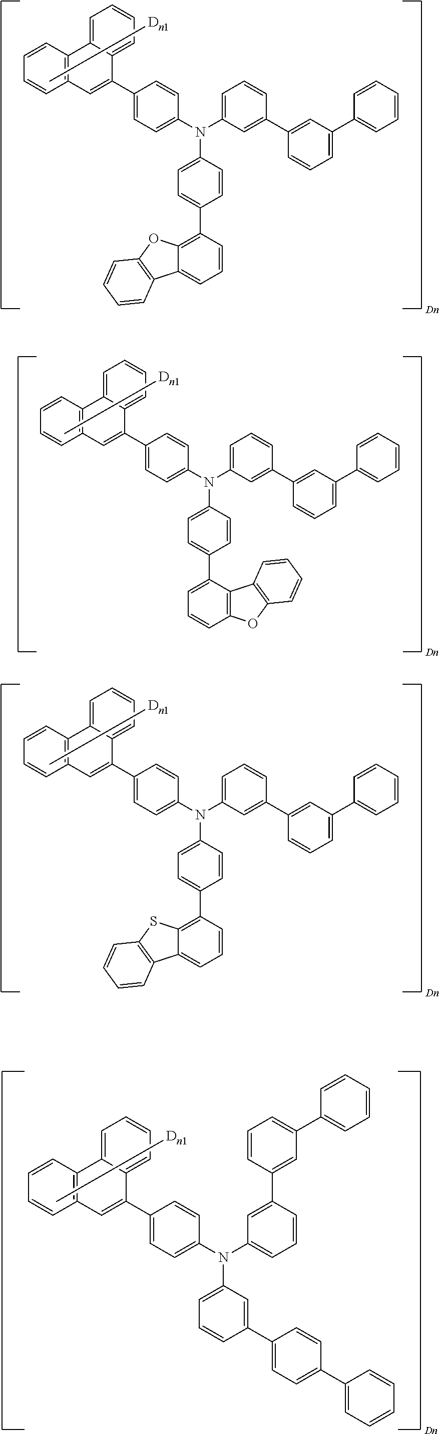

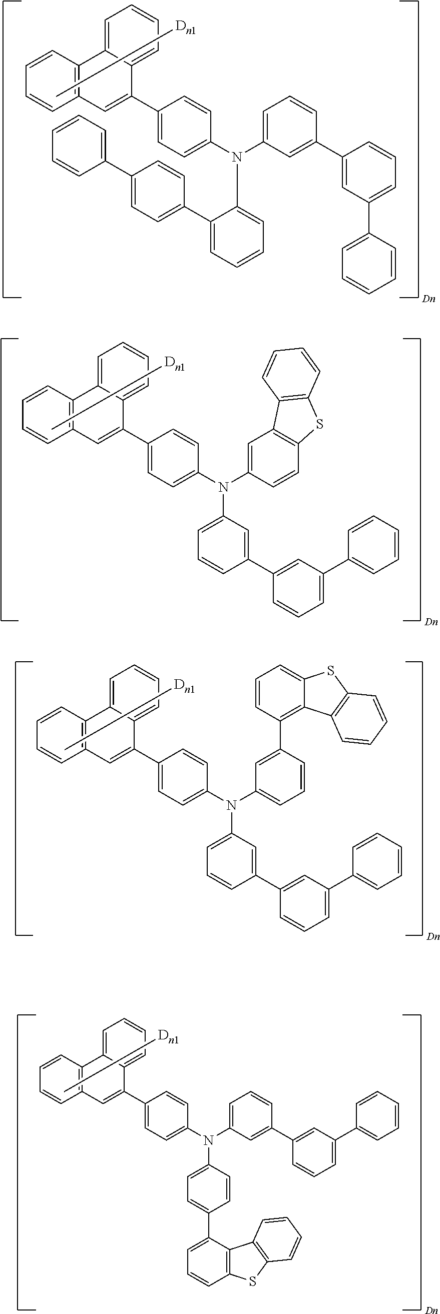

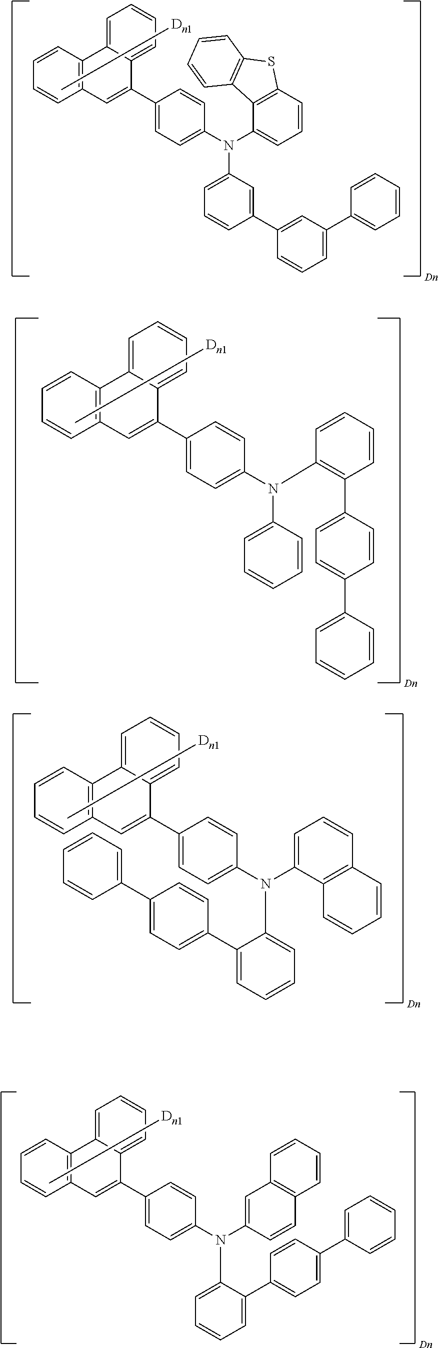

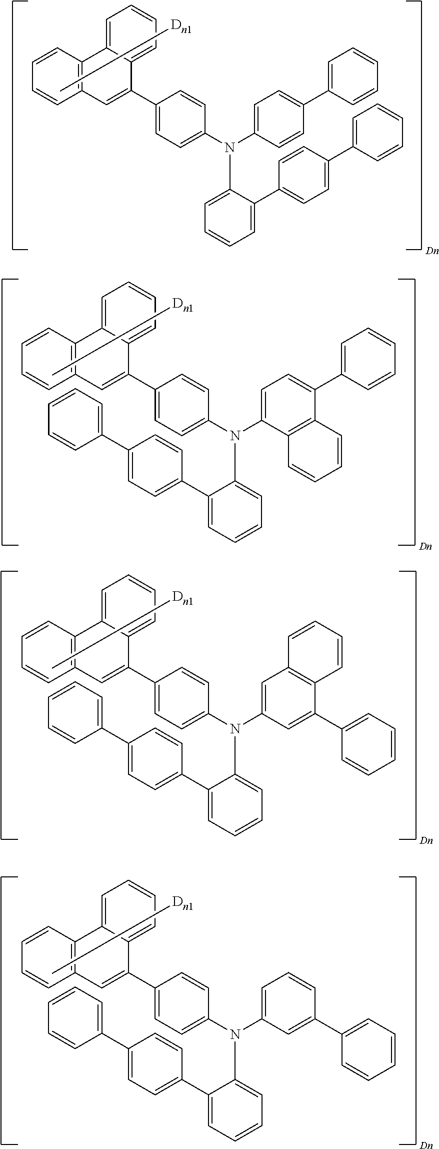

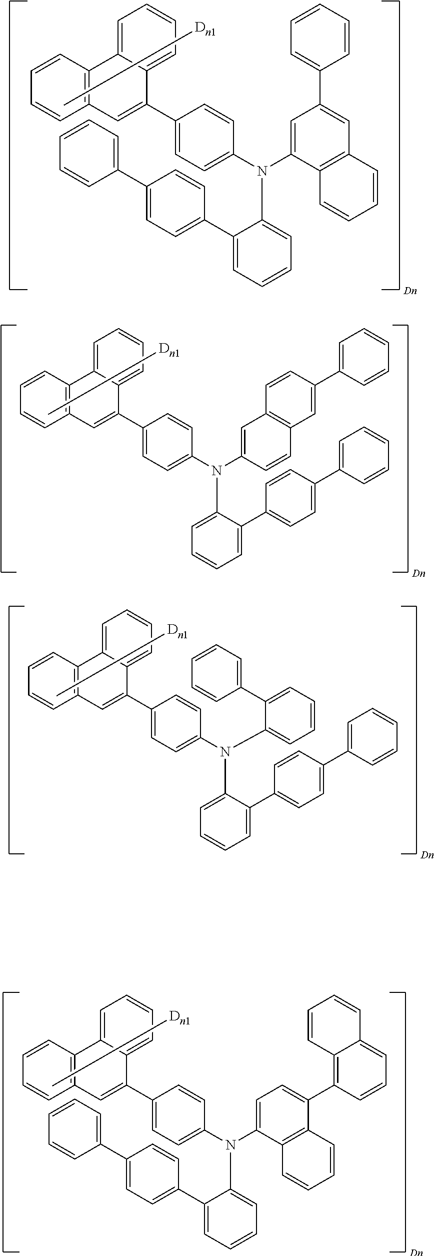

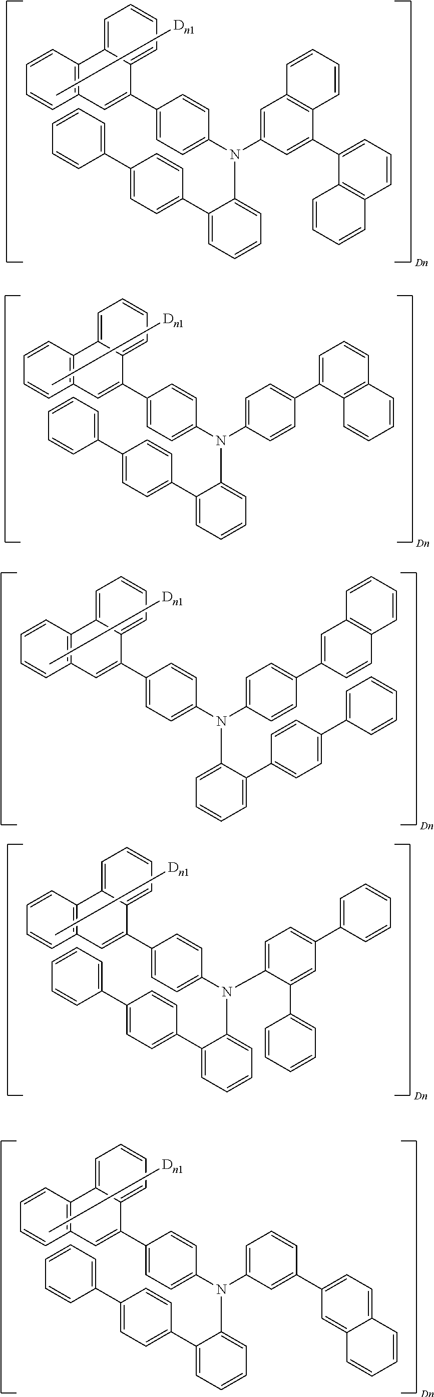

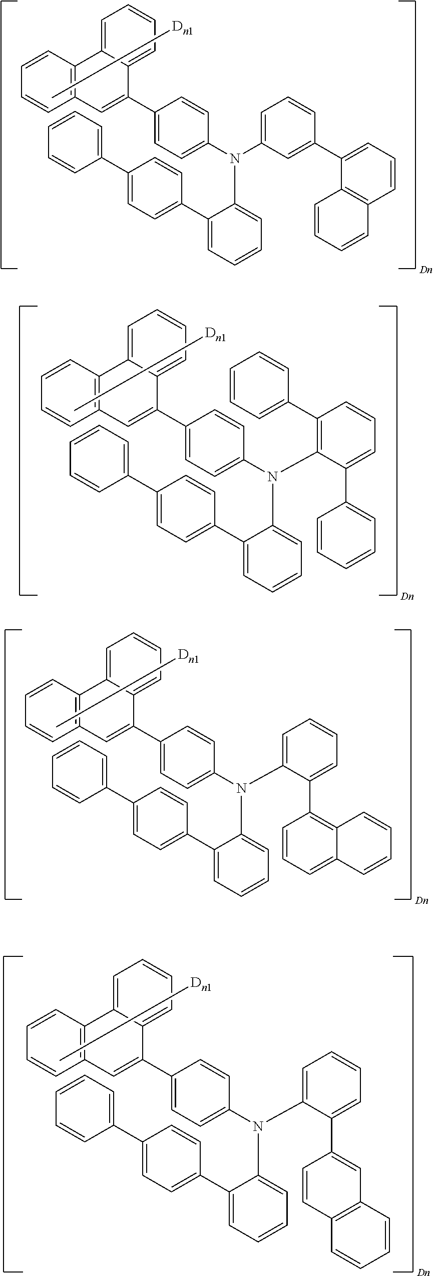

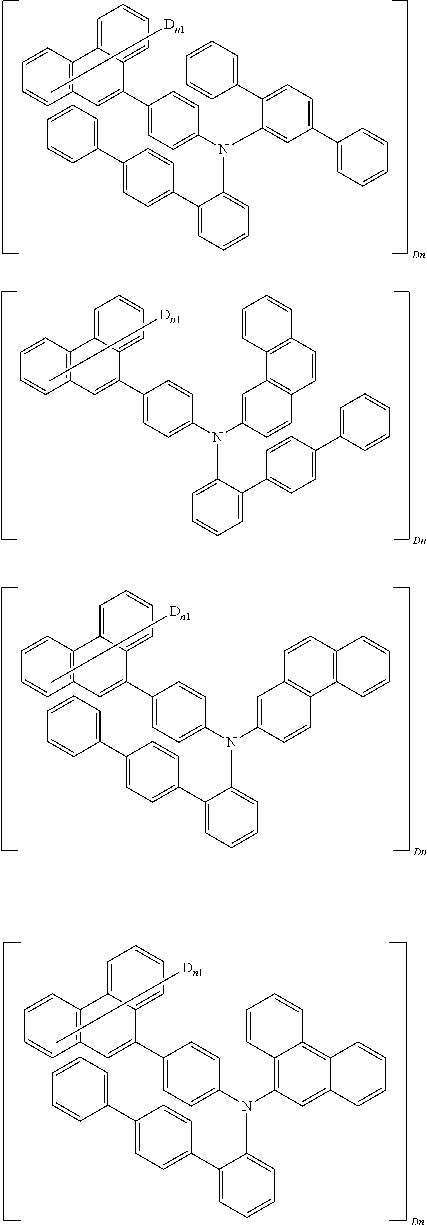

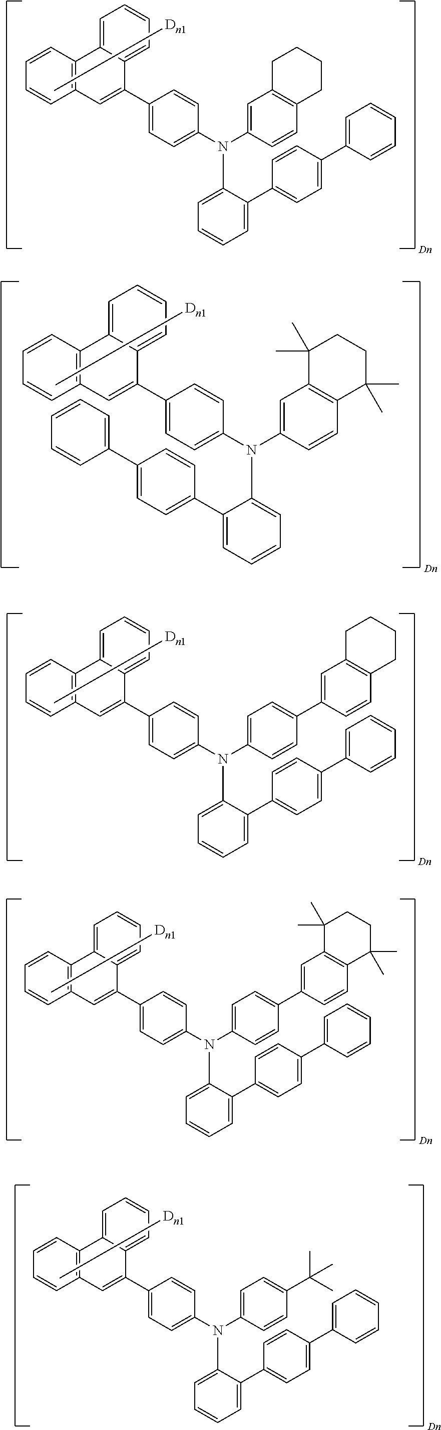

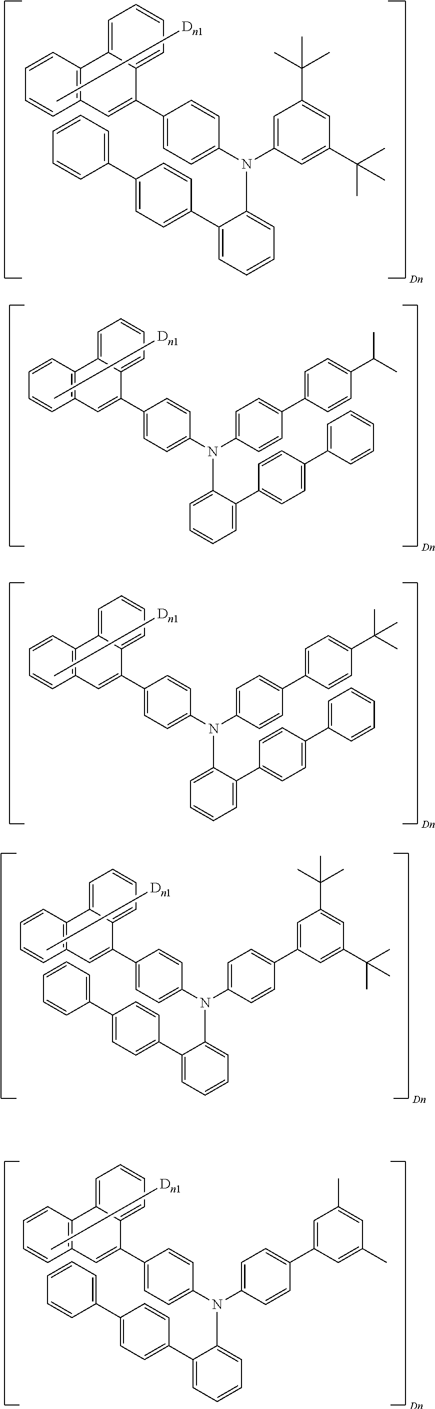

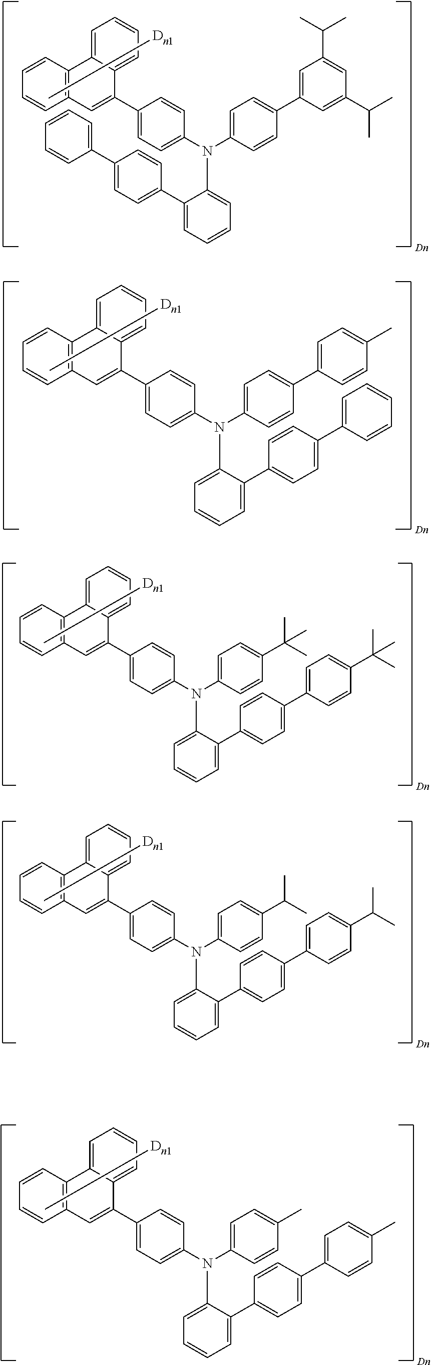

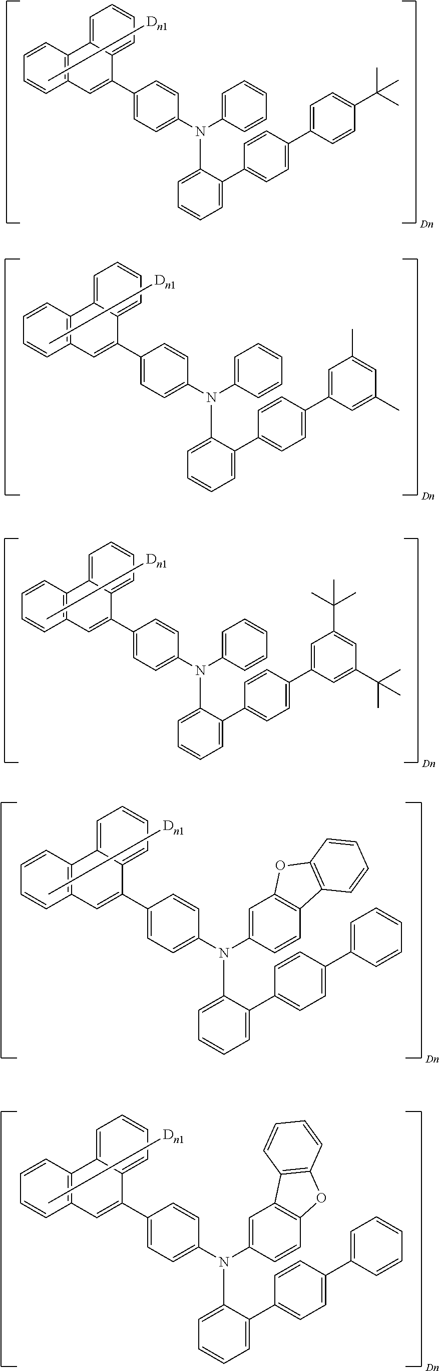

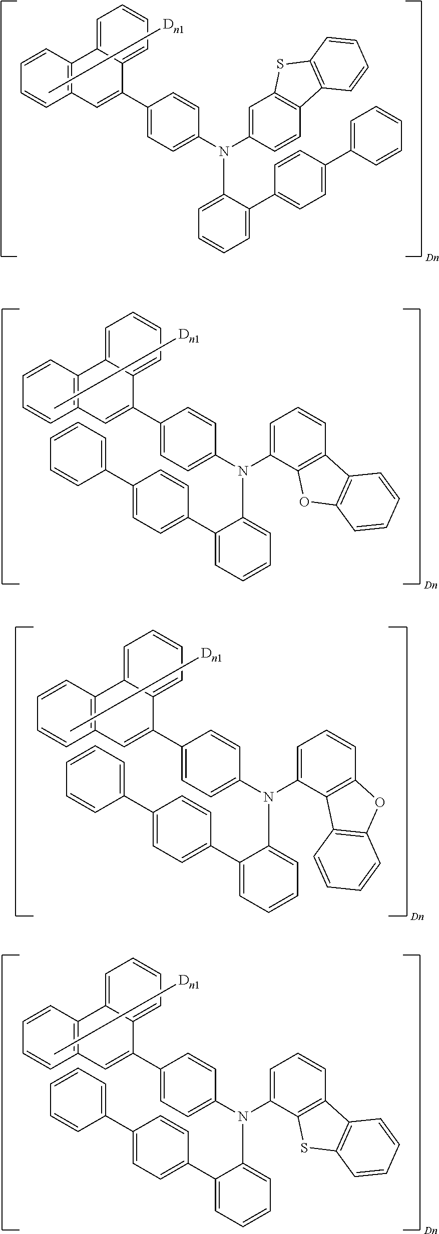

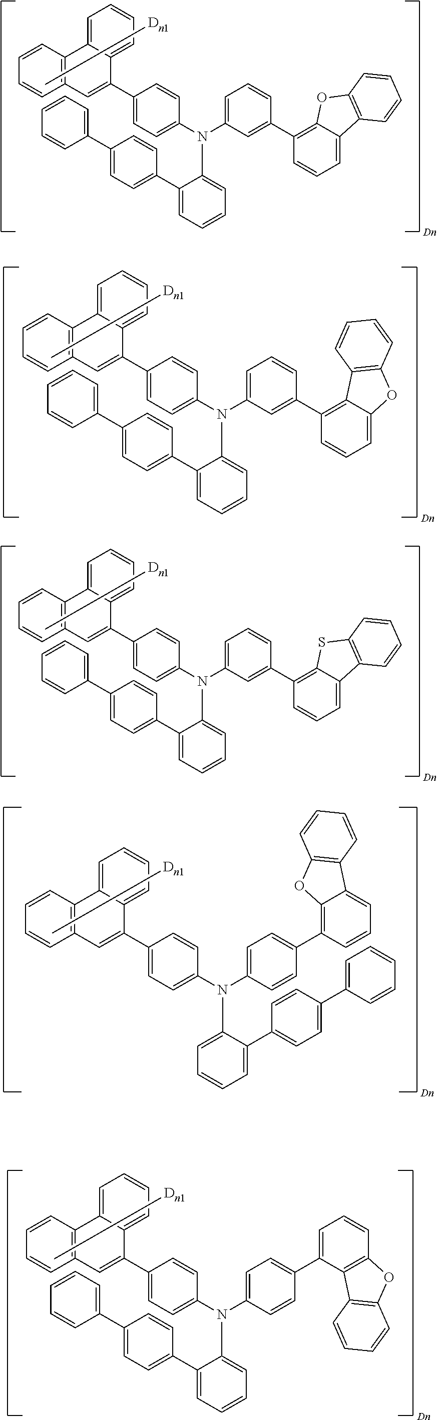

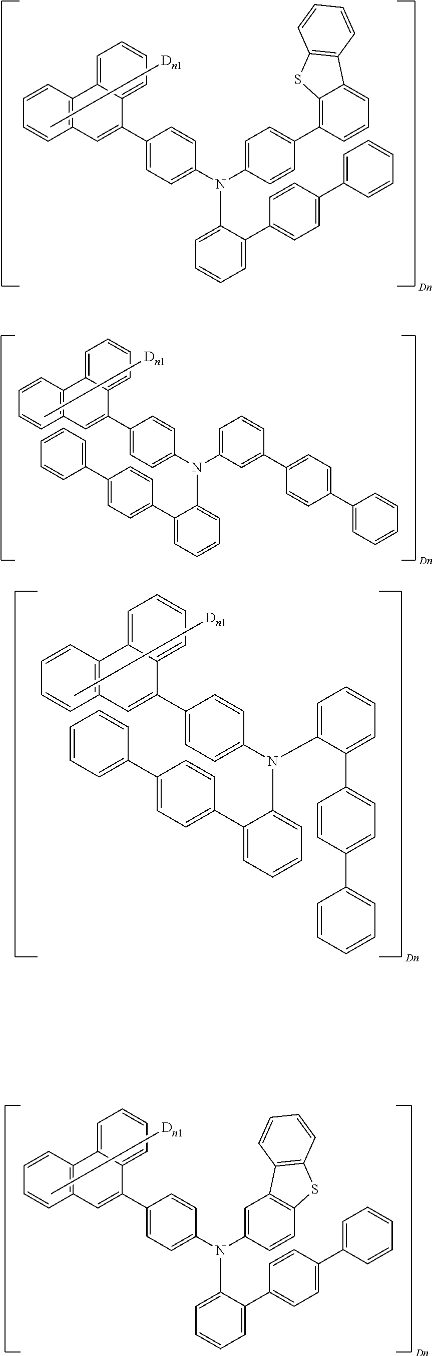

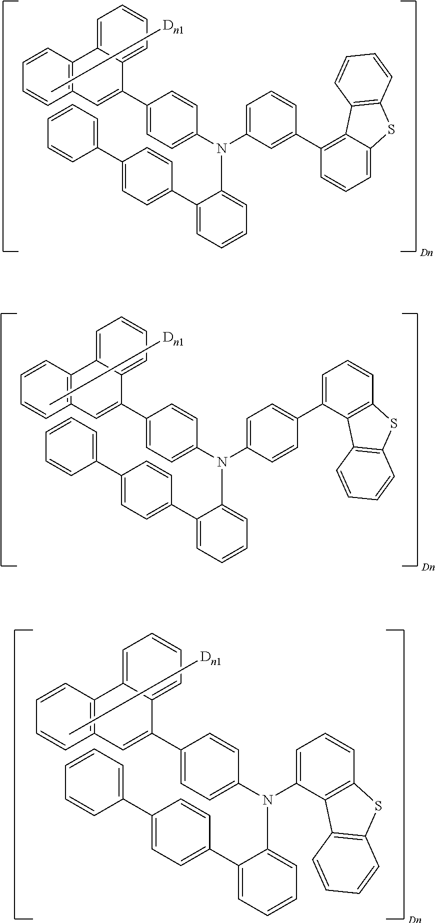

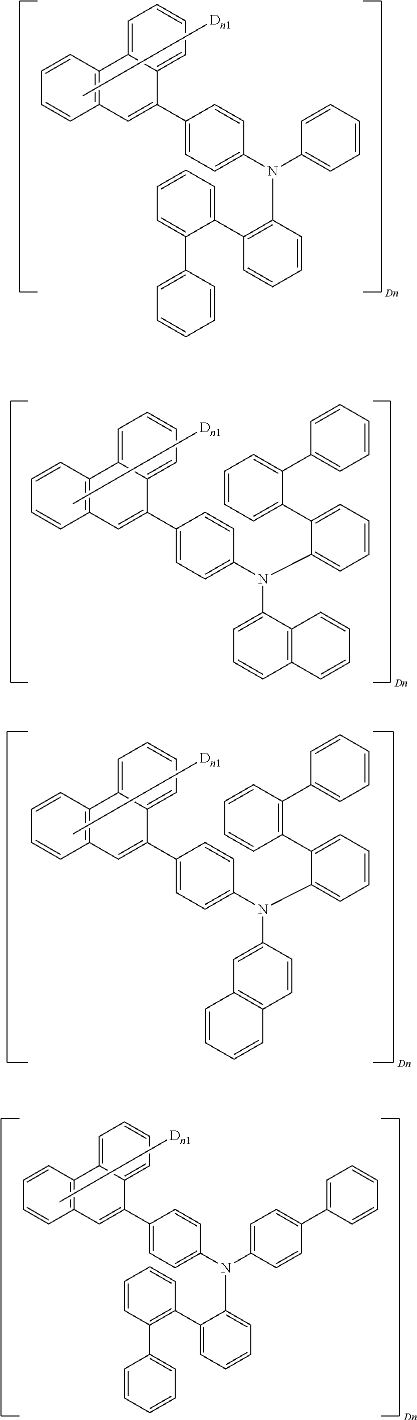

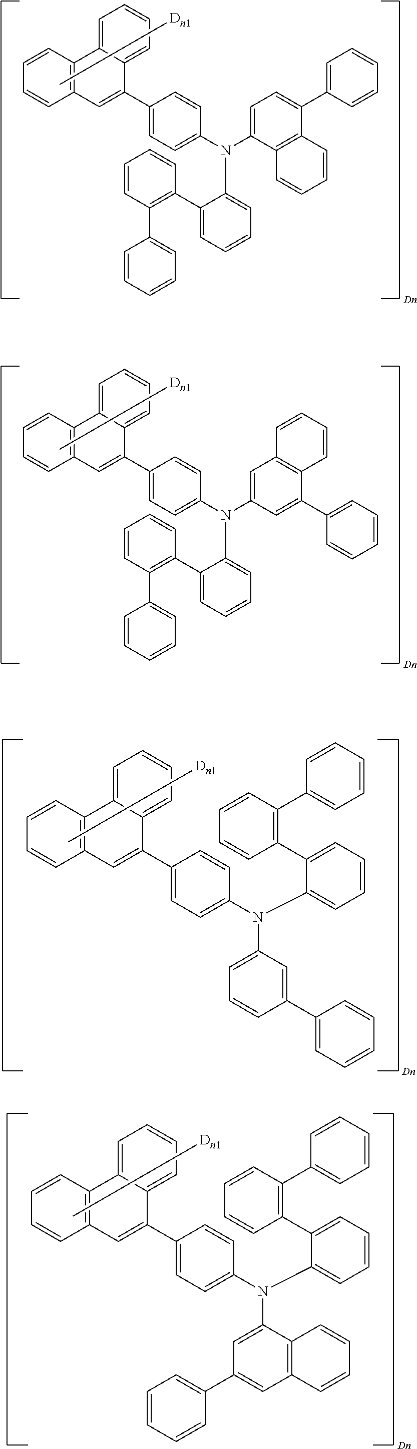

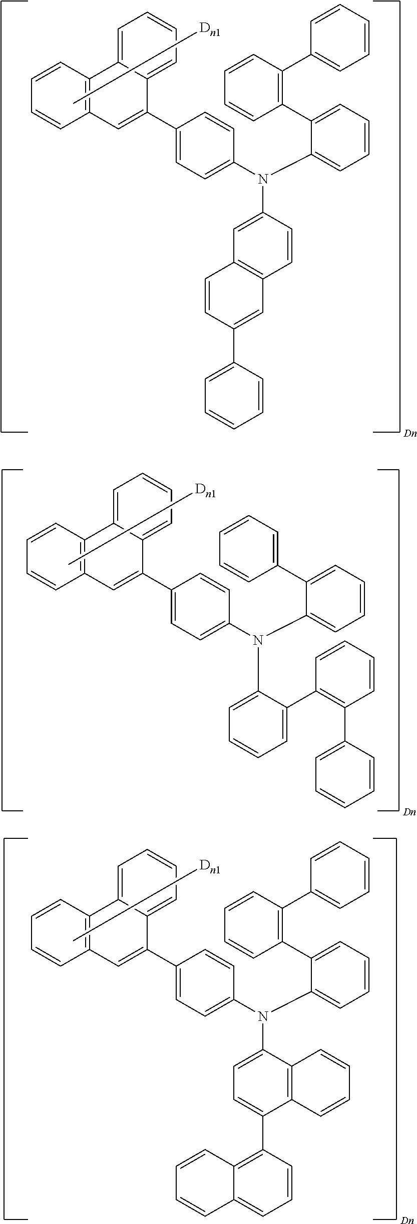

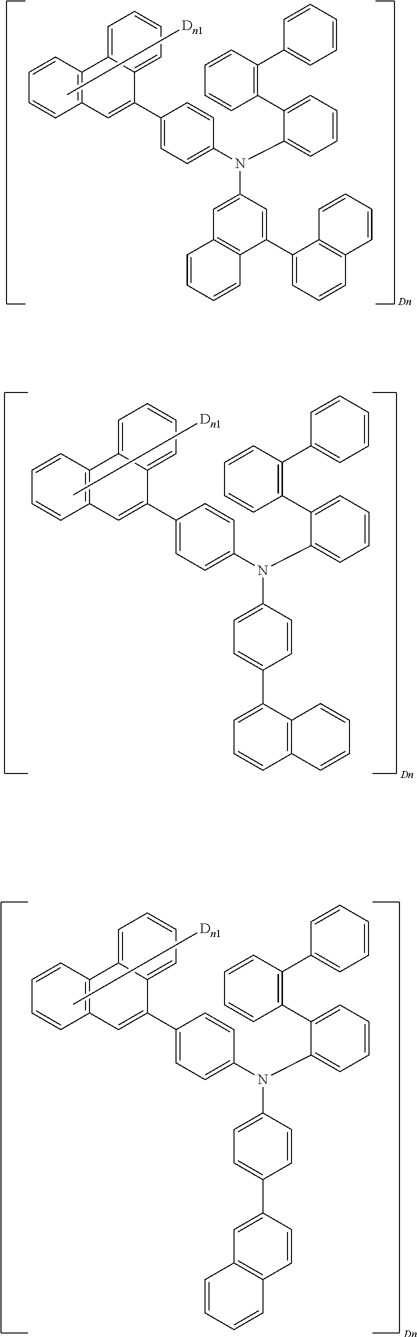

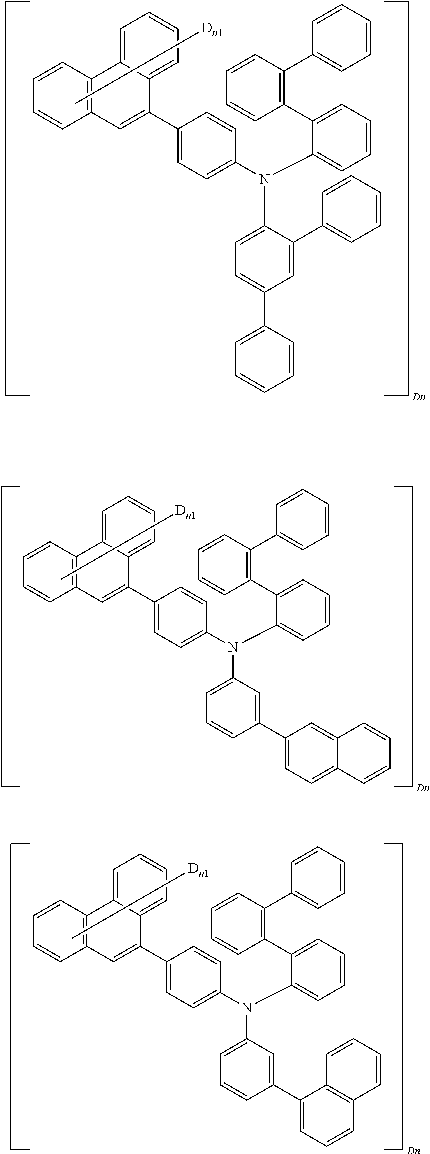

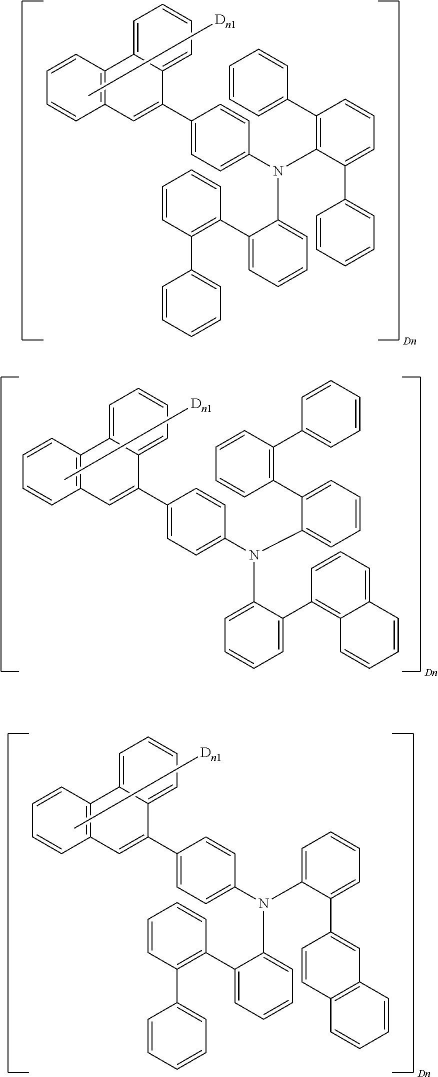

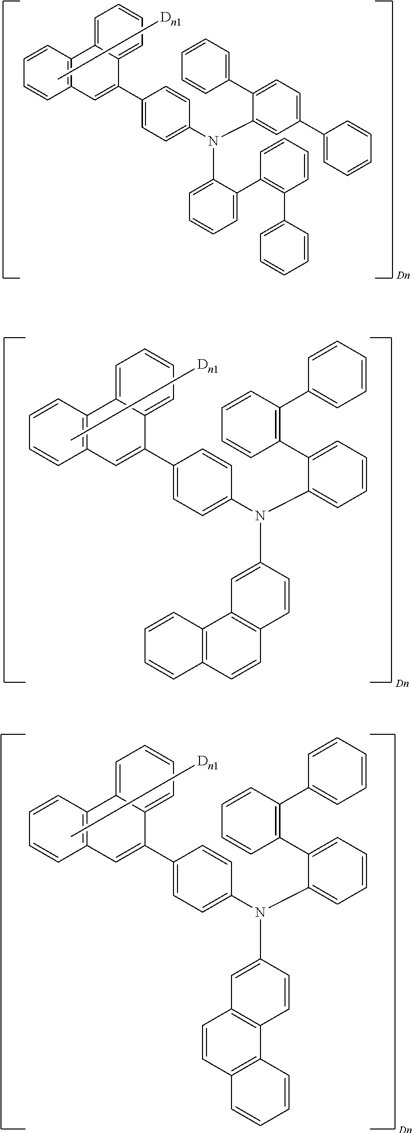

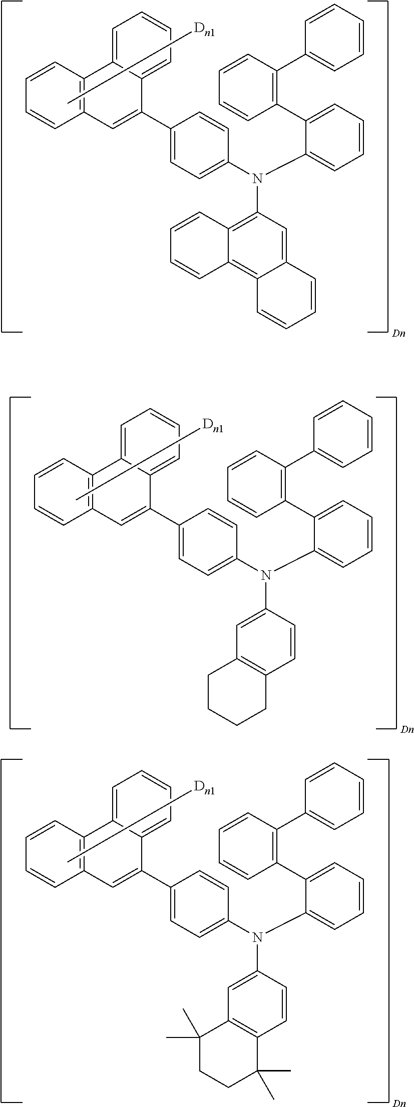

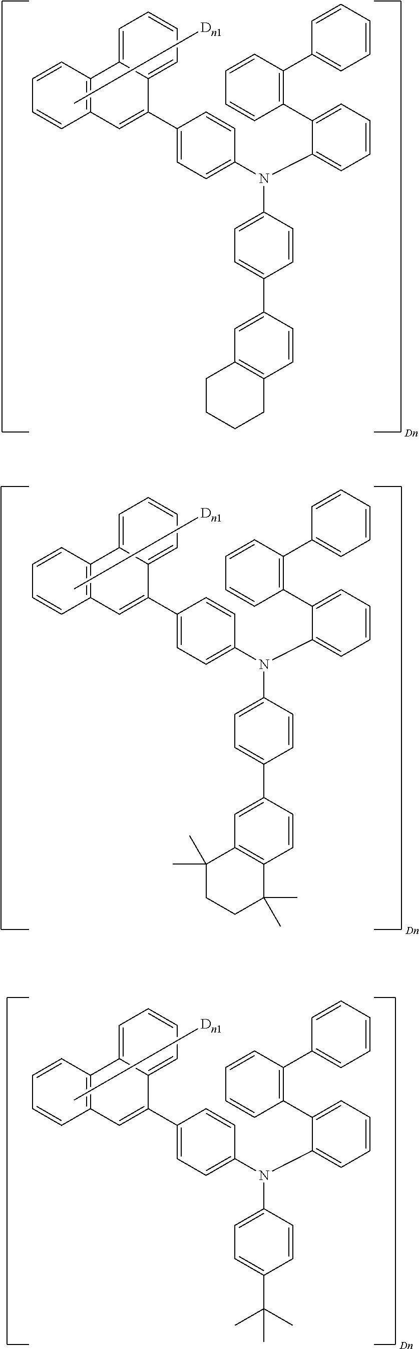

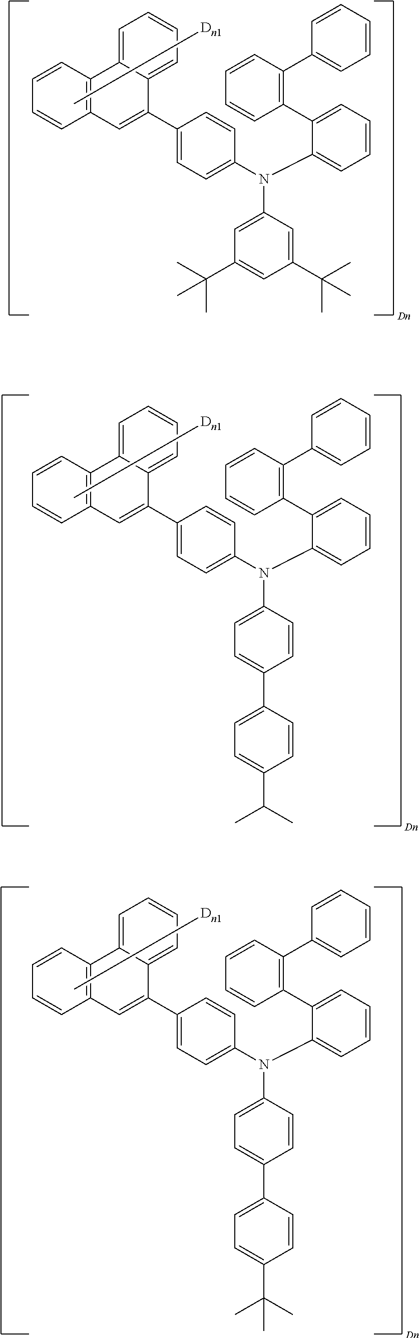

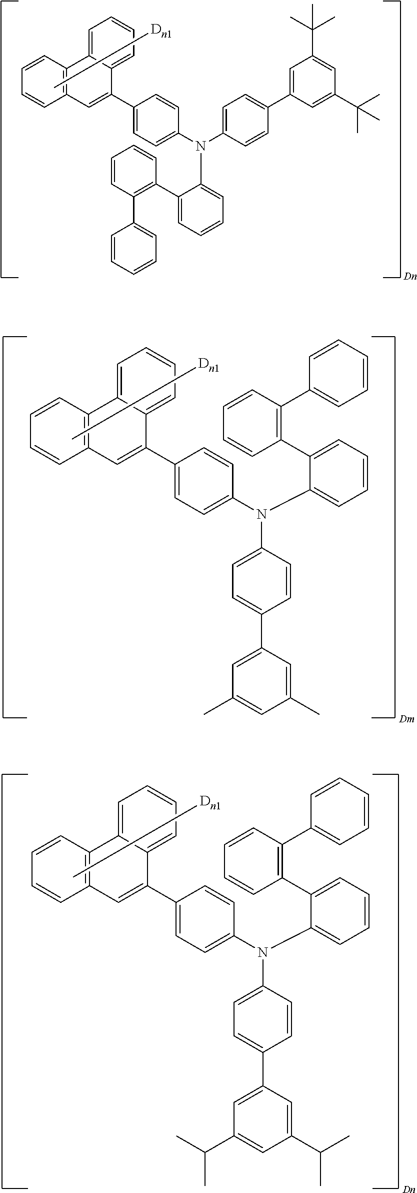

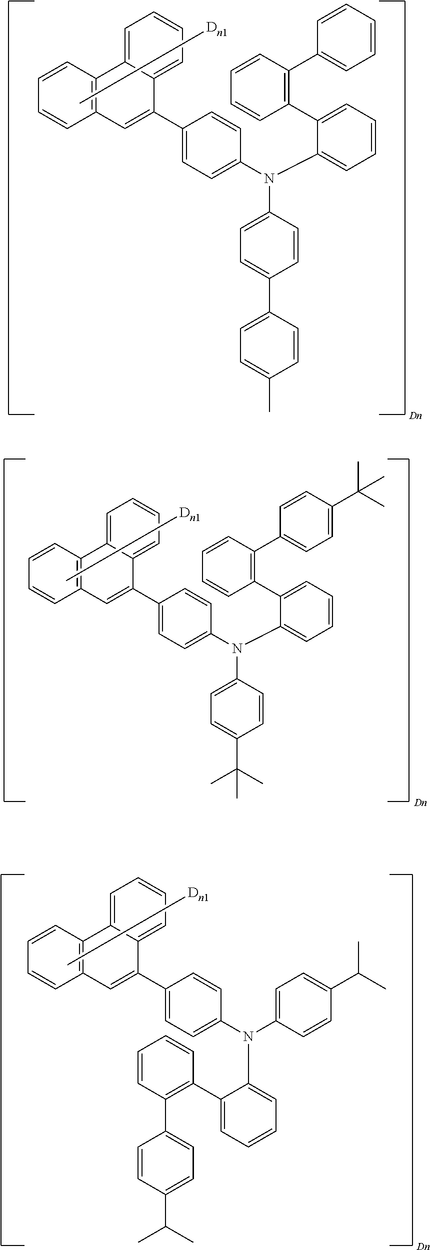

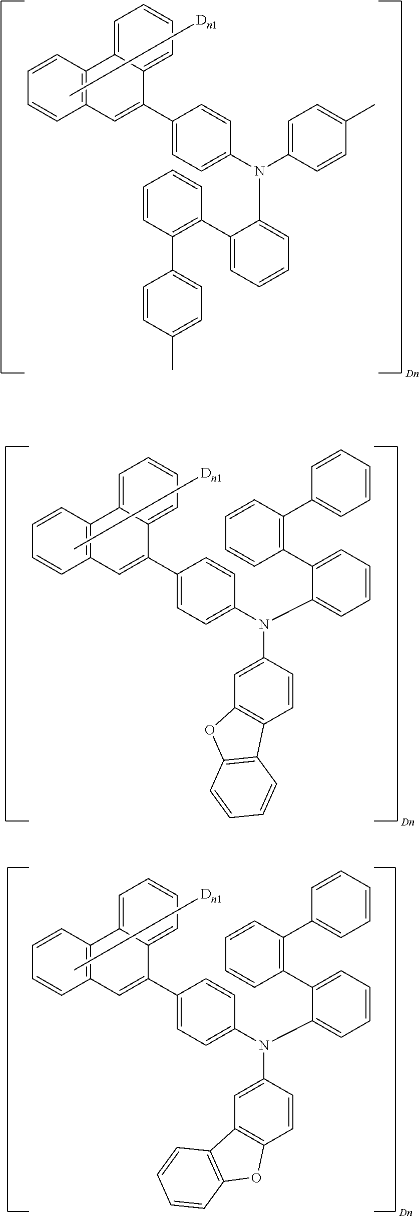

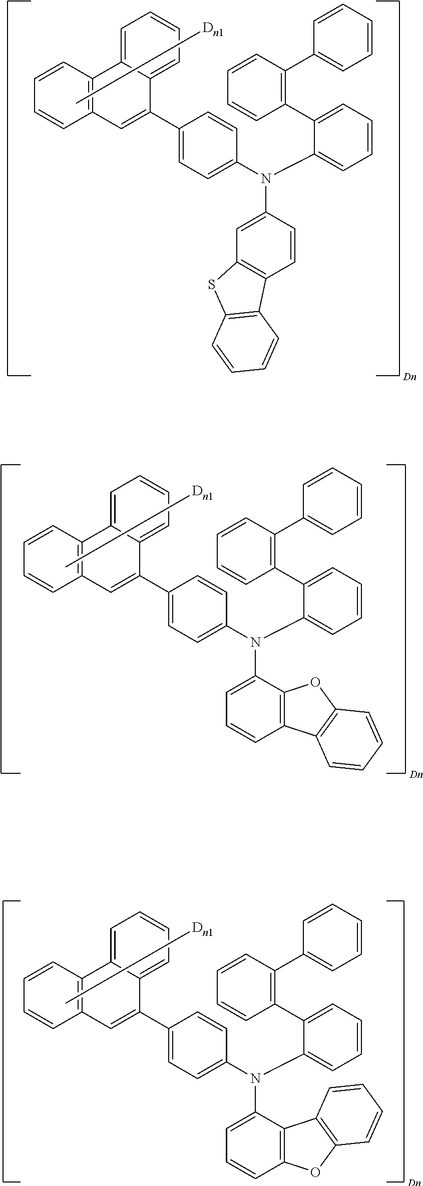

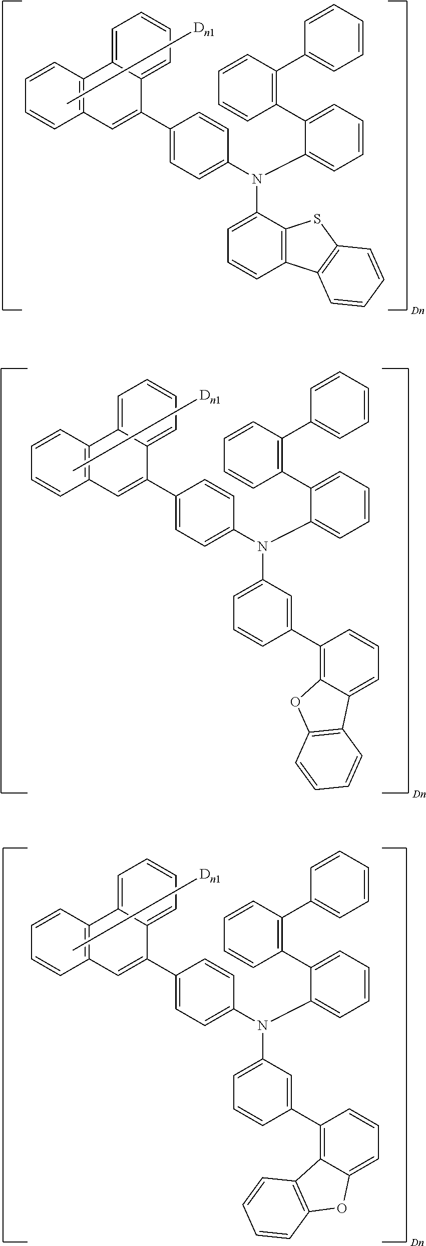

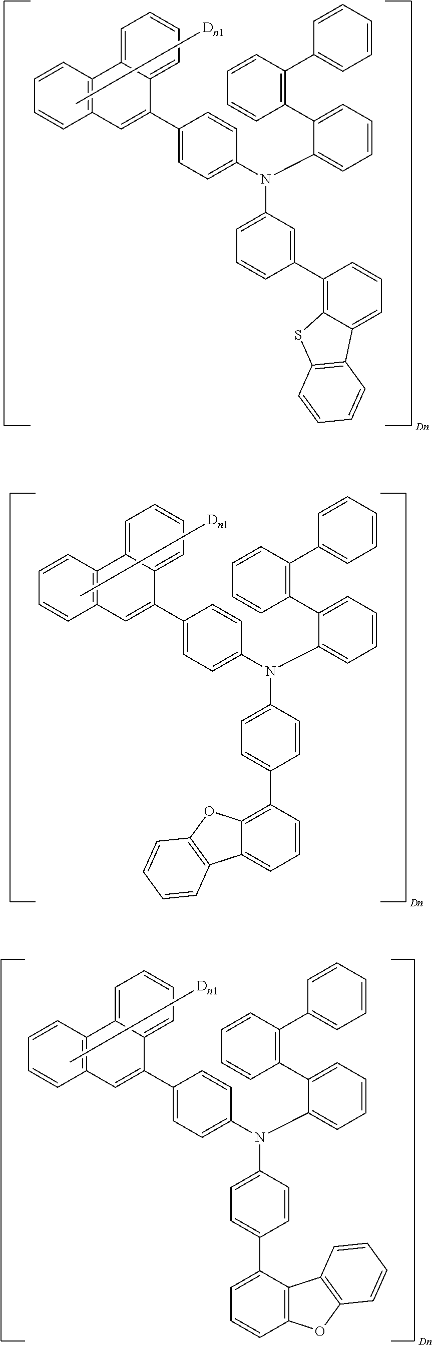

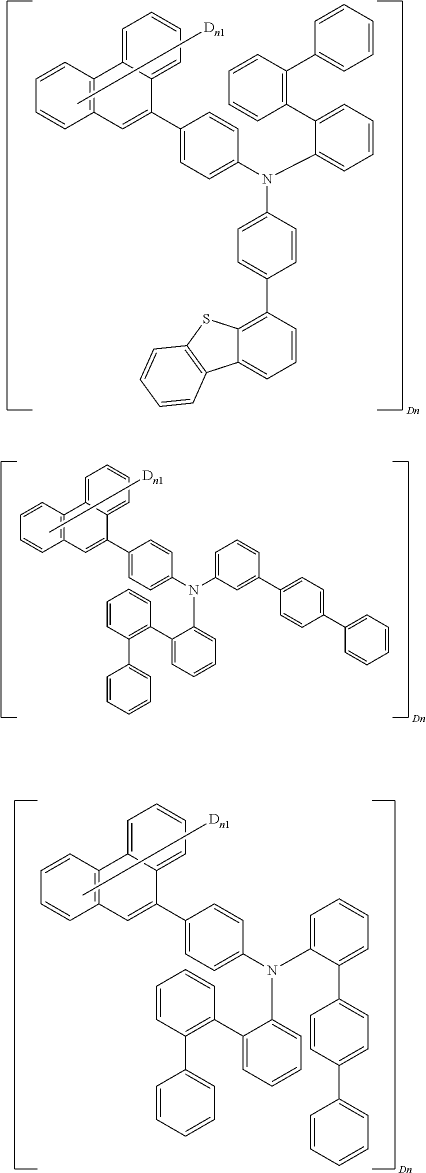

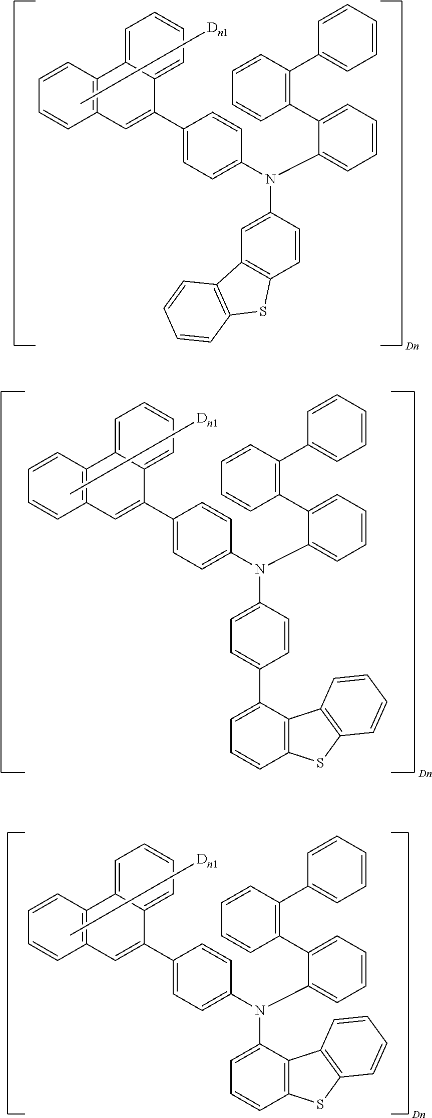

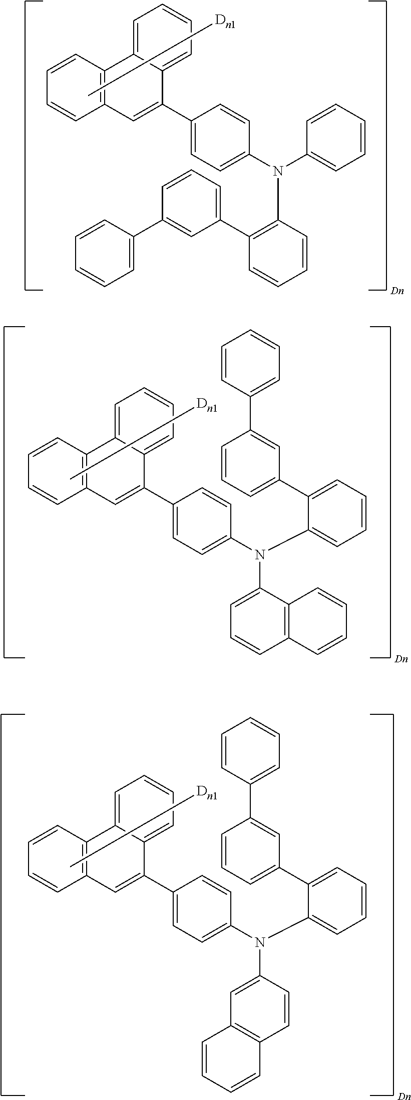

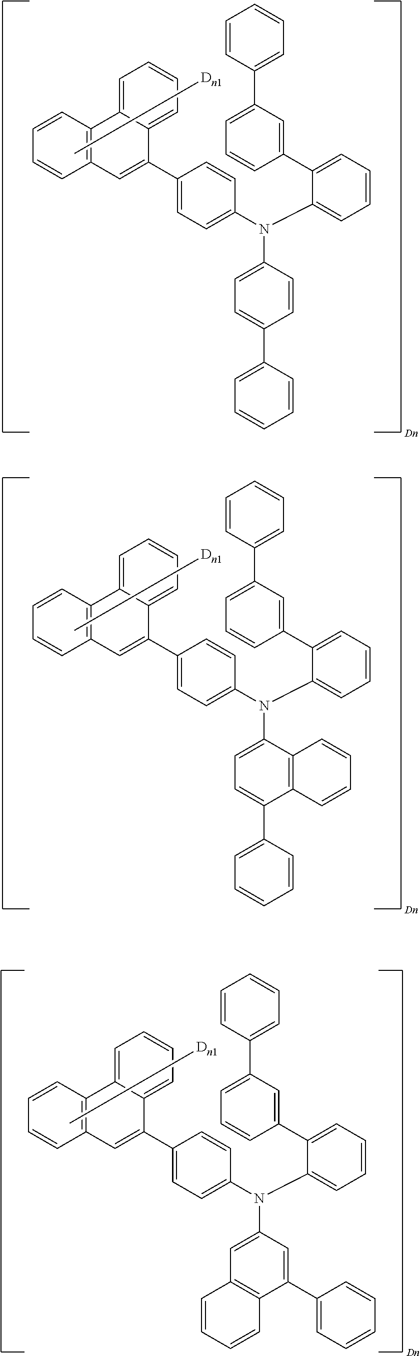

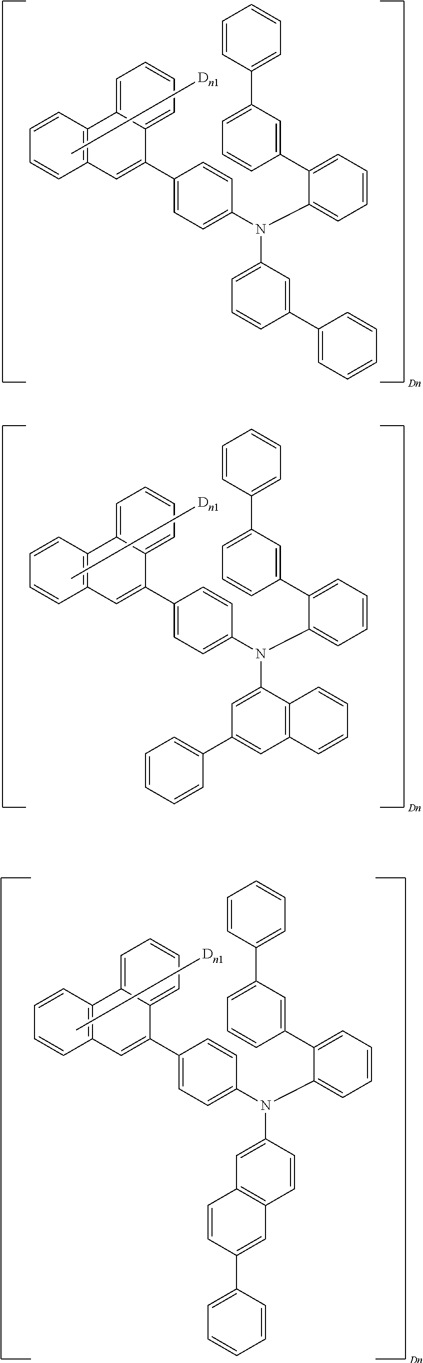

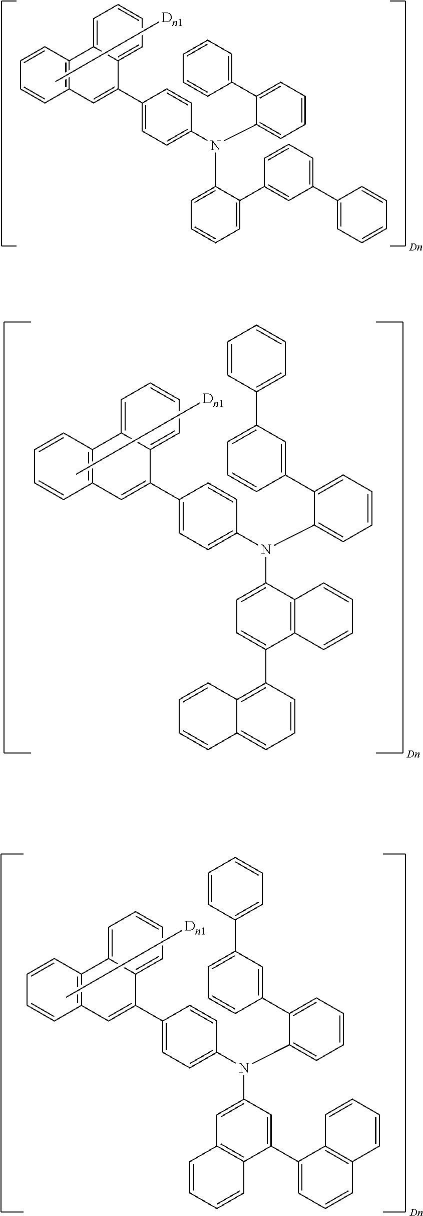

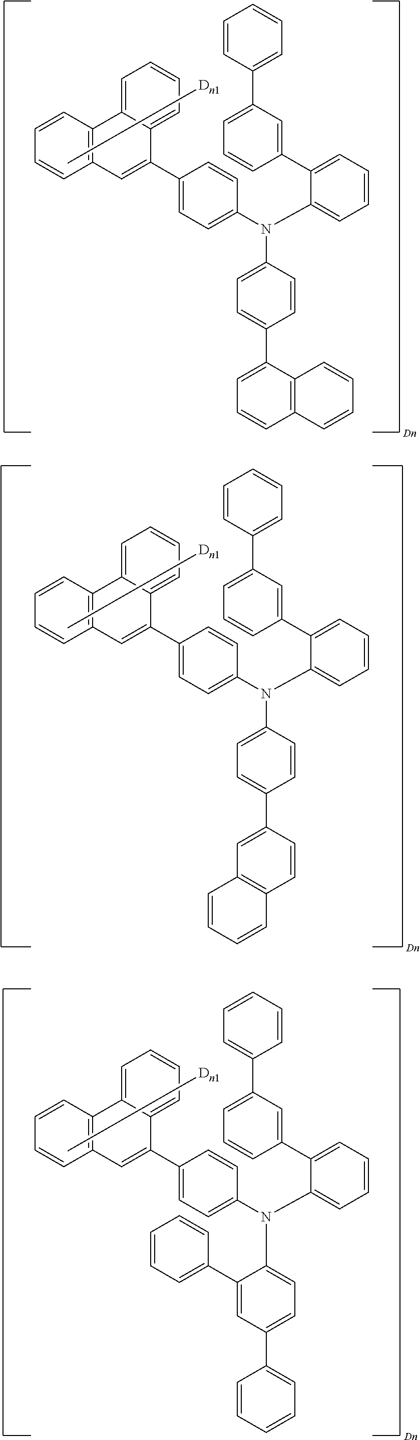

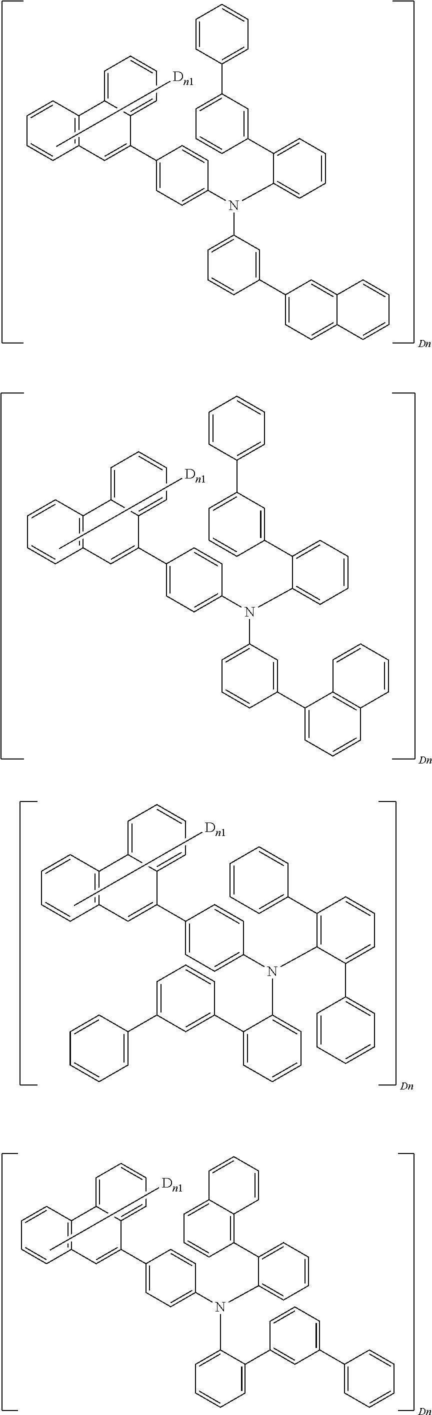

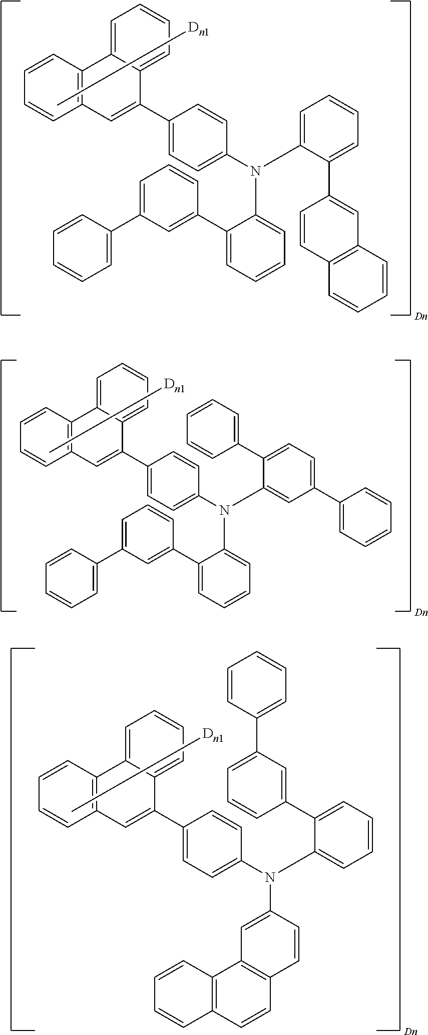

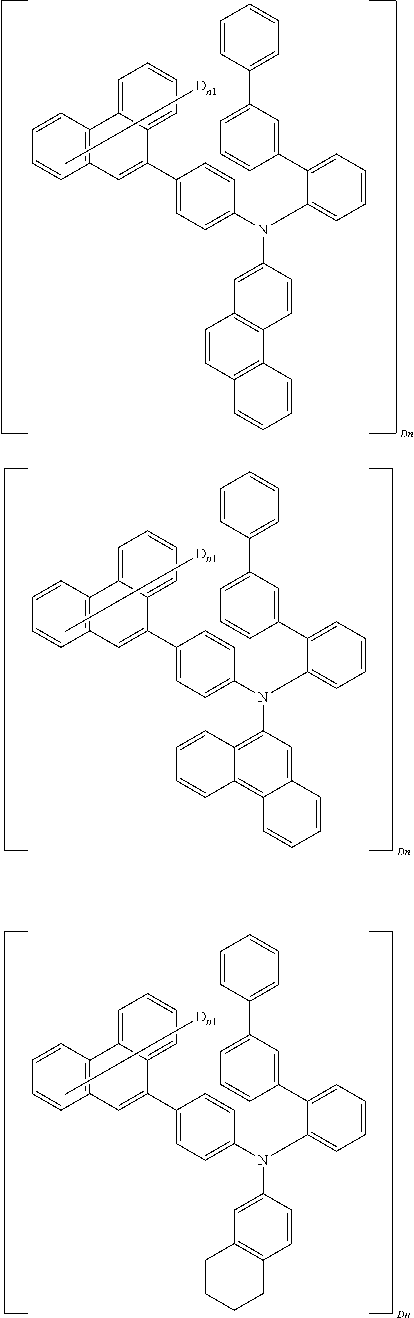

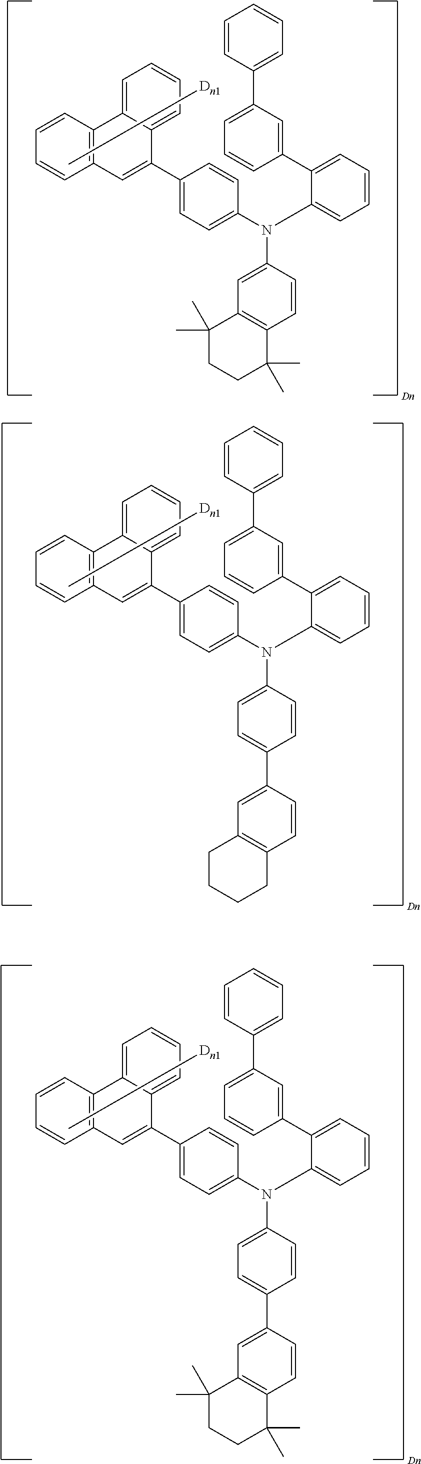

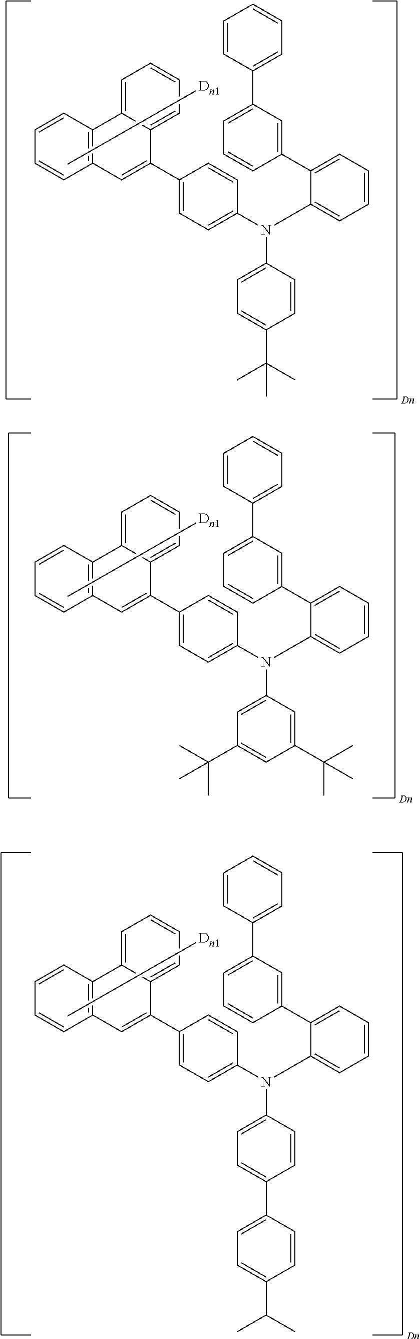

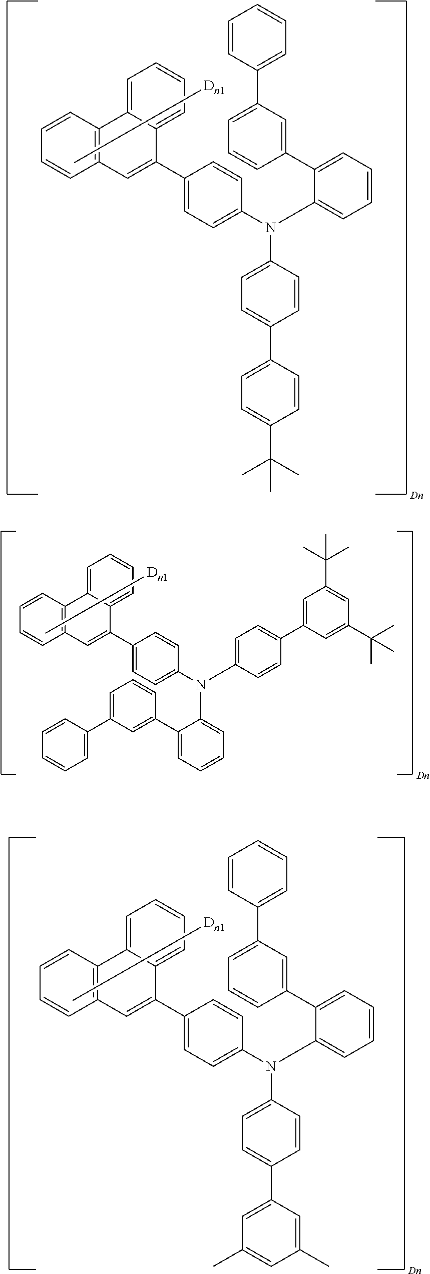

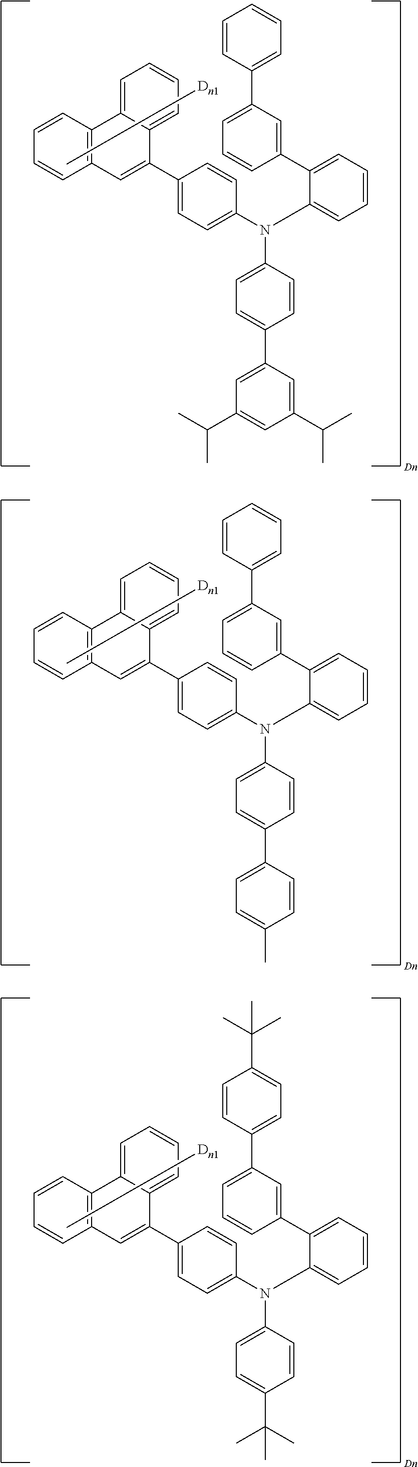

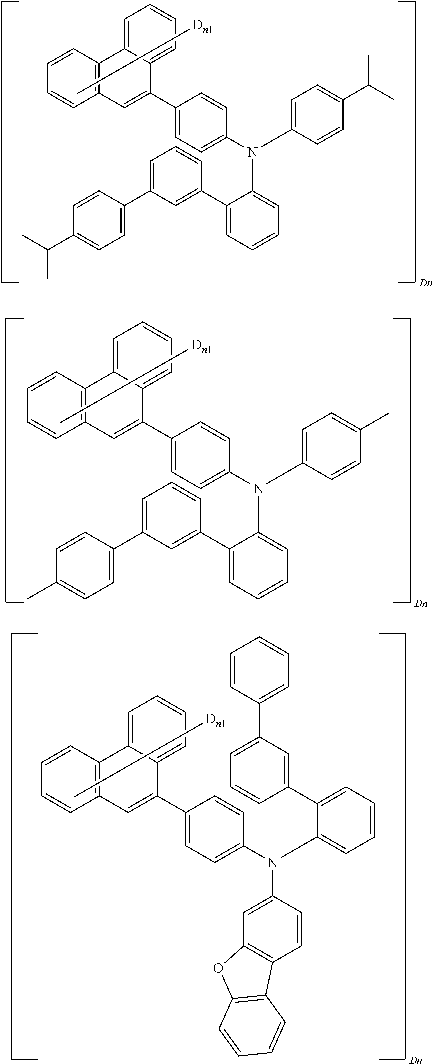

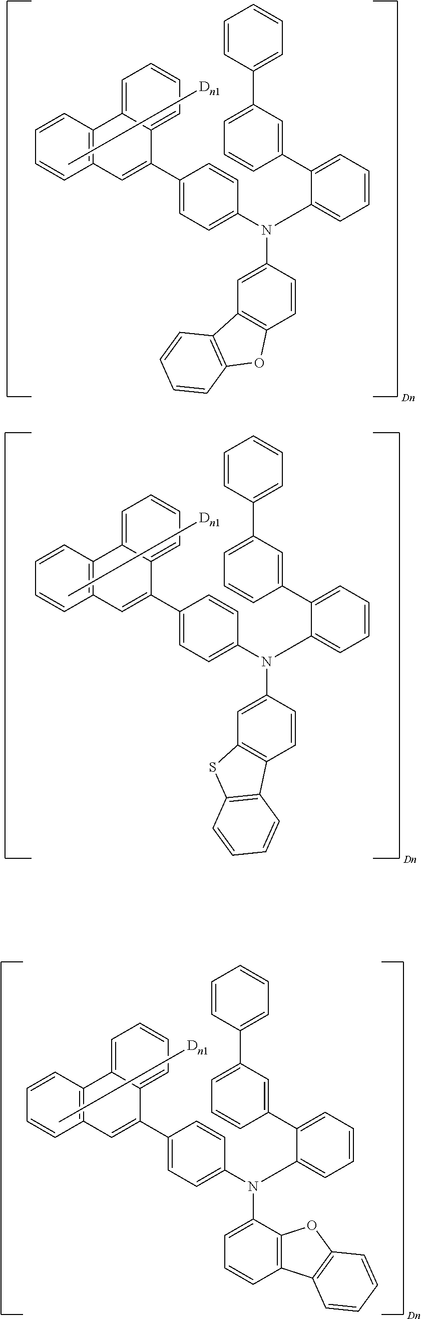

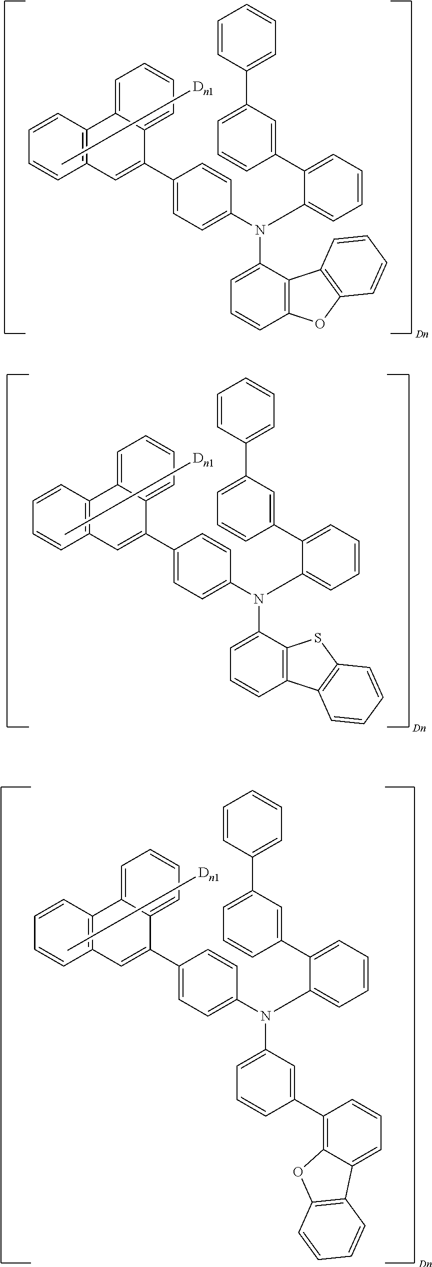

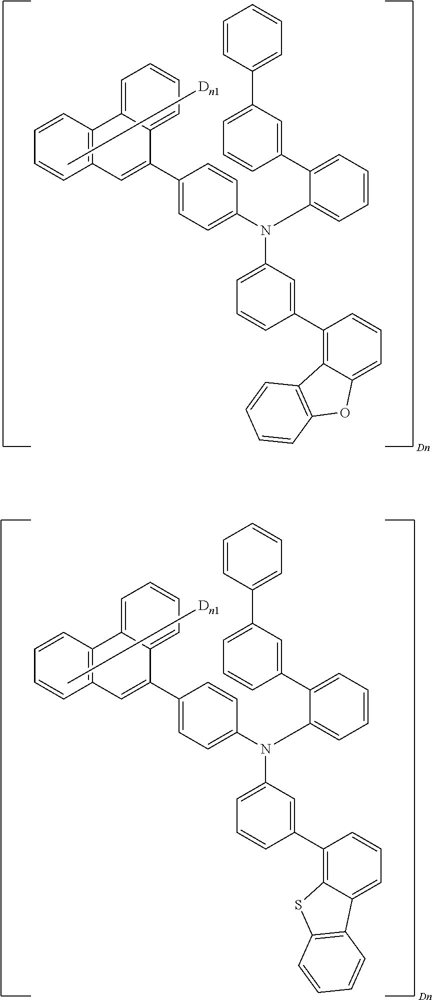

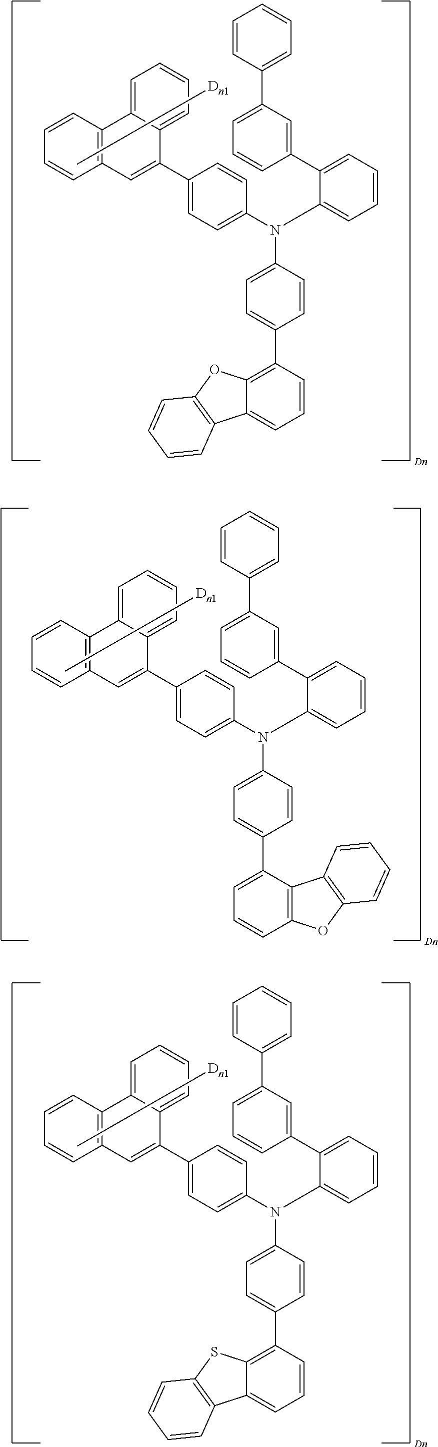

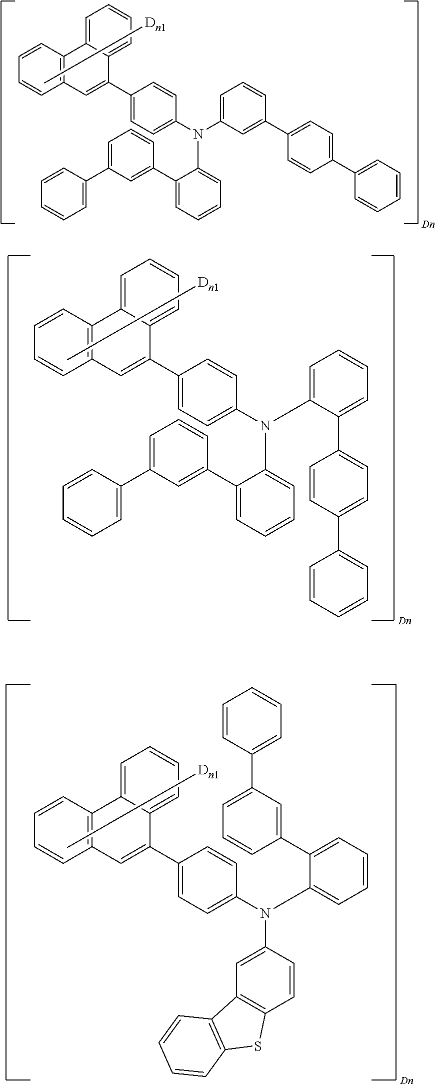

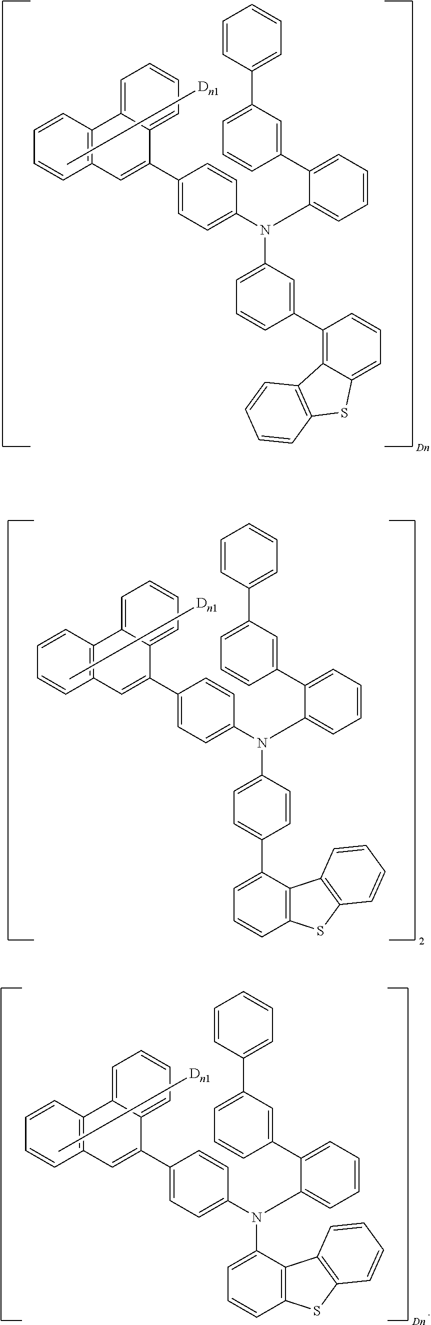

7. An organic light emitting device, comprising:
a first electrode;
a second electrode that is provided opposite to the first electrode; and
one or more organic material layers that are provided between the first electrode and the second electrode, wherein at least one layer of the one or more organic material layers comprises the compound of Chemical Formula 1 of claim 1.

8. An organic light emitting device as claimed in claim 7, wherein:
the organic material layer is a hole transport layer, a hole transport auxiliary layer, or a light emitting layer.
